SEQUENCE LISTING (13 sequences)

SEQ ID NO: 1 - protein, 15 aa, synthetic construct
  REGION 1..15: note = Variable light chain complementarity determining region 1
CTGSSSNIGAGYNVY

SEQ ID NO: 2 - protein, 7 aa, synthetic construct
  REGION 1..7: note = Variable light chain complementarity determining region 2
GNINRPS

SEQ ID NO: 3 - protein, 12 aa, synthetic construct
  REGION 1..12: note = Variable light chain complementarity determining region 3
CAAWDKSISGLV

SEQ ID NO: 4 - protein, 10 aa, synthetic construct
  REGION 1..10: note = Variable heavy chain complementarity determining region 1
GFTFSTYGMH

SEQ ID NO: 5 - protein, 26 aa, synthetic construct
  REGION 1..26: note = Variable heavy chain complementarity determining region 2
GKGLEWLSYISGGSSYIFYADSVRGR

SEQ ID NO: 6 - protein, 13 aa, synthetic construct
  REGION 1..13: note = Variable heavy chain complementarity determining region 3
CARILRGGSGMDL

SEQ ID NO: 7 - protein, 112 aa, synthetic construct
  REGION 1..112: note = Variable light chain amino acid sequence
QSVLTQPPSASGTPGQRVTISCTGSSSNIGAGYNVYWYQQLPGTAPKLLIYGNINRPSGVPDRFSGSKSGTSASLAISGLRSEDEADYYCAAWDKSISGLVFGGGTKLTVLG

SEQ ID NO: 8 - protein, 119 aa, synthetic construct
  REGION 1..119: note = Variable heavy chain amino acid sequence
EVQLLESGGGLVQPGGSLRLSCAASGFTFSTYGMHWVRQAPGKGLEWLSYISGGSSYIFYADSVRGRFTISRDNSENALYLQMNSLRAEDTAVYYCARILRGGSGMDLWGQGTLVTVSS

SEQ ID NO: 9 - DNA, 336 bp, synthetic construct
  misc_feature 1..336: note = source = /mol_type="unassigned DNA" /note="Variable light chain nucleotide sequence " /organism="Artificial Sequence"
  source 1..336: mol_type = other DNA
cagtctgtgctgactcagccaccctcagcgtctgggacccccgggcagagggtcaccatctcttgcactgggagcagctccaacatcggggcgggttacaatgtatactggtatcagcagctcccaggaacggcccccaaactcctcatctatggtaacatcaatcggccctcaggggtccctgaccgattctctggctccaagtctggcacctcagcctccctggccatcagtgggctccggtccgaggatgaggctgattattactgtgcagcatgggataagagcatttctggtctggttttcggcggaggaaccaagctgacggtcctaggt

SEQ ID NO: 10 - DNA, 357 bp, synthetic construct
  misc_feature 1..357: note = source = /mol_type="unassigned DNA" /note="Variable heavy chain nucleotide sequence" /organism="Artificial Sequence"
  source 1..357: mol_type = other DNA
gaggtgcagctgttggagtctgggggaggcttggtacagcctggggggtccctgagactctcctgtgcagcctctggattcaccttcagtacttatggcatgcactgggtccgccaggctccagggaaggggctggagtggctttcatatattagtggtggtagtagttacattttctacgcagactcagtgaggggccgattcaccatctccagagacaactccgagaacgcgctgtatctgcaaatgaacagcctgagagccgaggacacggccgtgtattactgtgcgagaatattaagaggcgggagcggtatggacctctggggccaaggtacactggtcaccgtgagctca

SEQ ID NO: 11 - protein, 105 aa, Homo sapiens
  REGION 1..105: note = Human Ig lambda light chain C2 region
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 12 - protein, 330 aa, Homo sapiens [sequence omitted]

SEQ ID NO: 13 - protein, 277 aa, Homo sapiens
  REGION 1..277: note = Human CD40 sequence
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

(19) United States
(12) Patent Application Publication
(10) Pub. No.: US 2026/0055200 A1
(43) Pub. Date: Feb. 26, 2026

(54) COMBINATION THERAPIES

(71) Applicant: Alligator Bioscience AB, Lund (SE)

(72) Inventors: Peter ELLMARK, Lund (SE); Karin Enell SMITH, Lund (SE); Malin CARLSSON, Lund (SE); David GOMEZ JIMENEZ, Lund (SE); Yago PICO DE COAÑA, Lund (SE); Sumeet AMBARKHANE, Lund (SE); Karin NORDBLADH, Lund (SE); Thomas HELBOE, Lund (SE); Charlotte Astrid RUSSEL, Lund (SE); Per NORLÉN, Lund (SE)

(21) Appl. No.: 18/874,865

(22) PCT Filed: Jun. 21, 2023
(86) PCT No.: PCT/EP23/66889

§ 371 Date: Dec. 13, 2024

(30) Foreign Application Priority Data
Jun. 23, 2022 (WO) PCT/EP2022/067261
Jan. 3, 2023 (GB) 2300007.8

(51) Int. Cl. C07K 16/28; A61K 31/475; A61K 31/513; A61K 31/519; A61K 31/555; A61K 38/19; A61K 39/00; A61K 45/06; A61P 35/00

(52) U.S. Cl. CPC C07K 16/2878; A61K 31/475; A61K 31/513; A61K 31/519; A61K 31/555; A61K 38/193; A61K 45/06; A61P 35/00; A61K 2039/505; A61K 2039/545

(57) ABSTRACT

The present invention relates to combination therapies for treating cancer, optionally chemotherapy-resistant cancers, in a subject. The combination therapies comprise (a) an antibody or antigen-binding portion thereof that specifically binds to CD40, and (b) chemotherapy. The invention also relates to pharmaceutical compositions, kits and methods of using such therapies.

### FIELD OF THE INVENTION

The present invention relates to anti-CD40 antibodies and chemotherapy, and their combined use in the treatment of cancer, such as chemotherapeutic-resistant cancer.

### BACKGROUND

Introduction to pancreatic ductal adenocarcinoma Pancreatic ductal adenocarcinoma (PDAC) is a type of exocrine pancreatic cancer. It is the most common type of pancreatic cancer with 95 out of 100 (95%) of all pancreatic cancers. Pancreatic cancer is the fourth leading cause of cancer death in both the United States and the European Union [1]. The incidence of pancreatic cancer is highest in Europe (7.7 per 100,000 people) and North America (7.6 per 100,000 people) followed by Oceania (6.4 per 100,000 people) [2]. In the United States, estimated new cases in 2020 are 57,600 and estimated deaths are 47,050 (Cancer facts and figures, American Cancer Society, 2020). The incidence is slightly higher for men than women.

The five-year life expectancy for pancreatic cancer is about 5%, a number which has not changed over the last two decades. Surgical resection is the only curative modality, but at best a fifth of patients are considered operable, and even in these cases, the five-year survival is on the order of 20%. More than three-quarters of patients present with advanced disease, about half have distant metastases at diagnosis, with the remaining quarter having inoperable local disease. In time, most of these locally advanced cancers will also metastasize [3].

Consequently, there is a need for effective treatment of metastatic pancreatic cancer. Until the last decade, the standard first-line treatment for metastatic pancreatic cancer was gemcitabine, which yielded a median overall survival (mOS) of about 6 months. A major advance came in 2011, with the introduction of the FOLFIRINOX regimen, which yielded an objective response rate (ORR) of 31% and mOS of 11.1 months compared to gemcitabine monotherapy with an ORR of 9.4% and mOS of 6.8 months in the randomized study PRODIGE 4 with 342 patients enrolled [4]. Two years later, the MPACT study comparing gemcitabine plus nab-paclitaxel to gemcitabine also demonstrated superior activity of the combination with an ORR of 23% and mOS of 8.5 months relative to gemcitabine monotherapy with an ORR 7% and mOS 6.7 months [5].

### Mitazalimab, a CD40 Agonist

Mitazalimab (also known as JNJ-64457107 and ADC-1013) is an agonistic, human monoclonal (IgG1) antibody targeting CD40. The agent has been investigated for the treatment of advanced stage solid tumours in two Phase 1 studies: A-14-1013-C-01 (EudraCT No. 2014-004556-56) and JNJ-64457107CAN1001 (EudraCT No. 2016-000969-23).

### Introduction to CD40

CD40 is a co-stimulatory receptor belonging to the tumour necrosis factor receptor (TNFR) superfamily [6]. CD40 is expressed in a multitude of cell types and can be detected on the surface of antigen presenting cells (APCs), including dendritic cells (DCs), B cells, and macrophages. In addition, CD40 is expressed on granulocytes, endothelial cells, smooth muscle cells, fibroblasts, and epithelial cells [6-8]. CD40 is also present on the membranes of a wide range of malignant cells, including non-Hodgkin and Hodgkin lymphomas, myeloma, and some carcinomas including those of the nasopharynx, bladder, cervix, kidney, and ovary [6, 9]. CD40 interacts with a single ligand, CD40L (or CD154), a transmembrane protein that is expressed by activated T cells, B cells, platelets, mast cells, macrophages, basophils, natural killer (NK) cells, and non-hematopoietic cells (smooth muscle cells, endothelial cells, and epithelial cells) [6, 7].

The molecular consequences of CD40 signalling depend on the cell type expressing CD40 and the microenvironment in which the CD40 signal is provided [10]. CD40 ligation and cross-linking is required for the adaptive immune response through the ‘licensing’ of APCs and especially DCs by inducing the upregulation of costimulatory receptors and major histocompatibility complex molecules as well as the production of pro-inflammatory cytokines. Thus, CD40 is involved in the functional maturation of APCs and consequently the activation of antigen-specific T lymphocytes [11-13]. CD40 also plays a role in humoral immunity by activating resting B lymphocytes and by increasing their antigen-presenting function [10, 14]. Moreover, CD40 is involved in the induction of innate immunity through stimulation of cytotoxic myeloid cells such as NK cells, macrophages, and granulocytes [10, 14, 15].

### Summary of Non-Clinical Data

The CD40 agonistic properties of mitazalimab have been validated in vitro in human monocyte-derived DC cultures. Ligation of CD40 by mitazalimab leads to up-regulation of activation markers on the surface of DCs, such as CD80 and CD86, and the release of cytokines such as IL-12. Mitazalimab has also been demonstrated to polarize tumour associated macrophages (TAMs) isolated from human prostate and ovarian tumour samples, from a more immune-suppressive phenotype into a more immune-inflammatory phenotype by upregulation of e.g., CD83.

The agonistic effect of mitazalimab is critically dependent on the binding of the Fc-portion of the antibody to Fcγ-receptors (FcγR). The ability of mitazalimab to activate DCs (i.e., upregulate CD86) was significantly decreased when an aglycosylated variant of mitazalimab which contained a N297Q mutation in the γ chain and thus that does not bind to FcγR was used. The CD40 agonistic effect was again restored upon crosslinking of this aglycosylated mitazalimab variant with an anti-human IgG, conclusively demonstrating that mitazalimab is dependent on FcγR-crosslinking for optimal activity. Mitazalimab showed immune-mediated and antibody-dependent cell-mediated cytotoxicity (ADCC)-dependent anti-tumour efficacy in vivo in human tumours transplanted into NSG mice and human CD40-transgenic (hCD40tg) mice. Moreover, mitazalimab induced a T-cell dependent and tumour-specific immunological memory to bladder cancer cells in hCD40tg mice, with immunity to tumour re-challenge for at least 5 months. Mitazalimab has also demonstrated a synergistically enhanced effect on tumour growth and survival when combined with other immunotherapies such as immune checkpoint inhibitors (e.g., PD-1), vaccination or chemotherapy (e.g., mFOLFIRINOX) in experimental tumour models in hCD40tg mice. For additional and detailed information of non-clinical data, see the mitazalimab Investigator's Brochure.

### Summary of Clinical Data

### Mitazalimab Single-Agent Studies

First-In-Human Study with Intratumoural Administration of Mitazalimab

Study A-14-1013-C-01 (NCT02379741) was a first-in-human, multicentre, non-randomized, open-label, multiple ascending dose escalation, phase-1 study of mitazalimab (ADC-1013) in patients with advanced stage solid malignancies.

Mitazalimab was administered as an intratumoural bolus injection into the same tumour lesion (intratumoural administration) every 14 days in 18 patients and as an intravenous (IV) infusion over 2 hours every 14 days in 5 patients.

No maximum tolerated dose (MTD) was identified. Increases in liver enzymes or bilirubin were observed in 9 of 18 patients given the intratumoural administration and 3 of 5 patient given IV administration. Only two of the patients had normal liver enzymes and bilirubin values at baseline. Two dose-limiting toxicities (DLTs) were observed, one patient with grade 3 abdominal pain and one patient with grade 3 cholecystitis, both received the intratumoural administration. The best response was stable disease (SD) in one patient with renal cell carcinoma (400 μg/kg intratumoural). No patients with pancreatic cancer were enrolled in this study.

Dose Escalation Study with Intravenous Administration of Mitazalimab

Study 64457107CAN1001 (NCT02829099) was a multicentre, non-randomized, open-label, ascending dose escalation phase 1 study with mitazalimab (JNJ-64457107) conducted in patients with advanced stage solid tumours. Mitazalimab was administered every 14 days as an IV infusion at doses ranging from 75 μg/kg to 2000 μg/kg with corticosteroid included in the premedication, and up to 1200 μg/kg without corticosteroid.

In total, 95 patients were exposed to mitazalimab. One patient remains on treatment. No MTD was identified. Two patients experienced a DLT, one patient with headache lasting 5 days and one patient had grade 3 liver enzyme elevation together with grade 2 bilirubin elevation. Seven pancreatic cancer patients were enrolled in the study, one of whom experienced SD for more than 6 months. A partial response (PR) was observed in a patient with renal cell cancer, who received fourteen cycles (i.e., 28 doses) at the 1200 μg/kg dose level and was on study for 9.2 months [16].

### Pharmacodynamic Activity

The pharmacodynamic biomarker data in Study 644577107CAN1001 were studied after the first mitazalimab administration and are consistent with CD40-mediated immune cell activation. Following IV administration of mitazalimab, margination of B cells was observed, with dose-dependent B-cell recovery, at all doses tested. NK cells and T cells also decreased in the peripheral blood following infusion of mitazalimab at all doses tested, with the exception of the lowest dose (75 μg/kg). The levels of both cell types were fully recovered by study Day 8.

CD40 receptor occupancy was assessed on B cells and shown to be dose dependent. CD40 remained engaged through Day 8 post mitazalimab administration. Peripheral levels of MCP-1, IP-10 and MIP-1B chemokines peaked 1-4 hours post-infusion, consistent with myeloid cell activation. Other chemokines (such as MIP-1α and IL-8) and cytokines (such as IFN-γ, TNF-α and IL12p70) were also observed, but to a lesser extent. IL-6 levels, which can be highly induced in subjects with cytokine release syndrome, were not elevated following infusion of mitazalimab. The pattern of biomarker changes was consistent with the proposed mechanism of mitazalimab as a CD40 agonist.

CD40 Agonists Administered to Patients with Pancreatic Cancer

### Selicrelumab

Selicrelumab (CP-870.893, RO7009789) is a CD40 agonistic monoclonal antibody, with an IgG2 format that activates CD40 independently from cross-linking with Fcγ receptors [17]. It is a general belief with agonistic CD40 antibodies that systemic delivery with cross-linking independent antibodies may increase the risk for toxicities which can reduce the therapeutic window [18]. Selicrelumab has been evaluated in combination with gemcitabine in patients with previously untreated pancreatic cancer [19]. The combination was well tolerated up to 0.2 mg/kg. One DLT, a cerebrovascular accident, occurred at the 0.2 mg/kg dose level. Four patients out of 22 had a PR, no complete responses (CR) were observed (response rate 19%). Cytokine release syndrome in relation to the selicrelumab infusion was observed in 20 of the 22 patients, one event was grade 3, all other events were grade 1 or 2. Immune activation with an increase in inflammatory cytokines, increase in expression of co-stimulatory molecules on B cells, and transient depletion of B cells were observed in all patients. Liver enzyme elevations occurred in approximately ⅔ of the patients, all were grade 1-2, while hyperbilirubinemia occurred in a few patients.

### APX005M

APX005M is a CD40 agonistic monoclonal antibody, with a mutated IgG1 format for improved binding to FcγR2b, which depends on cross-linking with FcγRs for its function. APX005M has a profile similar to mitazalimab, with the aim to improve efficacy and safety compared with previous CD40 antibodies. APX-005M is in clinical development for several malignancies.

Early clinical data with APX005M in combination with gemcitabine plus nab-paclitaxel with or without nivolumab in patients with previously untreated metastatic pancreatic cancer were presented in 2019, see Table 1. Gemcitabine plus nab-paclitaxel was given 3 times, nivolumab was given twice and APX005M once in each 28-days cycle. Immune profiling of the PBMCs demonstrated remodelling of the myeloid compartment in response to treatment, with rapid activation of DCs in most patients. Analysis of circulating mutant KRAS DNA showed marked and rapid decrease with therapy. Preliminary efficacy data from 24 patients showed encouraging data for the combinations and the randomized study continues [20].

TABLE 1
Preliminary efficacy data for APX005M in combination with gemcitabine
plus nab-paclitaxel (Gem-NabPac) with or without nivolimab (Nivo)

| | Gem- | Gem- | | | |
|---|---|---|---|---|---|
| | NabPac + | NabPac + | | Gem- | |
| | APX005M | APX005M | Gem-NabPac/ | NabPac/ | |
| | 0.1 | 0.3 | Nivo + APX005M | Nivo + APX005M | |
| | mg/kg | mg/kg | 0.1 mg/kg | 0.3 mg/kg | Total |
| | (N = 6) | (N = 6) | (N = 6) | (N = 6) | (N = 24) |
| Complete | 0 | 0 | 0 | 0 | 0 |
| Response | | | | | |
| Partial | 3 (50%) | 2 (33%) | 4 (67%) | 4 (67%) | 13 (54%) |
| Response | | | | | |
| Stable | 3 (50%) | 3 (50%) | 1 (17%) | 2 (33%) | 9 (38%) |
| Disease | | | | | |
| Progressive | 0 | 1 (17%) | 0 | 0 | 1 (4%) |
| Disease | | | | | |
| Not | 0 | 0 | 1 (17%) | 0 | 1 (4%) |
| Evaluable | | | | | |

The listing or discussion of an apparently prior-published document in this specification should not necessarily be taken as an acknowledgement that the document is part of the state of the art or is common general knowledge.

### SUMMARY OF THE INVENTION

Combination chemotherapy for metastatic pancreatic cancer has been pushed to the brink of tolerability, but survival outcomes remain poor. Pancreatic cancer is classified as immunologically “cold” compared to other tumours characterized by immune infiltrates. The desmoplastic stroma that forms around pancreatic cancer, in addition to functioning as a physical barrier to chemotherapy, is host to tumour fibroblasts and suppressive myeloid cells that dampen the immune response in the tumour microenvironment. Moreover, because pancreatic cancer harbours relatively few nonsynonymous mutations compared to other cancers, the tumours are characterised by low expression of tumour neoantigens. These two factors contribute to the lack of activity seen with checkpoint inhibitors in pancreatic cancer.

It is therefore an object of the present invention to provide an improved combination therapy for treating cancer (e.g. pancreatic cancer), optionally chemotherapy-resistant cancer.

A first aspect of the invention provides a combination therapy for use in treating cancer, optionally chemotherapy-resistant cancer, in a subject comprising: an antibody or antigen-binding portion thereof that specifically binds to CD40, and chemotherapy.

A second aspect of the invention provides a combination therapy comprising an antibody or antigen-binding portion thereof and chemotherapy for use in a dosage regimen for treating cancer, optionally chemotherapeutic-resistant cancer, wherein the dosage regimen comprises the following steps: (a) administration of an antibody or antigen-binding portion thereof that specifically binds to CD40, and (b) administration of chemotherapy.

A third aspect of the invention provides an antibody or antigen-binding portion thereof that specifically binds to CD40 for use in treating cancer, optionally chemotherapy-resistant cancer, in a subject, wherein the antibody or antigen-binding portion thereof is for use in combination with chemotherapy.

In an alternative version of the third aspect, the invention provides a chemotherapy for use in treating cancer, optionally chemotherapy-resistant cancer, in a subject, wherein the chemotherapy is for use in combination with an antibody or antigen-binding portion thereof that specifically binds to CD40.

In some embodiments, the subject may be undergoing treatment with the antibody or antigen-binding portion thereof and is then treated with chemotherapy to achieve the combination therapy. In one embodiment, the subject may be undergoing treatment with the chemotherapy and is then treated with the antibody or antigen-binding portion thereof to achieve the combination therapy.

A fourth aspect of the invention provides a use of an antibody or antigen-binding portion thereof that specifically binds to CD40 in the preparation of a medicament for treating cancer, optionally chemotherapeutic-resistant cancer, wherein the antibody or antigen-binding portion thereof is for use in combination with chemotherapy.

In an alternative version of the fourth aspect, the invention provides a use of an antibody or antigen-binding portion thereof that specifically binds to CD40 in the preparation of a medicament in the combination therapy according to the first or second aspect of the invention.

A fifth aspect of the invention provides a method of treating cancer, optionally chemotherapeutic-resistant cancer, in a subject, the method comprising administering to the subject a therapeutically effective amount of an antibody or antigen-binding portion thereof that specifically binds to CD40 and chemotherapy.

In an alternative version of the fifth aspect, the invention provides a method of treating cancer, optionally chemotherapeutic-resistant cancer, in a subject, the method comprising administering to the subject a therapeutically effective amount of the combination therapy according to the first aspect.

In a further alternative version of the fifth aspect, the invention provides a method of treating cancer, optionally chemotherapeutic-resistant cancer, in a subject, the method comprising administering to the subject the combination therapy according to the second aspect.

A sixth aspect of the invention provides a pharmaceutical composition comprising an antibody or antigen-binding portion thereof that specifically binds to CD40 and chemotherapy. The pharmaceutical composition may comprise the antibody or antigen-binding portion thereof and/or the chemotherapy according to any preceding aspect of the invention. The pharmaceutical composition may comprise one or more components of the chemotherapy (for example, 1, 2, 3 or all 4 components of a FOLFIRINOX regimen), and optionally further comprise the antibody or antigen-binding portion thereof.

A seventh aspect of the invention provides a kit comprising an antibody or antigen-binding portion that specifically binds to CD40 and chemotherapy. The kit may comprise the antibody or antigen-binding portion thereof and/or the chemotherapy according to any preceding aspect of the invention.

The following paragraphs may be in relation to any of the preceding aspects. Preferences and options for a given aspect, feature or parameter of the invention should, unless the context indicates otherwise, be regarded as having been disclosed in combination with any and all preferences and options for all other aspects, features and parameters of the invention.

In some embodiments, the cancer, optionally chemotherapy-resistant cancer, is pancreatic cancer. In some embodiments, the pancreatic cancer is an exocrine tumour, such as an exocrine adenocarcinoma or pancreatic ductal adenocarcinoma (PDAC), or an endocrine tumour. In some embodiments, the cancer is a solid tumour. The solid tumour may be selected from the group consisting of a pancreatic tumour, an adenoma, a blastoma, a carcinoma, a desmoid tumour, a desmoplastic small round cell tumour, an endocrine tumour, a germ cell tumour, a lymphoma, a sarcoma, a Wilms tumour, a lung tumour, a colon tumour, a lymph tumour, a breast tumour and a melanoma.

In some embodiments, the chemotherapy is selected from the group consisting of FOLFIRINOX or variants thereof (such as mFOLFIRINOX), gemcitabine, nab-paclitaxel, and combinations thereof. In some embodiments, the chemotherapy is FOLFIRINOX or a variant thereof, and the antibody or antigen-binding portion thereof is mitazalimab.

In some embodiments, the FOLFIRINOX or variant thereof comprises oxaliplatin (such as Eloxatin® or generic drug) infusion, optionally wherein the oxaliplatin is administered at a dose of 85 mg/m², intravenously, and/or for 2 hours. In some embodiments, the FOLFIRINOX or variant thereof comprises a folinate (such as leucovorin, calcium folinate, calcium levofolinate, disodium folinate and disodium levofolinate) infusion, optionally wherein the leucovorin is administered at a dose of 400 mg/m², intravenously, and/or for 2 hours. In some embodiments, the FOLFIRINOX or variant thereof comprises irinotecan (such as Campto®) infusion, optionally wherein the irinotecan is administered at a dose of 150 mg/m², intravenously, and/or 30 minutes after the end of the leucovorin infusion. In some embodiments, the FOLFIRINOX or variant thereof comprises 5-fluorouracil infusion, optionally wherein the 5-fluorouracil is administered at a dose of 2400 mg/m², intravenously, and/or for a duration of 46-48 hours (e.g. 2.4 g/m²/day).

In some embodiments, the subject receives at least one treatment cycle of FOLFIRINOX (e.g. mFOLFIRINOX). A treatment cycle may correspond to the OPTIMIZE-1 study (see Example 2). In some embodiments, the subject receives 1, 2, 3, 4, 5, 6, 7, 8, 9, 10, 11, 12 or more treatment cycles. In some embodiments, the subject does not receive more than 12 treatment cycles. In some embodiments, the subject receives 4 treatment cycles. In some embodiments, the subject receives 8 treatment cycles. In some embodiments, the subject receives more than 12 treatment cycles, for example the subject may receive 13, 14, 15, 16, 17, 18, 19, 20 or more treatment cycles. The number of treatment cycles for the patient may be adapted based on the observation of responsiveness to the treatment and/or the level of toxicity in the patient. Such an adaptation is within the remit of a physician for the patient, and readouts for responsiveness and/or toxicity are a matter of routine (such as described herein).

In some embodiments, the oxaliplatin is administered over 2 hours, immediately followed by the folinate (e.g. leucovorin) over 2 hours; and the irinotecan is administered over 90 minutes, starting 30 minutes after the start of the leucovorin; following which the 5-fluorouracil is administered over 46-48 hours.

In some embodiments, the subject receives premedication. In some embodiments, the subject receives post-medication. In some embodiments, the subject receives premedication and post-medication. Premedication may comprise: (i) NK1-receptor antagonist, such as Aprepritant, 125 mg PO, 60 minutes prior to infusion and/or continuously during days with chemotherapy, (ii) 5-HT3 receptor antagonist, such as Ondansetron, 8 mg PO, 30 minutes prior to infusion and/or continuously during days with chemotherapy, and/or (iii) corticosteroid, such as dexamethasone, 8 mg IV or PO, 30 minutes prior to infusion. Post-medication may comprise G-CSF, such as Neulasta, 6 mg SC, on the fourth day following the start of the FOLFORINOX regimen or variant thereof, and/or at least 24 hours after the end of the continuous 5-fluorouracil infusion.

In some embodiments, the antibody or antigen-binding portion thereof is administered at a dose from 50 μg/kg to 1200 μg/kg, such as from 450 μg/kg to 900 μg/kg. In some embodiments, the antibody or antigen-binding portion thereof is administered at a dose of 50 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 950 μg/kg, 1000 μg/kg, 1050 μg/kg, 1100 μg/kg, 1150 μg/kg, 1200 μg/kg or higher. In some embodiments, the antibody or antigen-binding portion thereof is administered at a dose of 450 μg/kg. In some embodiments, the antibody or antigen-binding portion thereof is administered at a dose of 900 μg/kg.

In some embodiments, the antibody or antigen-binding portion thereof is administered more than once. For example, the antibody or antigen-binding portion thereof may be administered, in any one or more treatment cycle, two times, three times, four times, five times, six times, seven times or more.

In some embodiments, the antibody or antigen-binding portion thereof comprises the following CDRs:

V_{L} CDR1:
[SEQ ID NO: 1]
CTGSSSNIGAGYNVY;
V_{L} CDR2:
[SEQ ID NO: 2]
GNINRPS;
V_{L} CDR3:
[SEQ ID NO: 3]
CAAWDKSISGLV;
VH CDR1:
[SEQ ID NO: 4]
GFTFSTYGMH;
V_{H} CDR2:
[SEQ ID NO: 5]
GKGLEWLSYISGGSSYIFYADSVRGR;
and
V_{H} CDR3:
[SEQ ID NO: 6]
CARILRGGSGMDL.

In some embodiments, the antibody or antigen-binding portion thereof comprises: (a) the light chain variable region of SEQ ID NO: 7 and/or the heavy chain variable region of SEQ ID NO: 8; (b) the light chain constant region of SEQ ID NO: 11 and/or the heavy chain constant region of SEQ ID NO: 12; or (c) the light chain of SEQ ID NO: 7 plus SEQ ID NO: 11, and/or the heavy chain of SEQ ID NO: 8 plus SEQ ID NO: 12.

In some embodiments, the antibody or antigen-binding portion thereof that specifically binds to CD40 is mitazalimab.

In some embodiments, the antibody or antigen-binding portion thereof that specifically binds to CD40 comprises or consists of an intact antibody, such as an IgG1 antibody.

In some embodiments, the antibody or antigen-binding portion thereof comprises or consists of an antigen-binding fragment selected from the group consisting of: an Fv fragment (such as a single chain Fv fragment, or a disulphide-bonded Fv fragment), and a Fab-like fragment (such as a Fab fragment; a Fab′ fragment or a F(ab)2 fragment).

In some embodiments, the antibody or antigen-binding portion thereof is human or humanised.

In some embodiments, the antibody or antigen-binding portion thereof and the chemotherapy are administered simultaneously, sequentially, or subsequently to each other.

In some embodiments, the antibody or antigen-binding portion thereof and/or the chemotherapy are administered locally to the tumour site. In some embodiments, the antibody or antigen-binding portion thereof and/or the chemotherapy are administered systemically. In some embodiments, the antibody or antigen-binding portion thereof is administered locally, and the chemotherapy is administered systemically. In some embodiments, the chemotherapy is administered locally, and the antibody or antigen-binding portion thereof is administered systemically. In some embodiments, a portion of the chemotherapy may be administered systemically while a different portion is administered locally. Systemic administration may be, for example, intravenous and/or subcutaneous.

In some embodiments, the antibody or antigen-binding portion thereof is administered on multiple separate occasions and the chemotherapy is administered continuously for the duration of the method.

It will be appreciated by persons skilled in the art that the presence of the antibody or antigen-binding portion thereof and the chemotherapy may provide a synergistic benefit in the treatment of cancer, optionally chemotherapy-resistant cancer, in a subject. By “synergistic” we include that the therapeutic effect in combination (e.g. as determined by reference to the rate of growth or the size of the tumour) is greater than the additive therapeutic effect of the two treatments administered separately. Such synergism can be identified by testing the antibody or antigen-binding portion thereof and the chemotherapy, alone and in combination, in a relevant cell line model of the cancer.

Optionally, the combination therapy further comprises a further agent with efficacy in the treatment of cancer, in addition to the antibody or antigen-binding portion thereof and the chemotherapy.

In some embodiments, the subject is a human.

### BRIEF DESCRIPTION OF THE FIGURES

FIG. 1: Study design for a phase 1b/2, open-label, multicentre study designed to evaluate the safety, tolerability, and efficacy of mitazalimab in combination with chemotherapy in patients with metastatic pancreatic ductal adenocarcinoma.

FIG. 2: Exemplary dose escalation schedule.

FIG. 3: Exemplary dosing regimen for mitazalimab and mFOLFIRINOX.

FIG. 4: Exemplary dosing regimen for mitazalimab and gemcitabine plus nab-paclitaxel.

FIG. 5: Exemplary dosing regimen for mitazalimab and FOLFIRINOX in mouse studies.

FIG. 6: Study of tumor volume (A) and survival (B) of hCD40tg bearing MB49-FFX-ACQ. Mitazalimab (mita), FOLFIRINOX (FFX).

FIG. 7: A. MB49 tumor-bearing hCD40tg mice which received treatment with FOLFIRINOX (oxaliplatin, irinotecan, folinic acid and 5-fluorouracil) on days 7-8, 14-15 and 21-22 had reduced tumor growth compared with vehicle treatment. B. Chemotherapy resistant MB49 cells, developed by continuous low dose treatment of FOLFIRINOX in vitro, were inoculated in tumor-bearing hCD40tg mice which received treatment with B. FOLFIRINOX on days 7-8, 14-15 and 21-22 alone or, C. in combination with 100 μg mitazalimab on days 10, 17 and 24 (**, p<0.01 mitazalimab+FOLFIRINOX vs vehicle, Mann-Whitney, non-parametric, 2-tail).

FIG. 8: Treatment duration for a number of patients (denoted as SC-##). Patient SC-13 withdrew from the trial for administration reasons after the first mitazalimab infusion, prior to receiving mFOLFIRINOX, and was not included in RP2D determination.

FIGS. 9 and 10: Peripheral blood assessments for IFN-γ (A), MCP-1 (B), B cell margination (C), and B cell activation (D). The square points correspond to 900 μg/kg, and the circle points correspond to 450 μg/kg.

FIG. 11: Dosing regimen for combination of Mitazalimab (Mita) and folfirinox (FFX/F) in MB49-hCD40 mice.

FIG. 12: Anti-tumour efficacy (A) and survival (B) for mitazalimab given days 10, 17 and 24. Mice were treated with vehicle (Dextrose), FOLFIRINOX (administered days 7-8, 14-15 and 21-22) and/or mitazalimab. (10, 17 and 24). Differences in tumour volume were calculated at day 17 using Mann-Whitney test. * p<0.05, ** p<0.01 and, *** p<0.005).

FIG. 13: Principal Component Analysis (PCA) plot displaying full transcriptome of whole blood samples collected at day 5 from the MB49 hCD40 mice tumour model.

FIG. 14: Heatmap displaying 3452 Differentially Expressed Genes (DEGs) from multigroup comparison (p.value<0.05, FoldChange>2).

FIG. 15: Targets of gene expression related to Mode of Action (MoA).

FIG. 16: Radar chart displaying the Gene Set Variation Analysis (GSVA) scores of selected pathways from the pathway enrichment analysis.

FIG. 17: (A) Key to show the categorisation of spots in the volcano plots based on the positions determined by the dotted lines on the plots. Volcano plots of: Mita vs Untreated (B); Folfirinox vs Untreated (C); Mita vs Folfirinox (D); Mita+Folfirinox vs Folfirinox (E); Mita vs Mita+Folfirinox (F); and Mita+Folfirinox vs Untreated (G).

FIG. 18: Top ten enriched pathways for: Folfirinox vs Untreated (A); Mita vs Folfirinox (B); Mita+Folfirinox vs Folfirinox (C); Mita vs Untreated (D); Mita+Folfirinox vs Untreated (E); and Mita vs Mita+Folfirinox (F).

FIG. 19: Treatment schedule describing KPCY tumour cell inoculation and administration of FOLFIRINOX and mitazalimab. D=day; hCD40tg=human CD40 transgenic; s.c.=subcutaneous.

FIG. 20: Anti-tumor efficacy of mitazalimab in combination with FOLFIRINOX KPCY pancreatic tumour model. A) tumour growth curve and B) changes in tumour volume Day 31 after tumour inoculation. Change in tumour volume was calculated as percent change from baseline tumour volume at Day 6, one day before initiation of treatment, with tumour volume at Day 31. *** indicates p<0.001; ** indicates p<0.01.

FIG. 21: A Swimmer's plot of the patients from Example 15. Symbols show Complete Response (CR), Partial Response (PR), Stable Disease (SD), Progressive Disease (PD), Not Evaluable (NE), Death, and ongoing treatment at cut-off date. 900 μg/kg Mitazalimab, N=57

FIG. 22: A Waterfall plot of bast percentage change from baseline of the patients used in Example 15. 900 μg/kg Mitazalimab, N=56.

FIG. 23: A Waterfall plot of percentage change from baseline at last available CT scan of the patients used in Example 15. 900 μg/kg Mitazalimab, N=56.

FIG. 24: A Spider plot of the percentage change in target lesions (sum of longest diameters) over time of the patients used in Example 15. 900 μg/kg Mitazalimab, N=57.

FIG. 25: Kaplan-Meier plot of Duration of Response (FAS), 900 μg/kg Mitazalimab, N=18

FIG. 26: Kaplan-Meier plot of Time to progression (FAS), 900 μg/kg Mitazalimab, N=57

FIG. 27: Kaplan-Meier plot of Progression-free survival (FAS), 900 μg/kg Mitazalimab, N=57

FIG. 28: Kaplan-Meier plot of Overall survival (FAS), 900 μg/kg Mitazalimab, N=57

### DETAILED DESCRIPTION OF THE INVENTION

All publications, patents and patent applications cited herein, whether supra or infra, are hereby incorporated by reference in their entirety.

It is to be understood that different applications of the disclosed combination therapies, uses, methods, pharmaceutical compositions and kits may be tailored to the specific needs in the art. It is also to be understood that the terminology used herein is for the purpose of describing particular embodiments of the invention only and is not intended to be limiting.

In addition, as used in this specification and the appended claims, the singular forms “a”, “an”, and “the” include plural referents unless the content clearly dictates otherwise. Thus, for example, reference to “an antibody” includes “antibodies”, reference to “an antigen” includes two or more such antigens, reference to “a subject” includes two or more such subjects, and the like.

The terms “combination therapy” or “combined treatment” or “in combination” as used herein denotes any form of concurrent or parallel treatment with at least two distinct arms of treatment. For example, a first arm of treatment may be with an antibody or antigen-binding portion thereof as described herein, and a second arm of treatment may be with chemotherapy.

An “antibody or antigen-binding portion thereof” may be referred to as an immunotherapy or an immunotherapeutic agent. Therefore, the combination therapy may be considered a combination of immunotherapy and chemotherapy. The term “antibody” as referred to herein includes whole antibodies and any antigen binding portion (i.e. “antigen-binding fragment”) or single chains thereof. An antibody refers to a glycoprotein comprising at least two heavy (H) chains and two light (L) chains inter-connected by disulfide bonds, or an antigen-binding portion thereof. Each heavy chain is comprised of a heavy chain variable region (abbreviated herein as VH) and a heavy chain constant region. Each light chain is comprised of a light chain variable region (abbreviated herein as VL) and a light chain constant region. The variable regions of the heavy and light chains contain a binding domain that interacts with an antigen. The VH and VL regions can be further subdivided into regions of hypervariability, termed complementarity determining regions (CDR), interspersed with regions that are more conserved, termed framework regions (FR). The constant regions of the antibodies may mediate the binding of the immunoglobulin to host tissues or factors, including various cells of the immune system (e.g. effector cells) and the first component (Clq) of the classical complement system.

The term “immunotherapeutic agent” is intended to include any antibody or antigen-binding portion thereof which can stimulate a host immune system to generate an immune response to a tumour or cancer in the subject. The term “immune response” includes T cell mediated and/or B cell mediated immune responses. Exemplary immune responses include T cell responses, e.g. cytokine production and cellular cytotoxicity. In addition, the term immune response includes immune responses that are indirectly affected by T cell activation, e.g. antibody production (humoral responses) and activation of cytokine responsive cells, e.g. macrophages.

Heavy chains can be of any isotype, including IgG (IgG1, IgG2, IgG3 and IgG4 subtypes), IgA (IgA1 and IgA2 subtypes), IgM and IgE.

Light chains include kappa chains and lambda chains.

Of relevance are antibodies and their antigen-binding fragments that have been “isolated” so as to exist in a physical milieu distinct from that in which it may occur in nature or that have been modified so as to differ from a naturally occurring antibody in amino acid sequence.

An antibody or antigen-binding portion thereof may be a polyclonal antibody or a monoclonal antibody. The antibody or antigen-binding portion thereof may be produced by any suitable method. For example, suitable methods for producing monoclonal antibodies are disclosed in “Monoclonal Antibodies; A manual of techniques”, H Zola (CRC Press, 1988) and in “Monoclonal Hybridoma Antibodies: Techniques and Application”, SGR Hurrell (CRC Press, 1982). Recombinant techniques may also be used.

The term “antigen-binding portion” or “antigen-binding fragment” of an antibody refers to one or more fragments of an antibody that retain the ability to specifically bind to an antigen, such as CD40. It has been shown that the antigen-binding function of an antibody can be performed by fragments of a full-length antibody. Examples of binding fragments encompassed within the term “antigen-binding portion” of an antibody include a Fab fragment, a F(ab′)₂ fragment, a Fab′ fragment, a Fd fragment, a Fv fragment, a dAb fragment and an isolated complementarity determining region (CDR). Single chain antibodies such as scFv and heavy chain antibodies such as VHH and camel antibodies are also intended to be encompassed within the term “antigen-binding portion” of an antibody. These antibody fragments may be obtained using conventional techniques known to those of skill in the art, and the fragments may be screened for utility in the same manner as intact antibodies.

An antibody for use in the methods of the invention may be a human antibody. The term “human antibody”, as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g. mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences—such antibodies are typically referred to as chimeric or humanised.

A human antibody for use the methods of the invention is typically a human monoclonal antibody. Such a human monoclonal antibody may be produced by a hybridoma which includes a B cell obtained from a transgenic nonhuman animal, e.g. a transgenic mouse, having a genome comprising a human heavy chain transgene and a light chain transgene fused to an immortalised cell. Human antibodies may also be prepared by in vitro immunisation of human lymphocytes followed by transformation of the lymphocytes with Epstein-Barr virus. The term “human antibody derivatives” refers to any modified form of the human antibody, e.g. a conjugate of the antibody and another agent or antibody.

An antibody or antigen-binding portion thereof according to the invention may alternatively be a humanised antibody.

The term “humanised” refers to an antibody molecule, generally prepared using recombinant techniques, having an antigen binding site derived from an immunoglobulin from a non-human species and a remaining immunoglobulin structure based upon the structure and/or sequence of a human immunoglobulin. The antigen-binding site may comprise either complete non-human antibody variable domains fused to human constant domains, or only the complementarity determining regions (CDRs) of such variable domains grafted to appropriate human framework regions of human variable domains. The framework residues of such humanised molecules may be wild type (e.g. fully human) or they may be modified to contain one or more amino acid substitutions not found in the human antibody whose sequence has served as the basis for humanisation. Humanisation lessens or eliminates the likelihood that a constant region of the molecule will act as an immunogen in human individuals, but the possibility of an immune response to the foreign variable region remains (LoBuglio, A. F. et al. (1989) “Mouse/Human Chimeric Monoclonal Antibody In Man: Kinetics And Immune Response,” Proc. Natl. Acad. Sci. (U.S.A.) 86:4220-4224).

Another approach focuses not only on providing human-derived constant regions, but modifying the variable regions as well so as to reshape them as closely as possible to human form. It is known that the variable regions of both heavy and light chains contain three complementarity-determining regions (CDRs) which vary in response to the antigens in question and determine binding capability, flanked by four framework regions (FRs) which are relatively conserved in a given species and which putatively provide a scaffolding for the CDRs. When nonhuman antibodies are prepared with respect to a particular antigen, the variable regions can be “reshaped” or “humanised” by grafting CDRs derived from nonhuman antibody on the FRs present in the human antibody to be modified. Application of this approach to various antibodies has been reported by Sato, K. et al. (1993) Cancer Res 53:851-856. Riechmann, L. et al. (1988) “Reshaping Human Antibodies for Therapy,” Nature 332:323-327; Verhoeyen, M. et al. (1988) “Reshaping Human Antibodies: Grafting An Antilysozyme Activity,” Science 239:1534-1536; Kettleborough, C. A. et al. (1991) “Humanization Of A Mouse Monoclonal Antibody By CDR-Grafting: The Importance Of Framework Residues On Loop Conformation,” Protein Engineering 4:773-3783; Maeda, H. et al. (1991) “Construction Of Reshaped Human Antibodies With HIV-Neutralizing Activity,” Human Antibodies Hybridoma 2:124-134; Gorman, S. D. et al. (1991) “Reshaping A Therapeutic CD4 Antibody,” Proc. Natl. Acad. Sci. (U.S.A.) 88:4181-4185; Tempest, P. R. et al. (1991) “Reshaping A Human Monoclonal Antibody To Inhibit Human Respiratory Syncytial Virus Infection in vivo,” Bio/Technology 9:266-271; Co, M. S. et al. (1991) “Humanized Antibodies For Antiviral Therapy,” Proc. Natl. Acad. Sci. (U.S.A.) 88:2869-2873; Carter, P. et al. (1992) “Humanization Of An Anti-p185her2 Antibody For Human Cancer Therapy,” Proc. Natl. Acad. Sci. (U.S.A.) 89:4285-4289; and Co, M. S. et al. (1992) “Chimeric And Humanized Antibodies With Specificity For The CD33 Antigen,” J. Immunol. 148:1149-1154.

In some embodiments, humanised antibodies preserve all CDR sequences (for example, a humanised mouse antibody which contains all six CDRs from the mouse antibodies). In other embodiments, humanised antibodies have one or more CDRs (one, two, three, four, five, six) which are altered with respect to the original antibody, which are also termed one or more CDRs “derived from” one or more CDRs from the original antibody. The ability to humanise an antigen is well known (see, e.g., U.S. Pat. Nos. 5,225,539; 5,530,101; 5,585,089; 5,859,205; 6,407,213; 6,881,557).

Any antibody referred to herein may be provided in isolated form or may optionally be provided linked (directly or indirectly) to another moiety. The other moiety may be a therapeutic molecule such as a cytotoxic moiety or a drug.

The therapeutic molecule may be directly attached, for example by chemical conjugation, to an antibody of the invention. Methods for conjugating molecules to an antibody are known in the art. For example, carbodiimide conjugation (Bauminger & Wilchek (1980) Methods Enzymol. 70, 151-159) may be used to conjugate a variety of agents, including doxorubicin, to antibodies or peptides. The water-soluble carbodiimide, 1-ethyl-3-(3-dimethylaminopropyl) carbodiimide (EDC) is particularly useful for conjugating a functional moiety to a binding moiety.

Other methods for conjugating a moiety to antibodies can also be used. For example, sodium periodate oxidation followed by reductive alkylation of appropriate reactants can be used, as can glutaraldehyde cross-linking. However, it is recognised that, regardless of which method of producing a conjugate of the invention is selected, a determination must be made that the antibody maintains its targeting ability and that the functional moiety maintains its relevant function.

A cytotoxic moiety may be directly and/or indirectly cytotoxic. By “directly cytotoxic” it is meant that the moiety is one which on its own is cytotoxic. By “indirectly cytotoxic” it is meant that the moiety is one which, although is not itself cytotoxic, can induce cytotoxicity, for example by its action on a further molecule or by further action on it. The cytotoxic moiety may be cytotoxic only when intracellular and is preferably not cytotoxic when extracellular.

The antibody or antigen-binding portion thereof may be linked to a cytotoxic moiety which is a directly cytotoxic chemotherapeutic agent. Optionally, the cytotoxic moiety is a directly cytotoxic polypeptide. Cytotoxic chemotherapeutic agents are well known in the art. In this context of the present invention, the antibody or antigen-binding portion thereof with a cytotoxic chemotherapeutic agent is for use in combination with a distinct chemotherapy. In some embodiments, the antibody or antigen-binding portion thereof is not conjugated to a cytotoxic chemotherapeutic agent.

Cytotoxic chemotherapeutic agents, whether for conjugated to the antibody or antigen-binding-portion thereof or for separate use as chemotherapy, such as anticancer agents, include: alkylating agents including nitrogen mustards such as mechlorethamine (HN2), cyclophosphamide, ifosfamide, melphalan (L-sarcolysin) and chlorambucil; ethylenimines and methylmelamines such as hexamethylmelamine, thiotepa; alkyl sulphonates such as busulfane; nitrosoureas such as carmustine (BCNU), lomustine (CCNU), semustine (methyl-CCNU) and streptozocin (streptozotocin); and triazenes such as decarbazine (DTIC; dimethyltriazenoimidazole-carboxamide); Antimetabolites including folic acid analogues such as methotrexate (amethopterin); pyrimidine analogues such as fluorouracil (5-fluorouracil; 5-FU), floxuridine (fluorodeoxyuridine; FUdR) and cytarabine (cytosine arabinoside); and purine analogues and related inhibitors such as mercaptopurine (6-mercaptopurine; 6-MP), thioguanine (6-thioguanine; TG) and pentostatin (2′-deoxycoformycin). Natural Products including vinca alkaloids such as vinblastine (VLB) and vincristine; epipodophyllotoxins such as etoposide and teniposide; antibiotics such as dactinomycin (actinomycin D), daunorubicin (daunomycin; rubidomycin), doxorubicin, bleomycin, plicamycin (mithramycin) and mitomycin (mitomycin C); enzymes such as L-asparaginase; and biological response modifiers such as interferon alphenomes. Miscellaneous agents including platinum coordination complexes such as cisplatin (cis-DDP) and carboplatin; anthracenedione such as mitoxantrone and anthracycline; substituted urea such as hydroxyurea; methyl hydrazine derivative such as procarbazine (N-methylhydrazine, MIH); and adrenocortical suppressant such as mitotane (o,p′-DDD) and aminoglutethimide; taxol and analogues/derivatives; and hormone agonists/antagonists such as flutamide and tamoxifen.

The cytotoxic moiety may be a cytotoxic peptide or polypeptide moiety which leads to cell death. Cytotoxic peptide and polypeptide moieties are well known in the art and include, for example, ricin, abrin, Pseudomonas exotoxin, tissue factor and the like. Methods for linking them to targeting moieties such as antibodies are also known in the art. Other ribosome inactivating proteins are described as cytotoxic agents in WO 96/06641. Pseudomonas exotoxin may also be used as the cytotoxic polypeptide. Certain cytokines, such as TNFα and IL-2, may also be useful as cytotoxic agents.

Certain radioactive atoms may also be cytotoxic if delivered in sufficient doses. Thus, the cytotoxic moiety may comprise a radioactive atom which, in use, delivers a sufficient quantity of radioactivity to the target site so as to be cytotoxic. Suitable radioactive atoms include phosphorus-32, iodine-125, iodine-131, indium-111, rhenium-186, rhenium-188 or yttrium-90, or any other isotope which emits enough energy to destroy neighbouring cells, organelles or nucleic acid. Preferably, the isotopes and density of radioactive atoms in the agents of the invention are such that a dose of more than 4000 cGy (preferably at least 6000, 8000 or 10000 cGy) is delivered to the target site and, preferably, to the cells at the target site and their organelles, particularly the nucleus.

The radioactive atom may be attached to the antibody, antigen-binding fragment, variant, fusion or derivative thereof in known ways. For example, EDTA or another chelating agent may be attached to the binding moiety and used to attach 111In or 90Y. Tyrosine residues may be directly labelled with 1251 or 1311.

The cytotoxic moiety may be a suitable indirectly cytotoxic polypeptide. The indirectly cytotoxic polypeptide may be a polypeptide which has enzymatic activity and can convert a non-toxic and/or relatively non-toxic prodrug into a cytotoxic drug. With antibodies, this type of system is often referred to as ADEPT (Antibody-Directed Enzyme Prodrug Therapy). The system requires that the antibody locates the enzymatic portion to the desired site in the body of the patient and after allowing time for the enzyme to localise at the site, administering a prodrug which is a substrate for the enzyme, the end product of the catalysis being a cytotoxic compound. The object of the approach is to maximise the concentration of drug at the desired site and to minimise the concentration of drug in normal tissues. The cytotoxic moiety may be capable of converting a non-cytotoxic prodrug into a cytotoxic drug.

The enzyme and prodrug of the system using a targeted enzyme as described herein may be any of those previously proposed. The cytotoxic substance may be any existing anti-cancer drug such as an alkylating agent; an agent which intercalates in DNA; an agent which inhibits any key enzymes such as dihydrofolate reductase, thymidine synthetase, ribonucleotide reductase, nucleoside kinases or topoisomerase; or an agent which effects cell death by interacting with any other cellular constituent. Etoposide is an example of a topoisomerase inhibitor.

Reported prodrug systems include those listed in Table 2.

TABLE 2

| Enzyme | Prodrug |
|---|---|
| Carboxypeptidase G2 | Derivatives of L-glutamic acid and benzoic acid |
| | mustards, aniline mustards, phenol mustards and |
| | phenylenediamine mustards; fluorinated derivatives of |
| | these |
| Alkaline phosphatase | Etoposide phosphate |
| | Mitomycin phosphate |
| Beta-glucuronidase | p-Hydroxyaniline mustard-glucuronide |
| | Epirubicin-glucuronide |
| Penicillin-V-amidase | Adriamycin-N phenoxyacetyl |
| Penicillin-G-amidase | N-(4′-hydroxyphenyl acetyl) palytoxin |
| | Doxorubicin and melphalan |
| Beta-lactamase | Nitrogen mustard-cephalosporin |
| | p-phenylenediamine; doxorubicin derivatives; |
| | vinblastine derivative-cephalosporin, cephalosporin |
| | mustard; a taxol derivative |
| Beta-glucosidase | Cyanophenylmethyl-beta-D-gluco-pyranosiduronic acid |
| Nitroreductase | 5-(Azaridin-1-yl-)-2,4-dinitrobenzamide |
| Cytosine deaminase | 5-Fluorocytosine |
| Carboxypeptidase A | Methotrexate-alanine |

Suitable enzymes for forming part of an enzymatic portion include: exopeptidases, such as carboxypeptidases G, G1 and G2 (for glutamylated mustard prodrugs), carboxypeptidases A and B (for MTX-based prodrugs) and aminopeptidases (for 2-α-aminocyl MTC prodrugs); endopeptidases, such as e.g. thrombolysin (for thrombin prodrugs); hydrolases, such as phosphatases (e.g. alkaline phosphatase) or sulphatases (e.g. aryl sulphatases) (for phosphylated or sulphated prodrugs); amidases, such as penicillin amidases and arylacyl amidase; lactamases, such as β-lactamases; glycosidases, such as β-glucuronidase (for β-glucuronomide anthracyclines), α-galactosidase (for amygdalin) and β-galactosidase (for β-galactose anthracycline); deaminases, such as cytosine deaminase (for 5FC); kinases, such as urokinase and thymidine kinase (for gancyclovir); reductases, such as nitroreductase (for CB1954 and analogues), azoreductase (for azobenzene mustards) and DT-diaphorase (for CB1954); oxidases, such as glucose oxidase (for glucose), xanthine oxidase (for xanthine) and lactoperoxidase; DL-racemases, catalytic antibodies and cyclodextrins.

Preferably, the prodrug is relatively non-toxic compared to the cytotoxic drug. Typically, it has less than 10% of the toxicity, preferably less than 1% of the toxicity as measured in a suitable in vitro cytotoxicity test.

It is likely that the moiety which is able to convert a prodrug to a cytotoxic drug will be active in isolation from the rest of the agent of the invention but it is necessary only for it to be active when (a) it is in combination with the rest of the agent of the invention and (b) the agent of the invention is attached to, adjacent to or internalised in target cells.

When each moiety is a polypeptide, the two portions may be linked together by any of the conventional ways of cross-linking polypeptides. For example, the antibody or antigen-binding portion thereof may be enriched with thiol groups and the further moiety reacted with a bifunctional agent capable of reacting with those thiol groups, for example the N-hydroxysuccinimide ester of iodoacetic acid (NHIA) or N-succinimidyl-3-(2-pyridyldithio)propionate (SPDP). Amide and thioether bonds, for example achieved with m-maleimidobenzoyl-N-hydroxysuccinimide ester, are generally more stable in vivo than disulphide bonds.

The cytotoxic moiety may be a radiosensitiser. Radiosensitisers include fluoropyrimidines, thymidine analogues, hydroxyurea, gemcitabine, fludarabine, nicotinamide, halogenated pyrimidines, 3-aminobenzamide, 3-aminobenzodiamide, etanixadole, pimonidazole and misonidazole. Also, delivery of genes into cells can radiosensitise them, for example delivery of the p53 gene or cyclin D. The further moiety may be one which becomes cytotoxic, or releases a cytotoxic moiety, upon irradiation. For example, the boron-10 isotope, when appropriately irradiated, releases a particles which are cytotoxic. Similarly, the cytotoxic moiety may be one which is useful in photodynamic therapy such as photofrin.

By “therapeutically effective amount”, “effective amount” or “therapeutically effective”, it is meant that a given substance is administered to a subject suffering from a condition, in an amount sufficient to cure, alleviate or partially arrest the condition or one or more of its symptoms. Such therapeutic treatment may result in a decrease in severity of disease symptoms, or an increase in frequency or duration of symptom-free periods. Effective amounts for a given purpose and a given agent will depend on the severity of the disease or injury as well as the weight and general state of the subject. This may be a predetermined quantity of active antibody calculated to produce a desired therapeutic effect in association with the required additive and diluent, i.e. a carrier or administration vehicle. Further, it is intended to mean an amount sufficient to reduce or prevent a clinically significant deficit in the activity, function and response of the host. Alternatively, a therapeutically effective amount is sufficient to cause an improvement in a clinically significant condition in a host. As is appreciated by those skilled in the art, the amount of a compound may vary depending on its specific activity. Suitable dosage amounts may contain a predetermined quantity of active composition calculated to produce the desired therapeutic effect in association with the required diluent. A therapeutically effective amount can be determined by the ordinary skilled medical or veterinary worker based on patient characteristics, such as age, weight, sex, condition, complications, other diseases, etc., as is well known in the art.

By “cancer”, we include solid tumours and blood-based cancers (e.g. leukaemia). The cancer may be malignant and/or metastatic. Solid tumours are classically defined by the tissue from which they originate, e.g. pancreas, breast, colon etc. However, since immunotherapy acts on the immune system, and not the tumour itself, the immune status of the tumour may be more predictive of the response than the origin of the tumour.

The cancer may be immunogenic. Such cancers are characterised by infiltration of immune cells, such as T cells and cells of myeloid origin. It has been demonstrated that infiltration of CD8 T cells, i.e. a more immunogenic cancer profile, correlates with a good prognosis following therapy, for example in colon cancer, (Galon et al., 2014, J. Pathol. 232(2):199-209).

The cancer may be non-immunogenic or poorly immunogenic. Poorly immunogenic tumours often have low or absent MHC Class I expression and are characterised by a lower number of infiltrating immune cells, such as T cells and cells of myeloid origin (Lechner et al., 2013, J Immunotherapy 36(9):477-89). The tumour may be a pancreatic tumour, an adenoma, an adenocarcinoma, a blastoma, a carcinoma, a desmoid tumour, a desmoplastic small round cell tumour, an endocrine tumour, a germ cell tumour, a lymphoma, a sarcoma, a Wilms tumour, a lung tumour, a colon tumour, a lymph tumour, a breast tumour or a melanoma.

In some embodiments, the cancer is a CD40-positive cancer. By “CD40-positive cancer”, we include any cancer that expresses CD40, albeit at different levels.

Types of pancreatic tumour includes an exocrine tumour, such as an exocrine adenocarcinoma or pancreatic ductal adenocarcinoma (PDAC), or an endocrine tumour. Types of blastoma include hepatoblastoma, glioblastoma, neuroblastoma or retinoblastoma. Types of carcinomas include colorectal carcinoma or hepatocellular carcinoma, pancreatic, prostate, gastric, oesophageal, cervical, and head and neck carcinomas, and adenocarcinoma. Types of sarcomas include Ewing sarcoma, osteosarcoma, rhabdomyosarcoma, or any other soft tissue sarcoma. Types of melanomas include Lentigo maligna, Lentigo maligna melanoma, Superficial spreading melanoma, Acral lentiginous melanoma, Mucosal melanoma, Nodular melanoma, Polypoid melanoma, Desmoplastic melanoma, Amelanotic melanoma, Soft-tissue melanoma, Melanoma with small nevus-like cells, Melanoma with features of a Spitz nevus and Uveal melanoma. Types of lymphoma include Precursor T-cell leukaemia/lymphoma, Follicular lymphoma, Diffuse large B cell lymphoma, Mantle cell lymphoma, B-cell chronic lymphocytic leukaemia/lymphoma, MALT lymphoma, Burkitt's lymphoma, Mycosis fungoides, Peripheral T-cell lymphoma, Nodular sclerosis form of Hodgkin lymphoma, Mixed-cellularity subtype of Hodgkin lymphoma. Types of lung tumour include tumours of non-small-cell lung cancer (adenocarcinoma, squamous-cell carcinoma and large-cell carcinoma) and small-cell lung carcinoma.

Each one of the above-described cancers is well-known, and the symptoms and cancer diagnostic markers are well described, as are the therapeutic agents used to treat those cancers. Accordingly, the symptoms, cancer diagnostic markers, and therapeutic agents used to treat the above-mentioned cancer types would be known to those skilled in medicine.

Clinical definitions of the diagnosis, prognosis and progression of a large number of cancers rely on certain classifications known as staging. Those staging systems act to collate a number of different cancer diagnostic markers and cancer symptoms to provide a summary of the diagnosis, and/or prognosis, and/or progression of the cancer. It would be known to the person skilled in oncology how to assess the diagnosis, and/or prognosis, and/or progression of the cancer using a staging system, and which cancer diagnostic markers and cancer symptoms should be used to do so.

By “cancer staging”, we include the Rai staging, which includes stage 0, stage I, stage II, stage III and stage IV, and/or the Binet staging, which includes stage A, stage B and stage C, and/or the Ann Arbour staging, which includes stage I, stage II, stage III and stage IV.

It is known that cancer can cause abnormalities in the morphology of cells. These abnormalities often reproducibly occur in certain cancers, which means that examining these changes in morphology (otherwise known as histological examination) can be used in the diagnosis or prognosis of cancer. Techniques for visualising samples to examine the morphology of cells, and preparing samples for visualisation, are well known in the art; for example, light microscopy or confocal microscopy.

By “chemotherapy-resistant cancer”, we include that the cancer is unresponsive to chemotherapy. For example, the cancer may continue to grow and/or metastasise despite treatment with chemotherapy. The selection of a particular chemotherapy can be made by a physician following a cancer diagnosis of a subject or patient. Cancer may be initially responsive to chemotherapy and subsequently develop resistance to the chemotherapy. Therefore, a subject may be undergoing treatment with a chemotherapy and subsequently develop resistance to the chemotherapy, following which the subject may be treated with the antibody or antigen-binding portion thereof, as described herein, which results in a combination therapy for treating chemotherapy-resistant cancer.

In some embodiments, the cancer is one that is resistant to treatment with a therapeutic anti-cancer antibody. Such resistant cancer may be a relapsed and/or refractory cancer. A relapsed cancer is a cancer that has previously been treated and, as a result of that treatment, the subject made a complete or partial recovery (i.e. the subject is said to be in remission), but that after the cessation of the treatment the cancer returned or worsened. Put another way, a relapsed cancer is one that has become resistant to a treatment, after a period in which it was effective, and the subject made a complete or partial recovery. A refractory cancer is a cancer that has been treated but which has not responded to that treatment, and/or has been treated but which has progressed during treatment. Put another way, a refractory cancer is one that is resistant to a treatment. It will be appreciated that a cancer may be a refractory cancer due to an intrinsic resistance. By “intrinsic resistance”, we include the meaning that the cancer and/or the subject and/or the target cell is resistant to a particular treatment from the first time at which it is administered, or before it is administered at all. A relapsed cancer and/or refractory cancer would be readily diagnosed by one skilled in the art of medicine.

The term “subject”, (which herein is used interchangeably with “patient”) includes any animal, including a human, that is in need of treatment with an antibody or antigen-binding portion thereof that specifically binds to CD40 and/or chemotherapy. The subject or patient may be mammalian or non-mammalian. Preferably, the subject is mammalian, such as a horse, or a cow, or a sheep, or a pig, or a camel, or a dog, or a cat. Most preferably, the mammalian patient is a human.

Preferably, the subject is one that has been diagnosed as having cancer, optionally chemotherapy-resistant cancer, or that has been identified as likely to have cancer, optionally chemotherapy-resistant cancer, and/or that exhibits symptoms of cancer, optionally chemotherapy-resistant cancer. By “exhibits”, we include that the subject displays a cancer symptom and/or a cancer diagnostic marker, and/or the cancer symptom and/or a cancer diagnostic marker can be measured, and/or assessed, and/or quantified. It would be readily apparent to the person skilled in medicine what the cancer symptoms and cancer diagnostic markers would be and how to measure and/or assess and/or quantify whether there is a reduction or increase in the severity of the cancer symptoms, or a reduction or increase in the cancer diagnostic markers; as well as how those cancer symptoms and/or cancer diagnostic markers could be used to form a prognosis for the cancer.

The term “regimen”, as used herein is synonymous with regime or regiment. By “dosage regimen” we include the meaning that the antibody or antigen-binding portion thereof and the chemotherapy are administered in steps, wherein multiple steps form a regime.

In some embodiments, the dosage regimens described herein can be repeated as many times as necessary in a particular subject. For instance, this dosage regimen can be employed each and every time the antibody or antigen-binding portion thereof that specifically binds to CD40 is administered to the subject. In some embodiments, the exact format of the dosage regimen (in terms of timing and amounts of doses) may be varied between repeat administrations to the subject. The advantage of using the dosage regimens described herein repeatedly is that it reinforces the anti-cancer effects.

However, as a person skilled in the art will appreciate, repeat dosing could also utilise higher or lower total doses as guided by patient tolerability. Analogous flat dosing-based, or receptor-occupancy guided, dosing regimens could be used.

It will be appreciated that the doses and dosage regimens of each of the therapeutic antibodies discussed and contemplated herein would be dependent on the approved doses/regimens for these therapeutic antibodies, and would also vary depending on the indication (for example type of cancer/stage) and/or subject (for example BMI or age).

The term “chemotherapy” includes the meaning of a chemotherapy regimen (which may comprise a treatment period, treatment cycles and an overall treatment time), chemotherapeutic agents (and combinations thereof), chemotherapeutic drugs (and combinations thereof), chemotherapy agents (and combinations thereof), and chemotherapy drugs (and combinations thereof).

A “treatment period” with a specific preparation or treatment as used herein means the period of time in which said specific preparation or treatment is administered to the patient. For example, if chemotherapy (e.g. a chemotherapy drug) is administered for 8 consecutive days, followed by 2 days of no administration of the chemotherapy, then the treatment period with the chemotherapy is 8 days.

The term “treatment cycle” as used herein means a course of one or more treatments or treatment periods that is repeated on a regular schedule and may encompass a period of rest. For example, a treatment given for 8 days followed by 2 days of rest is 1 treatment cycle. The treatment cycle may be repeated, either identically or in an amended form, e.g. with a different dose and/or schedule, or with different additional treatments. A “treatment interval” is the interval between starting and completing a treatment cycle.

The “overall treatment time” means the time period comprising all treatment cycles. As described above, treatment cycles may comprise time periods of no treatment (intervals in which no treatment is administered to the patient, i.e. no chemotherapy and no antibody, and optionally no other drug). Thus, as used herein, the overall treatment time may also comprise said intervals of no treatment within treatment cycles. For example, if the patient receives 8 treatment cycles of 10 days, then the overall treatment time is 80 days. The overall treatment time may comprise at least 1, or 2 or more cycles, or up to 12 cycles. In one embodiment, the overall treatment time comprises 3, 4, 5, 6, 7, 8, 9, 10, or 11 cycles.

Within a given treatment cycle, the antibody or antigen-binding portion thereof and the chemotherapy may be administered simultaneously, sequentially, or separately. As used herein “simultaneously” includes the meaning that the agents are to be taken together on at least one treatment day and may or may not be formulated as a single composition. “Simultaneously” also encompasses a partial overlap in treatment days upon which the agents are administered. For example, the chemotherapy may be administered for one or more consecutive days, and then both the chemotherapy and the antibody or antigen-binding portion thereof may be administered on subsequent consecutive days. “Sequentially” includes the meaning that the drugs are administered on consecutive treatment days, but not on the same treatment day. For example, the chemotherapy may be administered for one or more consecutive days, and the antibody or antigen-binding portion thereof may be administered for the immediately following one or more consecutive days. As used herein, “separate” administration means that the antibody or antigen-binding portion thereof and the chemotherapy are administered as part of the same overall dosing regimen, but they are not administered on the same day. For example, the chemotherapy may be administered for one or more consecutive days, then there may be one or more days during which neither the chemotherapy nor the antibody or antigen-binding portion thereof are administered, and then on one or more subsequent days, the antibody or antigen-binding portion thereof may be administered. Typically, the antibody or antigen-binding portion thereof is administered simultaneously with the chemotherapy (or a portion thereof), more typically the treatment periods of the antibody or antigen-binding portion thereof and the chemotherapy are partially overlapping.

In some embodiments, the chemotherapy is a form of FOLFIRINOX. FOLFIRINOX is a chemotherapy regimen that comprises oxaliplatin, a folinate (also known as folinic acid; e.g. leucovorin), irinotecan, and 5-fluorouracil, each of which may be administered as an infusion, for example an IV infusion. FOLFIRINOX regimens are known in the art, and modifications may be made to any one or more of the components, thereby resulting in a modified FOLFIRINOX (mFOLFIRINOX) regimen (also referred to herein as FOLFIRINOX variants). The modifications may be made prior to commencing a FOLFIRINOX regimen, during a FOLFIRINOX regimen (for example, to mitigate side effects of a particular components of the FOLFIRINOX regimen), and/or after a treatment cycle of a FOLFIRINOX regimen (for example, to mitigate side effects of a particular components of the FOLFIRINOX regimen should the treatment cycle be repeated).

The term “FOLFIRINOX”, as used herein, is replaceable with the term “mFOLFIRINOX”.

An exemplary, and preferred, mFOLFIRINOX corresponds to (as shown in Example 2):

| Agent | Dose | Route | Schedule |
|---|---|---|---|
| Oxaliplatin | 85 mg/m² | IV, 2 h infusion | Every 14 days, (Day 8 in |
| Leucovorin | 400 mg/m² | IV, 2 h infusion | Cycle 1, and Day 1 in |
| Irinotecan | 150 mg/m² | IV, 90 minute infusion | subsequent cycles) |
| | | started 30 minutes | |
| | | after end of the | |
| | | leucovorin infusion | |
| 5-Fluorouracil | 2400 mg/m² | IV, 46-48 hour infusion | Every 14 days (Day 8-10 in |
| infusion | | IV | Cycle 1 and Day 1-3 in |
| | | | subsequent cycles) |

Modified versions of FOLFIRINOX are known in the art. For example, as follows (full details of the authors can be found in the references section herein, see references 41-51; as derived from Tong et al., 2018):

| Regimen | Chemotherapy regimens | | | | |
|---|---|---|---|---|---|
| (Author) | Oxaliplatin | Folinic acid | Irinotecan | 5-FU bolus | 5-FU |
| 1 | 85 mg/m² | 400 mg/m² | 135 mg/m² | 300 mg/m² | 2400 mg/m² |
| (Stein) | | | | | |
| 2 | 85 mg/m² | 200 mg/m² | 150 mg/m² | None | 2800 mg/m² |
| (Vivaldi) | 85 mg/m² | 200 mg/m² | 165 mg/m² | None | 3200 mg/m² |
| 3 | 85 mg/m² | 400 mg/m² | 180 mg/m² | None | 2400 mg/m² |
| (Mahaseth) | | | | | |
| 4 | 85 mg/m² | 400 mg/m² | 130-135 | None | 2400 mg/m² |
| (Ghorani) | | | mg/m² | | |
| 5 | 85 mg/m² | 400 mg/m² | 180 mg/m² | None | 2400 mg/m² |
| (Nanda) | | | | | |
| 6 | 63.75 | 300 mg/m² | 135 mg/m² | 300 mg/m² | 1800 mg/m² |
| (Vočka) | mg/m² | | | | |
| 7 | 68 mg/m² | 400 mg/m² | 135 mg/m² | None | 2400 mg/m² |
| (Li) | | | | | |
| 8 | No specific regimen | | | | |
| (Chllamma) | | | | | |
| 9 | 85 mg/m² | 200 mg/m² | 150 mg/m² | None | 2400 mg/m² |
| (Takeda) | | | | | |
| 10 | 85 mg/m² | 200 mg/m² | 150 mg/m² | None | 2400 mg/m² |
| (Yoshida) | | | | | |
| 11 | 85 mg/m² | None | 165 mg/m² | None | 2400 mg/m² |
| (Blazer) | | | | | |

Accordingly, in some embodiments, the FOLFIRINOX (or mFOLFIRINOX) is selected from Regimen 1, Regimen 2, Regimen 3, Regimen 4, Regimen 5, Regimen 6, Regimen 7, Regimen 8, Regimen 9, Regimen 10 and/or Regimen 11, as described in the above table. In some embodiments, a treatment cycle may commence on a particular regimen, but then swap to an alternative regimen.

Further variations of FOLFIRINOX include the following (LV=leucovorin, an exemplary folinic acid); OX=oxaliplatin; Irino=irinotecan; Freq=frequency of cycles; q2W=2 weeks (i.e. the FOLFIRINOX is repeated every 2 weeks); mCyc=number of cycles of FOLFIRINOX; Met=cohort of metastatic pancreatic cancer subjects; LA=cohort of locally advanced pancreatic cancer subjects):

| Regimen | 5-FU | LV | OX | Irino | Freq | mCyc |
|---|---|---|---|---|---|---|
| FOLFIRINOX | 400 mg bolus, | 400 | 85 | 180 | q2W | 11 |
| | then 2400 | mg/m² | mg/m² | mg/m² | | |
| | mg/m² | | | | | |
| | over 46 h | | | | | |
| mFOLFIRINOX | 300 mg bolus, | 400 | 85 | 135 | q2W | Met 9 |
| (Yale) | then 2400 | mg/m² | mg/m² | mg/m² | | LA 8 |
| | mg/m² | | | | | |
| | over 46 h | | | | | |
| mFOLFIRINOX | 80% intensity | | | | q2W | Met 7 |
| (MSKCC) | | | | | | LA 6 |
| mFOLFIRINOX | 400 mg bolus, | 400 | 65 | 140 | q2W | Pending |
| (AVENGER 500) | then 2400 | mg/m² | mg/m² | mg/m² | | |
| | mg/m² | | | | | |
| | over 46 h | | | | | |
| mFOLFIRINOX | No bolus. | 400 | 85 | 150 | q2W | 12 |
| (Adjuvant) | 2400 mg/m² | mg/m² | mg/m² | mg/m² | | |
| | over 46 h | | | | | |

In some embodiments, FOLFIRINOX comprises of consists of oxaliplatin at a dose of 85 mg per square meter, given as a 2-hour intravenous infusion, immediately followed by leucovorin at a dose of 400 mg per square meter, given as a 2-hour intravenous infusion, with the addition, after 30 minutes, of irinotecan at a dose of 180 mg per square meter, given as a 90-minute intravenous infusion through a Y-connector. This treatment was immediately followed by fluorouracil at a dose of 400 mg per square meter, administered by intravenous bolus, followed by a continuous intravenous infusion of 2400 mg per square meter over a 46-hour period every 2 weeks.

In some embodiments, patients may receive palonosetron, aprepitant and dexamethasone for emesis prophylaxis. 12 cyc planned in met, 8 in LAPC; and/or pegylated filgrastim with each cycle on day 3 or 4 in the absence of severe leukocytosis.

In some embodiments, the FOLFIRINOX or variant thereof comprises oxaliplatin (such as Eloxatin® or generic drug) infusion, optionally wherein the oxaliplatin is administered at a dose of at least 85 mg/m², intravenously, and/or for 2 hours. In some embodiments, the oxaliplatin is administered at a dose of at least 40 mg/m², for example at least 45 mg/m², at least 50 mg/m², at least 55 mg/m², at least 60 mg/m², at least 65 mg/m², at least 70 mg/m², at least 75 mg/m², and/or at least 80 mg/m². In some embodiments, the oxaliplatin is administered at a dose from 40 mg/m² to 85 mg/m². The dose of oxaliplatin may vary between treatment cycles and/or be adapted during a treatment cycle.

In some embodiments, the FOLFIRINOX or variant thereof comprises a folinate (such as leucovorin, calcium folinate, calcium levofolinate, disodium folinate and disodium levofolinate) infusion, optionally wherein the folinate (e.g. leucovorin) is administered at a dose of at least 400 mg/m², intravenously, and/or for 2 hours. The dose of folinate may vary between treatment cycles and/or be adapted during a treatment cycle.

In some embodiments, the FOLFIRINOX or variant thereof comprises irinotecan (such as Campto®) infusion, optionally wherein the irinotecan is administered at a dose of at least 150 mg/m², intravenously, and/or 30 minutes after the end of the leucovorin infusion. In some embodiments, the irinotecan is administered at a dose of at least 80 mg/m², for example, 90 mg/m², 100 mg/m², 110 mg/m², 120 mg/m², 130 mg/m², 135 mg/m², 140 mg/m², 150 mg/m², 160 mg/m², 170 mg/m², or 180 mg/m². In some embodiments, the irinotecan is administered at a dose from 80 mg/m² to 180 mg/m². The dose of irinotecan may vary between treatment cycles and/or be adapted during a treatment cycle.

In some embodiments, the FOLFIRINOX or variant thereof comprises 5-fluorouracil (“5-FU”) infusion, optionally wherein the 5-fluorouracil is administered at a dose of at least 2400 mg/m², intravenously, and/or for a duration of 46-48 hours (e.g. 2.4 g/m²/day). In some embodiments, the 5-FU is administered at a bolus dose of at least 300 mg/m², for example 400 mg/m², 500 mg/m², 600 mg/m², 700 mg/m², 800 mg/m², 900 mg/m², 1000 mg/m², 1100 mg/m², 1200 mg/m², 1300 mg/m², 1360 mg/m², 1400 mg/m², 1500 mg/m², 1600 mg/m², 1700 mg/m², 1800 mg/m², 1900 mg/m², or 1920 mg/m². In some embodiments, the 5-FU is administered at a dose from 1360 mg/m² to 2400 mg/m². The dose of 5-FU may vary between treatment cycles and/or be adapted during a treatment cycle.

Side effects to chemotherapy (including each component associated with FOLFIRINOX) are well-known. It is a matter of routine for a physician to identify the onset of side effects associated with a chemotherapy (or component thereof), and adjust the concentration of the chemotherapy (or component thereof) to a lower dose that reduces the risk of side effects while maintaining an appropriate level of efficacy. Furthermore, additional agents that compensate for the side effects of particular chemotherapy (or components thereof) are known and can be included as premedication and/or post-medication.

By “premedication” we include the meaning that a form of medication is administered before a treatment or procedure. For example, premedication may be prior to treatment with the antibody or antigen-binding portion thereof, prior to chemotherapy or a portion thereof, or prior to both. The premedication selected may be to counter or alleviate known side effects of the antibody or antigen-binding portion thereof, or of the chemotherapy, prior to symptoms of the side effects occurring in a subject.

By “post-medication” we include the meaning that a form of medication is administered after a treatment or procedure. For example, post-medication may be after treatment with the antibody or antigen-binding portion thereof, after chemotherapy or a portion thereof, or after both. The post-medication selected may be to counter or alleviate known side effects of the antibody or antigen-binding portion thereof, or of the chemotherapy, after symptoms of the side effects are observed in a subject.

A further medication may be prior to one agent of a combination therapy (or dosage regimen thereof) and after a different agent, in which case the medication may be considered a premedication and post-medication. The presence of the medication may be such in a subject that it could be considered to be present during the combination therapy (or dosage regimen thereof). Accordingly, premedication and/or post-medication may be considered as being administered during a particular step of a combination therapy (or dosage regimen thereof).

Treatment with chemotherapy is often associated with cytopenia. Depending on the cell lineage, affected patients may suffer from different symptoms. Neutropenia is associated with an increased risk for infections, which may be severe and even fatal. Thrombocytopenia may lead to increased risk for bleeding. Anaemia can cause fatigue, dyspnoea, and tachycardia. Subjects can be monitored with regular blood tests to assess these laboratory parameters, and dose modifications of the chemotherapy can be made accordingly. Patients treated with mFOLFIRINOX may receive primary prophylaxis with G-CSF as a post-medication. If the gemcitabine plus nab-paclitaxel chemotherapy regimen is used, use of G-CSF is permitted, but its use is at investigator discretion. High grade neutropenia and febrile neutropenia should be managed according to institutional standards, e.g., with regard to antibiotic coverage. Blood component transfusion and administration of erythropoietin are alternative post-medications that may be authorised at investigator discretion. Red blood cell transfusion can be considered for hemoglobin<9.5 g/dL or significant symptoms of anemia.

Subjects treated with gemcitabine plus nab-paclitaxel are at increased risk for non-neutropenic sepsis. In the phase 3 study at the first occurrence of fever≥38.5° C. (regardless of neutrophil count), institution of ciprofloxacin (500 mg orally, twice daily)—or amoxicillin/clavulanate (Augmentin®, 500 mg orally, 2-3 times daily) in patients with allergy to fluoroquinolones may be initiated as a post-medication.

In general, chemotherapy-induced diarrhoea results from the death of rapidly dividing enterocytes and consequent loss of absorptive function coupled with inflammation and an altered gastro-intestinal osmotic gradient resulting in secretory loss of fluids and electrolytes. Treatment is generally supportive and in severe or persistent cases can require hospitalisation, with administration of parental fluids as post-medication. When coincident with neutropenia, patients are at elevated risk for infectious complications and coverage with broad-spectrum antibiotics according to institutional standard practices may be considered. In particular, the irinotecan component of mFOLFIRINOX causes both acute (within 24 hours) and delayed (2 to 14-day post-administration) diarrhoea.

Cytotoxic chemotherapy targets rapidly dividing cells such as the gastrointestinal epithelium including the oral mucosa, resulting in tissue damage and inflammation, mucositis. This can be severe enough to limit intake and compromise nutrition, so symptomatic supportive care and nutritional monitoring may be ensued as a post-medication. Of the components in mFOLFIRINOX, 5-fluorouracil (5-FU) is the strongest contributor to mucositis.

Both taxane and platinum-containing chemotherapy are associated with nerve damage attributed primarily to altered microtubular transport interfering with axonal function. Typically, this affects peripheral neurons, more distant than proximal and more often sensory than motor. The sensory neuropathy may begin with paresthesia but can progress to impair activities of daily living. The toxicity is cumulative over time and may persist or even worsen after dose modification. Predisposing factors include medical history of peripheral neuropathy, diabetes, advanced age or prior exposure to neurotoxic drugs. The main contributor to neuropathy in the gemcitabine/nab-paclitaxel regimen is the paclitaxel component, whereas oxaliplatin is responsible for most of the neurotoxicity in the mFOLFIRINOX regimen. In addition to this generalized peripheral neuropathy, a specific acute neuropathy, laryngopharyngeal dysesthesia, occurs infrequently in patients within hours of treatment with oxaliplatin. This can manifest as an uncomfortable sensation in the area of the laryngopharynx and patients can experience dyspnea and anxiety. For patient with a history of oxaliplatin-associated laryngospasm, it is recommended to administer a tranquilizer prior to oxaliplatin infusion and to infuse over six hours.

mFOLFIRINOX is considered to be a moderately emetogenic regimen, and prophylaxis with anti-emetics is recommended per investigator's discretion and institutional standard. Gemcitabine plus nab-paclitaxel is considered to be of low emetogenic potential. Note, that use of corticosteroids should be restricted when given as prophylaxis. Nausea as a symptom for infusion-related reaction has been observed with mitazalimab therapy and anti-emetics may be part of the pre- and/or post-medication in relation to mitazalimab administration.

If chemotherapy leaks into tissue surrounding the intended blood vessel, severe local tissue necrosis can occur; this has been reported for irinotecan and oxaliplatin. If this occurs, the infusion should be stopped immediately. Leaving the needle or catheter in place, as much of the agent as possible can be aspirated as a post-medication. Ice may be applied as an alternative or a further post-medication to the infiltrated area, for example for 15-20 minutes every 4 to 6 hours for a 72-hour period. Alternatively, or additionally, local corticotherapy may be applied.

In some embodiments, the subject receives at least one treatment cycle of FOLFIRINOX. A FOLFIRINOX treatment cycle may be as defined in the Examples. In some embodiments, the subject receives 1, 2, 3, 4, 5, 6, 7, 8, 9, 10, 11, or 12 FOLFIRINOX treatment cycles in total. In some embodiments, the subject does not receive more than 12 FOLFIRINOX treatment cycles. The number of FOLFIRINOX cycles may vary between individuals, and so the requirement for additional iterations may be assessed on a case-by-case basis. The competent physician administering the FOLFIRINOX cycle can therefore determine based on responsiveness (e.g. assessed by a reduction/prevention of cancer growth and/or metastasis) and/or risk of side effects (e.g. if particular side effects become problematic for the recipient of the FOLFIRINOX, or a component thereof, then a decision may be made to cease further treatment cycles).

In some embodiments, a FOLFIRINOX treatment cycle may be as follows:
Oxaliplatin IV over 2 hours immediately followed by;
Leucovorin or similar approved folinates over 2 hours;
Irinotecan administered over 90 minutes (starting 30 minutes after start of the leucovorin infusion), followed by;
5-FU infusion over 46-48 hours, for example over 46 hours, over 47 hours, or over 48 hours.

Local administration to the tumour site is preferred and includes peritumoural, juxtatumoural, intratumoural, intralesional, perilesional, intracranial and intravesicle administration by any suitable means, such as injection. Local administration may also include intra cavity infusion and inhalation, depending on the site of the tumour.

Systemic administration of any agent described herein (such as the antibody or antigen-binding portion thereof and/or the chemotherapy) means administration into the circulatory system of the subject, including the vascular and/or lymphatic system. Such administration may be by any suitable route, but is typically parenteral.

The phrase “parenteral administration” as used herein means modes of administration other than enteral and topical administration, and is typically achieved by injection, infusion or implantation. Suitable routes include intravenous, intramuscular, intradermal, intraperitoneal, subcutaneous, spinal, intracerebral, intrathecal, intraosseous or other parenteral routes of administration.

In some embodiments, the antibody or antigen-binding portion thereof is formulated and/or adapted for delivery by a route selected from the group comprising: intravenous; intramuscular; and subcutaneous. Preferably, the antibody or antigen-binding portion thereof is formulated and/or adapted for intravenous (i.e. “i.v” or “IV”) delivery.

In some embodiments, the antibody or antigen-binding portion thereof is delivered to the subject by a route selected from the group comprising: intravenous; intramuscular; and subcutaneous. Preferably, the antibody or antigen-binding portion thereof is delivered intravenously.

Thus, in preferred embodiments, the first and/or second and/or further doses of the antibody or antigen-binding portion thereof are formulated for intravenous delivery to the subject and/or are delivered by intravenous delivery to the subject.

Methods and formulations for intravenous administration of antibody or antigen-binding portions thereof are well known in the art. In the present invention, any type of intravenous administration may be used, such as injection or infusion.

In some embodiments, the chemotherapy is formulated and/or adapted for delivery by a route selected from the group comprising: intravenous; intramuscular; and subcutaneous.

In embodiments of the invention, the chemotherapy is delivered to the subject by a route selected from the group comprising: intravenous; intramuscular; and subcutaneous.

Thus, in preferred embodiments, the first and/or second and/or further doses of the chemotherapy are formulated for intravenous delivery to the subject and/or are delivered by intravenous delivery to the subject.

Depending on the route of administration, the antibody or antigen-binding portion thereof and/or the chemotherapy may be coated in a material to protect the agent(s) from the action of acids and other natural conditions that may inactivate or denature the antibody or antigen-binding portion thereof and/or chemotherapy. Preferred pharmaceutically acceptable carriers comprise aqueous carriers or diluents. Examples of suitable aqueous carriers that may be employed in the pharmaceutical compositions of the invention include water, buffered water and saline. Examples of other carriers include ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, using coating materials, such as lecithin, by the maintenance of the required particle size in the case of dispersions, and using surfactants. In many cases, it will be preferable to include isotonic agents, for example, sugars, polyalcohols such as mannitol, sorbitol, or sodium chloride in the composition.

Methods and formulations for various routes of administration are well known in the art.

The combination therapies and methods of the invention utilise an antibody that binds immunospecifically to CD40, that is an “anti-CD40 antibody”. In one embodiment, said antibody is retained at the tumour site following administration to a subject. The antibody preferably specifically binds to CD40, that is it binds to CD40 but does not bind, or binds at a lower affinity (e.g. a 10-fold lower affinity), to other molecules. Unless otherwise specified, the term CD40 as used herein refers to human CD40. The sequence of human CD40 is set out in SEQ ID NO: 13. An anti-CD40 antibody of the present invention may have some binding affinity for CD40 from other mammals, for example primate or murine CD40. The antibody preferably binds to human CD40 when localised on the surface of a cell.

In particular, the anti-CD40 antibodies used in the combination therapies of the invention compete for binding to human CD40 with a ‘reference antibody’ which comprises the light chain variable region of SEQ ID NO: 7 and the heavy chain variable region of SEQ ID NO: 8 (optionally together with light and heavy constant regions of SEQ ID NO: 11 and SEQ ID NO: 12, respectively). Such competitive binding inhibition can be determined using assays and methods well known in the art, for example using BIAcore chips with immobilised human CD40 and incubating in the presence of the reference antibody, with and without an antibody polypeptide to be tested. Alternatively, a pair-wise mapping approach can be used, in which the reference antibody is immobilised to the surface of the BIAcore chip, human CD40 is bound to the immobilised antibody, and then a second antibody is tested for simultaneous binding ability to human CD40 (see ‘BIAcore Assay Handbook’, GE Healthcare Life Sciences, 29-0194-00 AA 05/2012; the disclosures of which are incorporated herein by reference).

Exemplary anti-CD40 antibodies are disclosed in WO 2013/034904 and WO 2016/023960 to Alligator Bioscience AB (the disclosures of which are incorporated herein by reference).

The antibody preferably has the ability to bind to CD40 in its native state and in particular to CD40 localised on the surface of a cell. Preferably, an antibody will bind specifically to CD40. That is, an antibody used in the methods of invention will preferably bind to CD40 with greater binding affinity than that at which it binds to another molecule.

By “localised on the surface of a cell” it is meant that CD40 is associated with the cell such that one or more region of CD40 is present on the outer face of the cell surface. For example, CD40 may be inserted into the cell plasma membrane (i.e. orientated as a transmembrane protein) with one or more regions presented on the extracellular surface. This may occur in the course of expression of CD40 by the cell. Thus, in one embodiment, “localised on the surface of a cell” may mean “expressed on the surface of a cell”. Alternatively, CD40 may be outside the cell with covalent and/or ionic interactions localising it to a specific region or regions of the cell surface.

An anti-CD40 antibody described herein may induce and/or enhance ADCC-mediated lysis of a cell expressing CD40 and/or enhance apoptosis of a cell expressing CD40. The cell is typically a tumour cell. By “enhance” it is meant that the number of cells lysed or induced to undergo apoptosis increases in the presence of an antibody of the invention, relative to the number of cells lysed or induced to undergo apoptosis in the presence of an appropriate control substance. Methods for determining the level of ADCC-mediated lysis or apoptosis in a sample of cells are well-known in the art. For example, a chromium-51 release assay, europium release assay or sulphur-35 release assay may be used. In such assays, a previously labelled target cell line expressing the antigen (in this case CD40) is incubated with an antibody to be tested. After washing, effector cells (typically expressing Fc receptor CD16) are co-incubated with the antibody-labelled target cells. Target cell lysis is subsequently measured by release of intracellular label by a scintillation counter or spectrophotometry.

Preferably, the antibody, antigen-binding portion thereof, comprises an antibody Fc-region. It will be appreciated by skilled person that the Fc portion may be from an IgG antibody, or from a different class of antibody (such as IgM, IgA, IgD or IgE). For example, the Fc region may be from an IgG1, IgG2, IgG3 or IgG4 antibody. Advantageously, however, the Fc region is from an IgG1 antibody.

The Fc region may be naturally occurring (e.g. part of an endogenously produced antibody) or may be artificial (e.g. comprising one or more point mutations relative to a naturally occurring Fc region). Fc-regions with point mutations improving their ability to bind FcR may be advantageous, e.g. by altering serum half-life or improving binding to Fcγ receptors (FcγR) involved in ADCC and CDC. In particular, mutations that enhance binding to FcγRIIB, e.g. S267E (Strohl et al., 2009, Curr Opin Biotechnol, 20:685-691) may be advantageous for the invention given the link between FcγRIIB binding and functional activity of CD40 antibodies (Li et al., 2011, Science, 333: 1030-1034).

As an alternative to the labelling with radioisotopes required in such assays, methods may be used in which lysis is detected by measuring the release of enzymes naturally present in the target cells. This may be achieved by detection (for example bioluminescent detection) of the products of an enzyme-catalysed reaction. No previous labelling of the cells is required in such an assay. A typical cellular enzyme detected with such an assay is GAPDH.

An anti-CD40 antibody described herein may modulate the activity of a cell expressing CD40, wherein said modulation is an increase or decrease in the activity of said cell. The cell is typically a dendritic cell or a B cell.

Professional APCs, such as dendritic cells, are activated when signalling via CD40 occurs, which triggers several biological events, including immune cell activation, proliferation, and production of cytokines and chemokines. Methods for determining dendritic cell activation associated with CD40 are known in the art (discussed, for example, in Schonbeck et al., 2001, Cell Mol Life Sci., 58:40-43; van Kooten et al., 2000, J. Leuk., Biol., 67: 2-17) and are described further below.

Stimulation of human B cells with recombinant CD40L or anti-CD40 antibodies induces up-regulation of surface markers, such as CD23, CD30, CD80, CD86, Fas and MHC II, secretion of soluble cytokines, e.g. IL-6, TNF-γ and TNF-α, and homotypic aggregation. Methods for determining CD40-related B cell activation are known in the art (discussed, for example, in Schonbeck et al., 2001, supra) and are described further below.

Methods and assays for determining the ability of an antibody to modulate the activity of dendritic cells and B cells are well known in the art. For example, the activation of dendritic cells may be assessed by measuring the level of cell surface markers such as CD86 and CD80 and/or by measuring anti-CD40 antibody-induced secretion of IFNγ from T cells, wherein in an increase in any of these parameters indicates increased activation and a decrease represents decreased activation. Similarly, the ability of an antibody to modulate the activity of B cells may be assessed by measuring the level of cell surface markers (such as CD86) and/or by measuring anti-CD40 antibody-induced B cell proliferation (see Example 3 of WO 2016/023960), wherein in an increase in any of these parameters indicates increased activation and a decrease represents decreased activation.

Preferably, an anti-CD40 antibody described herein, which increases the activation of dendritic cells or B cells, has a potency for dendritic cell or B cell activation. Cell activation may typically be measured as an EC50 level in an assay which involves incubating isolated dendritic or B cells with the test stimulator and then detecting cell proliferation as the measure of activation.

The terms “binding activity” and “binding affinity” are intended to refer to the tendency of an antibody molecule to bind or not to bind to a target. Binding affinity may be quantified by determining the dissociation constant (Kd) for an antibody and its target. Similarly, the specificity of binding of an antibody to its target may be defined in terms of the comparative dissociation constants (Kd) of the antibody for its target as compared to the dissociation constant with respect to the antibody and another, non-target molecule.

Typically, the Kd for the antibody with respect to the target will be 2-fold, preferably 5-fold, more preferably 10-fold less than Kd with respect to the other, non-target molecule such as unrelated material or accompanying material in the environment. More preferably, the Kd will be 50-fold less, even more preferably 100-fold less, and yet more preferably 200-fold less.

The value of this dissociation constant can be determined directly by well-known methods and can be computed even for complex mixtures by methods such as those, for example, set forth in Caceci et al. (Byte 9:340-362, 1984). For example, the Kd may be established using a double-filter nitrocellulose filter binding assay such as that disclosed by Wong & Lohman (Proc. Natl. Acad. Sci. USA 90, 5428-5432, 1993). Other standard assays to evaluate the binding ability of ligands such as antibodies towards targets are known in the art, including for example, ELISAs, Western blots, RIAs, and flow cytometry analysis. The binding kinetics (e.g., binding affinity) of the antibody also can be assessed by standard assays known in the art, such as by BIAcore™ system analysis.

A competitive binding assay can be conducted in which the binding of the antibody to the target is compared to the binding of the target by another, known ligand of that target, such as another antibody. The concentration at which 50% inhibition occurs is known as the Ki. Under ideal conditions, the Ki is equivalent to Kd. The Ki value will never be less than the Kd, so measurement of Ki can conveniently be substituted to provide an upper limit for Kd.

An anti-CD40 antibody described herein is preferably capable of binding to its target with an affinity that is at least two-fold, 10-fold, 50-fold, 100-fold or greater than its affinity for binding to another non-target molecule.

An antibody used in the combination therapies and methods of the invention will typically exhibit the ability to:
(i) specifically bind to human CD40 when localised on the surface of a cell;
(ii) enhance antibody dependent cellular cytotoxicity (ADCC)-mediated lysis of a cell expressing CD40;
(iii) enhance apoptosis of a cell expressing CD40; and/or
(iv) modulate the activity of a cell expressing CD40, wherein said modulation is an increase or decrease in the activity of said cell.

In some embodiments, the combination therapies and methods of the invention will exhibit and improvement on survival of the recipient and/or on tumour volume control versus a relevant control. By “relevant control”, we include the meaning of an individual that: (i) is not undergoing treatment with a combination therapy as described herein; (ii) is undergoing treatment with a chemotherapy (e.g. mFOLFIRINOX) as described herein but is not being administered an anti-CD40 antibody (e.g. mitazalimab), preferably wherein the chemotherapy is the same as used in the combination therapy; and/or (iii) is undergoing treatment with an anti-CD40 antibody (e.g. mitazalimab) but is not undergoing treatment with a chemotherapy (e.g. mFOLFIRINOX) as described herein, preferably wherein the anti-CD40 antibody is the same as used in the combination therapy.

The antibody may be or may comprise a variant or a fragment of one of the specific anti-CD40 antibodies disclosed herein, provided that said variant or fragment retains specificity for CD40, and at least one of functional characteristics (i) to (iv).

A fragment is preferably an antigen-binding portion of a said antibody. A fragment may be made by truncation, e.g. by removal of one or more amino acids from the N and/or C-terminal ends of a polypeptide. Up to 10, up to 20, up to 30, up to 40 or more amino acids may be removed from the N and/or C terminal in this way. Fragments may also be generated by one or more internal deletions.

A variant may comprise one or more substitutions, deletions or additions with respect to the sequences of a specific anti-CD40 antibody disclosed herein. A variant may comprise 1, 2, 3, 4, 5, up to 10, up to 20, up to 30 or more amino acid substitutions and/or deletions from the specific sequences disclosed herein. “Deletion” variants may comprise the deletion of individual amino acids, deletion of small groups of amino acids such as 2, 3, 4 or 5 amino acids, or deletion of larger amino acid regions, such as the deletion of specific amino acid domains or other features. “Substitution” variants preferably involve the replacement of one or more amino acids with the same number of amino acids and making conservative amino acid substitutions. For example, an amino acid may be substituted with an alternative amino acid having similar properties, for example, another basic amino acid, another acidic amino acid, another neutral amino acid, another charged amino acid, another hydrophilic amino acid, another hydrophobic amino acid, another polar amino acid, another aromatic amino acid or another aliphatic amino acid.

Some properties of the 20 main amino acids which can be used to select suitable substituents are as follows:

| | | | |
|---|---|---|---|
| Ala | aliphatic, hydrophobic, neutral | Met | hydrophobic, neutral |
| Cys | polar, hydrophobic, neutral | Asn | polar, hydrophilic, neutral |
| Asp | polar, hydrophilic, charged (−) | Pro | hydrophobic, neutral |
| Glu | polar, hydrophilic, charged (−) | Gln | polar, hydrophilic, neutral |
| Phe | aromatic, hydrophobic, neutral | Arg | polar, hydrophilic, charged (+) |
| Gly | aliphatic, neutral | Ser | polar, hydrophilic, neutral |
| His | aromatic, polar, hydrophilic, | Thr | polar, hydrophilic, neutral |
| | charged (+) | | |
| Ile | aliphatic, hydrophobic, neutral | Val | aliphatic, hydrophobic, neutral |
| Lys | polar, hydrophilic, charged (+) | Trp | aromatic, hydrophobic, neutral |
| Leu | aliphatic, hydrophobic, neutral | Tyr | aromatic, polar, hydrophobic |

Preferred “variants” include those in which instead of the naturally occurring amino acid the amino acid which appears in the sequence is a structural analog thereof. Amino acids used in the sequences may also be derivatized or modified, e.g. labelled, providing the function of the antibody is not significantly adversely affected.

Variants may be prepared during synthesis of the antibody or by post-production modification, or when the antibody is in recombinant form using the known techniques of site-directed mutagenesis, random mutagenesis, or enzymatic cleavage and/or ligation of nucleic acids.

Preferably variant antibodies have an amino acid sequence which has more than 60%, or more than 70%, e.g. 75% or 80%, preferably more than 85%, e.g. more than 90% or 95% amino acid identity to the VL or VH domain of an antibody disclosed herein. This level of amino acid identity may be seen across the full length of the relevant SEQ ID NO sequence or over a part of the sequence, such as across 20, 30, 50, 75, 100, 150, 200 or more amino acids, depending on the size of the full length polypeptide.

In connection with amino acid sequences, “sequence identity” refers to sequences which have the stated value when assessed using ClustalW (Thompson et al., 1994, supra) with the following parameters:
Pairwise alignment parameters—Method: accurate, Matrix: PAM, Gap open penalty: 10.00, Gap extension penalty: 0.10;
Multiple alignment parameters—Matrix: PAM, Gap open penalty: 10.00, % identity for delay: 30, Penalize end gaps: on, Gap separation distance: 0, Negative matrix: no, Gap extension penalty: 0.20, Residue-specific gap penalties: on, Hydrophilic gap penalties: on, Hydrophilic residues: GPSNDQEKR. Sequence identity at a particular residue is intended to include identical residues which have simply been derivatized.

An anti-CD40 antibody of the invention may bind to the same epitope as a specific antibody as disclosed herein, since such an antibody is likely to mimic the action of the disclosed antibody. Whether or not an antibody binds to the same epitope as another antibody may be determined by routine methods. For example, the binding of each antibody to a target may be using a competitive binding assay. Methods for carrying out competitive binding assays are well known in the art. For example, they may involve contacting together an antibody and a target molecule under conditions under which the antibody can bind to the target molecule. The antibody/target complex may then be contacted with a second (test) antibody and the extent to which the test antibody is able to displace the first antibody from antibody/target complexes may be assessed. Such assessment may use any suitable technique, including, for example, Surface Plasmon Resonance, ELISA, or flow cytometry. The ability of a test antibody to inhibit the binding of a first antibody to the target demonstrates that the test antibody can compete with said first antibody for binding to the target and thus that the test antibody binds to the same epitope or region on the target as the first antibody, and may therefore mimic the action of the first antibody.

An anti-CD40 antibody of the invention may be an antibody comprising one, two or all three of the CDR sequences of SEQ ID NOs: 1 to 3 and/or one, two, or all three of the CDR sequences of SEQ ID NOs: 4 to 6. The antibody may comprise all six CDR sequences of SEQ ID NOs: 1 to 6.

The antibody may comprise the light chain variable region sequence of SEQ ID NO: 7 and/or the heavy chain variable region sequence of SEQ ID NO: 8.

The antibody may be, or may bind to the same epitope as, an antibody comprising the light chain variable region sequence of SEQ ID NO: 7 and the heavy chain variable region sequence of SEQ ID NO: 8. In addition, the antibody may comprise the light chain constant region sequence of SEQ ID NO: 11 and/or the heavy chain constant region sequence of SEQ ID NO: 12.

The anti-CD40 antibody or any variant or fragment thereof used in the combination therapies and methods of the invention preferably has a theoretical isoelectric point (pI) of 9.0 or above, preferably 9.1 or above, more preferably 9.2 or above or 9.25 or above, most preferably 9.3 or above.

According to certain embodiments, the antibody or antigen-binding portion thereof and the chemotherapy are administered simultaneously, either in the same composition or in separate compositions. According to other embodiments, the antibody or antigen-binding portion thereof and the chemotherapy are administered sequentially, i.e. the antibody or antigen-binding portion thereof is administered either prior to, during and/or after the administration of the chemotherapy. In some embodiments, the administration of the antibody or antigen-binding portion thereof and the chemotherapy are concurrent, i.e. the administration period of the antibody or antigen-binding portion thereof, and that of the chemotherapy overlap with each other. In some embodiments, the administration of the antibody or antigen-binding portion thereof and the chemotherapy are non-concurrent. For example, in some embodiments, the administration of the antibody or antigen-binding portion thereof is terminated before the chemotherapy is administered. In some embodiments, the administration of chemotherapy is terminated before the antibody or antigen-binding portion thereof is administered.

In some embodiments, the antibody or antigen-binding portion thereof and the chemotherapy are administered within a single therapeutic composition (e.g. a pharmaceutical composition). In some embodiments, the therapeutic composition further comprises therapeutically acceptable diluents or carrier. In some embodiments, the antibody or antigen-binding portion thereof is administered as a pharmaceutical composition, and the chemotherapy is not administered as a pharmaceutical composition.

The invention also provides a kit for treating cancer, optionally chemotherapy-resistant cancer in a subject, the kit comprising a combination therapy as defined above. For example, the kit may comprise (a) a therapeutically effective amount of an antibody or antigen-binding portion thereof that specifically binds to CD40 as described herein, and optionally (b) a therapeutically effective amount of chemotherapy that is suitable for administration (e.g. systemic administration) to a subject. The antibody or antigen-binding portion thereof is preferably provided in a form suitable for local administration to a tumour site.

The kits of the invention may additionally comprise one or more other reagents or instruments which enable any of the embodiments mentioned above to be carried out. Such reagents or instruments include one or more of the following: suitable buffer(s) (aqueous solutions) and means to administer the anti-CD40 antibody and/or the chemotherapy (such as a vessel or an instrument comprising a needle). The kit may include instructions for performing a combination therapy or method as described herein.

The anti-CD40 antibody and the chemotherapy described herein, or provided in the kits of the invention, may each be provided as a separate pharmaceutical composition formulated together with a pharmaceutically acceptable carrier. The chemotherapy, if composed of multiple agents, may each be provided as separate pharmaceutical compositions formulated together with a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible and are also compatible with the required routes of administration.

A pharmaceutical composition may include a pharmaceutically acceptable antioxidant. These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminium monostearate and gelatin.

Pharmaceutical compositions typically must be sterile and stable under the conditions of manufacture and storage. The composition can be formulated as a solution, microemulsion, liposome, or other ordered structure suitable to high drug concentration. Sterile injectable solutions can be prepared by incorporating the active agent (e.g. antibody) in the required amount in an appropriate solvent with one or a combination of ingredients enumerated above, as required, followed by sterilization microfiltration. Generally, dispersions are prepared by incorporating the active agent into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active agent plus any additional desired ingredient from a previously sterile-filtered solution thereof. Pharmaceutical compositions may comprise additional active ingredients as well as those mentioned above.

Suitable pharmaceutically acceptable buffers, diluents, carriers and excipients are well-known in the art (see Remington's Pharmaceutical Sciences, 18th edition, A. R Gennaro, Ed., Mack Publishing Company (1990) and handbook of Pharmaceutical Excipients, 3rd edition, A. Kibbe, Ed., Pharmaceutical Press (2000), the disclosures of which are incorporated herein by reference).

The term “buffer” is intended to include an aqueous solution containing an acid-base mixture with the purpose of stabilising pH. Examples of buffers are Trizma, Bicine, Tricine, MOPS, MOPSO, MOBS, Tris, Hepes, HEPBS, MES, phosphate, carbonate, acetate, citrate, glycolate, lactate, borate, ACES, ADA, tartrate, AMP, AMPD, AMPSO, BES, CABS, cacodylate, CHES, DIPSO, EPPS, ethanolamine, glycine, HEPPSO, imidazole, imidazolelacetic acid, PIPES, SSC, SSPE, POPSO, TAPS, TABS, TAPSO and TES.

The term “diluent” is intended to include an aqueous or non-aqueous solution with the purpose of diluting the agent in the pharmaceutical preparation. The diluent may be one or more of saline, water, polyethylene glycol, propylene glycol, ethanol or oils (such as safflower oil, corn oil, peanut oil, cottonseed oil or sesame oil).

The term “adjuvant” is intended to include any compound added to the formulation to increase the biological effect of the agent of the invention. The adjuvant may be one or more of zinc, copper or silver salts with different anions, for example, but not limited to fluoride, chloride, bromide, iodide, tiocyanate, sulfite, hydroxide, phosphate, carbonate, lactate, glycolate, citrate, borate, tartrate, and acetates of different acyl composition. The adjuvant may also be cationic polymers such as cationic cellulose ethers, cationic cellulose esters, deacetylated hyaluronic acid, chitosan, cationic dendrimers, cationic synthetic polymers such as poly(vinyl imidazole), and cationic polypeptides such as polyhistidine, polylysine, polyarginine, and peptides containing these amino acids.

The excipient may be one or more of carbohydrates, polymers, lipids and minerals. Examples of carbohydrates include lactose, glucose, sucrose, mannitol, and cyclodextrins, which are added to the composition, e.g., for facilitating lyophilisation. Examples of polymers are starch, cellulose ethers, cellulose carboxymethylcellulose, hydroxypropylmethyl cellulose, hydroxyethyl cellulose, ethylhydroxyethyl cellulose, alginates, carrageenans, hyaluronic acid and derivatives thereof, polyacrylic acid, polysulphonate, polyethylenglycol/polyethylene oxide, polyethyleneoxide/polypropylene oxide copolymers, polyvinylalcohol/polyvinylacetate of different degree of hydrolysis, and polyvinylpyrrolidone, all of different molecular weight, which are added to the composition, e.g., for viscosity control, for achieving bioadhesion, or for protecting the lipid from chemical and proteolytic degradation. Examples of lipids are fatty acids, phospholipids, mono-, di-, and triglycerides, ceramides, sphingolipids and glycolipids, all of different acyl chain length and saturation, egg lecithin, soy lecithin, hydrogenated egg and soy lecithin, which are added to the composition for reasons similar to those for polymers. Examples of minerals are talc, magnesium oxide, zinc oxide and titanium oxide, which are added to the composition to obtain benefits such as reduction of liquid accumulation or advantageous pigment properties.

The active antibody-based agents of the invention, and/or the chemotherapy, may be formulated into any type of pharmaceutical composition known in the art to be suitable for the delivery thereof.

In one embodiment, the pharmaceutical compositions of the invention may be in the form of a liposome, in which the agent is combined, in addition to other pharmaceutically acceptable carriers, with amphipathic agents such as lipids, which exist in aggregated forms as micelles, insoluble monolayers and liquid crystals. Suitable lipids for liposomal formulation include, without limitation, monoglycerides, diglycerides, sulfatides, lysolecithin, phospholipids, saponin, bile acids, and the like. Suitable lipids also include the lipids above modified by poly(ethylene glycol) in the polar headgroup for prolonging bloodstream circulation time. Preparation of such liposomal formulations can be found in for example U.S. Pat. No. 4,235,871, the disclosures of which are incorporated herein by reference.

The pharmaceutical compositions of the invention may also be in the form of biodegradable microspheres. Aliphatic polyesters, such as poly(lactic acid) (PLA), poly(glycolic acid) (PGA), copolymers of PLA and PGA (PLGA) or poly(caprolactone) (PCL), and polyanhydrides have been widely used as biodegradable polymers in the production of microspheres. Preparations of such microspheres can be found in U.S. Pat. No. 5,851,451 and in EP 0 213 303, the disclosures of which are incorporated herein by reference.

In a further embodiment, the pharmaceutical compositions of the invention are provided in the form of nanoparticles, for example based on poly-gamma glutamic acid. Details of the preparation and use of such nanoparticles can be found in WO 2011/128642, the disclosures of which are incorporated herein by reference. It will be appreciated by persons skilled in the art that one or more of the active components of the combination therapies of the present invention may be formulated in separate nanoparticles, or both active components may be formulated in the same nanoparticles.

In a further embodiment, the pharmaceutical compositions of the invention are provided in the form of polymer gels, where polymers such as starch, cellulose ethers, cellulose carboxymethylcellulose, hydroxypropylmethyl cellulose, hydroxyethyl cellulose, ethylhydroxyethyl cellulose, alginates, carrageenans, hyaluronic acid and derivatives thereof, polyacrylic acid, polyvinyl imidazole, polysulphonate, polyethylenglycol/polyethylene oxide, polyethyleneoxide/polypropylene oxide copolymers, polyvinylalcohol/polyvinylacetate of different degree of hydrolysis, and polyvinylpyrrolidone are used for thickening of the solution containing the agent. The polymers may also comprise gelatin or collagen.

Alternatively, the agents may simply be dissolved in saline, water, polyethylene glycol, propylene glycol, ethanol or oils (such as safflower oil, corn oil, peanut oil, cottonseed oil or sesame oil), tragacanth gum, and/or various buffers.

It will be appreciated that the pharmaceutical compositions of the invention may include ions and a defined pH for potentiation of action of the active agent. Additionally, the compositions may be subjected to conventional pharmaceutical operations such as sterilisation and/or may contain conventional adjuvants such as preservatives, stabilisers, wetting agents, emulsifiers, buffers, fillers, etc.

The pharmaceutical compositions according to the invention may be administered via any suitable route known to those skilled in the art. Thus, possible routes of administration include parenteral (intravenous, subcutaneous, and intramuscular), topical, ocular, nasal, pulmonary, buccal, oral, parenteral, vaginal and rectal. Also, administration from implants is possible.

Advantageously, the pharmaceutical composition is suitable for administration at or near the site of a tumour, e.g. intra-tumourally or peri-tumourally.

In some embodiments, the pharmaceutical composition is suitable for parenteral administration. Methods for formulating an antibody into a pharmaceutical composition will be well-known to those skilled in the arts of medicine and pharmacy.

The combination therapy of the invention may be delivered using an injectable sustained-release drug delivery system. These are designed specifically to reduce the frequency of injections. An example of such a system is Nutropin Depot which encapsulates recombinant human growth hormone (rhGH) in biodegradable microspheres that, once injected, release rhGH slowly over a sustained period. Preferably, delivery is performed intra-muscularly (i.m.) and/or subcutaneously (s.c.) and/or intravenously (i.v.).

The combination therapy of the invention can be administered by a surgically implanted device that releases the drug directly to the required site. For example, Vitrasert releases ganciclovir directly into the eye to treat CMV retinitis. The direct application of this toxic agent to the site of disease achieves effective therapy without the drug's significant systemic side-effects.

Electroporation therapy (EPT) systems can also be employed for the administration of the combination therapy of the invention. A device which delivers a pulsed electric field to cells increases the permeability of the cell membranes to the drug, resulting in a significant enhancement of intracellular drug delivery.

The combination therapy of the invention can also be delivered by electro-incorporation (EI). EI occurs when small particles of up to 30 microns in diameter on the surface of the skin experience electrical pulses identical or similar to those used in electroporation. In EI, these particles are driven through the stratum corneum and into deeper layers of the skin. The particles can be loaded or coated with drugs or genes or can simply act as “bullets” that generate pores in the skin through which the drugs can enter.

An alternative combination therapy of the invention is the ReGel injectable system that is thermo-sensitive. Below body temperature, ReGel is an injectable liquid while at body temperature it immediately forms a gel reservoir that slowly erodes and dissolves into known, safe, biodegradable polymers. The active substance is delivered over time as the biopolymers dissolve.

The combination therapy of the invention can also be delivered orally. The process employs a natural process for oral uptake of vitamin B₁₂ and/or vitamin D in the body to co-deliver proteins and peptides. By riding the vitamin B₁₂ and/or vitamin D uptake system, the agents, medicaments and pharmaceutical compositions of the invention can move through the intestinal wall. Complexes are synthesised between vitamin B₁₂ analogues and/or vitamin D analogues and the drug that retain both significant affinity for intrinsic factor (IF) in the vitamin B₁₂ portion/vitamin D portion of the complex and significant bioactivity of the active substance of the complex.

The combination therapy of the invention can be introduced to cells by “Trojan peptides”. These are a class of polypeptides called penetratins which have translocating properties and can carry hydrophilic compounds across the plasma membrane. This system allows direct targeting of oligopeptides to the cytoplasm and nucleus and may be non-cell type specific and highly efficient. See Derossi et al. (1998), Trends Cell Biol. 8, 84-87.

The combination therapy of the invention may be a unit dosage containing a daily dose or unit, daily sub-dose or an appropriate fraction thereof, of the active ingredient.

The combination therapy of the invention will normally be administered orally or by any parenteral route, in the form of a pharmaceutical composition comprising the active ingredient, optionally in the form of a non-toxic organic, or inorganic, acid, or base, addition salt, in a pharmaceutically acceptable dosage form. Depending upon the disorder and patient to be treated, as well as the route of administration, the compositions may be administered at varying doses.

In human therapy, the combination therapy of the invention can be administered alone but will generally be administered in admixture with a suitable pharmaceutical excipient, diluent or carrier selected with regard to the intended route of administration and standard pharmaceutical practice.

For example, the combination therapy of the invention can be administered orally, buccally or sublingually in the form of tablets, capsules, ovules, elixirs, solutions or suspensions, which may contain flavouring or colouring agents, for immediate-, delayed- or controlled-release applications. The agents, medicaments and pharmaceutical compositions of the invention may also be administered via intracavernosal injection.

Such tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxy-propylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, cellulose, milk sugar or high molecular weight polyethylene glycols. For aqueous suspensions and/or elixirs, the agents, medicaments and pharmaceutical compositions of the invention may be combined with various sweetening or flavouring agents, colouring matter or dyes, with emulsifying and/or suspending agents and with diluents such as water, ethanol, propylene glycol and glycerin, and combinations thereof.

The combination therapy of the invention can be administered parenterally, for example, intravenously, intraarterially, intraperitoneally, intrathecally, intraventricularly, intrasternally, intracranially, intra-muscularly or subcutaneously, or they may be administered by infusion techniques. They are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

Medicaments and pharmaceutical compositions suitable for parenteral administration include aqueous and non-aqueous sterile injection solutions which may contain antioxidants, buffers, bacteriostats and solutes which render the formulation isotonic with the blood of the intended recipient; and aqueous and non-aqueous sterile suspensions which may include suspending agents and thickening agents. The medicaments and pharmaceutical compositions may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

The combination therapy of the invention can also be administered intranasally or by inhalation and are conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A3 or 1,1,1,2,3,3,3-heptafluoropropane (HFA 227EA3), carbon dioxide or other suitable gas. In the case of a pressurised aerosol, the dosage unit may be determined by providing a valve to deliver a metered amount. The pressurised container, pump, spray or nebuliser may contain a solution or suspension of the active agent, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of an agent of the invention and a suitable powder base such as lactose or starch.

Alternatively, the combination therapy of the invention can be administered in the form of a suppository or pessary, or they may be applied topically in the form of a lotion, solution, cream, gel, ointment or dusting powder. The agents, medicaments and pharmaceutical compositions of the invention may also be transdermally administered, for example, by the use of a skin patch. They may also be administered by the ocular route, particularly for treating diseases of the eye.

For ophthalmic use, the combination therapy of the invention can be formulated as micronised suspensions in isotonic, pH adjusted, sterile saline, or, preferably, as solutions in isotonic, pH adjusted, sterile saline, optionally in combination with a preservative such as a benzalkonium chloride. Alternatively, they may be formulated in an ointment such as petrolatum.

For application topically to the skin, the combination therapy of the invention can be formulated as a suitable ointment containing the active agent suspended or dissolved in, for example, a mixture with one or more of the following: mineral oil, liquid petrolatum, white petrolatum, propylene glycol, polyoxyethylene polyoxypropylene agent, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

Formulations suitable for topical administration in the mouth include lozenges comprising the active ingredient in a flavoured basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Generally, in humans, local administration of the combination therapy of the invention at or near the site of a tumour is the preferred route, in particular intra-tumoural or peri-tumoural administration.

For veterinary use, the combination therapy of the invention is administered as a suitably acceptable formulation in accordance with normal veterinary practice and the veterinary surgeon will determine the dosing regimen and route of administration which will be most appropriate for a particular animal.

An eighth aspect of the invention relates to a method of prognosing or predicting responsiveness to a combination therapy described herein. In some embodiments, the method comprises determining a biomarker signature of a test sample, which enables a prognosis or prediction to be reached in respect of the individual from which the sample is obtained. The biomarker signature may be based on one or more of the biomarkers selected from the group consisting of Ccl2, Ccl3, Ccl4, Cxcl10 and Ifng, and the biomarkers listed in Table 36, Table 37 and/or Table 38.

For example, this aspect of the invention provides a method of prognosing responsiveness to a combination therapy described herein, comprising or consisting of the steps of:
(a) providing a sample (such as an ex vivo sample) from an individual to be tested; and
(b) determining a biomarker signature of the test sample by measuring the presence and/or amount in the test of sample of one or more biomarkers selected from one or more of the biomarkers selected from the group consisting of Ccl2, Ccl3, Ccl4, Cxcl10 and Ifng, and the biomarkers listed in Table 36, Table 37 and/or Table 38;
wherein the presence and/or amount in the test sample of the one or more biomarkers is indicative of responsiveness to the combination therapy in the individual.

It is intended that the method encompasses situations that do not necessitate a surgical step performed on the human or animal body. Therefore, by “providing a sample”, we include the meaning that the sample has been provided, and no active step (e.g. surgical step) was required to obtain such a sample. In this case, the sample may be referred to as an ex vivo sample.

In some embodiments, the sample is a blood sample. In some preferred embodiments, the sample is a serum or plasma sample derived from an unfractionated blood sample. In some preferred embodiments, the sample is a serum sample.

By “biomarker”, we include any naturally occurring biological molecule, or component or fragment thereof, the measurement of which can provide information useful in the diagnosis of cancer (e.g. pancreatic cancer), optionally chemotherapy-resistant cancer. Thus, in the context of Ccl2, Ccl3, Ccl4, Cxcl10 and Ifng and Tables 36-38, the biomarker(s) may be a nucleic acid molecule, such as a mRNA or cDNA, which encodes the protein or part thereof. Alternatively, the biomarker(s) may be the protein, or a polypeptide fragment or carbohydrate moiety thereof.

By “prognosing” or “prognostic”, we include predicting the likely course (i.e. responsiveness) of a medical condition. The responsiveness may be determined based on a positive outcome for the medical condition. For example, in cancer (e.g. pancreatic cancer), optionally chemotherapy-resistant cancer, responsiveness may be determined based on increased survival compared with a relevant control, and/or decreased tumour size/volume compared with a relevant control.

The methods of the invention are also suitable for testing a sample from any individual who is suspected of having, or at risk of developing, cancer (e.g. pancreatic cancer), optionally chemotherapy-resistant cancer. The methods of the invention are suitable for testing a sample from any individual who is undergoing treatment for cancer (e.g. pancreatic cancer), optionally chemotherapy-resistant cancer. The treatment may be chemotherapy as described herein alone (e.g. mFOLFIRINOX), anti-CD40 antibody (e.g. mitazalimab) alone, or a combination therapy thereof.

In some preferred embodiments, the treatment is a combination of mFOLFIRINOX and Mitazalimab.

It will be appreciated by persons skilled in the art that, in addition to measuring the biomarkers in a sample from an individual to be tested, the methods of the invention may also comprise measuring those same biomarkers in one or more control samples. By “relevant control” with respect to the methods of the invention, we include the meaning of an individual that: (i) is not undergoing treatment with a combination therapy as described herein; (ii) is undergoing treatment with a chemotherapy (e.g. mFOLFIRINOX) as described herein but is not being administered an anti-CD40 antibody (e.g. mitazalimab), preferably wherein the chemotherapy is the same as used in the combination therapy; and/or (iii) is undergoing treatment with an anti-CD40 antibody (e.g. mitazalimab) but is not undergoing treatment with a chemotherapy (e.g. mFOLFIRINOX) as described herein, preferably wherein the anti-CD40 antibody is the same as used in the combination therapy.

Thus, in some embodiments, the method further comprises or consists of the steps of:
(c) providing one or more (negative) relevant control samples as described herein; and
(d) determining a biomarker signature of the one or more control samples by measuring the presence and/or amount in the control sample(s) of the one or more biomarkers measured in step (b);
   wherein responsiveness to the combination therapy is identified in the event that the presence and/or amount in the test sample of the one or more biomarkers measured in step (b) is different from the presence and/or amount in the control sample(s) of the one or more biomarkers measured in step (d).

By “is different to the presence and/or amount in a control sample” we include that the presence and/or amount of the one or more biomarker(s) in the test sample differs from that of the one or more control sample(s) (or to predefined reference values representing the same). Preferably, the presence and/or amount in the test sample differs from the presence or amount in one or more control sample(s) (or mean of the control samples) by at least ±5%, for example, at least ±6%, ±7%, ±8%, ±9%, ±10%, ±11%, ±12%, ±13%, ±14%, ±15%, ±16%, ±17%, ±18%, ±19%, ±20%, ±21%, ±22%, ±23%, ±24%, ±25%, ±26%, ±27%, ±28%, ±29%, ±30%, ±31%, ±32%, ±33%, ±34%, ±35%, ±36%, ±37%, ±38%, ±39%, ±40%, ±41%, ±42%, ±43%, ±44%, ±45%, ±46%, ±47%, ±48%, ±49%, ±50%, ±51%, ±52%, ±53%, ±54%, ±55%, ±56%, ±57%, ±58%, ±59%, ±60%, ±61%, ±62%, ±63%, ±64%, ±65%, ±66%, ±67%, ±68%, ±69%, ±70%, ±71%, ±72%, ±73%, ±74%, ±75%, ±76%, ±77%, ±78%, ±79%, ±80%, ±81%, ±82%, ±83%, ±84%, ±85%, ±86%, ±87%, ±88%, ±89%, ±90%, ±91%, ±92%, ±93%, ±94%, ±95%, ±96%, ±97%, ±98%, ±99%, ±100%, ±125%, ±150%, ±175%, ±200%, ±225%, ±250%, ±275%, ±300%, ±350%, ±400%, ±500% or at least ±1000% of the one or more control sample(s) (e.g., the negative control sample).

Alternatively or additionally, the presence or amount in the test sample differs from the mean presence or amount in the control samples by at least ≥1 standard deviation from the mean presence or amount in the control samples, for example, 1.5, ≥2, ≥3, ≥4, ≥5, ≥6, ≥7, ≥8, ≥9, ≥10, ≥11, ≥12, ≥13, ≥14 or 15 standard deviations from the mean presence or amount in the control samples. Any suitable means may be used for determining standard deviation (e.g., direct, sum of square, Welford's), however, in one embodiment, standard deviation is determined using the direct method (i.e., the square root of [the sum the squares of the samples minus the mean, divided by the number of samples]).

Alternatively, or additionally, by “is different to the presence and/or amount in a control sample” we include that the presence or amount in the test sample does not correlate with the amount in the control sample in a statistically significant manner. For example, the presence or amount in the test sample may differ in a statistically significant manner with that of the control sample by an adjusted p-value of ≤0.05, for example, ≤0.04, ≤0.03, ≤0.02, ≤0.01, ≤0.005, ≤0.004, ≤0.003, ≤0.002, ≤0.001, ≤0.0005 or ≤0.0001. In some other embodiments, the presence or amount in the test sample may differ in a statistically significant manner with that of the control sample by an adjusted p-value of ≤0.05, for example, ≤0.04, ≤0.03, ≤0.02, ≤0.01, ≤0.005, ≤0.004, ≤0.003, ≤0.002, ≤0.001, ≤0.0005 or ≤0.0001. In some embodiments, the −Log₁₀ pvalue is >1.0, for example, >1.1, >1.2, >1.3, >1.4, >1.5, >1.6, >1.7, >1.8, >1.9, >2.0, >2.1, >2.2, >2.3, >2.4, >2.5, >2.6, >2.7, >2.8, >2.9, or >3.0. For example, in some embodiments, the −Log₁₀ pvalue is >1.87.

Any suitable means for determining p-value known to the skilled person can be used, including z-test, t-test, Student's t-test, f-test, Mann-Whitney U test, Wilcoxon signed-rank test and Pearson's chi-squared test.

Alternatively, or additionally, by “is different to the presence and/or amount in a control sample” we include that the presence or amount in the test sample does not correlate with the amount in the control sample when determined as described in Example 12. For example, we mean or include that the presence or amount in the test sample may differ in a statistically significant manner with that of the control sample with a −Log₁₀ pvalue of >1.0, for example, >1.1, >1.2, >1.3, >1.4, >1.5, >1.6, >1.7, >1.8, >1.9, >2.0, >2.1, >2.2, >2.3, >2.4, >2.5, >2.6, >2.7, >2.8, >2.9, or >3.0; preferably with a −Log₁₀ p-value of >1.87; and/or the presence or amount in the test sample differs from that of the control sample with a Log₂ fold change of >1 (i.e. more than 1), for example, >1.1, >1.2, >1.3, >1.4, >1.5, >1.6, >1.7, >1.8, >1.9, or >2, or a Log₂ fold change of <−1 (i.e. less than −1, meaning that the fold change value is departing further from 0), for example, <−1.1, <−1.2, <−1.3, <−1.4, <−1.5, <−1.6, <−1.7, <−1.8, <−1.9, or <−2. In some preferred embodiments, the log 2 fold change is greater than 1.

In some embodiments, the biomarker signature is based on biomarkers that are upregulated by the combination therapy in comparison with a relevant control (as described herein), wherein the biomarker is selected from the group consisting of Ccl2, Ccl3, Ccl4, Cxcl10 and Ifng.

In some embodiments, the biomarker signature is based on biomarkers that are upregulated by the combination therapy in comparison with chemotherapy (e.g. mFOLFIRINOX) alone, preferably an equivalent chemotherapy as used for the combination therapy. For example, the biomarker signature may be based on the upregulation of a biomarker selected from the group consisting of Adar, Parp9, Dhx58, Dhx60, Slfn4, Ifit3, Ifit3b, Herc6, Usp18, Ifi44, Hmgcs2, Orm1, and combinations thereof.

In some embodiments, the biomarker signature is based on biomarkers that are upregulated by the combination therapy in comparison with anti-CD40 antibody (e.g. mitazalimab) administration alone, preferably an equivalent anti-CD40 antibody as used for the combination therapy. For example, the biomarker signature may be based on the upregulation of a biomarker selected from the group consisting of Acer2, Mapre3, Lancl3, Eda2r, Hmgcs2, Car3, and combinations thereof. In some embodiments, the biomarker signature is based on biomarkers that are downregulated by the combination therapy in comparison with anti-CD40 antibody (e.g. mitazalimab) administration alone, preferably an equivalent anti-CD40 antibody as used for the combination therapy. For example, the biomarker signature may be based on the downregulation of a biomarker selected from the group consisting of Bud31, Gm33111, Ing2, Asf1a, Atf4, Gpbp1, Cd200r3, Zbtb44, Mtss2, Mospd1, Supt4a, Eif1, Gm15931, Ms4a2, Mcpt8, Rag1, and combinations thereof.

In some embodiments, the biomarker signature is based on biomarkers that are upregulated by the combination therapy in comparison with an untreated control (i.e. an individual that has not been subjected to a combination therapy as disclosed herein, or to chemotherapy or anti-CD40 antibody monotherapies). For example, the biomarker signature may be based on the upregulation of a biomarker selected from the group consisting of Zfp750, Eda2r, Psrc1, Cdkn1a, Ifit3, Ifit3b, Usp18, Mapre3, Upp1, Psme1, Parp9, Clca3a1, Gbp7, Ddx60, Ligp1, Herc6, Slfn4, Ifi44, and combinations thereof. In some embodiments, the biomarker signature is based on biomarkers that are downregulated by the combination therapy in comparison with an untreated control (i.e. an individual that has not been subjected to a combination therapy as disclosed herein, or to chemotherapy or anti-CD40 antibody monotherapies). For example, the biomarker signature may be based on the downregulation of a biomarker selected from the group consisting of Cd209a, Abca9, Hpgd, Fcgrt, and combinations thereof.

In some embodiments, the biomarker signature is based on biomarkers that are upregulated by the combination therapy in comparison with either corresponding monotherapy, preferably an equivalent chemotherapy (e.g. mFOLFIRINOX) or anti-CD40 antibody (e.g. mitazalimab) as used in the combination therapy. For example, the biomarker signature may be based on the upregulation of Hmgcs2.

In some embodiments, the biomarker signature is based on biomarkers that are upregulated by the combination therapy in comparison with either an anti-CD40 antibody (e.g. mitazalimab) monotherapy, preferably an equivalent anti-CD40 antibody as used for the combination therapy, or an untreated control as described herein. For example, the biomarker signature may be based on the upregulation of Mapre3 and/or Eda2r.

In one embodiment, the method of the invention may further comprise or consist of the steps of:
(e) providing one or more (positive) control sample from an individual who is undergoing or has undergone treatment with the combination therapy; and
(f) determining a biomarker signature of the control sample by measuring the presence and/or amount in the control sample of the one or more biomarkers measured in step (b);
wherein responsiveness to the combination therapy is identified in the event that the presence and/or amount in the test sample of the one or more biomarkers measured in step (b) corresponds to the presence and/or amount in the control sample of the one or more biomarkers measured in step (f).

Thus, the methods of the invention may comprise steps (c)+(d) and/or steps (e)+(f).

By “corresponds to the presence and/or amount in a control sample”, we include that the presence and/or amount is identical to that of a positive control sample(s); or closer to that of one or more positive control sample(s) than to one or more negative control sample(s) (or to predefined reference values representing the same). Preferably the presence and/or amount is within ±40% of that of the one or more positive control sample(s) (or mean of the positive control samples), for example, within ±39%, ±38%, ±37%, ±36%, ±35%, ±34%, ±33%, ±32%, ±31%, ±30%, ±29%, ±28%, ±27%, ±26%, ±25%, ±24%, ±23%, ±22%, ±21%, ±20%, ±19%, ±18%, ±17%, ±16%, ±15%, ±14%, ±13%, ±12%, ±11%, ±10%, ±9%, ±8%, ±7%, ±6%, ±5%, ±4%, ±3%, ±2%, ±1%, ±0.05% or within 0% of the one or more positive control sample(s).

Alternatively or additionally, the difference in the presence or amount in the test sample is ≤5 standard deviation from the mean presence or amount in the positive control sample(s), for example, ≤4.5, ≤4, ≤3.5, ≤3, ≤2.5, ≤2, ≤1.5, ≤1.4, ≤1.3, ≤1.2, ≤1.1, ≤1, ≤0.9, ≤0.8, ≤0.7, ≤0.6, ≤0.5, ≤0.4, ≤0.3, ≤0.2, ≤0.1 or 0 standard deviations from the from the mean presence or amount in the control sample(s), provided that the standard deviation ranges for differing and corresponding biomarker expressions do not overlap (e.g., abut, but no not overlap).

Alternatively, or additionally, by “corresponds to the presence and/or amount in a control sample” we include that the presence or amount in the test sample correlates with the amount in the control sample in a statistically significant manner. For example, the presence or amount in the test sample does not differ from that of the control sample by a statistical difference with a p-value of >0.05, for example >0.06, >0.07, >0.08, >0.09 or >0.1.

Alternatively, or additionally, by “corresponds to the presence and/or amount in a control sample” we include that the presence or amount in the test sample correlates with the amount in the control sample when determined as described in Example 12. For example, we mean or include that the presence or amount in the test sample correlates with that of the control sample with a Log₁₀ p-value of <3.0, for example, <2.9, <2.8, <2.7, <2.6, <2.5, <2.4, <2.3, <2.2, <2.1, <2.0, preferably <2.5; and/or the presence or amount in the test example correlates with that of the control sample with a Log₂ fold change of <2 (i.e. less than 2), for example, <1.9, <1.8, <1.7, <1.6, <1.5, <1.4, <1.3, <1.2, <1.1, or <1, or a Log₂ fold change of >−2 (i.e. more than −2, meaning that the fold change value is moving closer to 0), for example, >−1.9, >−1.8, >−1.7, >−1.6, >−1.5, >−1.4, >−1.3, >−1.2, >−1.1, or >−1.

Differential expression (upregulation or downregulation) of biomarkers, or lack thereof, can be determined by any suitable means known to a skilled person. Differential expression is determined to a p value of a least less than 0.05 (p=<0.05), for example, at least <0.04, <0.03, <0.02, <0.01, <0.009, <0.005, <0.001, <0.0001, <0.00001 or at least <0.000001.

It will be appreciated by persons skilled in the art that differential expression may relate to a single biomarker or to multiple biomarkers considered in combination (i.e., as a biomarker signature). Thus, a p value may be associated with a single biomarker or with a group of biomarkers. Indeed, biomarkers having a differential expression p value of greater than 0.05 when considered individually may nevertheless still be useful as biomarkers in accordance with the invention when their expression levels are considered in combination with one or more other biomarkers.

In one preferred embodiment of the eighth aspect of the invention, the method is repeated on the individual. Thus, steps (a) and (b) may be repeated using a sample from the same individual taken at different time to the original sample tested (or the previous method repetition). Such repeated testing may enable responsiveness to the combination therapy to be assessed, and (if appropriate) to select an alternative regime to be adopted.

Thus, in one embodiment, the method is repeated using a test sample based on the treatment cycles and/or regimes described herein. For example, the method may be repeated using a test sample taken every period from the group consisting of: 1 day, 2 days, 3 days, 4 days, 5 days, 6 days, 7 days, 8 days, 9 days, 10 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, or more.

Alternatively, or additionally, the method may be repeated at least once, for example, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 21 times, 22 times, 23 times, 24 times, 25 times, or more.

Alternatively, or additionally, the method is repeated continuously.

In some embodiments, the method is repeated until responsiveness to the combination therapy is observed in the individual using the methods of the present invention and/or conventional clinical methods.

### EXAMPLES

### Example 1—Sequence Information

### Anti-CD40 Antibody Clone G12 (Antibody ADC-1013)

This clone is described in WO 2016/023960. An exemplary antibody that comprises the CDR sequences of ADC-1013 is known as mitazalimab.

(a) CDR Sequences (Defined According to the IMGT Numbering, with Core CDR Sequences Underlined Therein)

V_{L} CDR1:
[SEQ ID NO: 1]
CTGSSSNIGAGYNVY;
V_{L} CDR2:
[SEQ ID NO: 2]
GNINRPS;
V_{L} CDR3:
[SEQ ID NO: 3]
CAAWDKSISGLV;
VH CDR1:
[SEQ ID NO: 4]
GFTFSTYGMH;
V_{H} CDR2:
[SEQ ID NO: 5]
GKGLEWLSYISGGSSYIFYADSVRGR;
and
V_{H} CDR3:
[SEQ ID NO: 6]
CARILRGGSGMDL.

### (b) Variable Region Sequences

Variable light chain (V_{L}) amino acid sequence-(CDRs underlined)
SEQ ID NO: 7
QSVLTQPPSASGTPGQRVTISCTGSSSNIGAGYNVYWYQQLPGTAPKLLIYGNINRPSGVP
DRFSGSKSGTSASLAISGLRSEDEADYYCAAWDKSISGLVFGGGTKLTVLG
Variable heavy chain (V_{H}) amino acid sequence-(CDRs underlined)
SEQ ID NO: 8
EVQLLESGGGLVQPGGSLRLSCAASGFTFSTYGMHWVRQAPGKGLEWLSYISGGSSYIFYA
DSVRGRFTISRDNSENALYLQMNSLRAEDTAVYYCARILRGGSGMDLWGQGTLVTVSS
Variable light chain (V_{L}) nucleotide sequence-
SEQ ID NO: 9
CAGTCTGTGCTGACTCAGCCACCCTCAGCGTCTGGGACCCCCGGGCAGAGGGTCACCA
TCTCTTGCACTGGGAGCAGCTCCAACATCGGGGGGGGTTACAATGTATACTGGTATCAG
CAGCTCCCAGGAACGGCCCCCAAACTCCTCATCTATGGTAACATCAATCGGCCCTCAGG
GGTCCCTGACCGATTCTCTGGCTCCAAGTCTGGCACCTCAGCCTCCCTGGCCATCAGTG
GGCTCCGGTCCGAGGATGAGGCTGATTATTACTGTGCAGCATGGGATAAGAGCATTTCT
GGTCTGGTTTTCGGCGGAGGAACCAAGCTGACGGTCCTAGGT
Variable heavy chain (V_{H}) nucleotide sequence-
SEQ ID NO: 10
GAGGTGCAGCTGTTGGAGTCTGGGGGAGGCTTGGTACAGCCTGGGGGGTCCCTGAGAC
TCTCCTGTGCAGCCTCTGGATTCACCTTCAGTACTTATGGCATGCACTGGGTCCGCCAGG
CTCCAGGGAAGGGGCTGGAGTGGCTTTCATATATTAGTGGTGGTAGTAGTTACATTTTCT
ACGCAGACTCAGTGAGGGGCCGATTCACCATCTCCAGAGACAACTCCGAGAACGCGCT
GTATCTGCAAATGAACAGCCTGAGAGCCGAGGACACGGCCGTGTATTACTGTGCGAGAA
TATTAAGAGGCGGGAGCGGTATGGACCTCTGGGGCCAAGGTACACTGGTCACCGTGAG
CTCA

### (c) Exemplary Constant Region Amino Acid Sequences

Human Ig lambda light chain C2 region (NCBI AAA59107.1)-
SEQ ID NO: 11
QPKAAPSVTLFPPSSEELQANKATLVCLISDFYPGAVTVAWKADSSPVKAGVETTTPSKQSN
NKYAASSYLSLTPEQWKSHRSYSCQVTHEGSTVEKTVAPTECS
Human Ig gamma-1 heavy chain constant region (UniProt P01857.1)-
SEQ ID NO: 12
ASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTLPPSRDELTKNQV
SLTCLVKGFYPSDIAVEWESNGQPENNYKTTPPVLDSDGSFFLYSKLTVDKSRWQQGNVFS
CSVMHEALHNHYTQKSLSLSPGK
Human CD40 Sequence-
>gi|117606560|gb|ABK41937.1|CD40 molecule, TNF receptor superfamily
member 5 [Homo sapiens]
SEQ ID NO: 13
MVRLPLQCVLWGCLLTAVHPEPPTACREKQYLINSQCCSLCQPGQKLVSDCTEFTETECLPC
GESEFLDTWNRETHCHQHKYCDPNLGLRVQQKGTSETDTICTCEEGWHCTSEACESCVLH
RSCSPGFGVKQIATGVSDTICEPCPVGFFSNVSSAFEKCHPWTSCETKDLVVQQAGTNKTD
VVCGPQDRLRALVVIPIIFGILFAILLVLVFIKKVAKKPTNKAPHPKQEPQEINFPDDLPGSNT
AAPVQETLHGCQPVTQEDGKESRISVQERQ

### Example 2—OPTIMIZE-1 Clinical Study

### General Information

Study title: A phase 1b/2, open-label, multi-centre study assessing the clinical efficacy of mitazalimab in combination with chemotherapy in metastatic pancreatic ductal adenocarcinoma.
Region: Europe.
Estimated number of sites: 6-8 sites for Part 1 and up to 15 sites for Part 2.
Number of patients estimated: Up to 80 patients.
Patient population: Patients with previously untreated metastatic pancreatic ductal adenocarcinoma, measurable and histologically confirmed. Patients must be 18 years of age or older and have an Eastern Cooperative Oncology Group performance (ECOG) status of 0 or 1 and good organ function by laboratory assessment.

### Investigational Medicinal Product(s), Dosage and Administration

Mitazalimab was assessed in combination with the chemotherapy modified FOLFIRINOX (mFOLFIRINOX). Mitazalimab and mFOLFIRINOX were administered by intravenous infusions following a 14-day cycle schedule, except for the first treatment cycle of 21 days where mitazalimab is administered at Day 1 and 10 and infusion of mFOLFIRINOX start Day 8.
Mitazalimab: An agonistic human monoclonal (IgG1) antibody targeting CD40, developed for cancer immunotherapy. In Part 1 (Phase 1b) of the study, the dose of mitazalimab was escalated from a starting dose of 450 μg/kg up to 900 μg/kg (2 dose levels evaluated) to obtain the recommended phase 2 dose (RP2D) of mitazalimab in combination with mFOLFIRINOX. In Part 2 of the study, the RP2D of mitazalimab was administered in combination with mFOLFIRINOX to all patients.

The chemotherapy was administered as background treatment as it is standard of care for first line treatment of advanced pancreatic cancer recommended by the European Society of Medical Oncology (ESMO) and the National Comprehensive Cancer Network (NCCN). Therefore, mFOLFIRINOX and gemcitabine plus nab-paclitaxel are classified as Non-Investigational Medicinal Products.
mFOLFIRINOX: The mFOLFIRINOX treatment consisted of oxaliplatin (85 mg/m²), leucovorin (400 mg/m²), irinotecan (150 mg/m²) and 5-fluorouracil (5-FU, 2400 mg/m²). In case of supply issues with leucovorin, or to adhere to local standard of care practice, other approved similar folinates may be used under the supervision of an experienced clinician. In case mFOLFIRINOX is not found safe and tolerable together with the lowest dose of mitazalimab, mFOLFIRINOX will be exchanged to gemcitabine plus nab-paclitaxel.
Gemcitabine plus nab-paclitaxel (if applicable): Gemcitabine in combination with nab-paclitaxel is approved in Europe for the first line treatment of adult patients with metastatic adenocarcinoma of the pancreas. Gemcitabine plus nab-paclitaxel was only administered in this study in case mFOLFIRINOX was not found safe and tolerable in combination with mitazalimab at the lowest dose-level.

### Study Design

A phase 1b/2, open-label, multicentre study designed to evaluate the safety, tolerability, and efficacy of mitazalimab in combination with chemotherapy in patients with metastatic pancreatic ductal adenocarcinoma.

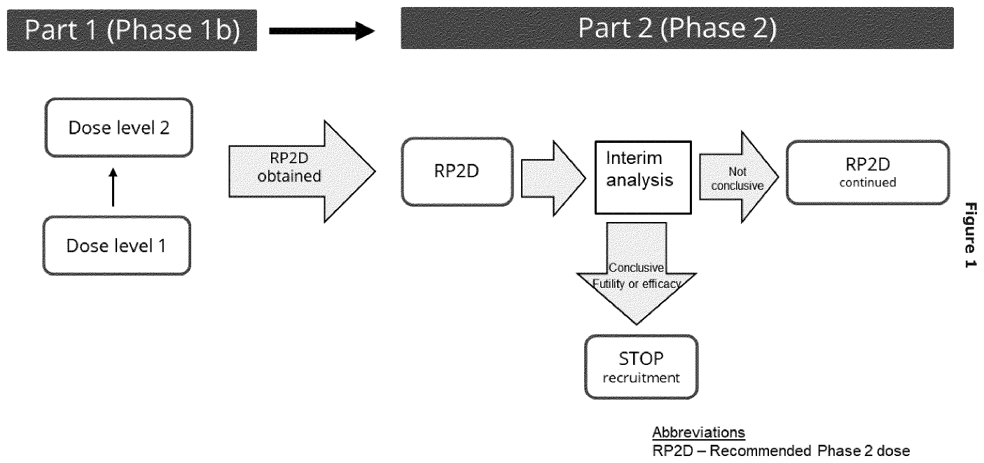

The study consists of 2 parts (as illustrated in FIG. 1):
Part 1 (Phase 1b): Two dose levels of mitazalimab in combination with mFOLFIRINOX were evaluated to determine a tolerable mitazalimab dose (RP2D) for Part 2 of the study. Part 1 followed a Bayesian optimal interval (BOIN) design with at least 3 patients enrolled at each dose level. As a minimum, 6 patients were evaluated at the RP2D in Part 1. If the lowest dose of mitazalimab (450 μg/kg) in combination with mFOLFIRINOX was not found safe and tolerable, the mFOLFIRINOX treatment could be exchanged to gemcitabine plus nab-paclitaxel.
Part 2 (Phase 2): Up to 60 enrolled patients were administered mitazalimab and mFOLFIRINOX (or gemcitabine plus nab-paclitaxel) to explore the clinical efficacy of mitazalimab in combination with chemotherapy in Part 2 of the study. A Simon's two-stage design with an interim analysis for stop for futility or efficacy was performed when 23 patients in total (including patients from both Part 1 and Part 2) were evaluable for objective response rate (ORR).

In Part 1, there was staggered dosing with at least 11 days between the first dose of mitazalimab administered to the first patient and the first dose of mitazalimab administered to the second patient at each dose level.

All patients were monitored for at least 4 hours after the end of the first infusion of mitazalimab, and for at least 2 hours after the second mitazalimab infusion. If infusion-related reactions had not been observed at the latest infusion (the 2nd or later infusion), the monitoring of the patient could be reduced to 1 hour for subsequent infusions. The monitoring period may be prolonged for all patients by the Data Review Committee (DRC) based on emerging safety data.

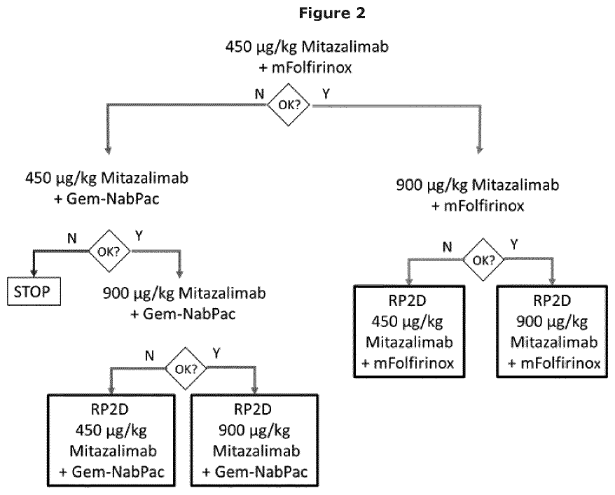

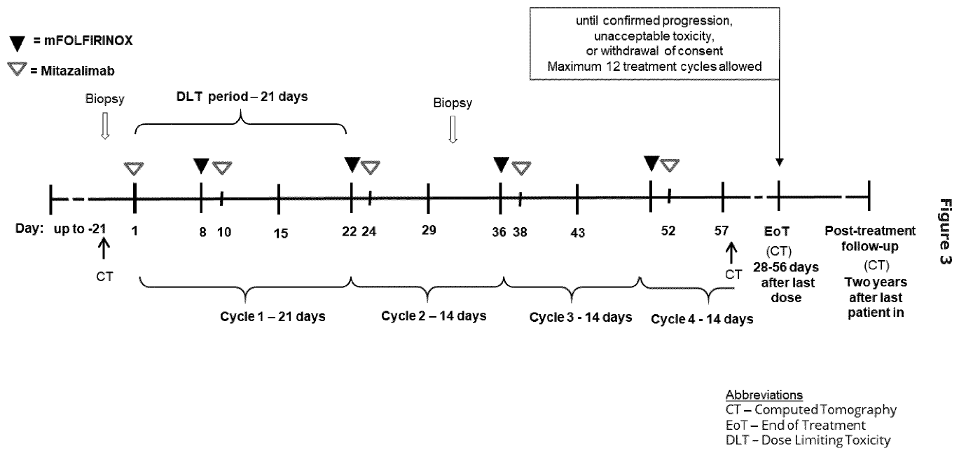

Referring to FIG. 2, in case the lowest planned dose level of mitazalimab (450 μg/kg) in combination with mFOLFIRINOX in Part 1 was found not safe or tolerable, mFOLFIRINOX could be exchanged to gemcitabine plus nab-paclitaxel, and the same mitazalimab dose escalation schedule followed. If the lowest planned dose level of mitazalimab in combination with gemcitabine plus nab-paclitaxel in Part 1 was found not safe or tolerable, the study terminated.

When the last patient in Part 1 completed the DLT evaluation period, and data had been collected and reviewed by the DRC, a decision to continue to Part 2 was taken. Up to 60 patients were planned to be enrolled in Part 2.

An interim analysis to allow stopping for futility or efficacy was performed when efficacy data became available for a total of 23 patients at RP2D (Part 1 and Part 2). The interim analysis was based on objective response rate (ORR) observed for all patients dosed at the mitazalimab RP2D. The interim analysis included efficacy data up to 17 weeks of study treatment (8 cycles for mFOLFIRINOX and 4 cycles for gemcitabine plus nab-paclitaxel). Depending on the outcome of the interim analysis, it was decided if the study should stop further enrollment or continue to enroll 37 additional patients.

The dosage schedule and visit assessment schedules were the same for Part 1 and Part 2 in the study for each respective combination of chemotherapy.

### Duration of Treatment

The patients may receive treatment with mitazalimab and mFOLFIRINOX for a maximum of 12 treatment cycles (˜6 months; one 21-day treatment period/dose-limiting toxicity (DLT) evaluation period (Cycle 1) followed by eleven 14-day treatment cycles). However, it is possible to extend beyond 12 treatment cycles until progression an unacceptable level of toxicity is observed in the patient.

The patients may receive treatment with mitazalimab and gemcitabine plus nab-paclitaxel for a maximum of 6 treatment cycles (˜6 months; one 35-day treatment period (Cycle 1) whereof the first 28 days will be the DLT evaluation period followed by five 28-day treatment cycles) (if applicable).

Patients may continue study treatment within above defined limits until progressive disease (PD), or clear clinical deterioration, according to Investigator's judgment, as long as the patients are tolerating the treatment and agree to continue. After study treatment completion the patients perform an End of treatment visit and continue to a treatment follow up period assessing disease and survival status.

### Doses, Dosage Schedule and Dose Escalation

Both mFOLFIRINOX and gemcitabine plus nab-paclitaxel are well-established and effective chemotherapy regimens for treatment of advanced pancreatic cancer. A central principle in the dose-finding portion of the study is to never compromise the intensity of this standard chemotherapy. If an adverse event is considered related to chemotherapy based on the investigators' clinical judgement, treatment can be adjusted in the standard manner as summarized in the dose adjustment sections in Example 4 (for mFOLFIRINOX) and Example 5 (for gemcitabine plus nab-paclitaxel) to this protocol. However, when an unexpected toxicity or toxicity of unexpected intensity with regard to these chemotherapies is encountered during the escalation portion of this study and the investigator considers that this toxicity could undermine the effectiveness of the chemotherapy, the investigator could discontinue mitazalimab. If this occurred during the DLT period of the study and the adverse event was not considered a DLT, the patient could be replaced.

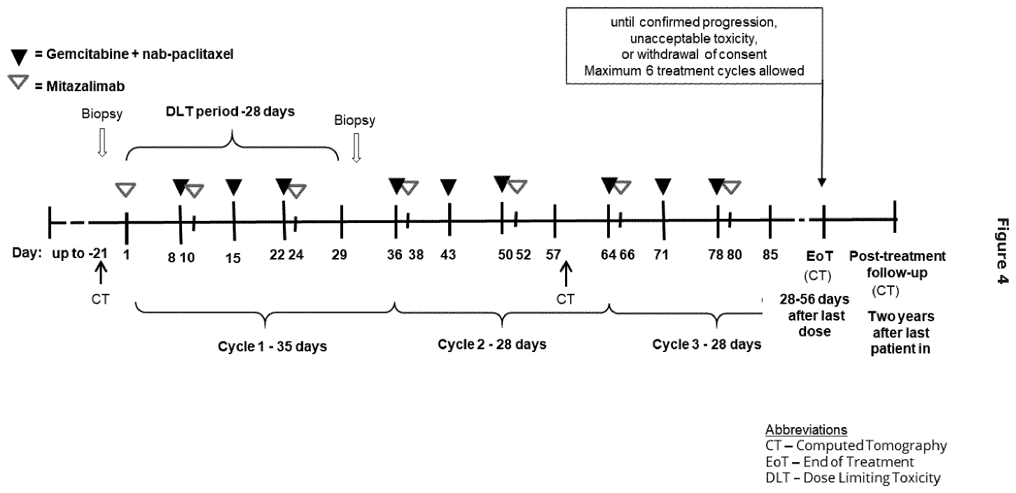

Two dose levels, 450 μg/kg and 900 μg/kg, of mitazalimab were planned to be administered in combination with mFOLFIRINOX in Part 1. During the first treatment cycle (the first 21 days) for each patient, mitazalimab was administered on Day 1 and Day 10 and mFOLFIRINOX infusion started on Day 8. During the following 14-day treatment cycles, mFOLFIRINOX was administered on Day 1 and mitazalimab on Day 3 of each cycle, see FIG. 3. In case the number of DLTs for a dose level fulfilled the stop criteria, see Table 3 Escalation rules based on number of patients with DLTs, at the lowest dose level of mitazalimab, the chemotherapy mFOLFIRINOX could be exchanged to the chemotherapy gemcitabine plus nab-paclitaxel, as illustrated in FIG. 2. If changing the chemotherapy from mFOLFIRINOX to gemcitabine plus nab-paclitaxel, see dosage schedule in Example 5.

The dose escalation in Part 1 follows a BOIN design with at least 3 evaluable patients per dose level. According to the BOIN design, each time a cohort of patients has been completed, a decision is made whether the next cohort should remain on the same dose, escalate to the next higher dose level or de-escalate to the next lower dose level, according to the decision rules presented in Table 3 below. A range of 3-9 patients was included at each dose level. In this study, the de-escalation from 450 μg/kg mitazalimab in combination with mFOLFIRINOX was a change of chemotherapy to gemcitabine plus nab-paclitaxel as previously described.

TABLE 3
Escalation rules based on number of patients with DLTs

| | Number of patients evaluable for | | | | | | |
|---|---|---|---|---|---|---|---|
| Decision, based on the number | DLT at the current dose level | | | | | | |
| of patients with DLTs (N_{DLT}) | 3 | 4 | 5 | 6 | 7 | 8 | 9 |
| Escalate, if N_{DLT}≤ | 0 | 0 | 1 | 1 ^{a} | 1 | 1 | 2 |
| Remain on dose- level, if N_{DLT}= | 1 | 1 | NA | 2 | 2 | 2 | 3 |
| De-escalate, if N_{DLT}≥ | 2 | 2 | 2 | 3 | 3 | 3 | 4 |
| Disallow dose level, if N_{DLT}≥ | 3 | 3 | 4 | 4 | 5 | 5 | 5 |

NA = Not applicable.
^{a} Stop if higher dose level has been evaluated and the number of DLTs on that higher dose level led to a de-escalation.

The dose escalation was determined by the DRC upon review of safety data obtained during the DLT evaluation period as well as available data beyond the DLT evaluation period from all patients in the study. Dose escalation was allowed when the DRC defined the dose and the dosage schedule for the next dose level. Based on available data, the DRC could decide to introduce intermediate dose levels in between the pre-planned dose level as well as to change the dosing frequency.

Mitazalimab in combination with mFOLFIRINOX: The DLT evaluation period is defined as the time from the first dose of mitazalimab (Day 1) until Day 21 in the first treatment cycle (Cycle 1). Mitazalimab in combination with gemcitabine plus nab-paclitaxel: The DLT evaluation period is defined as the time from the first dose of mitazalimab (Day 1) until Day 28 within the first treatment cycle of 35 days (Cycle 1). This period enabled the DRC to evaluate for early safety of mitazalimab together with the chemotherapy combination.

A DLT was defined as one of the following toxicities (i.e., drug-related AEs) graded by CTCAE version 5.0:
Grade 4 neutropenia lasting for more than 7 days
Grade 4 thrombocytopenia lasting for more than 7 days
Grade 4 infusion-related reaction
Grade 3 infusion-related reaction which does not resolve to a lower grade within 24 hours after onset
Grade 4 AST, ALT and/or bilirubin
Grade 3 AST, ALT and/or bilirubin not resolved to a lower grade within 7 days
Any grade≥3 non-hematologic toxicity except for:
   Laboratory abnormalities that have no clinical consequence and resolve to grade 52 within 14 days (including electrolyte abnormalities responding to medical intervention
   Fatigue
   Nausea and/or vomiting lasting less than 48 hours
   Diarrhea which does resolve to less than grade 3 by the end of the DLT period

The same DLT criteria applied for mitazalimab in combination with mFOLFIRINOX and gemcitabine plus nab-paclitaxel. A DLT was considered related to study treatment unless there is a clear, well-documented, alternative explanation for the adverse event (AE). AEs that meet the above criteria but occur after the DLT evaluation period were not defined as DLTs, unless the onset of the event was during the DLT evaluation period. The event could be reported as AEs/Serious adverse events (SAEs), as applicable.

The RP2D of mitazalimab in combination with mFOLFIRINOX or gemcitabine plus nab-paclitaxel, as obtained in Part 1, was administered to all patients in Part 2. Modifications in administration of mFOLFIRINOX or gemcitabine plus nab-paclitaxel, were allowed as described in Example 4 and Example 5, respectively. The same dosage schedule for mitazalimab in combination with each respective chemotherapy applies for both Part 1 and Part 2 of the study.

### Objectives and Endpoints

PART 1

| | |
|---|---|
| OBJECTIVES | ENDPOINTS |

Primary

| | |
|---|---|
| To determine the recommended Phase 2 | Incidence of DLTs |
| dose (RP2D) of mitazalimab in combination | |
| with chemotherapy | |

Secondary

| | |
|---|---|
| To assess the safety and tolerability of | Type, frequency and severity of |
| mitazalimab in combination with | adverse events (AEs) |
| chemotherapy | |
| To assess the immunogenicity of | Detection and characterization of anti- |
| mitazalimab | drug antibody (ADA) titers in serum |
| To assess pharmacokinetics (PK) of | PK parameters will include Cₘₐₓ, Tₘₐₓ, |
| mitazalimab after single and repeated | and AUC_{(0-T)}. Additional parameters |
| administrations | may be calculated depending on data |
| | obtained |
| To assess the clinical activity of mitazalimab | Objective response rate (ORR) |
| in combination with chemotherapy (i.e., | Best Overall Response (BOR), with |
| anti-tumour activity as per RECIST v. 1.1 | response categories CR, PR, SD and |
| guideline) | PD |
| | Duration of response (DoR) |
| | Duration of SD |
| | Disease control rate |
| | Time to next anti-cancer therapy |
| To assess survival outcomes following | Progression-free survival (PFS) |
| repeated administrations of mitazalimab in | Overall survival (OS) |
| combination with chemotherapy | |

Exploratory

| | |
|---|---|
| To characterize the impact of mitazalimab | Change in cytokines and chemokines |
| in combination with chemotherapy on the | Quantification of changes in innate |
| immune response both systemically and in | and adaptive immune cell populations |
| the tumour microenvironment | and immune cell activation status |
| | Change in gene expression and |
| | immune signatures, assessing |
| | differentially expressed genes and |
| | gene signatures for specific immune |
| | cells |
| To characterize the impact on CA19-9 and | Change in CA19-9 |
| ctDNA of mitazalimab in combination with | Change in ctDNA and differentially |
| chemotherapy | expressed genes of interest |

PART 2

| | |
|---|---|
| OBJECTIVES | ENDPOINTS |

Primary

| | |
|---|---|
| To assess the clinical activity of mitazalimab | Objective response rate (ORR) |
| in combination with chemotherapy (i.e., | |
| anti-tumour activity as per RECIST v. 1.1 | |
| guideline) | |

Secondary

| | |
|---|---|
| To assess the clinical activity of mitazalimab | Best Overall Response (BOR), with |
| in combination with chemotherapy (i.e., | response categories CR, PR, SD and |
| anti-tumour activity as per RECIST v. 1.1 | PD |
| guideline) | Duration of response (DoR) |
| | Duration of SD |
| | Disease control rate |
| | Time to next anti-cancer therapy |
| To assess survival outcomes following | Progression-free survival (PFS) |
| repeated administrations of mitazalimab in | Overall survival (OS) |
| combination with chemotherapy | |
| To assess the safety and tolerability of | Type, frequency and severity of AEs |
| mitazalimab in combination with | |
| chemotherapy | |
| To assess the immunogenicity of | Detection and characterization of anti- |
| mitazalimab | drug antibody (ADA) titers in serum |
| To assess PK of mitazalimab after single and | PK parameters will include Cₘₐₓ, Tₘₐₓ, |
| repeated administrations | and AUC_{(0-T)}. Additional parameters |
| | may be calculated depending on data |
| | obtained |

Exploratory

| | |
|---|---|
| To characterize the impact of mitazalimab | Change in cytokines and chemokines |
| in combination with chemotherapy on the | Quantification of changes in innate |
| immune response both systemically and in | and adaptive immune cell populations |
| the tumour microenvironment | and immune cell activation status |
| | Change in gene expression and |
| | immune signatures, assessing |
| | differentially expressed genes and |
| | gene signatures for specific immune |
| | cells |
| To characterize the impact on CA19-9 and | Change in CA19-9 |
| ctDNA of mitazalimab in combination with | Change in ctDNA and differentially |
| chemotherapy | expressed genes of interest |

### Assessments

Assessments included demographics, medical history (including previous anti-cancer treatments), height and weight, vital signs (blood pressure, pulse rate, oxygen saturation and body temperature), physical examination, ECG, ECOG performance status, and clinical laboratory tests (clinical chemistry, haematology, urinalysis), concomitant medication and collection of AEs. Blood samples were taken for analysis of pharmacokinetics, pharmacodynamics, and immunogenicity analyses. Anti-tumour activity was evaluated by assessing CT scans according to RECIST v. 1.1 guideline. A post-treatment follow-up period included assessment of disease/survival status, subsequent cancer-related therapy and anti-tumour activity (CT-scan). The post-treatment follow-up period will continue for up to 2 years after last patient in (LPI).

### Statistics

### Statistical Methodology

No formal statistical hypothesis was defined for Part 1 (Phase 1b). For Part 2, the primary endpoint ORR was compared between mitazalimab and historical control using a one-sided exact binomial test performed on the 10% significance level. All secondary and exploratory endpoints were summarized using appropriate descriptive statistics. Patients in Part 1 who are on the same dose regimen as patients in Part 2 were pooled together with patients enrolled in Part 2 for statistical analyses and data summaries.

### Sample Size

In Part 1, it was estimated that at least 9 patients would be enrolled for the 2 dose levels planned, 3 patients at the lower level and 3+3 patients at the higher dose level. The actual number of patients in Part 1 was dependent on data obtained during the study.

For Part 2, the sample size was based on extended Simon's two-stage design including an interim analysis for futility and efficacy and assumed a 15% drop-out rate. A total of 23 patients at RP2D (Part 1+Part 2) with data evaluable for ORR were included in the futility and efficacy analysis. To achieve this number, an estimate of 27 patients needed to be enrolled. An additional 37 patients may be enrolled if the study continues, depending on the outcome of the interim analysis, to include a total of 54 evaluable patients.

List of Abbreviations and Terms

| Abbreviation/term | Definition |
|---|---|
| ADA | Anti-drug antibody (i.e., antibodies against |
| | mitazalimab) |
| ADCC | Antibody-dependent cellular cytotoxicity |
| AE | Adverse event |
| AESI | Adverse event of special interest |
| ALT | Alanine aminotransferase |
| aPTT | activated partial thromboplastin time |
| AST | Aspartate aminotransferase |
| AUC | Area under the serum concentration-time curve |
| AR | Adverse reaction |
| BSA | Body surface area |
| Cₘₐₓ | Maximum serum concentration |
| CA 19-9 | Carbohydrate antigen 19-9 |
| CD40 | Cluster of differentiation 40 |
| CL | Clearance |
| CNS | Central nervous system |
| CR | Complete response |
| CRO | Contract Research Organization |
| CT | Computed tomography |
| CTCAE | Common Terminology Criteria for Adverse Events |
| ctDNA | Circulating tumour DNA |
| DC | Dendritic cells |
| DLT | Dose-limiting toxicity |
| DPD | Dihydropyrimidine dehydrogenase |
| DRC | Data Review Committee |
| EC | Ethics Committee |
| ECG | Electrocardiogram |
| ECOG | Eastern Cooperative Oncology Group |
| EMA | European Medicines Agency |
| ESMO | European Society for Medical Oncology |
| FDA | Food and Drug Administration (US) |
| 5-FU | 5-Fluorouracil |
| G-CSF | Granulocyte Colony-Stimulating Factor |
| GDPR | General Data Protection Regulation (EU) 2016/679 |
| Gem-NabPac | Gemcitabine plus nab-paclitaxel |
| GLP | Good Laboratory Practice |
| ICH | International Conference on Harmonization |
| IFNγ | Interferon gamma |
| IL-2 (IL-6, IL-8 etc) | Interleukin-2 (Interleukin-6, Interleukin-8 etc) |
| IMP | Investigational medicinal product |
| IMPD | Investigational Medicinal Product Dossier |
| IV | Intravenous |
| IWRS | Interactive Web Response System |
| LPI | Last patient in |
| mFOLFIRINOX | modified FOLFIRINOX |
| MTD | Maximum Tolerated Dose |
| NCCN | National Comprehensive Cancer Network |
| ORR | Objective Response Rate |
| PK | Pharmacokinetics |
| PO | Per oral |
| PD | Progressive disease |
| PR | Partial response |
| RSI | Reference safety information |
| SAE | Serious adverse event |
| SAR | Serious adverse reaction |
| SD | Stable disease |
| SUSAR | Suspected Unexpected Serious Adverse Reaction |
| Tₘₐₓ | Time to Cₘₐₓ |

### Visit Assessment Schedule

The visit assessments tables below are applicable when administering mitazalimab in combination with mFOLFIRINOX and includes all assessments to be performed during the study. Table 4 lists the study assessments to be performed during screening and Treatment cycle 1. Table 5 lists the assessments to be performed during Treatment cycle 2 and subsequent cycles, and at the End of treatment. The assessments to be performed during the post-treatment follow-up period, and at the End of study, are listed in Table 6. The timing of the different assessments is indicated with an “X”.

A number of pre- and post-medications can be administered with specific timing in relation to administration (up to 3 days prior to infusion) of mitazalimab and mFOLFIRINOX, respectively.

The assessment schedules are applicable for both Part 1 and Part 2 of the study.

Visit assessment schedules to be used when administering mitazalimab in combination with gemcitabine plus nab-paclitaxel are found in Example 5.

TABLE 4
Assessment schedule during screening period and Treatment cycle 1

| | Activity/Visit | | | | | | | | | |
|---|---|---|---|---|---|---|---|---|---|---|
| | Screening | Cycle 1 (21-day treatment cycle) | | | | | | | | |
| | Day | | | | | | | | | |
| | −21 to −1 | 1 | | | 2 | 8 ± 1 | | 9 ± 1 | 10 ± 1 | 10 ± 1³ |
| | Time in relation to study drug | | | | | | | | | |
| | | | | | 24 ± | | | | | |
| | | Pre | Mitazalimab | Post² | 2 h² | Pre | mFOLFIRINOX³ | | | Pre |
| Informed | X⁴ | | | | | | | | | |
| Consent | | | | | | | | | | |
| Eligibility | X | | | | | | | | | |
| criteria | | | | | | | | | | |
| Demographics | X | | | | | | | | | |
| Medical | X | | | | | | | | | |
| history | | | | | | | | | | |
| Body weight | X¹² | X⁵ | | | | | | | | |
| Height | X | | | | | | | | | |
| Vital signs | X | X | | 1 h ± | | X⁵ | | | | |
| | | | | 10 min | | | | | | |
| | | | | 2 h ± | | | | | | |
| | | | | 10 min | | | | | | |
| | | | | 4 h ± | | | | | | |
| | | | | 15 min | | | | | | |
| Physical | X | X⁵ | | | | X | | | | |
| examination | | | | | | | | | | |
| 12-lead ECG | X | X⁵ | | 1 h ± | | | | | | |
| | | | | 10 min | | | | | | |
| | | | | 4 h ± | | | | | | |
| | | | | 15 min | | | | | | |
| ECOG | X | X⁵ | | | | | | | | |
| Treatment | | X⁶ | | | | X⁶ | | | | X⁶ |
| discontinuation | | | | | | | | | | |
| criteria | | | | | | | | | | |
| Pre- and | | Note: Premedications are to be given up to 3 days | | | | | | | | |
| post- | | prior to administration of study treatment. | | | | | | | | |
| medication | | | | | | | | | | |
| Mitazalimab | | | X⁷ | | | | | | | |
| administration | | | | | | | | | | |
| mFOLFIRINOX | | | | | | | | | | |
| administration | | | | | | | | | | |
| Oxaliplatin | | | | | | | X | | | |
| Irinotecan | | | | | | | X | | | |
| Leucovorin¹³ | | | | | | | X | | | |
| 5-FU | | | | | | | X | X | X | |
| Adverse | Continuous from signing ICF until 28 days | | | | | | | | | |
| events | after last dose of study drug | | | | | | | | | |
| Concomitant | Continuous from signing of ICF | | | | | | | | | |
| medication | until End of treatment visit | | | | | | | | | |
| Concomitant | Continuous from signing of ICF | | | | | | | | | |
| medical | until End of treatment visit | | | | | | | | | |
| procedures | | | | | | | | | | |
| Clinical | | | | | | | | | | |
| laboratory tests | | | | | | | | | | |
| Pregnancy test | X | X⁸ | | | | | | | | |
| DPD | X¹¹ | | | | | | | | | |
| Clinical chemistry, | X | X⁵ | | | | X⁵ | | | | X |
| hematology and | | | | | | | | | | |
| coagulation | | | | | | | | | | |
| Urinalysis | X | X⁵ | | | | X⁵ | | | | |
| Pharmacokinetics | | X | | 5 min, | X | X | | | | X |
| | | | | 1 h ± | | | | | | |
| | | | | 10 min | | | | | | |
| | | | | 2 h ± | | | | | | |
| | | | | 10 min | | | | | | |
| | | | | 4 h ± | | | | | | |
| | | | | 20 min | | | | | | |
| Immunogenicity | | X⁵ | | | | | | | | |
| CT scan/Tumour | X⁹ | | | | | | | | | |
| response | | | | | | | | | | |
| evaluation | | | | | | | | | | |
| CA19-9 | X | X⁵ | | | | | | | | |
| Immunophenotyping | | X⁵ | | | X | X | | | | X |
| Cytokines and | | X⁵ | | 1 h ± | X | X | | | | X |
| chemokines | | | | 10 min | | | | | | |
| | | | | 4 h ± | | | | | | |
| | | | | 20 min | | | | | | |
| Whole | | X⁵ | | 1 h ± | X | X | | | | X |
| blood RNA | | | | 10 min | | | | | | |
| | | | | 4 h ± | | | | | | |
| | | | | 20 min | | | | | | |
| Whole | X | | | | | | | | | |
| blood DNA | | | | | | | | | | |
| Tumour | X¹⁰ | | | | | | | | | |
| biopsy | | | | | | | | | | |

| | Activity/Visit | | | | |
|---|---|---|---|---|---|
| | Cycle 1 (21-day treatment cycle) | | | | |
| | Day | | | | |
| | 10 ± 1³ | | 11 ± 1 | 17 ± 4 | |
| | Time in relation to study drug | | | | |
| | | | 24 ± | | Unscheduled |
| | Mitazalimab³ | Post² | 2 h² | | visit¹ |
| Informed | | | | | |
| Consent | | | | | |
| Eligibility | | | | | |
| criteria | | | | | |
| Demographics | | | | | |
| Medical | | | | | |
| history | | | | | |
| Body weight | | | | | X |
| Height | | | | | |
| Vital signs | | 1 h ± | | | X |
| | | 10 min | | | |
| | | 2 h ± | | | |
| | | 10 min | | | |
| Physical | | | | | X |
| examination | | | | | |
| 12-lead ECG | | 1h ± | | | X |
| | | 10 min | | | |
| ECOG | | | | | X |
| Treatment | | | | | X⁶ |
| discontinuation | | | | | |
| criteria | | | | | |
| Pre- and | Note: Premedications are to be given up to 3 days | | | | |
| post- | prior to administration of study treatment. | | | | |
| medication | | | | | |
| Mitazalimab | X⁷ | | | | |
| administration | | | | | |
| mFOLFIRINOX | | | | | |
| administration | | | | | |
| Oxaliplatin | | | | | |
| Irinotecan | | | | | |
| Leucovorin¹³ | | | | | |
| 5-FU | | | | | |
| Adverse | Continuous from signing ICF until 28 days | | | | |
| events | after last dose of study drug | | | | |
| Concomitant | Continuous from signing of ICF | | | | |
| medication | until End of treatment visit | | | | |
| Concomitant | Continuous from signing of ICF | | | | |
| medical | until End of treatment visit | | | | |
| procedures | | | | | |
| Clinical | | | | | |
| laboratory tests | | | | | |
| Pregnancy test | | | | | X |
| DPD | | | | | |
| Clinical chemistry, | | | X | X | X |
| hematology and | | | | | |
| coagulation | | | | | |
| Urinalysis | | | | | X |
| Pharmacokinetics | | 5 min, | X | X | X |
| | | 1 h ± | | | |
| | | 10 min | | | |
| | | 2 h ± | | | |
| | | 10 min | | | |
| | | 4 h ± | | | |
| | | 20 min | | | |
| Immunogenicity | | | | | X |
| CT scan/Tumour | | | | | X |
| response | | | | | |
| evaluation | | | | | |
| CA19-9 | | | | | X |
| Immunophenotyping | | | X | | X |
| Cytokines and | | 1 h ± | X | | X |
| chemokines | | 10 min | | | |
| | | 4 h ± | | | |
| | | 20 min | | | |
| Whole | | 1 h ± | X | | |
| blood RNA | | 10 min | | | |
| | | 4 h ± | | | |
| | | 20 min | | | |
| Whole | | | | | |
| blood DNA | | | | | |
| Tumour | | | | | |
| biopsy | | | | | |

¹The assessments to be performed during unscheduled visits should be based on Investigator's judgement.
²Assessments to be performed at specified time points after End of infusion. The End of infusion is defined as when the infusion of mitazalimab is completed/stopped (i.e., before rinsing if applicable). For example, 4 h post-dose means 4 hours after end of infusion.
³mFOLFIRINOX should be administered during 3 consecutive days. Mitazalimab should be administered on the same day as the day mFOLFIRINOX administration has ended or the day after.
⁴Informed consent must be obtained prior to performing any screening assessments
⁵The assessment may be performed within 72 hours (up to 3 days) prior to administration of study treatment. If the screening assessment were taken Day −1 to −3 prior to dosing, it does not need to be repeated.
⁶If any of the treatment discontinuation criteria apply, the patient should be discontinued from treatment and the End of treatment visit should be performed, see End of treatment visit in Table 4.
⁷If the infusion of mitazalimab is interrupted due to an AE, a PK sample and a sample for immunogenicity should be taken at the time of interruption, or as soon as is feasible considering the patient safety. An immunogenicity sample do not need to be taken if it is first administration of mitazalimab that is interrupted.
⁸The result of the pregnancy test must be available prior to dosing.
⁹The CT scan at screening may be obtained 28 days prior to first dose of mitazalimab.
¹⁰The baseline biopsy may be collected up to 28 days before first dose of mitazalimab. If a fresh biopsy cannot be taken during the screening period, archival biopsy material (most recent) could be used.
¹¹The DPD laboratory test may be collected up to 28 days before the first dose of mitazalimab.
¹²Body weight assessed before first dose of mitazalimab will be used for calculation of dose throughout study as long as the body weight do not change more than 10% from baseline.
¹³In case of supply issues with leucovorin, or to adhere to local standard of care practice, other approved similar folinates may be used under the supervision of an experienced clinician.

TABLE 5
Assessment schedule for Treatment cycle 2 and subsequent cycles

| | Activity/Visit | | | | | | | | | |
|---|---|---|---|---|---|---|---|---|---|---|
| | Cycle 2 and subsequent cycles (14-day treatment cycles) | | | | | | | | | End of treatment |
| | Cycle day | | | | | | | | | visit² |
| | 1³ | | 2 | 3 | 3 + 1 | | | 10 ± 4 | | 28-56 days |
| | Time in relation to study drug | | | | | | | | Unscheduled | after last |
| | Pre | mFOLFIRINOX⁴ | | | Pre | Mitazalimab⁴ | Post⁵ | | visit¹ | dose |
| Body weight | X⁶ | | | | | | | | X | |
| Vital signs | X | | | | X | | 1 h ± 10 min | X | X | X |
| Physical | X⁶ | | | | X⁶ | | | X | X | X |
| examination | | | | | | | | | | |
| 12-lead ECG | | | | | | | 1 h ± 10 min¹² | | X | X |
| ECOG | X⁶ | | | | | | | | X | X |
| Treatment | X⁷ | | | | X⁷ | | | | X⁷ | |
| discontinuation | | | | | | | | | | |
| criteria | | | | | | | | | | |
| Pre- and | Note: Premedications are to be given up to 3 days prior to administration of study treatment. | | | | | | | | | |
| post- | | | | | | | | | | |
| medication | | | | | | | | | | |
| mFOLFIRINOX | | | | | | | | | | |
| administration | | | | | | | | | | |
| Oxaliplatin | | X | | | | | | | | |
| Irinotecan | | X | | | | | | | | |
| Leucovorin¹³ | | X | | | | | | | | |
| 5-FU | | X | X | X | | | | | | |
| Mitazalimab | | | | | | X⁸ | | | | |
| administration | | | | | | | | | | |
| Adverse events | Continuous from signing ICF until 28 days after last dose of study treatment | | | | | | | | | |
| Concomitant | Continuous from signing of ICF until End of treatment visit | | | | | | | | | |
| medication | | | | | | | | | | |
| Concomitant | Continuous from signing of ICF until End of treatment visit | | | | | | | | | |
| medical | | | | | | | | | | |
| Procedures | | | | | | | | | | |
| Clinical | | | | | | | | | | |
| laboratory tests | | | | | | | | | | |
| Pregnancy test | X⁹ | | | | | | | | X | X |
| Clinical chemistry, | X⁶ | | | | X⁶ | | | X | X | X |
| hematology and | | | | | | | | | | |
| coagulation | | | | | | | | | | |
| Urinalysis | X⁶ | | | | | | | X | X | X |
| Pharmacokinetics | X | | | | | | 1 h ± 10 min | | X | X |
| | (Cycle 2, 4, 6, | | | | | | (Cycle 2, 4, 6, | | | |
| | 8, 10, 12) | | | | | | 8, 10, 12) | | | |
| Immunogenicity | X | | | | | | | | X | X |
| | (Cycle 2, 4, 6, | | | | | | | | | |
| | 8, 10, 12) | | | | | | | | | |
| CT scan/Tumour | | | | | | | | X | | X |
| response | | | | | | | | (Cycle | | |
| evaluation | | | | | | | | 4, 8, 12) | | |
| CA19-9 | X | | | | | | | | X | X |
| Immunophenotyping | X | | | | | | | | X | |
| | (Cycle 2, 4, 6, | | | | | | | | | |
| | 8, 10, 12) | | | | | | | | | |
| Cytokines and | X | | | | | | | | X | |
| chemokines | (Cycle 2, 4, 6, | | | | | | | | | |
| | 8, 10, 12) | | | | | | | | | |
| Whole | X | | | | | | | | | |
| blood RNA | (Cycle 2, 4, 6, | | | | | | | | | |
| | 8, 10, 12) | | | | | | | | | |
| Whole | X | | | | | | | | | |
| blood DNA | (Cycle 2, 4, 6, | | | | | | | | | |
| | 8, 10, 12) | | | | | | | | | |
| Tumour | | | | | | | | X¹⁰ | X¹⁰, ¹¹ | X^{10, 11} |
| biopsy | | | | | | | | (Cycle 2) | | |

¹The assessments to be performed during the unscheduled visits should be based on Investigator's judgement.
²After completion of the End of treatment visit, the patient will either enter the post-treatment follow-up period (please refer to Table 4), or if any of the study withdrawal criteria in Section Error! Reference source not found. apply, the patient will be withdrawn from the study and have the End of treatment visit and End of study visit assessments performed (please refer to the End of study visit in Table 4).
³Day 1 must be 14 ± 1 days after the start of the last mFOLFIRINOX administration.
⁴mFOLFIRINOX should be administered during 3 consecutive days. Mitazalimab should be administered on the same day as the day mFOLFIRINOX administration has ended or the day after.
⁵Assessments to be performed at specified time after End of infusion. The End of infusion is defined as when the infusion of IMP is completed/stopped (i.e. before rinsing if applicable). For example, 1 h post-dose means 1 hour after end of infusion.
⁶The assessment may be performed within 72 hours (i.e., up to 3 days) prior to administration of study treatment.
⁷If any of the treatment discontinuation criteria apply, the patient should be discontinued from treatment and the End of treatment visit should be performed.
⁸If the infusion of mitazalimab is interrupted due to an AE, a PK sample and a sample for immunogenicity should be taken at the time of interruption, or as soon as is feasible considering patient safety.
⁹The result of the pregnancy test must be available prior to dosing.
¹⁰Biopsy to be taken only if baseline biopsy (fresh or archival) was obtained at screening.
¹¹Biopsy not to be taken if already obtained in Cycle 2.
¹²Assessment to be obtained in Cycle 2 and 3 only.
¹³In case of supply issues with leucovorin, or to adhere to local standard of care practice, other approved similar folinates may be used under the supervision of an experienced clinician.

TABLE 6
Assessment schedule for post-treatment follow-up period and End of study visit

| | Activity/Visit | | |
|---|---|---|---|
| | Post-treatment follow-up | | End of study visit⁴ |
| | Time after End of treatment visit | | |
| | | 3 months, and thereafter | |
| | 1 month | every 3rd month ± 14 days | LPI + 2 years |
| Disease/Survival status | X¹ | X¹ | X¹ |
| Subsequent cancer-related therapy | X¹ | X¹ | X¹ |
| CT scan/Tumour response evaluation | | X² | X |
| Study withdrawal criteria | X³ | X³ | X |

¹Disease/Survival status and subsequent cancer-related therapy may be followed up via the patient's medical records (as allowed by local regulations) or phone contact.
²Assessment to be performed until new cancer-related therapy/continued treatment with chemotherapy backbone alone.
³If any of the study withdrawal criteria apply, the patient will be withdrawn from the study and have the End of study visit performed.
⁴End of study visit will be performed 2 years after last patient in (LPI), i.e. there will be a variation of duration of the treatment follow up period for individual patients.

### Inclusion Criteria

A patient is eligible to be included in the study if all the following criteria apply:
1. Has provided written informed consent
2. Is ≥18 years of age at the time of signing the informed consent form (ICF)
3. Has an Eastern Cooperative Oncology Group (ECOG) performance status of 0 or 1
4. Has a diagnosis of previously untreated metastatic pancreatic ductal adenocarcinoma (histologically documented)
5. Has measurable disease per RECIST v. 1.1
6. Has not received previous chemotherapy for pancreatic ductal adenocarcinoma
7. Has not received prior abdominal radiotherapy (except for palliative radiotherapy to non-target lesions)
8. Has a life expectancy of 3 months
9. Has acceptable hematologic laboratory values defined as:
   a. Neutrophils≥1.5×10⁹/L without growth factor stimulation within 3 weeks prior to the blood test
   b. Platelets≥100×10⁹/L
   c. Hemoglobin≥6.2 mmol/L (˜100 g/L) (may be after transfusion)
10. Has acceptable clinical chemistry laboratory values defined as:
   a. Bilirubin≤1.5×ULN (biliary drainage is permitted)
   b. AST≤3×ULN (irrespective of hepatic metastases)
   c. ALT≤3×ULN (irrespective of hepatic metastases)
   d. Creatinine≤1.5×ULN or glomerular filtration rate (GFR) of ≥45 mL/min (see Example 6 for calculation of GFR)
   e. INR≤1.5×ULN
   f. Albumin≥28 g/L
11. For women of childbearing potential¹:
   a. Has a negative highly sensitive serum (β-human chorionic gonadotropin [β-hCG]) pregnancy test at screening
   b. Is willing to use highly effective contraception methods during study treatment and for at least six months thereafter
12. Fertile men must practice effective contraceptive methods (i.e. surgical sterilization, or a condom used with a spermicide) during study treatment and for at least six months thereafter
13. Is willing to comply with all study procedures ¹ A woman is considered of childbearing potential (WOCBP), i.e. fertile, following menarche and until becoming post-menopausal unless permanently sterile. Permanent sterilisation methods include hysterectomy, bilateral salpingectomy and bilateral oophorectomy. A postmenopausal state is defined as no menses for 12 months without an alternative medical cause. A high follicle stimulating hormone (FSH) level in the postmenopausal range may be used to confirm a post-menopausal state in women not using hormonal contraception or hormonal replacement therapy. However, in the absence of 12 months of amenorrhea, a single FSH measurement is insufficient.

### Exclusion Criteria

A patient is excluded if any of the following criteria apply:
1. Has other types of non-ductal tumour of the pancreas, including endocrine tumours or acinar cell adenocarcinoma, cyst adenocarcinoma and ampullary carcinoma
2. Has other current cancer or history of cancer in the prior 3 years before signing the ICF other than in situ cervical cancer, or basal cell or squamous cell carcinoma treated with local excision only
3. Has known CNS metastases or carcinomatous meningitis
4. Has contraindication to any constituent of study treatment (mitazalimab and applicable chemotherapy)
5. Has a history of chronic diarrhea, inflammatory disease of the colon or rectum, or unresolved partial or complete intestinal obstruction
6. Has a history of myocardial infarction within 12 months of the first administration of mitazalimab, uncontrolled angina pectoris, unstable cardiac arrhythmias, or congestive heart failure of New York Heart Association class II or greater
7. Has QTc>450 msec
8. Has uncontrolled intercurrent illness, including active infection
9. Has a known history of HIV, hepatitis B or active hepatitis C infection
10. Is a female patient who is pregnant or nursing
11. Has received attenuated vaccine within 28 days before the first dose of study treatment
12. Any condition that, in the opinion of the Investigator, would place the patient at increased risk or preclude the patient's compliance with the study
13. Participates in another investigational drug or device study with any intervention within the previous 4 weeks prior to first dose of mitazalimab
   Additional Exclusion Criteria Only Applicable for mFOLFIRINOX Treatment:
14. Has received prior treatment with irinotecan or platinum-containing chemotherapy
15. Has pre-existing peripheral neuropathy greater than grade 1
16. Has known Gilbert's disease
17. Has known genotype UGT1A1*28/*28
18. Has known fructose intolerance (malabsorption)
19. Has complete dihydropyrimidine dehydrogenase (DPD) deficiency

### Additional Exclusion Criteria Only Applicable for Gemcitabine Plus Nab-Paclitaxel Treatment:

14. Has a history of slowly progressive dyspnea and unproductive cough, or of conditions such as sarcoidosis, silicosis, idiopathic pulmonary fibrosis, pulmonary hypersensitivity, pneumonitis or multiple allergies
15. Has a history of Peripheral Artery Disease (eg, claudication, Leo Buerger's disease)
16. Has a history of connective tissue disorders (eg, lupus, scleroderma, arteritis nodosa)

### Treatment

### Investigational Medicinal Product (IMP)—Mitazalimab

The IMP mitazalimab is an agonistic human monoclonal (IgG1) antibody targeting CD40, developed for cancer immunotherapy.

### Manufacturing

Mitazalimab is manufactured by Biogen (USA) according to GMP. The product is manufactured using a stable Chinese Hamster Ovarian (CHO) cell line in a fed batch bioreactor. The product does not contain any components of animal origin and has been reviewed to be safe from an adventitious agent perspective.

### Formulation, Packaging and Labelling

Mitazalimab may be supplied as a lyophilized cake in single-use glass vials, in which case it can be reconstituted using sterile water for injection (WFI) prior to use. Each vial requires the addition of 2 mL sterile WFI to fully dissolve the cake. Each vial contains 40 mg mitazalimab, and the resulting concentration after reconstitution will be 20 mg/mL. The exact concentration will be stated on the primary vial. The packaging, labelling and distribution to the clinical sites will be performed by Fisher Clinical Services (FCS). The packaging and labelling will be done at FCS site in Allentown, Pennsylvania USA and the distribution to the sites will be done by FCS in Germany according to Good Manufacturing Practice (GMP) under the responsibility of the Sponsor. For further details regarding formulation, packaging and labelling, please refer to the Mitazalimab Investigational Medicinal Product Dossier (IMPD).

### Handling, Preparation and Storage

The mitazalimab vials may be stored refrigerated at the local pharmacy at a temperature of 2-8° C. and protected from light in a secure area according to local regulations. The vials must not be used after the expiry date. The preparation of mitazalimab solution for infusion will be carried out at the local pharmacy according to instructions in the Pharmacy Manual. After reconstitution, mitazalimab is to be diluted in physiological saline or 5% dextrose in water. The prepared mitazalimab solution for infusion is preferably kept at room temperature protected from light in a secure area according to local regulations. The mitazalimab solution for infusion can be infused within 16 hours from the reconstitution of the lyophilized cake. For detailed guidance on study drug preparation, handling and storage, please refer to the Pharmacy Manual.

### Administration of Mitazalimab

Mitazalimab will be administered intravenously during a 2-hour rate-controlled infusion. Weight at baseline will be used to calculate the mitazalimab dose throughout the study. If the pre-dose assessment of weight assessed in the beginning of each treatment cycle change>10% from baseline, the mitazalimab dose can be recalculated and adjusted accordingly. All patients will be monitored for at least 4 hours after the end of the first infusion of mitazalimab, and for at least 2 hours after the second mitazalimab infusion. If infusion-related reactions have not been observed at the latest infusion (the 2^{nd} or later infusion), the monitoring of the patient can be reduced to 1 hour for subsequent infusions.

### Mitazalimab Dose Modifications

The dose and dosing frequency of mitazalimab for an individual patient may be modified in the following ways:
The dose may be reduced due to an AE:
   If an adverse event is possibly, probably or definitely related to mitazalimab and results in more than two weeks of treatment delay, the dose of mitazalimab will be reduced by 50%. If the dose has already been reduced once, mitazalimab treatment will stop; and/or
   If an adverse event is serious or greater than grade 2 and is possibly, probably or definitely related to mitazalimab, mitazalimab will be held until the AE resolves to the greater of the baseline grade or grade 1 or less; at that time mitazalimab may be resumed at the investigator's discretion either at the previous dose, or if not already dose reduced, at a 50% dose reduction; the investigator may also elect to forego rechallenge.
A pre-dose may be introduced due to an AE, e.g., infusion-related AEs.

### Handling of Infusion-Related Reactions

If clinically significant symptoms of an infusion-related reaction occur, the infusion can be temporarily stopped (interrupted). Upon recovery, the infusion can be resumed at 50% of the rate at which the reaction occurred for at least 30 minutes. If there is no reoccurrence of clinically significant symptoms after 30 minutes, the infusion rate can be increased according to the original infusion plan. Lower infusion rates may be selected, for example if clinically indicated.

Depending on the time of occurrence and the severity of the reaction, the Investigator may consider administering supportive medication, e.g., antihistamines, acetaminophen or corticosteroids, in addition to the mandatory pre- and post-medications described herein.

If severe infusion-related reactions are observed, the DRC may introduce changes to the mitazalimab infusion.

If AEs which are considered related to the infusion, e.g., infusion-related reactions, are not well controlled by the above schedule, the DRC can decide to introduce a pre-dose of mitazalimab on the day prior to first administration. The pre-dose will be a maximum of 10% of the full doses. A pre-dose could be set forth for all patients enrolled after the decision by the DRC.

Cytokine release syndrome may be indistinguishable from infusion-related reactions if the symptoms occur in relation to the infusion. Cytokine release syndrome may also have many symptoms that resemble an infection or even sepsis. Cytokine release syndrome as reported with CD3 targeting antibodies or CAR T cell infusion has not been observed with mitazalimab.

The preferred term when reporting reactions considered related to the infusion will be infusion-related reaction rather than cytokine release syndrome. Infusion-related reactions of grade 2 or higher should be reported as adverse events of special interest (AESIs).

### Handling of Impaired Liver Parameters

Hepatic injury is defined by the following alterations of liver parameters:
For patients with normal liver function (ALT, AST and bilirubin within normal limits) at baseline an elevation of AST and/or ALT>3-fold ULN combined with an elevation of bilirubin>2-fold ULN measured in the same blood draw sample; and/or
For patients with impaired liver function at baseline, i.e., above ULN for AST or ALT, an elevation of AST and/or ALT>5-fold ULN combined with an elevation of total bilirubin>2-fold ULN measured in the same blood draw sample.

If laboratory values consistent with hepatic injury as defined above are observed the following laboratory tests can be repeated within 48 to 72 hours: ALT, AST, and bilirubin (total and direct). If hepatobiliary toxicity is observed, treatment can be held to allow evaluation of alternative causes, e.g., biliary obstruction/stent malfunction. Other causes may need to be followed up according to Example 7 Drug-induced Liver Injury (DILI). Increased AST or ALT of grade 3 or higher, and bilirubin of grade 2 or higher should be reported as AESIs.

### Non-Investigational Medicinal Products

### Mitazalimab Pre- and Post-Medication

Pre- and post-medications may be given in connection with each mitazalimab administration. The premedication can start 3 days prior to mitazalimab infusion. The advantageous pre- and post-medications are listed in Table 7 below and include types of medication and timing of intake in relation to mitazalimab infusion.

Antihistamine H1 and leukotriene inhibitor can be given as premedication, for example starting 3 days prior to mitazalimab administration.

TABLE 7
Pre- and post-medication to be administered together with mitazalimab

| | | |
|---|---|---|
| Type of medication | Agent and dose | Exemplary schedule |

Premedication:

| | | |
|---|---|---|
| Antihistamine (H1) | E.g., Cetirizine 10 mg | Daily morning intake starting 3 days |
| | PO | prior to start of mitazalimab |
| | | infusion. On day of dosing 1-3 hours |
| | | prior to start of mitazalimab |
| | | infusion. |
| Leukotriene inhibitor | Montelukast 10 mg PO | Daily morning intake starting 3 days |
| | | prior to start of mitazalimab |
| | | infusion. On day of dosing 1-3 hours |
| | | prior to start of mitazalimab |
| | | infusion. |
| Antihistamine (H2) | E.g., Famotidine 40 mg | At least 30 minutes prior to start of |
| | PO (optional) | mitazalimab infusion. |
| Antipyretic | E.g., Acetaminophen | At least 30 min prior to start of |
| | 650-1000 mg PO | mitazalimab infusion. |
| Antiemetic | E.g., Ondansetron 8 mg | At least 30 minutes prior to start of |
| | PO or IV (optional) | mitazalimab infusion. |

Post-medication:

| | | |
|---|---|---|
| Leukotriene inhibitor | Montelukast 10 mg PO | Morning intake on the two following |
| | | days after mitazalimab |
| | | administration. |
| Antihistamine (H1) | E.g., Cetirizine 10 mg | Morning intake on the 2 following |
| | PO | days after mitazalimab |
| | | administration. |

mFOLFIRINOX

The mFOLFIRINOX used in this study is comprised of the constituents oxaliplatin, leucovorin, irinotecan and 5-fluorouracil (5-FU) and is considered standard of care for first line treatment of advanced pancreatic cancer by the European Society of Medical Oncology (ESMO) and the National Comprehensive Cancer Network (NCCN) based on evidence from large phase 3 trials.

The original “full” FOLFIRINOX regimen defined in ACCORD 11/0402, a 342-patient phase 3 study, consisted of a 400 mg bolus of 5-FU followed by 2400 mg/m² 5-FU over 46 hours, 400 mg/m² leucovorin, 85 mg/m² oxaliplatin, and 180 mg/m² irinotecan. Over the last decade, it has become common practice to administer less intense versions of this regimen, known collectively as mFOLFIRINOX, to reduce side effects. Current practice guidelines place mFOLFIRINOX regimens on par with full FOLFIRINOX as a preferred regimen for first line treatment of patients with metastatic pancreatic cancer and good performance status (NCCN Clinical Practice Guidelines in Oncology. Pancreatic Adenocarcinoma. Version 1. 2021. Accessible at the following webpage: https://www.nccn.org/professionals/physician_gls/pdf/pancreatic.pdf).

Based on phase 2 trials, other variations of FOLFIRINOX have been recognized as acceptable alternatives that retain comparable clinical efficacy with an improved side effect profile. These have included, for example, regimens reducing all components by 20% [30] or reducing the 5-FU bolus to 300 mg and reducing the irinotecan to 135 mg/m² [31].

Other clinical trials that have sought to combine novel agents with FOLFIRINOX have opted for similar modifications of FOLFIRINOX, for example, no 5-FU bolus was administered in a study combining FOLFIRINOX with Hedgehog inhibitor IPI-926 [32]. In the AVENGER 500 trial combining FOLFIRINOX with CPI-613, the 5-FU component was kept at full strength including the bolus, but oxaliplatin was reduced to 65 mg/m² and irinotecan to 140 mg/m² [33].

The mFOLFIRINOX regimen employed in the current study is identical to the one used in the phase 3 PRODIGE 24 trial adjuvant pancreatic study and differs from the original FOLFIRINOX regimen in that the 5-FU bolus is omitted, and the irinotecan dose is 150 mg/m² [34].

Administration of mFOLFIRINOX

Mitazalimab can be administered in combination with the chemotherapy regimen mFOLFIRINOX as first choice. mFOLFIRINOX may be comprised of the constituents oxaliplatin, leucovorin, irinotecan and/or 5-FU. The recommended parameters for timing and sequence of infusion of constituents of mFOLFIRINOX are described in Table 8 below.

Leucovorin is one of several folinates that can be used in combination with 5-FU in cytotoxic therapy. Leucovorin potentiates 5-FU's inhibition of thymidylate synthase, enhancing 5-FU's antimetabolic activity. Alternative folinates to leucovorin may be utilized based on local availability including but not limited to calcium folinate, calcium levofolinate, disodium folinate and disodium levofolinate. Dosing based on folinic acid as defined in the relevant SmPCs can be followed. Where leucovorin is referenced within this application it is acknowledged that similar approved folinates can be used.

Variations in the administration are permitted, which may be achievable based on drug dosing and modification guidelines, such as those described in Example 4.

Weight and height at baselines can be used to calculate the doses of mFOLFIRNOX constituents. Doses can be re-adjusted if the patient's body surface area (BSA) changes by >10% from baseline. If the patient's BSA changes by ≤10%, no adjustment is necessary unless the site has a standard procedure to adjust doses based upon current BSA according to institutional guidelines.

TABLE 8
mFOLFIRINOX administration
mFOLFIRINOX regimen

| Agent | Dose | Route | Schedule |
|---|---|---|---|
| Oxaliplatin | 85 | IV, 2 h infusion | Every 14 days, (Day 8 in |
| | mg/m² | | Cycle 1, and Day 1 in |
| Leucovorin | 400 | IV, 2 h infusion | subsequent cycles) |
| | mg/m² | | |
| Irinotecan | 150 | IV, 90 minute | |
| | mg/m² | infusion started 30 | |
| | | minutes after end | |
| | | of the leucovorin | |
| | | infusion | |
| 5-FU | 2400 | 46-48 hour | Every 14 days (Day 8-10 in |
| | mg/m² | infusion IV | Cycle 1 and Day 1-3 in |
| | | | subsequent cycles) |

### Recommended Infusion Timing and Sequence:

Oxaliplatin IV over 2 hours immediately followed by;
Leucovorin or similar approved folinates over 2 hours;
Irinotecan administered over 90 minutes (starting 30 minutes after start of the leucovorin infusion), followed by;
5-FU infusion over 46-48 hours.

If one of the mFOLFIRINOX constituents is stopped, one or more of the other constituents may be given. In case mFOLFIRINOX is not deemed safe and tolerable in combination with the lowest planned dose level of mitazalimab (450 μg/kg), mFOLFIRINOX may be exchanged to gemcitabine plus nab-paclitaxel, and/or a lower dose of mitazalimab may be used. The dosage schedule and dosing modifications allowed for gemcitabine plus nab-paclitaxel are described in Example 5.

mFOLFIRINOX Pre- and Post-Medications.

Pre- and post-medications may be given in connection with each mFOLFIRINOX administration. The suggested premedication will start an hour prior to administration of oxaliplatin, the first constituents of mFOLFIRINOX. The suggested pre- and post-medications are listed below.

The suggested premedication can include one or more of the following medications given 30-60 minutes prior to the infusion:
NK1-receptor antagonist, e.g., Aprepritant, 125 mg PO, 60 min prior to infusion and can be continued during days with chemotherapy. Aprepitant is a cytochrome 2C9 inducer and may inactivate some oral contraceptives. Therefore, women of child-bearing potential may use an alternative means of contraception if Aprepitant is administered.
5-HT3 receptor antagonist, e.g., Ondansetron, 8 mg PO, 30 min prior to infusion and can be continued during days with chemotherapy.
Corticosteroid, e.g., dexamethasone 8 mg IV or PO, 30 min prior to infusion.

The post-medication can include the following medications:
G-CSF, e.g., Neulasta, 6 mg SC, on the fourth day following start of mFOLFIRINOX regimen (at least 24 h after the end of the continuous 5-FU iv infusion).

Alternative pre- and post-medications to mFOLFIRINOX are allowed.

### Gemcitabine and Nab-Paclitaxel (if Applicable)

Gemcitabine and nab-paclitaxel are considered standard of care for first line treatment of advanced metastatic pancreatic cancer by the European Society of Medical Oncology (ESMO) and the National Comprehensive Cancer Network (NCCN) based on evidence from large, randomised phase 3 trials.

Participants should, prior to study treatment, be informed to seek advice about donation and cryopreservation of germlines because of the possibility of irreversible infertility caused by treatment with gemcitabine or nab-paclitaxel.

Gemcitabine plus nab-paclitaxel can be supplied/obtained according to Clinical Study Agreements and in accordance with local guidelines. Gemcitabine and nab-paclitaxel will be stored and handled according to package inserts and stored in a secure place under appropriate storage conditions.

### Administration of Gemcitabine and Nab-Paclitaxel

Gemcitabine plus nab-paclitaxel can be given in combination with mitazalimab in case mFOLFIRINOX is found not viable for a particular subject. The recommended (exemplary) parameters for timing and sequence of infusion for gemcitabine and nab-paclitaxel, respectively, are listed in Table 9 below. Variations in the administration are permitted, provided drug dosing and modification guidelines are followed, see Example 5.

TABLE 9
Gemcitabine and nab-paclitaxel administration

| Agent | Dose | | Route | Schedule |
|---|---|---|---|---|
| Nab-paclitaxel | 125 | mg/m² | IV | Day 8, 15 and 22 in |
| Gemcitabine | 1000 | mg/m² | IV | Treatment cycle 1 (35-day |
| | | | | treatment cycle) and Day |
| | | | | 1, 8, 15 of subsequent |
| | | | | treatment cycles (28-day |
| | | | | treatment cycles) |

### Recommended Infusion Timing and Sequence:

Nab-paclitaxel infused over 30-40 min;
Gemcitabine infused over 30 min, immediately after completion of nab-paclitaxel infusion.

### Study Assessments

### Demographics and Medical History

Age, gender, race and ethnic origin of study participants were recorded. Clinically significant abnormal findings observed during the physical examination and non-serious AEs occurring pre-treatment (i.e., after signing the ICF but before first dose) can be recorded as medical history.

Cancer Disease Status, Including Other Cancer than Study Disease

The date of initial histopathological diagnosis of pancreatic adenocarcinoma was recorded as well as the disease stage at time of diagnosis and the current disease stage. Surgery for the cancer can be recorded, including information if the surgery was of curative intention. Radiotherapy can be recorded, also when given for palliative purpose.

Any prior anti-cancer treatment for other cancer disease than pancreatic adenocarcinoma (if applicable) can be recorded, including:
Treatment identity, including surgery, radiotherapy and chemotherapy as applicable;
Start and end date of treatment;
Best response (CR, PR, SD, PD and NE);
Reason for discontinuation (for treatments stopped before initially planned).

### Body Weight and Height

Body weight (without overcoat and shoes) was measured at screening and during treatment and rounded to the nearest kilogram. The dose calculation of mitazalimab is based on the body weight. In case the body weight changes more than 10% from baseline measurement, the mitazalimab dose can be re-calculated. The dose calculation for constituents of mFOLFIRINOX, gemcitabine and nab-paclitaxel are based on BSA. In case the BSA change more than 10% based on calculation using baseline measurement of body weight, the dose of constituents of mFOLFIRINOX, gemcitabine and nab-paclitaxel can be re-calculated. Additional body weight assessments may be performed based on Investigator's judgement at regular visits or at additional (Unscheduled) visits. Height (without shoes) was measured at screening and rounded to the nearest centimetre.

### Vital Signs

The vital sign measurements included systolic and diastolic blood pressure, pulse rate, oxygen saturation and body temperature. Blood pressure determinations can, preferably using the same equipment within each visit, be made after the patient has rested for 10 minutes. Any new or aggravated clinically significant abnormal findings as compared with the pre-treatment assessment will be reported as an AE. Additional vital sign assessments may be performed based on Investigator's judgement at regular visits or at additional (Unscheduled) visits.

### Physical Examination

Physical examination can include general appearance and examination of the following body systems: eyes, mouth and throat, lymph node regions, respiratory, cardiovascular system, abdomen, extremities, and skin. An examination of the neurological system may be done based on the Investigator's judgement. Any new or aggravated clinically significant abnormal medical findings as compared with the pre-treatment assessment can be reported as an AE. Additional assessments of physical examination may be performed based on Investigator's judgement at regular visits or at additional (Unscheduled) visits.

### Electrocardiogram (ECG)

Standard 12-lead ECGs were recorded, and an overall interpretation of the ECGs was performed by the Investigator or, if applicable, the Investigator delegated this task to a cardiologist. The ECG printout was signed and dated following review and interpretation. For the ECG recordings, the patients must be resting and in horizontal or half laid position for at least 10 minutes. The same method of assessment can be used throughout the study. Any irregularity observed or occurring during the ECGs (e.g., vomiting, cough) can induce a repeat of the ECG. Any new or aggravated clinically significant abnormal ECG findings as compared with the pre-treatment assessment can be reported as an AE. Additional ECGs recordings may be performed based on Investigator's judgement at regular visits or at additional (Unscheduled) visits.

### ECOG Performance Status

The Eastern Cooperative Oncology Group (ECOG) performance status scale [35] provided in Table 10 below, was used by the Investigator to grade the patients' performance status of daily living activities.

TABLE 10
ECOG performance status

| Grade | ECOG |
|---|---|
| 0 | Fully active, able to carry on all pre-disease performance without |
| | restriction. |
| 1 | Restricted in physically strenuous activity but ambulatory and |
| | able to carry out work of a light or sedentary nature, e.g., light |
| | housework, office work. |
| 2 | Ambulatory and capable of all self-care but unable to carry out |
| | any work activities. Up and about more than 50% of waking |
| | hours. |
| 3 | Capable of only limited self-care, confined to bed or chair more |
| | than 50% of waking hours. |
| 4 | Completely disabled. Cannot carry on any self-care. Totally |
| | confined to bed or chair. |
| 5 | Dead |

### Clinical Laboratory Tests

The time points for the clinical laboratory assessments are specified in Section 0 VISIT ASSESSMENT SCHEDULE. However, more frequent tests may be performed if indicated by the clinical condition of the patient or by abnormalities that warrant more frequent monitoring as judged by the Investigator. The screening laboratory results must be available and reviewed by the Investigator before the first dose of mitazalimab. The results from the clinical laboratory tests were summarized in the Clinical Study Report. Deterioration as compared to pre-treatment in these parameters will therefore only be reported as AEs if they fulfil any of the criteria for a SAE or are the reason for modifying the study treatment. Deterioration of a laboratory value that is unequivocally due to disease progression will not be reported as an AE.

The procedures for blood and urine sample collection, preparation and handling were performed as per local procedures. The DPD test taken at screening can be analysed at a central laboratory or per local procedures. The clinical laboratory tests to be performed are listed in Table 11. All tests can be performed by the local laboratory, although the urinalysis tests can be performed and assessed by the site personnel. If an abnormal urinalysis result (urine dipstick) is regarded as clinically significant, microscopy can be used to measure sediment, i.e., red blood cells, white blood cells, epithelial cells, crystals, casts, and culture of bacteria.

TABLE 11
Clinical laboratory tests

| Dihydropyrimidine | DPD assessed during screening only when mitazalimab is given in | |
|---|---|---|
| dehydrogenase (DPD) | combination with mFOLFIRINOX. Analysed at central laboratory or | |
| | per local procedures. | |
| Pregnancy testing (for women of | Serum β-human chorionic gonadotropin (β-hCG) pregnancy test is | |
| childbearing potential only) | required at screening, at the other time points either serum or urine | |
| | pregnancy tests can be used. | |
| | The results of the pregnancy test must be available prior to each | |
| | drug administration. | |
| Clinical chemistry | Creatinine | Lactate dehydrogenase |
| | Total bilirubin | (LDH) |
| | Alanine aminotransferase (ALT) | Urea or Blood urea |
| | Aspartate aminotransferase (AST) | nitrogen (BUN) |
| | Alkaline phosphatase | Uric acid |
| | Albumin | Phosphorus |
| | Total protein | Bicarbonate |
| | Total calcium | Sodium |
| | Glucose | Potassium |
| | Creatine kinase | Magnesium |
| | C-reactive protein | Chloride (if feasible) |
| Hematology | White blood cell count with | Hemoglobin |
| | differential (absolute) counts | Platelet count |
| | (neutrophils, eosinophils, | |
| | lymphocytes, and monocytes, | |
| | basophils) | |
| Coagulation | International normalized ratio (INR) | D-dimer |
| | aPTT | Fibrinogen |
| Urinalysis (dipstick) | Protein | Ketones |
| | Hemoglobin/blood | Bilirubin |
| | Glucose | Urobilinogen |
| | Specific gravity | Nitrite |
| | pH | Leukocyte esterase |

### Immunogenicity (Anti-Drug Antibodies)

Blood samples (serum) were taken for immunogenicity testing according to Section 0 VISIT ASSESSMENT SCHEDULE for mitazalimab in combination with mFOLFIRINOX, and according to visit assessment tables in Example 5 for mitazalimab in combination with gemcitabine and nab-paclitaxel. Samples may also be collected at additional time points, at regular visits or at additional (Unscheduled) visits, based on Investigator's judgement. If the infusion of mitazalimab is interrupted due to an AE, a sample for immunogenicity can be collected (except during the first infusion) at the time of interruption or as soon as feasible considering patient safety together with a PK sample. The samples for immunogenicity testing can be used for anti-drug antibody (ADA) analysis (i.e., antibodies to mitazalimab). The samples analysed for immunogenicity and confirmed positive can be tested for neutralizing antibodies. Other analyses may be performed to further characterize the immunogenicity of mitazalimab. Immune response analysis may be conducted on PK samples collected at other timepoints noted in the visit assessment schedule if deemed necessary. Details regarding sample collection and processing will be provided in the Laboratory Manual.

### Pharmacokinetics (PK)

Blood samples (serum) were taken for analysis of mitazalimab concentrations and PK analysis according to Section 0 VISIT ASSESSMENT SCHEDULE for mitazalimab in combination with mFOLFIRINOX, and Example 5 for mitazalimab in combination with gemcitabine and nab-paclitaxel. PK samples may be collected at additional time points, at regular visits or at additional (Unscheduled) visits, based on Investigator's judgement. The samples for PK analysis are preferably taken from a peripheral vein contralateral to the arm into which mitazalimab is infused. If the infusion of mitazalimab is interrupted due to an AE, a PK sample and a sample for immunogenicity can be collected (except during the first infusion when no sample for immunogenicity needs to be taken) at the time of interruption or as soon as feasible considering patient safety.

The following PK parameters will be derived for mitazalimab:
Cₘₐₓ
Tₘₐₓ
AUC_{(0-T)}

Other PK parameters may be derived if data allows such as:
AUC_{0-∞}
AUCₜ
Elimination half-life (T_{1/2})
Total serum clearance (CL)
Volume of distribution (V_{d})

### Assessment of Anti-Tumour Activity

### Computed Tomography (CT) Scan

A CT scan of chest/abdomen/pelvis can be taken according to local practice. Other body areas may also be CT scanned if needed to assess the tumour(s) (e.g., a CT scan of neck would be needed for a patient having cervical nodes or a head and neck tumour). Additional CT scans may be taken based on Investigator's judgement at regular visits or at additional (Unscheduled) visits. The use of intravenous contrast is at the discretion of the radiologist performing the scanning, but imaging must be consistent per patient throughout the study.

If a CT scan is considered not feasible, as judged by the Investigator, a Magnetic Resonance Imaging (MRI) may be performed. The same scanning modality is preferably used throughout the study. The Investigator and/or radiologist identified the tumours to be followed throughout the study.

A CT scan is performed at screening (within 28 days of first dose), 9 weeks after start of study treatment (end of cycle 4 for mFOLFIRINOX combination and at end of Cycle 2 for gemcitabine plus nab-paclitaxel treatment) and thereafter at every 8^{th} week assuming no treatment delays. Additional CT scans may be performed at the discretion of the Investigator at unscheduled visits. The CT scans were evaluated according to RECIST v. 1.1. Patients with response (PR or CR) can have a confirmatory CT scan at least 4 weeks later to confirm the response. If the patients have progressive disease, the patients should discontinue treatment.

### Tumour Response Evaluation

The evaluation of tumour response was done according to RECIST v. 1.1 by the investigational sites using CT scans according to Example 3 RECIST v. 1.1 guideline. Patients with PD can discontinue study treatment. However, patients with suspected progression are allowed to continue on treatment if they are considered to be clinically stable in the opinion of the Investigator until clinical or radiological progression is documented.

### Pharmacodynamics

The aim with the pharmacodynamic biomarkers is to characterize changes in intratumoural and systemic immune activation associated with mode of action of mitazalimab and correlate pharmacodynamic effects with clinical response. Disease progression and treatment response will also be followed with liquid biopsies, analysing biomarkers such as Carbohydrate antigen 19-9 (CA19-9) and circulating tumour DNA (ctDNA). All biomarker analyses in blood and tumour biopsies may be exploratory in nature. All exploratory analyses were performed at a fit-for-purpose laboratory, as the data from the analyses are for scientific use and decision making only. The data may be included in a scientific publication but will not be included in the clinical study report.

### Blood

The blood samples will be taken according to Section 1 Visit Assessment Schedule, for mitazalimab in combination with mFOLFIRINOX, and according to Example 5 for mitazalimab in combination with gemcitabine plus nab-paclitaxel.

Different types of blood samples will be taken and the following pharmacodynamic biomarkers may be evaluated:
Cytokines and chemokines: Serum samples are to be analysed for levels of cytokines and chemokines involved in the immune activation of mitazalimab, including but not limited to IFN-γ, TNF-α, IL12p70, IL-6, MCP-1, IP-10, MIP-1α, MIP-1β and IL-8; e.g., using a 30-plex kit with Luminex, MSD or similar.
Immune phenotyping: Whole blood samples will be used for immunophenotyping of whole blood for quantification of immune cell populations and immune cell activation directly or indirectly involved with immune activation of mitazalimab using flow cytometry. The following biomarker panels may be analysed:
   T/NK/NKT cells (e.g., CD45, CD3, CD8, CD4, CD16, CD56, CCR7, CD45RA+activation markers CD25, Ki67);
   B cells (e.g., CD45, CD19, CD27, IgD+activation markers CD86, CD83, CD54, HL-DR); and/or
   Monocytes and DCs (CD45, CD14, CD16, CD11c, CD123+activation markers CD54, CD86, HLA-DR, CD83).
Whole blood RNA: Whole blood RNA samples will be collected for analyzation of gene signatures involved in the immune activation with mitazalimab (including but not limited to: immunoscore, IFNγ genes profile, APC gene profile and T cell gene profile) and prognostic tumour gene profiles (e.g., TMB and MSI-high) and can be analysed with e.g., Nanostring IO 360, RNAseq, or similar.
Whole blood DNA: Cell free whole blood DNA samples will be collected for evaluation of circulating tumour DNA (ctDNA). Changes in tumour specific genes e.g., KRAS will be followed as a biomarker to monitor treatment response. Whole blood DNA can be collected for evaluation of TCRb clonality.
CA19-9: Changes in the levels of pancreatic serum protein CA19-9 will be followed as a biomarker for monitoring disease progression and treatment response.

### Tumour Biopsy

Tumour biopsies were collected according to Section 1 Visit Assessment Schedule, for mitazalimab in combination with mFOLFIRINOX, and according to Example 5 for mitazalimab in combination with gemcitabine plus nab-paclitaxel. Collection of biopsies may be omitted in case the tumour is inaccessible or the biopsy procedure expose the patient to an increased risk, as judged by the Investigator. If a biopsy cannot be taken at screening, archival biopsy material (i.e., tissue slides or tissue block, preferably from the most recently collected tumour biopsy prior to enrolment) can be collected if possible. If no biopsy is obtained (fresh nor archival) at screening, no further biopsies will be collected during the study. Biopsies (core biopsy, 18-gauge needle) will be taken under local anaesthesia. The tumour biopsies are preferably taken from the same tumour throughout the study. Imaging can be used to guide the biopsies. Tumour tissue collected by punch biopsy or excisional biopsy are acceptable alternatives for cutaneous tumours. Anticoagulation medication can be paused as medically indicated prior to a tumour biopsy is performed. The biopsies can be formalin-fixed and paraffin-embedded.

Tumour biopsy analysis may include:
Immunohistochemistry: Archival or freshly collected tumour biopsies may be analysed for CD40 target expression and immune cell infiltration at baseline and correlate with clinical response. On treatment biopsies will be compared with baseline biopsies for analyzation of immune cell infiltration and immune activation induced by mitazalimab for proof of mechanism. The following immunohistochemistry (IHC) panels may be included:
   CD40 target expression;
   T cell infiltration and activation status;
   Macrophage infiltration with M1/M2 differentiation and activation status;
   Cell surface markers related to immune regulation e.g., PD-L1; and/or
   Other markers describing proof of mechanism such as e.g., fibrotic markers and stroma (e.g., fibronectin and collagen type I) macrophage functionality related to macrophage functionality.
Gene profiling: Baseline and on treatment tumour biopsies will be collected for analyzation of gene signatures involved in the immune activation with mitazalimab (including but not limited to: immunoscore, IFNγ genes profile, APC gene profile and T cell gene profile) and prognostic tumour gene profiles (e.g., TMB and MSI-high) and can be analysed with e.g., Nanostring IO 360, RNAseq. or similar.

Any tumour biopsy material remaining after the analysis described, was stored in a biobank, for possible future analyses.

### Disease/Survival Status (Post Treatment Follow-Up)

During the post-treatment follow-up, disease and survival status were collected, see Table 4 in Section 0 VISIT ASSESSMENT SCHEDULE for mitazalimab in combination with mFOLFIRINOX and Table 22 in Example 5 for mitazalimab in combination with gemcitabine plus nab-paclitaxel.

The following will be assessed:
Survival; and/or
Current disease stage.

If the patient died, the date and cause of death will be collected and documented. Disease/survival status may be followed up via the patient's medical records (as allowed by local regulations) or phone contact. Where allowed by local law, public records may be used to document death for the purpose of obtaining survival status.

### Subsequent Cancer-Related Therapy (Post-Treatment Follow-Up)

During the post-treatment follow-up, all subsequent cancer-related therapies were collected. Subsequent cancer-related therapy may be followed up via the patient's medical records (as allowed by local regulations) or phone contact.

### Statistics

### Study Hypotheses

No formal statistical hypothesis is defined for Part 1 (Phase 1b).

For Part 2 (Phase 2), the null hypothesis for the primary endpoint ORR is:

$${H}_{0} : ORR = 30 \%$$

The null hypothesis was tested against the alternative:

$${H}_{1} : ORR > 30 \%$$

### Sample Size Considerations

In Part 1, it was estimated that at least 9 patients would be included in two dose levels with at least 3 patients on each dose and 6 patients at the RP2D. For Part 2 the sample size calculation was based on an assumption of 30% ORR for mFOLFIRINOX [4]. Adding mitazalimab was assumed to increase the ORR to approximately 45%. Gemcitabine plus nab-paclitaxel was assumed to have similar ORR (29%) as mFOLFIRINOX [5] and all other assumptions were the same, hence, the sample size estimation was considered valid for both treatment options. The sample size estimation was based on an extended Simon's two-stage design with break for futility and efficacy [36]. The optimal design was selected as it minimized the expected sample size. There were two cut-offs for number of responders in the interim analysis: one for stopping for futility and one for stopping for efficacy. If the number of responders was between these cut-offs the study continues. Table 12 present the sample size.

TABLE 12
Extended Simon's two-stage design
Design restrictions: ORR ≥ 30% historic ORR = 30%, alpha (one-sided) = 0.10

| | | | | | | | Second | | Est. N | | |
|---|---|---|---|---|---|---|---|---|---|---|---|
| ORR | Power | | First stage | | | | stage | | given | PET_{H0} | PET_{H1} |
| (%) | (%) | Design | n₁ | r_{fut} [a] | r_{eff} [b] | ORR_{eff} [c] | n | r_{fut} [a] | H0 | (%) | (%) |
| 45 | 80 | Optimal | 23 | 7 | 11 | 52.2 | 54 | 20 | 34.17 | 63.96 | 42.87 |

[a] If number of responders is equal to or below this number the study is stopped for futility/not conclusive
[b] If number of responders is above this number the study is stopped for efficacy
[c] ORR needed in first stage to stop for efficacy (r_{eff} + 1/n₁)
PET_{H0} = Probability of Early Termination (both for futility and efficacy) given there is no increase in response rate when adding Mitazalimab
PET_{H1} = Probability of Early Termination (both for futility and efficacy) given there is an increase in response rate when adding Mitazalimab

Assuming a drop-out rate of 15% (not evaluable for response), the interim analysis required a total of 27 enrolled patients at RP2D to reach 23 evaluable patients. Patient recruitment continued during the interim analysis. If the study continued after interim analysis, a total number of 64 needed to be enrolled at the RP2D to reach 54 evaluable patients assuming the same drop-out rate as above.

### Statistical Methods

### Primary Efficacy

The primary endpoint is the Objective Response Rate (ORR) defined as the proportion of patients achieving a confirmed complete response (CR) or partial response (PR) on the RECIST v. 1.1 at any time during the study. For the interim analysis, the ORR was based on the response evaluation after 8 cycles for mitazalimab in combination with mFOLFIRINOX or 4 cycles for mitazalimab in combination with gemcitabine plus nab-paclitaxel (i.e., approximately 4 months after first dose). The response did not have to be confirmed as per RECIST v. 1.1 for the interim analysis. The primary endpoint ORR was compared between mitazalimab and historical control using a one-sided exact binomial test performed on the 10% significance level. Two-sided exact binomial 90% confidence intervals were also presented. In addition, ORR was presented for each study visit. Patients not evaluable for ORR and who completed at least 2 cycles were considered as non-responders.

### Secondary Efficacy

All secondary efficacy endpoint were presented using descriptive statistics. Best Overall Response (BOR) for each patient will be defined as the best response from RECIST v. 1.1 categories CR, PR, SD and PD at any time during the study. Disease control rate will be defined as either CR, PR or SD at each visit as determined by RECIST v. 1.1. The duration of response (DoR) was defined as number of days from initial response of CR or PR to progressive disease or death due to underlying disease, whichever came first. Duration of Stable Disease (SD) was defined as number of days from first dose of mitazalimab to progressive disease or death, whichever came first. Time to next anti-cancer therapy was defined as number of days from the time from first dose of mitazalimab to next treatment initiated. Kaplan-Meier curves were presented for DoR, duration of SD and time to next anti-cancer therapy. Progression-free survival (PFS) was defined as the number of days from first dose of mitazalimab to progressive disease or death due to any cause, whichever occurred first. Overall survival (OS) was defined as the number of days from first dose of mitazalimab to date of death from any cause. Kaplan-Meier curves were presented for PFS and OS. In addition, detection and characterization of anti-drug antibody (ADA) titers in serum were summarized.

### Safety Analysis

Number or patients experiencing AEs and number of events were summarized by SOC and preferred term. In addition, the following AEs were presented:
Incidence of DLT (Part 1);
AEs Grade 3 or higher;
Serious AEs;
AEs leading to discontinuation of study treatment; and
AEs leading to death.

### Analysis Populations

Patients in Part 1 who are on the same dose regimen as patients in Part 2 were pooled together with the Part 2 patients for the statistical analyses and data summaries.

### Full Analysis Set (FAS)

The FAS included all patients who had at least 1 post-baseline efficacy measurement. The FAS population was used for all efficacy and explorative endpoints.

### Safety Set

The safety set comprised all patients that received any study treatment (mitazalimab) and was used for all endpoints related to the safety objectives.

### Per Protocol Set (PP)

The PP set comprised all patients that completed at least one treatment cycle and had at least 1 post-baseline efficacy measurement and no critical protocol deviations. PP was used for all endpoints related to the efficacy objectives in addition to FAS.

### Dose Escalation in Part 1

Part 1 followed a Bayesian Optimal Interval (BOIN) design with at least 3 evaluable patients per dose level. The BOIN is described in statistical detail in reference [37] as well as in clinical application in reference [38]. It can be considered as a generalization of the 3+3, accelerated titration and 3+3+3 designs and is quite similar to these designs. According to the BOIN design each time a cohort of patients has been completed, it is evaluated whether the next cohort should remain on the same dose, escalate to the next higher dose level above or de-escalate to the next lower dose level, according to the decision rules presented in Table 3. The BOIN design shares the simplicity of the 3+3 design, which makes the decision of dose escalation/de-escalation by comparing p with 0/3, 1/3, 2/3, 0/6, 1/6, and 2/6. In the BOIN design this decision is based on a comparison of p with two pre-determined fixed boundaries, λₑ and λ_{d}. Where \hat{p} is the observed DLT-rate \hat{p} number of patients with DLT/number of patients treated) at the current dose level. In this study, the target toxicity level, i.e., the target DLT rate, is 30% and the boundaries λₑ=0.236 and λ_{d}=0.359.

### Interim Analysis in Part 2

An interim analysis was conducted when 23 evaluable patients at RP2D have completed 17 weeks treatment (8 treatment cycles for mitazalimab and mFOLFIRINOX combination or 4 cycles for the mitazalimab and gemcitabine plus nab-paclitaxel combination) according to the extended Simon's two-stage design. The cut-off limits for stopping for futility or efficacy are 57 or >12 responders (ORR), i.e., if there are 8-11 responders the study continued.

### Example 3: Appendix 1 Response Evaluation Criteria in Solid Tumours—RECIST v. 1.1 Guideline

The revised RECIST v. 1.1 guideline are available at:
https://ctep.cancer.gov/protocoldevelopment/docs/recist_guideline.pdf

### Definitions

Response and progression were evaluated in this trial using the international criteria (version 1.1) proposed by the Response Evaluation Criteria in Solid Tumours (RECIST) Committee [Eur J Cancer. 45 (2009) 228-247]. Changes in only the largest diameter (unidimensional measurement) of the tumour lesions are used in the RECIST v. 1.1 criteria. Note: Lesions are either measurable or non-measurable using the criteria provided below. The term “evaluable” in reference to measurability will not be used because it does not provide additional meaning or accuracy.

### Measurable Disease

Measurable disease is defined by the presence of at least one measurable lesion. Measurable lesions are defined as those that can be accurately measured in at least one dimension [longest diameter (LD) in the plane of measurement to be recorded] with a minimum size of:
10 mm by CT scan (CT scan slice thickness no greater than 5 mm);
10 mm caliper measurement by clinical exam (lesions which cannot be accurately measured with calipers should be recorded as non-measurable); and/or
20 mm by chest x-ray.

Malignant lymph nodes: To be considered pathologically enlarged and measurable, a lymph node must be ≥15 mm in short axis when assessed by CT scan (CT scan slice thickness no greater than 5 mm).

### Non-Measurable Disease

All other lesions (or sites of disease), including small lesions (longest diameter<10 mm or pathological lymph nodes with ≥10 to <15 mm short axis) are considered non-measurable disease. Lesions considered truly non-measurable include: leptomeningeal disease, ascites, pleural/pericardial effusions, lymphangitis cutis/pulmonis, inflammatory breast disease, abdominal masses/abdominal organomegaly identified by physical exam and not followed by CT or MRI.

Bone lesions, cystic lesions and lesions previously treated with local therapy may be considered as follows:

### Bone Lesions:

Bone scan, positron-emission tomography (PET) scan or plain films are not considered adequate imaging techniques to measure bone lesions. However, these techniques can be used to confirm the presence or disappearance of bone lesions.
Lytic bone lesions or mixed lytic-blastic lesions, with identifiable soft tissue components, that can be evaluated by cross sectional imaging techniques (i.e. CT or MRI) can be considered as measurable lesions if the soft tissue component meets the definition of measurability described above.
Blastic bone lesions are non-measurable.

### Cystic Lesions:

Lesions that meet the criteria for radiographically defined simple cysts should not be considered malignant lesions (neither measurable nor non-measurable) since they are, by definition, simple cysts.
‘Cystic lesions’ thought to represent cystic metastases can be considered measurable lesions, if they meet the definition of measurability described above. However, if non-cystic lesions are present in the same subject, these are preferred for selection as target lesions.
   Lesions with Prior Local Treatment:
Tumour lesions situated in a previously irradiated area, or in an area subjected to other loco-regional therapy, are usually not considered measurable unless there has been demonstrated progression in the lesion.

### Target Lesions

All measurable lesions up to a maximum of two lesions per organ and five lesions in total, representative of all involved organs, can be identified as target lesions and recorded and measured at baseline. Target lesions should be selected on the basis of their size (lesions with the longest diameter) and their suitability for accurate repeated measurements (either by imaging techniques or clinically). A sum of the diameters (longest for non-nodal lesions, short axis for nodal lesions) for all target lesions was calculated and reported as the baseline sum diameters. The baseline sum diameters was used as reference by which to characterize the objective tumour response.

### Lymph Node Assessment

For lymph nodes, measurements can be made of the short axis, which is defined as perpendicular to the LD of node assessed in the plane of measurement:
Target lesion if short axis≥15 mm;
Non-target lesion if short axis is ≥10 but <15 mm;
Normal if short axis<10 mm.

For baseline, the actual short axis measurement was added to the sum of LD of non-nodal lesions.

### Non-Target Lesions

All other lesions (or sites of disease) including pathological lymph nodes can be identified as non-target lesions and be recorded at baseline. Measurements of these lesions are not required, and these lesions can be followed as “present,” “absent,” or in rare cases “unequivocal progression”. In addition, it is possible to record multiple non-target lesions involving the same organ as a single item on the case report form (e.g. ‘multiple enlarged pelvic lymph nodes’ or ‘multiple liver metastases’).

### Guidelines for Evaluation of Measurable Disease

All measurements can be taken and recorded in metric notation using a ruler or calipers. All baseline evaluations can be performed as closely as possible to the beginning of treatment and never more than 4 weeks before the beginning of the treatment. The same method of assessment and the same technique is preferably used to characterize each identified and reported lesion at baseline and during follow-up. Imaging-based evaluation is preferred to evaluation by clinical examination when both methods have been used to assess the antitumour effect of a treatment.
Clinical lesions. Clinical lesions will only be considered measurable when they are superficial and ≥10 mm diameter as assessed using calipers (e.g., skin nodules). In the case of skin lesions, documentation by colour photography, including a ruler to estimate the size of the lesion, is recommended. When lesions can be evaluated by both clinical exam and imaging, imaging evaluation should be undertaken since it is more objective and may be reviewed at the end of the trial.
Chest x-ray. Chest CT is preferred over chest x-ray, particularly when progression is an important endpoint. Lesions on chest x-ray may be considered measurable if they are clearly defined and surrounded by aerated lung.
Conventional CT and MRI. This guideline has defined measurability of lesions on CT scan based on the assumption that CT slice thickness is 5 mm or less. When CT scans have slice thickness>5 mm, the minimum size for a measurable lesion should be twice the slice thickness. MRI is acceptable in certain situations (e.g., for body scans).
Ultrasound (US). US should not be used to measure tumour lesions. US examinations cannot be reproduced in their entirety for independent review at a later date because they are operator dependent. If new lesions are identified by US, confirmation by CT or MRI is advised. If there is concern about radiation exposure at CT, MRI may be used instead of CT.
Endoscopy, Laparoscopy. The utilization of these techniques for objective tumour evaluation is not advised. However, such techniques can be useful to confirm complete pathological response when biopsies are obtained or to determine relapse in trials where recurrence following complete response or surgical resection is an endpoint.
Tumour markers. Tumour markers alone cannot be used to assess objective tumour response. If markers are initially above the upper normal limit, they must normalize for a subject to be considered in complete clinical response.
Cytology, Histology. These techniques can be used to differentiate between partial responses (PR) and complete responses (CR) in rare cases (e.g., residual lesions in tumour types such as germ cell tumours, where known residual benign tumours can remain).

### Response Criteria

### Evaluation of Target Lesions

| | |
|---|---|
| Complete | Disappearance of all target lesions. Any pathological |
| Response (CR): | lymph nodes (whether target or non-target) must have |
| | reduction in short axis to <10 mm. |
| Partial | At least a 30% decrease in the sum of diameters of |
| Response (PR): | target lesions, taking as reference the baseline sum |
| | diameters. |
| Progressive | At least a 20% increase in the sum of diameters of target |
| Disease (PD): | lesions, taking as reference the smallest sum on trial (this |
| | includes the baseline sum if that is the smallest). In |
| | addition to the relative increase of 20%, the sum must |
| | also demonstrate an absolute increase of at least 5 mm. |
| | The appearance of one or more new lesions is also |
| | considered progression. |
| Stable Disease | Neither sufficient shrinkage to qualify for PR nor |
| (SD): | sufficient increase to qualify for PD, taking as reference |
| | the smallest sum diameters while in the trial. |

### Assessment of Target Lymph Nodes

Lymph nodes identified as target lesions can have the actual short axis measurement recorded (measured in the same anatomical plane as the baseline exam), even if the nodes regress to below 10 mm in the trial. In order to qualify for CR, each node can achieve a short axis<10 mm. For PR, SD and PD, the actual short axis measurement of the nodes is to be included in the sum of target lesions.

Target Lesions that Become “Too Small to Measure”

All lesions (nodal and non-nodal) recorded at baseline can have their actual measurements recorded at each subsequent evaluation, even when very small (e.g., 2 mm). If it is the opinion of the radiologist that the lesion has disappeared, the measurement can be recorded as 0 mm. If the lesion was believed to be present and was faintly seen but too small to measure, a default value of 5 mm can be assigned.

Lesions that Split or Coalesce on Treatment

When non-nodal lesions fragment, the longest diameters of the fragmented portions can be added together to calculate the target lesion sum. Similarly, as lesions coalesce, a plane between them may be maintained that would aid in obtaining diameter measurements of each individual lesion. If the lesions have truly coalesced such that they are no longer separable, the vector of the longest diameter can be the maximal longest diameter for the ‘coalesced lesion.’

### Evaluation of Non-Target Lesions

| | |
|---|---|
| Complete | Disappearance of all non-target lesions and normalization |
| Response (CR): | of tumour marker level. All lymph nodes must be non- |
| | pathological in size (<10 mm short axis). |
| Non-CR/ | Persistence of one or more non-target lesion(s) and/or |
| Non-PD: | maintenance of tumour marker level above the normal |
| | limits. |
| Progressive | Unequivocal progression of existing non-target lesions. |
| Disease (PD): | (The appearance of one or more new lesions is also |
| | considered progression.) To achieve ‘unequivocal |
| | progression’ on the basis of the non-target disease, there |
| | must be an overall level of substantial worsening in non- |
| | target disease such that, even in the presence of SD or PR |
| | in target disease, the overall tumour burden has increased |
| | sufficiently to merit discontinuation. |

### New Lesions

The finding of a new lesion can be unequivocal (i.e., not attributed to differences in scanning technique, change in imaging modality, or findings thought to represent something other than tumour, such as a ‘new’ healing bone lesion). A lesion identified on a follow-up trial in an anatomical location that was not scanned at baseline was considered a new lesion and indicated disease progression. If a new lesion was equivocal, continued therapy and follow-up evaluation clarified if it represents truly new disease. If repeat scans confirmed this is definitely a new lesion, then progression could be declared using the date of the initial scan.

### Evaluation of Best Overall Response

The best overall response is the best response recorded from the start of the treatment until disease progression/recurrence (taking as reference for progressive disease the smallest measurements recorded since the treatment started). The subject's best overall response assignment depended on findings of both target and non-target disease and took into consideration the appearance of new lesions. Furthermore, depending on the nature of the trial, it may also require confirmatory measurement. Specifically, in non-randomized trials where response is the primary endpoint, confirmation of PR or CR is preferred to deem either one the “best overall response”. Table 13 provides a summary of the overall response status calculation at each time point for subjects who have measurable disease at baseline.

TABLE 13
Time Point Response: Subjects with Target
(+/− Non-target) Disease

| | | New | Overall |
|---|---|---|---|
| Target Lesions | Non-target Lesions | Lesions | Response |
| CR | CR | No | CR |
| CR | Non-CR/non-PD | No | PR |
| CR | Not evaluated | No | PR |
| PR | Non-PD or not all evaluated | No | PR |
| SD | Non-PD or not all evaluated | No | SD |
| Not all evaluated | Non-PD | No | NE |
| PD | Any | Yes or No | PD |
| Any | PD | Yes or No | PD |
| Any | Any | Yes | PD |

CR = complete response,
PR = partial response,
SD = stable disease
PD = progressive disease,
NE = unevaluable

Best response determination for studies where confirmation of CR or PR is required: Complete or partial responses may be claimed only if the criteria for each are confirmed by a repeat assessment at least 4 weeks later. In this circumstance, the best overall response can be interpreted as in Table 14.

TABLE 14
Best Overall Response when Confirmation of CR and PR Required

| Overall | Overall | |
|---|---|---|
| response | response | |
| First time | Subsequent | |
| point | time point | BEST overall response |
| CR | CR | CR |
| CR | PR | SD, PD or PR* |
| CR | SD | SD provided minimum criteria for SD |
| | | duration met, otherwise PD |
| CR | PD | SD provided minimum criteria for SD |
| | | duration met, otherwise PD |
| CR | NE | SD provided minimum criteria for SD |
| | | duration met, otherwise NE |
| PR | CR | PR |
| PR | PR | PR |
| PR | SD | SD |
| PR | PD | SD provided minimum criteria for SD |
| | | duration met, otherwise PD |
| PR | NE | SD provided minimum criteria for SD |
| | | duration met, otherwise NE |
| NE | NE | NE |

CR = complete response;
PR = partial response;
SD = stable disease;
PD = progressive disease;
NE = unevaluable
* If CR is truly met at first time point, then any disease seen at a subsequent time point, even disease meeting PR criteria relative to baseline, makes the disease PD at that point (since disease must have reappeared after CR). Best response would depend on whether minimum duration for SD was met. However, sometimes ‘CR’ may be claimed when subsequent scans suggest small lesions were likely still present and in the fact subject had PR, not CR, at the first time point. Under these circumstances, the original CR should be changed to PR and the best response is PR.

### Confirmatory Measurement/Duration of Response

### Confirmation

To be assigned a status of PR or CR, changes in tumour measurements can be confirmed by repeat assessments that are preferably performed 4 weeks after the criteria for response are first met. In the case of SD, follow-up measurements preferably met the SD criteria at least once after trial entry at a minimum interval of 7 weeks.

### Duration of Overall Response

The duration of overall response was measured from the time measurement criteria were met for CR or PR (whichever is first recorded) until the first date that recurrent or progressive disease is objectively documented (taking as reference for progressive disease the smallest measurements recorded since the treatment started). The duration of overall CR was measured from the time measurement criteria were first met for CR until the first date that recurrent disease is objectively documented.

### Duration of Stable Disease

Stable disease was measured from the start of the treatment until the criteria for progression were met, taking as reference the smallest measurements recorded since the treatment started.

### Example 4: Appendix 2 mFOLFIRINOX and Permitted Dose Modifications

The guidelines for the permitted dose modification of mFOLFIRINOX during the study are described in this appendix. All other information related to mFOLFIRINOX treatment is described throughout the protocol.

### Rules for Dose Omissions and Modified Schedules

The dose modifications for the mFOLFIRINOX treatment are outlined below. Toxicities are graded based upon CTCAE v 5.0. Dose adjustments are to be made according to the system showing the greatest degree of toxicity. Doses will be reduced, one level at a time, for hematologic and non-hematological toxicities.
Three levels of dose modifications are permitted, for each constituent of mFOLFIRINOX, according to the criteria below (Table 15).
If a toxicity requiring dose modification occurs following the third dose reduction of any constituent, additional dose reductions are not permitted. However, further treatment can be discussed with the Medical Monitor.

TABLE 15
Dose modifications for mFOLFIRINOX

| Dose Level/ | Planned | | Dose modification steps | | | | | |
|---|---|---|---|---|---|---|---|---|
| Constituent | Dose | | Dose Level − 1 | | Dose Level − 2 | | Dose Level − 3 | |
| Oxaliplatin | 85 | mg/m² | 60 | mg/m² | 50 | mg/m² | 40 | mg/m² |
| Leucovorin | 400 | mg/m² | 400 | mg/m² | 400 | mg/m² | 400 | mg/m² |
| Irinotecan | 150 | mg/m² | 120 | mg/m² | 100 | mg/m² | 80 | mg/m² |
| 5-FU | 2400 | mg/m² | 1920 | mg/m² | 1600 | mg/m² | 1360 | mg/m² |

If treatment is held for more than 4 consecutive weeks for a treatment related toxicity, patients can discontinue mFOLFIRINOX and mitazalimab treatment. However, if a patient is clinically benefitting at the end of a 4-week hold, the Investigator may contact the Medical Monitor to potentially continue protocol-based therapy. Patients who discontinue study treatment can perform the End of treatment visit and proceed to the post-treatment follow-up period. After the End of treatment visit, patients will receive recommendation on standard of care treatment from Investigator.

Determination regarding the need for dose modification of 5-FU, irinotecan, oxaliplatin, and/or leucovorin can be made based on the guidelines according to system outlined below. Management of toxicities and supportive care, except where indicated below, can be performed as judged by the Investigator.

Dose Modifications for Neutropenia and/or Thrombocytopenia at the Start of a Cycle or within a Cycle

Blood count on

| the first day of | | Dose reduction | | |
|---|---|---|---|---|
| mFOLFIRINOX | | | | 5-FU and |
| dosing | Dose Delay | Irinotecan | Oxaliplatin | leucovorin |
| Neutrophils ≥1.5 × | No delay | No dose reduction | | |
| 10⁹/L | | | | |
| Thrombocytes ≥100 × | | | | |
| 10⁹/L | | | | |
| Neutrophils <1.5 × | Delay | 1^{st} episode: | 1^{st} episode: no | 1^{st} episode: |
| 10⁹/L | treatment until | dose reduction | dose reduction | no dose |
| | neutrophils ≥1.5 × | to 120 mg/m² | 2^{nd} episode: | reduction |
| | 10⁹/L | 2^{nd} episode: | dose reduction | |
| | | maintain dose | to 60 mg/m² | |
| | | at 120 mg/m² | 3^{rd} episode: | |
| | | 3^{rd} episode: | discuss | |
| | | discuss | treatment stop | |
| | | treatment stop | or maintain | |
| | | or maintain | only 5-FU and | |
| | | only 5-FU and | leucovorin | |
| | | leucovorin | | |
| Thrombocytes <100 × | Delay | 1^{st} episode: no | 1^{st} episode: | 1^{st} episode: |
| 10⁹/L | treatment until | dose reduction | dose reduction | no dose |
| | thrombocytes ≥100 × | 2^{nd} episode: | to 60 mg/m² | reduction |
| | 10⁹/L | dose reduction | 2^{nd} episode: | 2^{nd} episode: |
| | If no recovery | to 120 mg/m² | maintain 60 | reduce the |
| | within 14 days | 3^{rd} episode: | mg/m² | dose of iv |
| | discuss | discuss | 3^{rd} episode: | continuous |
| | treatment stop | treatment stop | discuss | infusion by |
| | | or maintain | oxaliplatin stop | 25% |
| | | only 5-FU and | | |
| | | leucovorin | | |

The use of prophylactic G-CSF is advised when there is a delay in treatment due to hematologic toxicity involving neutrophils. G-CSF can first be started 24 hours after end of the cytotoxic chemotherapy. Other hematologic toxicities do not require dose modification. However, red blood cell transfusion can be considered for haemoglobin<9.5 g/dL or significant symptoms of anaemia or per institutional guidelines.

### Measures for Hematologic Toxicity During the Cycle (Nadir Values)

| Events | Dose reduction for mFOLFIRINOX |
|---|---|
| Febrile neutropenia | 1^{st} episode: dose reduction of irinotecan to |
| Grade 4 | 120 mg/m² and add G-CSF if applicable |
| neutropenia >7 days | 2^{nd} episode: in addition, reduce oxaliplatin to |
| Infection with grade | 60 mg/m² |
| 3-4 neutropenia | 3^{rd} episode: discuss growth factor or further |
| | treatment reduction, maintain only 5-FU and |
| | leucovorin if necessary |
| Thrombocytopenia | 1^{st} episode: dose reduction of oxaliplatin to |
| grade 3-4 | 60 mg/m² |
| | 2^{nd} episode: in addition, reduce irinotecan to |
| | 120 mg/m² and reduce the dose of 5-FU iv |
| | continuous infusion by 25% |
| | 3^{rd} episode: stop oxaliplatin and irinotecan, continue |
| | 5-FU and leucovorin |

### Dose Modifications for Diarrhoea

| Events | Dose reduction for mFOLFIRINOX |
|---|---|
| Diarrhea grade 1-2 | No dose modification; Initiate/optimize supportive |
| | care |
| Diarrhea grade 3-4 | 1^{st} episode: dose reduction of irinotecan to |
| or Diarrhea + fever | 120 mg/m² |
| and/or grade 3-4 | 2^{nd} episode: dose reduction of oxaliplatin to |
| neutropenia | 60 mg/m² and reduce the dose of 5-FU iv |
| | continuous infusion by 25% |
| | 3^{rd} episode: stop irinotecan |
| Recurrent diarrhea | No dose reduction for irinotecan, oxaliplatin and |
| (>48 h) despite high | 5-FU after recovery except if Diarrhea grade 3-4 or |
| doses of loperamide | diarrhea + fever and/or grade 3-4 neutropenia |

For symptoms of diarrhoea (and/or abdominal cramping) that occur at any time during a treatment cycle, it is suggested that patients should be instructed to take an anti-diarrheal, such as loperamide (2 mg every 2 hours until diarrhoea resolves for 12 hours; 4 mg 4 hours at night is allowed) or diphenoxylate/atropine (Lomotil) as treatment for diarrhoea. For persisting diarrhoea (i.e., lasting more than 48 hours) treatment with broad spectrum antibiotics, fluoroquinolone orally for 7 days. If severe diarrhoea, hospitalization for parenteral rehydration and change to iv antibiotics should be considered. Acute diarrhoea and abdominal cramps, developing during or within 24 hours after irinotecan administration, may occur as part of a cholinergic syndrome. For irinotecan-related cholinergic reactions, the infusion time may be increased to mitigate these symptoms and prophylactic atropine per institutional guidelines is permitted.

### Dose Modifications for Drug Related Hepatic Toxicity

For all hepatobiliary toxicity, hold treatment and evaluate for non-drug causes, e.g. biliary obstruction/stent malfunction (see also Example 7). Once the underlying aetiology is corrected and improving, resume therapy (5-FU and oxaliplatin only without irinotecan) at the previous dose level and add irinotecan once toxicity improved to ≤Grade 1.

The following dose modification guidelines may be used for hyperbilirubinemia:
1) Grade 2 and Grade 3 hyperbilirubinemia: Omit irinotecan until Grade≤1 and resume at the same dose level;
2) Grade 4 hyperbilirubinemia: Hold therapy until Grade 1 and resume at the next dose level once underlying aetiology is corrected*.

*Note: If the aetiology of hyperbilirubinemia is from biliary obstruction (i.e., reversible, and non-therapy related), discussion with the Medical Monitor may permit the option to continue irinotecan at original levels once liver function tests are resolved to ≤Grade 1.

### Dose Modifications for Mucositis

Mucositis as a toxicity is caused by 5-FU. If grade 3-4 toxicity occurs, continuous 5-FU IV infusion can be reduced by 25% for the remaining courses.

### Dose Modifications for Peripheral Neuropathy

Only oxaliplatin is modified in the case of peripheral neuropathy:
1) Grade peripheral neuropathy: Continue monitoring at same dose level;
2) Grade 2 peripheral neuropathy persisting more than 14 days: Decrease oxaliplatin by one dose level;
3) Grade 3 peripheral neuropathy: Decrease oxaliplatin dose to 65 mg/m² if G3 peripheral neuropathy persists for more than 14 days, discontinue oxaliplatin;
4) Grade 4 peripheral neuropathy: Discontinue oxaliplatin, continue 5-FU, irinotecan and leucovorin at same dose level, if resolved to <Grade 1, therapy can be resumed on case-by-case basis after discussion with Medical Monitor.

### Hand-Foot Syndrome

Hand-Foot syndrome as a toxicity is caused by 5-FU. If grade 3-4 toxicity occurs, continuous 5-FU IV infusion can be reduced by 25% for the remaining courses.

Dose Modifications for Other Clinically Significant Non-Hematologic* Toxicities (Except Alopecia and Grade 3 Nausea and Vomiting Responding to Medical Treatment within 72 Hours)

| Toxicity and | Suggested Dose Modification | | | | |
|---|---|---|---|---|---|
| Grade | Occurrence | 5-FU | Irinotecan | Oxaliplatin | Leucovorin |
| Grade 3 | First | Reduce the suspected offending agent by one dose level | | | |
| Hold treatment | Second | Reduce the suspected offending agent by one or more | | | |
| until AE resolves | | dose level(s) | | | |
| to ≤ Grade 1. | Third | Discontinue the suspected offending agent | | | |
| | Fourth | Discuss with Medical Monitor | | | |
| Grade 4 | First | Discontinue the suspected offending agent | | | |
| Hold treatment | Second | Discuss with Medical Monitor | | | |
| until AE resolves | | | | | |
| to ≤ Grade 1. | | | | | |

*Determination of “clinically significant” AEs and “offending drug” is at the discretion of the treating physician and/or the Medical Monitor.

Gastrointestinal ulceration, regardless of whether it is haemorrhagic: hold 5-FU until symptoms resolved. Acute laryngopharyngeal dysesthesia is due to oxaliplatin and can be handled by institutional practices which may include prolonged oxaliplatin infusion duration to 6 hours and infusion of 1 g of calcium gluconate and 1 g of magnesium sulfate over 15 min before all subsequent oxaliplatin infusions.

### Dose Modifications for Infusion-Related Reactions

Either institutional guidelines or those described below can be followed in case of infusion-related reactions to any chemotherapy component given per protocol. Infusion reactions will be defined according to the National Cancer Institute CTCAE (version 5.0) definitions of an allergic reaction or anaphylaxis as noted below.

| | Suggested Dose Modification (any |
|---|---|
| Toxicity and Grade | constituent of mFOLFIRINOX) |
| Grade 1: | Reduce infusion rate of offending agent |
| Transient flushing or rash, drug | by 50% |
| fever <38° C. (100.4° F.); intervention | Monitor patient every 15 minutes for |
| not indicated | worsening of condition |
| | Future infusions may be administered at |
| | a reduced rate (e.g., over 60-120 |
| | minutes for irinotecan), at the discretion |
| | of the Investigator |
| | NOTE: Premedication with a combination of |
| | diphenhydramine hydrochloride 25-50 mg IV, |
| | dexamethasone 10-20 mg IV, and |
| | acetaminophen 650 mg orally or per |
| | institutional guidelines may be provided as part |
| | of subsequent treatments. |
| Grade 2: | Stop infusion of offending agent |
| Intervention or infusion interruption | Administer diphenhydramine hydrochloride |
| indicated; responds promptly to | 25-20 mg IV, acetaminophen 650 mg orally, |
| symptomatic treatment (e.g., | and oxygen |
| antihistamines, NSAIDS, narcotics); | Resume infusion at 50% of the prior rate |
| prophylactic medications indicated | once infusion reaction has resolved |
| for ≤24 hours. | Monitor patient every 15 minutes for |
| | worsening of condition |
| | For all subsequent infusions, pre-medicate |
| | with diphenhydramine hydrochloride 25-50 |
| | mg IV, dexamethasone 10-20 mg IV, and |
| | acetaminophen 650 mg orally |
| | Future infusions may be administered at a |
| | reduced rate (e.g., over 60-120 minutes for |
| | irinotecan), at the discretion of the |
| | Investigator. |
| Grade 3: | Stop infusion and disconnect infusion tubing |
| Prolonged (e.g., not rapidly | from patient |
| responsive to symptomatic | Administer diphenhydramine hydrochloride |
| medication and/or brief interruption | 25-50 mg IV, dexamethasone 10-20 mg IV, |
| of infusion); recurrence of symptoms | bronchodilators for bronchospasm, and |
| following initial improvement; | other medications or oxygen as medically |
| hospitalization indicated for clinical | necessary |
| sequelae (e.g., renal impairment, | No further treatment will be permitted |
| pulmonary infiltrates). | during this visit |
| | Consider consultation with allergist prior to |
| | subsequent treatment; de-sensitization to |
| | oxaliplatin as per institutional guidelines |
| | with premedication with a combination of |
| | diphenhydramine hydrochloride 25-50 mg |
| | IV, dexamethasone 10-20 mg IV, |
| | montelukast 10 mg and acetaminophen 650 |
| | mg orally |
| Grade 4: | Stop the infusion and disconnect infusion |
| Life-threatening consequences; | tubing from patient |
| urgent intervention indicated. | Administer epinephrine, bronchodilators or |
| | oxygen as indicated for bronchospasm |
| | Administer diphenhydramine hydrochloride |
| | 50 mg IV, dexamethasone 10-20 mg IV and |
| | other medications as medically necessary |
| | Consider hospital admission for observation |
| | No further treatment will be permitted |
| | during this visit |

### Cardiac Toxicity

In case of cardiac pain, e.g., angina pectoris or myocardial infarction, 5-FU treatment can be stopped.

### Extravasation

Severe reactions due to irinotecan or oxaliplatin extravasation have been reported [39]. General recommendations in case of extravasation are as follow:
stop infusion immediately,
do not remove the needle or the catheter,
suck/aspirate the maximum of infiltrated product through the needle,
apply ice on the infiltrated area for 15 to 20 minutes every 4 to 6 hours for a period of 72 hours,
apply local corticotherapy.

Regularly check the infiltrated site during the following days, to verify whether more treatment is needed. Do not hesitate to require a surgical consultation in case of doubt.

### Example 5: Appendix 3 Gemcitabine Plus Nab-Paclitaxel Administration Including Dosing Schedule, Visit Assessment Tables and Potential Dose Modifications (if Applicable)

Please note that gemcitabine plus nab-paclitaxel treatment only applies for this study in case the Data Review Committee (DRC) has taken a formal decision during Part 1 of the study to change the chemotherapy mFOLFIRINOX to gemcitabine plus nab-paclitaxel.

### Dose Modifications

Dose adjustments are to be made according to the system showing the greatest degree of toxicity. Doses may be reduced, one level at a time, for hematologic and non-haematological toxicities.

At least two levels of dose modifications are permitted, for each drug, according to the criteria below, see Table 16. If a toxicity requiring dose modification occurs following the second, or further, dose gemcitabine and nab-paclitaxel reduction of either drug, additional dose reductions are not permitted.

TABLE 16
Dose modifications for gemcitabine and nab-paclitaxel

| Dose Level/ | Planned | | Dose modification steps | | |
|---|---|---|---|---|---|
| Agent | Dose | | Dose Level −1 | Dose Level −2 | |
| Nab-paclitaxel | 125 | mg/m² | 100 mg/m² | 75 | mg/m² |
| Gemcitabine | 1000 | mg/m² | 800 mg/m² | 600 | mg/m² |

If treatment is held for >3 consecutive weeks for a treatment related toxicity, patients can stop all study treatment. However, if a patient is clinically benefitting at the end of a 3-week hold, treating physicians may contact the Medical Monitor to potentially extend therapy. Patients who discontinue study treatment can perform End of treatment visit and enter post-treatment follow up period, as per Table 21 and Table 22, respectively. In situations where toxicity justifies discontinuation of an agent, only the individual offending agent needs to be removed from the regimen, and treatment can continue otherwise per protocol. Determination regarding the need for dose modifications of nab-paclitaxel and/or gemcitabine can be made based on the following guidelines.

TABLE 17
Dose Modifications for Neutropenia and/or Thrombocytopenia
at the start of a cycle or within a cycle

| | | | | Nab- | |
|---|---|---|---|---|---|
| Cycle | | | | paclitaxel | Gemcitabine |
| Day | Neutrophils | | Thrombocytes | Dose | Dose |
| Day 1 | ≥1.5 × 10⁹/L | AND | ≥100 × 10⁹/L | Treat on time at current dose | |
| | | | | levels | |
| | <1.5 × 10⁹/L | OR | <100 × 10⁹/L | Delay doses until recovery | |
| Day 8 | ≥1 × 10⁹/L | AND | ≥75 × 10⁹/L | Treat on time at current dose | |
| | | | | levels | |
| | ≥0.5 × 10⁹/L | OR | ≥50 × 10⁹/L | Reduce to DL-1 | |
| | to <1 × 10⁹/L | | to <100 × 10⁹/L | | |

| | | | |
|---|---|---|---|
| <0.5 × 10⁹/L | OR | <50 × 10⁹/L | Withhold doses |

Day 15, if Day 8 doses were given without modification or reduced:

| Day | ≥1 × 10⁹/L | AND | ≥75 × 10⁹/L | Treat on time at current dose |
|---|---|---|---|---|
| 15 | | | | levels |
| | ≥0.5 × 10⁹/L | OR | ≥50 × 10⁹/L | Reduce 1 dose level from |
| | to <1 × 10⁹/L | | to 75 × 10⁹/L | Day 8 |
| | <0.5 × 10⁹/L | OR | <50 × 10⁹/L | Withhold doses |
| Day | If Day 8 doses were withheld | | | |
| 15: | | | | |
| | >1 × 10⁹/L | AND | ≥75 × 10⁹/L | Reduce 1 dose level from |
| | | | | Day 8 |
| | ≥0.50 × 10⁹/L | OR | ≥50 × 10⁹/L | Reduce 2 dose levels from |
| | to ≤1 × 10⁹/L | | to <75 × 10⁹/L | Day 1 |
| | <0.5 × 10⁹/L | OR | <50 × 10⁹/L | Withhold doses |

The use of prophylactic G-CSF is advised when there is a delay in treatment due to hematologic toxicity involving neutrophils. G-CSF can first be started 24 hours after end of the cytotoxic chemotherapy. If hematologic toxicity is restricted to platelet counts alone, dose modification of only gemcitabine could be considered. Other hematologic toxicities do not necessarily require dose modification. However, red blood cell transfusion can be considered for haemoglobin<9.5 g/dL or significant symptoms of anaemia or per institutional guidelines.

TABLE 18
Dose Modifications for Other Clinically Significant Non-Hematologic* Toxicities

| | | Gemcitabine |
|---|---|---|
| Adverse Drug Reaction | Nab-paclitaxel Dose | Dose |
| Febrile Neutropenia: Grade 3 | Withhold doses until fever resolves and ANC is ≥1.5 × | |
| or 4 | 10⁹/L resume at next lower dose level for both agents | |
| Peripheral Neuropathy: | Withhold dose until improvement to ≤ | Treat with same |
| Grade 3 or 4 | Grade 1; Resume at next lower | dose |
| | dose level | |
| Cutaneous Toxicity: | Reduce doses to next lower dose level; discontinue | |
| Grade 2 or 3 | treatment if toxicity persists | |
| Gastrointestinal Toxicity: | Withhold until improves to ≤ Grade 1; Resume at next | |
| Grade 3 mucositis or | lower dose level | |
| diarrhoea | | |

For all other ≥Grade 3 non-hematologic toxicities (*except nausea, vomiting, alopecia and pulmonary embolism and Adverse events of special interest (AESIs) described below).
Withhold dose of either or both agent(s) until improvement to ≤Grade 1;
Resume at next lower dose level.

### Hepatotoxicity and Dose Reduction for Gemcitabine Plus Nab-Paclitaxel

If the AST or ALT or both increase is less than 5×ULN, gemcitabine can be continued without dose reduction;
If the AST or ALT or both increase is more than 5×ULN, but less than 20×ULN, gemcitabine dose can be reduced by 25%;
If the AST or ALT or both increase is more 20×ULN, gemcitabine can be stopped.

### Dosage Schedule

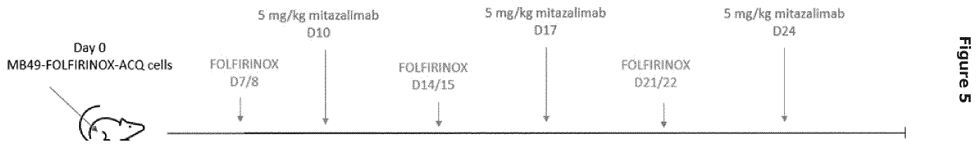

The first treatment cycle with mitazalimab and gemcitabine plus nab-paclitaxel will last 35-days, mitazalimab will be administered on Day 1, Day 10 and 24 and gemcitabine plus nab-paclitaxel on Day 8, 15 and 22. During the following 28-day treatment cycles, mitazalimab will be administered on Day 3 and 17 and gemcitabine plus nab-paclitaxel on Day 1, 8 and 15. A maximum of 6 treatment cycles will be allowed for mitazalimab and gemcitabine plus nab-paclitaxel combination, see FIG. 4.

TABLE 19
Assessment schedule during screening period and up to Day 14 of Treatment cycle 1

| | Activity/Visit | | | | | | | | | | |
|---|---|---|---|---|---|---|---|---|---|---|---|
| | Screening | Cycle 1 (35-day treatment cycle)¹⁰ - Day 1 to 14 | | | | | | | | | |
| | Day | | | | | | | | | | |
| | −21 to −1 | 1 | | | 2 | 8 ± 1 | | 10 ± 1 | | | 11 ± 1 |
| | Time in relation to study drug | | | | | | | | | | |
| | | | | | | | Gem- | | | | |
| | | Pre | Mitazalimab | Post¹ | 24 ± 2 h¹ | Pre | NabPac | Pre | Mitazalimab² | Post¹ | 24 ± 2 h¹ |
| Informed | X³ | | | | | | | | | | |
| Consent | | | | | | | | | | | |
| Eligibility | X | | | | | | | | | | |
| criteria | | | | | | | | | | | |
| Demographics | X | | | | | | | | | | |
| Medical history | X | | | | | | | | | | |
| Body weight | X | X⁴ | | | | | | | | | |
| Height | X | | | | | | | | | | |
| Vital signs | X | X | | 1 h ± | | X | | | | 1 h ± | |
| | | | | 10 min | | | | | | 10 min | |
| | | | | 2 h ± | | | | | | 2 h ± | |
| | | | | 10 min | | | | | | 10 min | |
| | | | | 4 h ± | | | | | | | |
| | | | | 15 min | | | | | | | |
| Physical | X | X⁴ | | | | X | | | | | |
| examination | | | | | | | | | | | |
| 12-lead ECG | X | X⁴ | | 1 h ± | | | | | | 1 h ± | |
| | | | | 10 min | | | | | | 10 min | |
| | | | | 4 h ± | | | | | | | |
| | | | | 15 min | | | | | | | |
| ECOG | X | X⁴ | | | | | | | | | |
| Treatment | | X⁵ | | | | X⁵ | | X⁵ | | | |
| discontinuation | | | | | | | | | | | |
| criteria | | | | | | | | | | | |
| Pre- and | | Note: Premedications are to be given up to 3 days | | | | | | | | | |
| post- | | prior to administration of mitazalimab. | | | | | | | | | |
| medication | | | | | | | | | | | |
| Mitazalimab | | | X⁶ | | | | | | X⁶ | | |
| administration | | | | | | | | | | | |
| Gem-NabPac | | | | | | | | | | | |
| administration | | | | | | | | | | | |
| Nab-paclitaxel | | | | | | | X | | | | |
| Gemcitabine | | | | | | | X | | | | |
| Adverse | Continuous from signing ICF until 28 | | | | | | | | | | |
| events | days after last dose of IMP | | | | | | | | | | |
| Concomitant | Continuous from signing of ICF | | | | | | | | | | |
| medication | until End of treatment visit | | | | | | | | | | |
| Concomitant | Continuous from signing of ICF | | | | | | | | | | |
| medical | until End of treatment visit | | | | | | | | | | |
| procedures | | | | | | | | | | | |
| Clin laboratory tests | | | | | | | | | | | |
| Pregnancy test | X | X⁷ | | | | | | | | | |
| Clinical chemistry, | X | X⁴ | | | | X⁴ | | X | | | |
| hematology and | | | | | | | | | | | |
| coagulation | | | | | | | | | | | |
| Urinalysis | X | X⁴ | | | | X⁴ | | | | | |
| Pharmacokinetics | | X⁴ | | 5 min, | X | X | | X | | 5 min, | X |
| | | | | 1 h ± | | | | | | 1 h ± | |
| | | | | 10 min | | | | | | 10 min | |
| | | | | 2 h ± | | | | | | 2 h ± | |
| | | | | 10 min | | | | | | 10 min | |
| | | | | 4 h ± | | | | | | 4 h ± | |
| | | | | 20 min | | | | | | 20 min | |
| Immunogenicity | | X⁴ | | | | | | | | | |
| CT scan/Tumour | X⁸ | | | | | | | | | | |
| response | | | | | | | | | | | |
| evaluation | | | | | | | | | | | |
| CA19-9 | X | X⁴ | | | | | | | | | X |
| Immunophenotyping | | X⁴ | | | X | X | | | | | X |
| Cytokines and | | X⁴ | | 1 h ± | X | X | | X | | 1 h ± | X |
| chemokines | | | | 10 min, | | | | | | 10 min, | |
| | | | | 4 h ± | | | | | | 4 h ± | |
| | | | | 20 min | | | | | | 20 min | |
| Whole | | X⁴ | | 1 h ± | X | X | | X | | 1 h ± | |
| blood RNA | | | | 10 min, | | | | | | 10 min, | |
| | | | | 4 h ± | | | | | | 4 h ± | |
| | | | | 20 min | | | | | | 20 min | |
| Whole | X | | | | | | | | | | |
| blood DNA | | | | | | | | | | | |
| Tumour | X⁹ | | | | | | | | | | |
| biopsy | | | | | | | | | | | |

* The sections referred to in the table are the sections in the main body of the protocol.
¹Assessments to be performed at specified time after End of infusion. The End of infusion is defined as when the infusion of IMP is completed/stopped (i.e., before rinsing if applicable). For example, 4 h post-dose means 4 hours after end of infusion.
²Mitazalimab can be administered 2 days following gemcitabine administration.
³Informed consent must be obtained before or at screening prior to performing any screening assessments.
⁴The assessment may be performed within 72 hours (up to 3 days) prior to administration of study treatment. If the screening assessment were taken Day −1 to −3 prior to dosing, it does not need to be repeated at Day 1.
⁵If any of the treatment discontinuation criteria apply, the patient can be discontinued from treatment and the End of treatment visit can be performed, see End of treatment visit in Table 21.
⁶If the infusion of mitazalimab is interrupted due to an AE, a PK sample and a sample for immunogenicity can be taken at the time of interruption, or as soon as is feasible considering the patient safety. An immunogenicity sample does not need to be taken if it is first administration of mitazalimab that is interrupted.
⁷The result of the pregnancy test must be available prior to dosing.
⁸The CT scan at screening may be obtained 28 days prior to first dose of mitazalimab.
⁹The baseline biopsy may be collected up to 28 days before first dose. If a fresh biopsy cannot be taken during screening period, archival biopsy material (most recent) could be used.
¹⁰The DLT evaluation period includes Day 1 to 28 of the full 35-day treatment cycle.

TABLE 20
Assessment schedule for Day 15-35 of Treatment cycle 1

| | Activity/Visit | | | | | | | | |
|---|---|---|---|---|---|---|---|---|---|
| | Cycle 1 (35-day treatment cycle)¹¹ - Day 15 to 35 | | | | | | | | |
| | Day | | | | | | | | |
| | 15 ± 1 | | 22 ± 1 | | 24 ± 1 | | | | |
| | Time in relation to study drug | | | | | | | 29 ± 4 | Unscheduled |
| | Pre | Gem-NabPac | Pre | Gem-NabPac | Pre | Mitazalimab³ | Post² | 24 ± 2 h² | visit¹ |
| Body weight | X⁴ | | | | | | | | X |
| Height | | | | | | | | | |
| Vital signs | X | | X | | | | 1 h ± 10 min | | X |
| Physical | X⁴ | | X⁴ | | | | | | X |
| examination | | | | | | | | | |
| 12-lead ECG | | | | | | | 1 h ± 10 min | | X |
| ECOG | | | | | | | | | X |
| Treatment | X⁵ | | X⁵ | | X⁵ | | | | X⁵ |
| discontinuation | | | | | | | | | |
| criteria | | | | | | | | | |
| Pre- and | Note: Premedications are to be given up to 3 days prior to administration of mitazalimab. | | | | | | | | |
| post- | | | | | | | | | |
| medication | | | | | | | | | |
| Mitazalimab | | | | | | X⁶ | | | |
| administration | | | | | | | | | |
| Gem-NabPac | | | | | | | | | |
| administration | | | | | | | | | |
| Nab-paclitaxel | | X | | X | | | | | |
| Gemcitabine | | X | | X | | | | | |
| Adverse events | Continuous from signing ICF until 28 days after last dose of study treatment | | | | | | | | |
| Concomitant medication | Continuous from signing of ICF until End of treatment visit | | | | | | | | |
| Concomitant medical procedures | Continuous from signing of ICF until End of treatment visit | | | | | | | | |
| Clin laboratory tests | | | | | | | | | |
| Pregnancy test | | | | | | | | | X |
| Clinical chemistry, | X⁴ | | X⁴ | | X | | | | X |
| hematology and | | | | | | | | | |
| coagulation | | | | | | | | | |
| Urinalysis | X⁴ | | X⁴ | | | | | | X |
| Pharmacokinetics | | | | | X | | 5 min | X | X |
| | | | | | | | 1 h ± 10 min | | |
| | | | | | | | 2 h ± 10 min | | |
| | | | | | | | 4 h ± 20 min | | |
| Immunogenicity | | | | | | | | | X |
| CT scan/Tumour | | | | | | | | | X |
| response | | | | | | | | | |
| evaluation | | | | | | | | | |
| CA19-9 | | | | | | | | | X |
| Immunophenotyping | X | | | | | | | | X |
| Cytokines and chemokines | X | | | | | | | | X |
| Whole blood RNA | X | | | | | | | | |
| Whole blood DNA | | | | | | | | | |
| Tumour biopsy | | | | | | | | X⁷ | X^{7, 8} |

* The sections referred to in the table are the sections in the main body of the protocol.
¹The assessments to be performed during the Unscheduled visit can be based on Investigator's judgement.
²Assessments to be performed at specified time after End of infusion. The End of infusion is defined as when the infusion of IMP is completed/stopped (i.e., before rinsing if applicable). For example, 4 h post-dose means 4 hours after end of infusion.
³Mitazalimab can be administered 2 days after gemcitabine administration
⁴The assessment may be performed within 72 hours (up to 3 days) prior to administration of study treatment.
⁵If any of the treatment discontinuation criteria apply, the patient can be discontinued from treatment and the End of treatment visit can be performed, see End of treatment visit in Table 21.
⁶If the infusion of mitazalimab is interrupted due to an AE, a PK sample and a sample for immunogenicity can be taken at the time of interruption, or as soon as is feasible considering the patient safety. An immunogenicity sample does not need to be taken if it is first administration of mitazalimab that is interrupted.
⁷Biopsy to be taken only if baseline biopsy (fresh or archival) was obtained at screening and second biopsy not obtained during Treatment cycle 1. Biopsy not to be taken if already obtained in Cycle 1.

TABLE 21
Assessment schedule for Treatment cycle 2 and subsequent cycles

| | Activity/Visit | | | | | | | | | | |
|---|---|---|---|---|---|---|---|---|---|---|---|
| | Cycle 2 and subsequent cycles (28-day treatment cycles) | | | | | | | | | | |
| | Cycle day | | | | | | | | | | |
| | 1³ | | 3 + 1 | | | 8 ± 1 | | 15 ± 1 | | 17 ± 1 | |
| | Time in relation to study drug administration | | | | | | | | | | |
| | | Gem- | | | | | Gem- | | Gem- | | |
| | Pre | NabPac | Pre | Mitazalimab4 | Post⁵ | Pre | NabPac | Pre | NabPac | Pre | Mitazalimab4 |
| Body weight | X⁶ | | | | | | | | | | |
| Vital signs | X | | X | | 1 h ± | X | | X | | X | |
| | | | | | 10 min | | | | | | |
| Physical | X⁶ | | X⁶ | | | X⁶ | | X⁶ | | X⁶ | |
| examination | | | | | | | | | | | |
| 12-lead ECG | | | | | 1 h ± | | | | | | |
| | | | | | 10 min¹² | | | | | | |
| ECOG | X⁶ | | | | | | | | | | |
| Treatment | X⁷ | | X⁷ | | | X⁷ | | X⁷ | | X⁷ | |
| discontinuation | | | | | | | | | | | |
| criteria | | | | | | | | | | | |
| Pre- and | Note: Premedications are to be given up to 3 days | | | | | | | | | | |
| post- | prior to administration of mitazalimab. | | | | | | | | | | |
| medication | | | | | | | | | | | |
| Gem-NabPac | | | | | | | | | | | |
| administration | | | | | | | | | | | |
| Nab-paclitaxel | | X | | | | | X | | X | | |
| Gemcitabine | | X | | | | | X | | X | | |
| Mitazalimab | | | | X⁸ | | | | | | | X⁸ |
| administration | | | | | | | | | | | |
| Adverse events | Continuous from signing ICF until 28 days | | | | | | | | | | |
| | after last dose of study treatment | | | | | | | | | | |
| Concomitant | Continuous from signing of ICF | | | | | | | | | | |
| medication | until End of treatment visit | | | | | | | | | | |
| Concomitant | Continuous from signing of ICF | | | | | | | | | | |
| medical | until End of treatment visit | | | | | | | | | | |
| procedures | | | | | | | | | | | |
| Clin | | | | | | | | | | | |
| laboratory tests | | | | | | | | | | | |
| Pregnancy test | X⁹ | | | | | | | | | | |
| Clinical chemistry, | X⁶ | | X⁶ | | | X⁶ | | X⁶ | | X⁶ | |
| hematology and | | | | | | | | | | | |
| coagulation | | | | | | | | | | | |
| Urinalysis | X⁶ | | | | | | | | | | |
| Pharmacokinetics | X | | | | 1 h ± | | | | | | |
| | | | | | 10 min | | | | | | |
| Immunogenicity | X | | | | | | | | | | |
| CT scan/Tumour | | | | | | | | | | | |
| response | | | | | | | | | | | |
| evaluation | | | | | | | | | | | |
| CA19-9 | X | | | | | | | | | | |
| Immunophenotyping | X | | | | | | | | | | |
| Cytokines and | | | | | | | | | | | |
| chemokines | | | | | | | | | | | |
| Whole | X | | | | | | | | | | |
| blood RNA | | | | | | | | | | | |
| Whole | X | | | | | | | | | | |
| blood DNA | | | | | | | | | | | |
| Tumour biopsy | | | | | | | | | | | |

| | Activity/Visit | | | |
|---|---|---|---|---|
| | | | | End of |
| | Cycle 2 and subsequent cycles (28-day treatment cycles) | | | treatment |
| | Cycle day | | | visit² |
| | 17 ± 1 | | | 28-56 |
| | Time in relation to study drug administration | | Unscheduled | days after |
| | Post⁵ | 24 ± 2 | visit¹ | last dose |
| Body weight | | | X | |
| Vital signs | 1 h ± | X | X | X |
| | 10 min | | | |
| Physical | | X | X | X |
| examination | | | | |
| 12-lead ECG | | | X | X |
| ECOG | | | X | X |
| Treatment | | | X⁷ | |
| discontinuation | | | | |
| criteria | | | | |
| Pre- and | Note: Premedications are to be given up to 3 days | | | |
| post- | prior to administration of mitazalimab. | | | |
| medication | | | | |
| Gem-NabPac | | | | |
| administration | | | | |
| Nab-paclitaxel | | | | |
| Gemcitabine | | | | |
| Mitazalimab | | | | |
| administration | | | | |
| Adverse events | Continuous from signing ICF until 28 days | | | |
| | after last dose of study treatment | | | |
| Concomitant | Continuous from signing of ICF | | | |
| medication | until End of treatment visit | | | |
| Concomitant | Continuous from signing of ICF | | | |
| medical | until End of treatment visit | | | |
| procedures | | | | |
| Clin | | | | |
| laboratory tests | | | | |
| Pregnancy test | | | X | X |
| Clinical chemistry, | | X | X | X |
| hematology and | | | | |
| coagulation | | | | |
| Urinalysis | | X | X | X |
| Pharmacokinetics | | | X | X |
| Immunogenicity | | | X | X |
| CT scan/Tumour | | X | X | X |
| response | | (Cycle | | |
| evaluation | | 2, 4, 6 | | |
| CA19-9 | | | X | X |
| Immunophenotyping | | | X | |
| Cytokines and | | | X | |
| chemokines | | | | |
| Whole | | | | |
| blood RNA | | | | |
| Whole | | | | |
| blood DNA | | | | |
| Tumour biopsy | | | X^{10, 11} | X^{10, 11} |

* The sections referred to in the table are the sections in the main body of the protocol.
¹The assessments to be performed during the unscheduled visit can be based on Investigator's judgement.
²After completion of the End of treatment visit, the patient will either enter the post-treatment follow-up period (see Table), or if any of the study withdrawal criteria apply, the patient will be withdrawn from the study and have the End of treatment visit and End of study visit assessments performed, see End of study visit in Table 22).
³Day 1 can be at least 14 days after the last gemcitabine plus nab-paclitaxel administration.
⁴Mitazalimab can be administered 2 days after gemcitabine administration.
⁵Assessments to be performed at specified time after End of infusion. The End of infusion is defined as when the infusion of study treatment is completed/stopped (i.e., before rinsing if applicable). For example, 1 h post-dose means 1 hour after end of infusion.
⁶The assessment may be performed within 72 hours (i.e., up to 3 days) prior to administration of study treatment.
⁷If any of the treatment discontinuation criteria apply, the patient can be discontinued from treatment and the End of treatment visit can be performed.
⁸If the infusion of mitazalimab is interrupted due to an AE, a PK sample and a sample for immunogenicity can be taken at the time of interruption, or as soon as is feasible considering patient safety.
⁹The result of the pregnancy test must be available prior to dosing.
¹⁰Biopsy to be taken only if baseline biopsy (fresh or archival) was obtained at screening.
¹¹Biopsy not to be taken if already obtained in Cycle 1 or 2.
¹²Assessment to be obtained in Cycle 2 only.

TABLE 22
Assessment schedule for post-treatment follow-up period and End of study visit

| | Activity/Visit | | |
|---|---|---|---|
| | Post-treatment follow-up | | End of study visit⁴ |
| | Time after End of treatment visit | | |
| | | 3 months, and thereafter | |
| | 1 month | every 3rd month ± 14 days | 2 years after LPI |
| Disease/Survival status | X¹ | X¹ | X¹ |
| Subsequent cancer-related therapy | X¹ | X¹ | X¹ |
| CT scan/Tumour response evaluation | | X² | X |
| Study withdrawal criteria | X³ | X³ | X |

* The sections referred to in the table are the sections in the main body of the protocol.
¹Disease/Survival status and subsequent cancer related therapy may be followed up via the patient's medical records or phone contact.
²Assessment to be performed until new cancer-related therapy/continued treatment with chemotherapy backbone alone.
³If any of the study withdrawal criteria apply, the patient will be withdrawn from the study and have the End of study visit performed.
⁴End of study visit will be performed 2 years after LPI, i.e., there will be a variation of duration of the treatment follow-up period for individual patients

### Example 6: Appendix 4 Calculation of Glomerular Filtration Rate

Glomerular filtration rate (GFR) may be estimated based on commonly used and accepted formulae, i.e., one of the below formulae.

### Cockcroft Gault Formula:

$$GFR = \frac{( 140 - age ) \times weight \times Fs}{Serum Creatinine \times 72} \\ Units : GFR [ ml / min ] , age [ years ] , weight [ kg ] , serum creatinine [ mg / dl ] , Fs is a correction Factor for Sex : in males Fs = 1 , in females Fs = 0.85$$

### Modification of Diet in Renal Disease (MDRD) Formula:

$$GFR = 170 \times Serum {Creatinine}^{- 0.999} \times {Age}^{- 0 , 176} \times {BUN}^{- 0.17} \times {Albumin}^{+ 0.318} \times Fs Units : GFR [ ml / min ] , age [ years ] , serum creatinine [ mg / dl ] , Fs is a correction Factor for Sex : in males Fs = 1 , in females Fs = 0.762$$

### Variations of the MDRD Formula:

$$GFR = 186 \times Serum {Creatinine}^{- 1.154} \times {Age}^{- 0 , 203} \times Fs Units : GFR [ ml / min ] , age [ years ] , serum creatinine [ mg / dl ] , Fs is a correction Factor for Sex : in males Fs = 1 , in females Fs = 0.742$$

### Example 7: Appendix 5 Drug Induced Liver Injury (DILI)

Alterations of liver laboratory parameters may be further evaluated using the following procedures:

### Procedures

Repeat the following laboratory tests:
ALT, AST, and bilirubin (total and direct)—within 48 to 72 hours. If ALT and/or AST>3-fold ULN combined with an elevation of total bilirubin>2-fold ULN are confirmed (if normal values at baseline/screening), or ALT and/or AST>5-fold ULN combined with an elevation of total bilirubin>2-fold ULN are confirmed (if elevated values at baseline/screening) results of the laboratory parameters described below must be made available to the investigator and to the Sponsor as soon as possible.
In addition, the following was obtained:
   a detailed history of current symptoms and concurrent diagnoses and medical history;
   a history of concomitant drug use (including non-prescription medications, herbal and dietary supplement preparations), alcohol use, recreational drug use, and special diets;
   a history of exposure to environmental chemical agents (consider home and workplace exposure).

Provide abdominal ultrasound or other appropriate imaging to rule out biliary tract, pancreatic or intrahepatic pathology, e.g., bile duct stones or neoplasm.

### Clinical Chemistry

alkaline phosphatase, albumin, PT or INR, CK, CK-MB, ceruloplasmin, α-1 antitrypsin, transferrin, amylase, lipase, fasting glucose, cholesterol, triglycerides, glutamate-dehydrogenase, D-dimers, C-reactive protein, gamma-glutamyl-transpeptidase.

### Serology

Hepatitis A (RNA), Hepatitis B (HbsAg, Anti-HBs, DNA), Hepatitis C (Anti-HCV, RNA), Hepatitis D (Anti-IgM, Anti-IgG), Hepatitis E (Anti-HEV, Anti-HEV IgM, RNA if Anti-HEV IgM positive), Cytomegalovirus (repeat CMV DNA), Anti-Smooth Muscle antibody (titer), Anti-nuclear antibody (titer), Anti-LKM (liver-kidney microsomes) antibody, Anti-mitochondrial antibody.

### Hormone

Thyroid stimulating hormone.

### Haematology

White blood count+differential, haemoglobin, thrombocytes.

In case AST/ALT remain elevated and the previous testing does not provide a likely cause for the elevation, the following tests can be performed: Epstein Barr Virus (VCA IgG, VCA IgM), herpes simplex virus (IgG, IgM), varicella (IgG, IgM), parvovirus (IgG, IgM), toxoplasmosis (IgG, IgM).

Initiate close observation of patients by repeat testing of ALT, AST, and total bilirubin (with fractionation by total and direct) at least weekly until the laboratory ALT and/or AST abnormalities stabilize or return to normal, then according to the protocol. Depending on further laboratory changes or additional parameters identified, follow-up can be based on medical judgement and Good Clinical Practice (GCP).

### Example 8: Anti-Tumor Efficacy of Mitazalimab and Chemotherapy (FOLFIRINOX) in a Chemotherapy-Resistant Preclinical Tumor Model MB-49

### Summary

The aim of this study was to explore the anti-tumor efficacy of the CD40 antibody mitazalimab in chemotherapy-resistant preclinical tumor models as an addition to chemotherapy (FOLFIRINOX). The combination of mitazalimab and FOLFIRINOX demonstrated a strong anti-tumor response in mice with established FOLFIRINOX resistant MB-49 tumors. These data support the basic concept that the combination of chemotherapy and immuno-oncology is well tolerated and has a very potent anti-tumor effect in vivo on chemotherapy-resistant cancer cells. The data further validate the potential of mitazalimab in combination with standard of care chemotherapy such as FOLFIRINOX.

### Material and Methods

### Drubs and Drub Candidates Evaluated

Note: All mice were dosed with flat doses and mg/kg doses reported herein are based on an average mouse weight of 20 g (0.02 kg).

TABLE 23
List of antibodies and controls referred to in this report

| Antibody | | Dose, | Administration | Administration | |
|---|---|---|---|---|---|
| or vehicle ID | Batch | mg/kg | volume, μl | route | Vendor |
| 10% DMSO | RNBH4175 | — | 200 | oral | Sigma |
| 10% Kolliphor | BCCD0310 | | | | Aldrich |
| EL | AF29584116 | | | | Sigma |
| 80% PBS | | | | | Aldrich |
| | | | | | HyClone |
| Mitazalimab | AG6954C | 5 | 100 | intraperitoneal | Alligator |
| Oxaliplatin | PY07218 | 2.5 | 100 | intraperitoneal | Apoteket |
| (Accord)* | PY09029 | 25 | 100 | | |
| Irinotecan | 19K28LC | 50 | 200 | | |
| (Accord)* | PY05245 | 25 | 100 | | |
| Calcium | | | | | |
| folinate | | | | | |
| (Sandoz)* | | | | | |
| 5-fluorouracil | | | | | |
| (Accord)* | | | | | |
| 5% glucose** | 19E07G65 | — | 100 | intraperitoneal | Baxter |

*Together these four components make up the chemotherapy known as FOLFIRINOX.
**Vehicle for mitazalimab and FOLFIRINOX.

### Dosing of FOLFIRINOX

As FOLFIRINOX is composed of four individual components, it was dosed over the course of two days, for a total of three cycles. Mice first received oxaliplatin followed by irinotecan, followed the day after by folinic acid and 2 hours after that, mice were dosed with 5-fluorouracil.

### Dosing of Mitazalimab

Three days following the initiation of the dosing of FOLFIRINOX, mitazalimab was administered in 5% glucose (dextrose buffer) at 5 mg/kg.

### Dosing Regimen

The dosing regimen applied in the two efficacy studies detailing number of doses and their relation to one another is outlined in FIG. 5.

### Cell Lines

Scandion Oncology generated MB49 cell lines resistant to three cytostatic components of the combined chemotherapy FOLFIRINOX (Irinotecan, Oxaliplatin and 5-fluorouracil). Two chemotherapy-resistant MB49 bladder carcinoma cell lines were produced, namely MB49-FOLFIRINOX-de novo and MB49-FOLFIRNINOX-acquired (MB49-FFX-ACQ). Methods of making a chemotherapy-resistant cell lines (such as with MB49) are known to the skilled person (as described in Amaral et al., 2019, Establishment of Drug-resistant Cell Lines as a Model in Experimental Oncology: A Review, Anticancer Research, 36:6443-6455). The de novo cell line was later disregarded for in vivo titration due to its poor growth in culture, and the experiments described in this report have been performed with the MB49 cell line with acquired chemotherapy resistance.

The MB49 mouse bladder carcinoma cell line was used as a starting point for the FOLFIRINOX-resistant model. Resistance can be established using the following approaches:
1. Acquired resistance: cancer cells were exposed to gradually increasing concentrations of drugs until a resistant cell population is generated. Based on previous experience (PMID: 26801902, 25759163, 25596703; Jandu et al., 2016, Jensen et al., 2015, and Hansen et al., 2015), a starting concentration 50-fold below the IC50 values of each drug was used, gradually increasing 2-fold for every third passaging of the cells (approximately every third week) until a difference in IC50 for each drug, when compared with drug sensitive cells, was increased at least by 5-fold. This process results in MB49 cell lines with an increased resistance to FOLFIRINOX.
2. De novo resistance: cancer cells were exposed to an initial high drug concentration, varying from 10-fold below to 10-fold above the IC50 values of each drug. The objective of this approach was to kill most of the cancer cells. However, a few cancer cells with “de novo” resistance will survive, being then cultured, and expanded to establish a drug resistant cell population.

Initially, only MB49-FFX-ACQ cultured without FOLFIRINOX before cell banking were titrated in vivo (ELN 148285), and a small-scale follow-up titration was later added to enable comparison to the cell line grown with FOLFIRINOX before master cell banking (ELN 148525).

Experiments in Tumor-Bearing hCD40tg Mice

The hCD40tg mouse strain on C57Bl/6 background has previously been generated by Alligator Bioscience (DOCID-1084249735-8226; Mangsbo et al., 2015, The human agonistic CD40 antibody ADC-1013 eradicates bladder tumors and generates T-cell-dependent tumor immunity, Clin Cancer Res; 21(5):1115-26).

On day 0, ten- to twelve-week-old female hCD40tg mice were injected subcutaneously on the right hind flank with 0.4×10⁶ MB49-FOLFIRINOX-ACQ in a volume of 100 μl PBS. Cells were cultured either with (study 1) or without FOLFIRINOX (study 2) before inoculation in mice. Controls or antibodies were administered as outlined in Table 24, and mice were monitored shortly following each treatment to detect potential signs of toxicity. Tumor volume was measured three times weekly with a calliper and calculated as: ((width/2×length/2×height/2)×4n/3). Mice were weighed weekly, and weight loss exceeding 25% was considered an ethical endpoint, as well as tumor volume exceeding 2 cm³, tumor ulceration or affected health.

TABLE 24
Group allocation and experimental setup

| | No. of | | Treatment | |
|---|---|---|---|---|
| Group | mice | Treatment | route | Treatment days |
| 1 | 10 | Vehicle | i.p. | 7-8, 14-15, 21-22 |
| | | | | 10, 17, 24 |
| 2 | 10 | mitazalimab | i.p. | 10, 17, 24 |
| 3 | 10 | FOLFIRINOX | i.p. | 7-8, 14-15, 21-22 |
| 4 | 10 | mitazalimab | i.p. | 10, 17, 24 |
| | | FOLFIRINOX | i.p. | 7-8, 14-15, 21-22 |

### Statistical Analysis

Collected data was analyzed using Kaplan Meier and Log-rank (Mantel-Cox) for survival and Mann-Whitney, non-parametric, 2-tail for FACS data and tumor growth using GraphPad Prism program (*, p<0.05; **, p<0.01).

### Results

Study Using MB49-FFX-ACQ Co-Cultured with FOLFIRINOX

The MB49-FFX-ACQ cells were grown with addition of 0.3 μM 5-fluorouracil, 4.5 μM oxaliplatin and 0.12 μM irinotecan (SN38) to the culture media up until three passages before master cell banking.

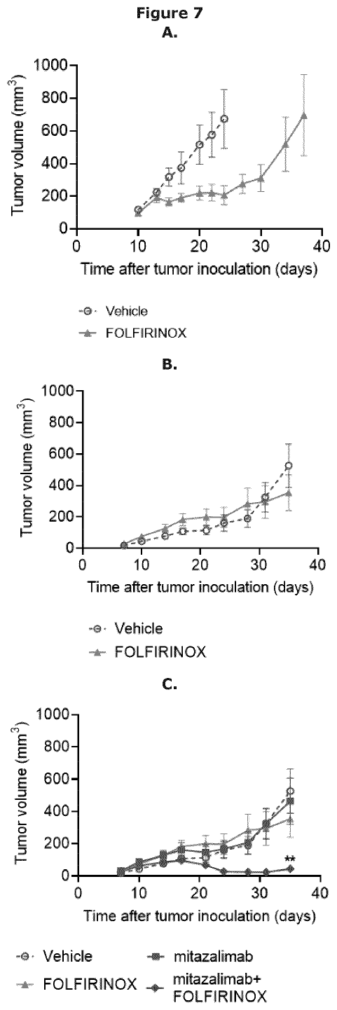

FIG. 7A demonstrates that FOLFIRINOX reduces tumour growth in chemo-sensitive tumours. FIG. 7B demonstrates no effect of FOLFIRINOX in chemo-resistant tumours. FIG. 7C demonstrates mitazalimab synergises with FOLFIRINOX in chemo-resistant tumours.

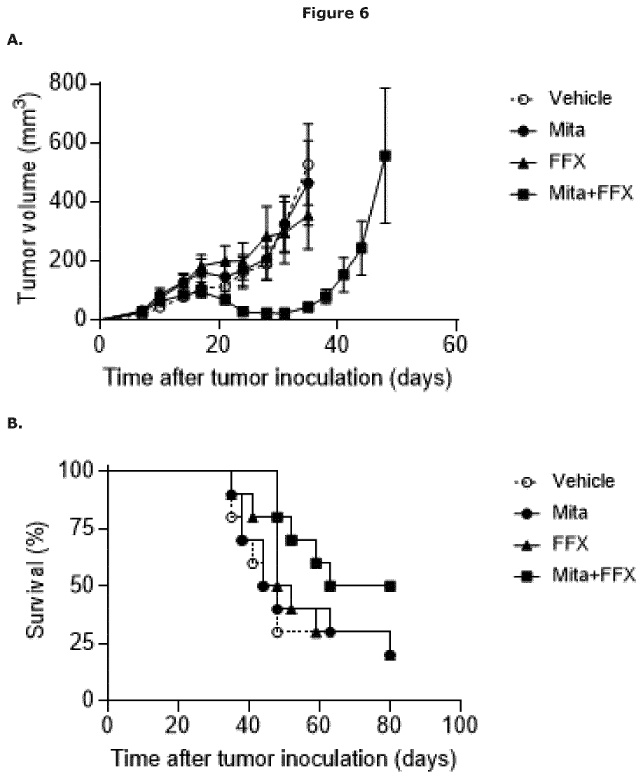

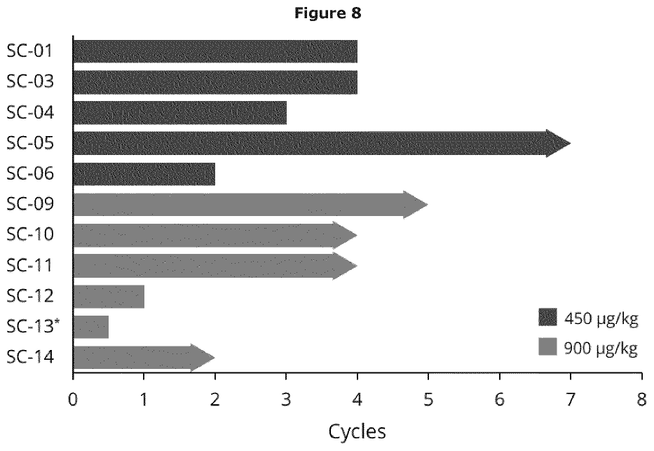

The combination of mitazalimab+FOLFIRINOX induced statistically significant tumor growth inhibition compared to vehicle, and cured tumor-bearing mice (complete responders: 5/10 for mitazalimab+FOLFIRINOX), see FIGS. 6 and 8.

### Evaluation and Conclusions

In the performed efficacy study, the combination of mitazalimab and FOLFIRINOX demonstrated a strong anti-tumor response in mice with established FOLFIRINOX resistant MB49 tumors. These data support the basic concept that the combination of chemotherapy and immuno-oncology is well tolerated and has a very potent anti-tumor effect in vivo on chemotherapy resistant cancer cells. The data further validate the potential of mitazalimab in combination with standard of care chemotherapy such as FOLFIRINOX.

### Example 9: Safety Data from OPTIMIZE-1, a Phase 1b/2 Study of Mitazalimab in Combination with mFOLFIRINOX in Patients with Metastatic Pancreatic Ductal Adenocarcinoma (PDAC): Mitazalimab 900 μg/kg Determined as Safe and Recommended Dose for Phase 2 (RP2D) Part of the Study

Mitazalimab is a human CD40 agonistic IgG1 antibody being developed as cancer immunotherapy. Targeting CD40 kickstarts the cancer immunity cycle by licensing DCs leading to tumor-specific T cell priming and activation. Furthermore, in PDAC, CD40 agonists activates myeloid cells and promote degradation of the desmoplastic tumor stroma, improving influx of T cells and chemotherapeutic agents into the tumor.

Mitazalimab has shown to be safe and well tolerated (at doses up to 1200 μg/kg) with signs of clinical activity in solid tumors in a Phase I study (NCT02829099). Most drug related adverse events (AE) were grade 1 or 2.

OPTIMIZE-1 (NCT04888312) is a Phase 1b/2, open-label, multicenter study designed to evaluate safety, tolerability, and efficacy of mitazalimab in combination with mFOLFIRINOX in adults diagnosed with previously untreated metastatic PDAC.

The objective of the first (Phase 1b) part of the study was to determine the RP2D of mitazalimab+mFOLFIRINOX. Mitazalimab was escalated from 450 μg/kg to 900 μg/kg following a Bayesian optimal interval design with at least 3 patients enrolled per dose level. In the first 21-day treatment cycle (Dose Limiting Toxicity assessment period), mitazalimab was administered intravenously on days 1 and 10 and mFOLFIRINOX starts on day 8. In the second and subsequent cycles, treatment followed a 14-day cycle schedule where mitazalimab was administered 2 days after mFOLFIRINOX.

In part 2 of the study (Phase 2), mitazalimab at the RP2D was administered in combination with mFOLFIRINOX. Primary endpoint was RECIST-defined overall response rate. Progression-free survival and overall survival was assessed as secondary endpoints.

In the Phase 1b (dose escalation) part of this study, 11 patients were treated with mitazalimab: 5 at 450 μg/kg and 6 at 900 μg/kg mitazalimab doses. One patient in the 900 μg/kg dose cohort withdrew from the trial for administrative reasons after the first mitazalimab infusion, prior to receiving mFOLFIRINOX and was not included in RP2D determination. Key baseline characteristics included: 7 female, 4 male; median age 63 (range 57-70); ECOG 0-1; median time since PDAC diagnosis 25 days.

Mitazalimab related AEs were reported in 9/11 patients. Treatment related AEs occurring in >1 patient were fever (60%), muscle pain (50%) and fatigue (20%). At the 450 μg/kg dose, all mitazalimab related AEs were grade 1-2. At the 900 μg/kg dose, 4 patients (67%) experienced grade 1-2 mitazalimab related AEs. One patient in the 900 μg/kg dose experienced mitazalimab related grade 3 fatigue and grade 3 headache that led to treatment discontinuation after the first cycle. There were no mitazalimab related grade 4 or 5 AEs. 1/10 patients required mFOLFIRINOX dose reduction and at the cutoff date the range of treatment length was 1-14 weeks.

Mitazalimab combined with mFOLFIRINOX was safe and well tolerated. The 900 μg/kg dose of mitazalimab was selected as the RP2D and patient enrolment at the RP2D is ongoing.

### OPTIMIZE-1 Safety Summary

### DLTs:

SC-12 (001-009): G3 Headache (related to Mitazalimab). 59-year-old female h/o NSCLC; progression on C4 scan, also new lung lesion. Off study.

### SAEs:

SC-01 (002-001): SAE G3 New Lung Cancer (unrelated). 60-year-old female h/o NSCLC; progression on C4 scan, also new lung lesion. Off study.
SC-05 (103-001): SAE G3 Anorexia (unrelated). 65-year-old male h/o abd pain, ascites. Hospitalization for pain management. Resolved. Study treatment ongoing.
SC-06 (001-001): Nullified SAE G2 Alteration of General Status. 66-year-old male. Alternative cause: progression.
SC-14 (001-011): SAE G3 Supraventricular Tachycardia (unrelated). 60-year-old female. Resolved. Study treatment ongoing.

### AESIs:

None (Infusion-Related Reactions G2; Cytokine Release Syndrome G2; LFTs>5×ULN; Bilirubin 1.5×ULN).

### Mitazalimab-Related (of 11 Patients at 2 Dose Levels):

Grade 3: Fatigue (2); Headache (1); Hypokalemia (1).
Grade 1-2 (most common): Fever or similar (6); Flu/muscle ache (5); Fatigue (3).

### Example 10: OPTIMIZE-1 Futility Analysis

As mentioned in the study design of Example 2, an interim futility analysis was performed on 23 patients in total (results summarised in Table 28). The patient disposition for the safety set is shows in Table 25, in which the number of screened subjects and their inclusion or exclusion (including reasons for exclusion) are outlined. The patient disposition for the Full Analysis Set (FAS) at the 900 μg/kg dose is shown in Table 26, with the respective patient demographics shown in Table 27.

TABLE 25
Patient disposition (Safety set)

| | | 450 μg/kg | | 900 μg/kg | | | |
|---|---|---|---|---|---|---|---|
| | | Mitazalimab | | Mitazalimab | | Overall | |
| | | (N = 5) | | (N = 38) | | (N = 43) | |
| Screened subjects | | 5 | (100.0) | 38 | (100.0) | 43 | (100.0) |
| Included in safety set | | 5 | (100.0) | 38 | (100.0) | 43 | (100.0) |
| Excluded from safety set | | 0 | (0.0) | 0 | (0.0) | 0 | (0.0) |
| Included in full analysis set (Efficacy) | | 0 | (0.0) | 23 | (60.5) | 23 | (53.5) |
| Excluded from full analysis set | | 5 | (100.0) | 15 | (39.5) | 20 | (46.5) |
| 1. | Beyond required sample size of 23 patients | 0 | (0.0) | 9 | (23.7) | 9 | (20.9) |
| | for futility analysis | | | | | | |
| 2. | Did not receive the combination of | 5 | (100.0) | 0 | (0.0) | 5 | (11.6) |
| | mitazalimab at the RP2D | | | | | | |
| 3. | Did not complete two cycles of treatment | 0 | (0.0) | 6 | (15.8) | 6 | (14.0) |
| Ongoing treatment | | 0 | (0.0) | 27 | (71.1) | 27 | (62.8) |
| Discontinued treatment | | 5 | (100.0) | 11 | (28.9) | 16 | (37.2) |
| 1. | Progressive disease | 2 | (40.0) | 3 | (7.89) | 5 | (11.6) |
| 2. | Unacceptable toxicity | 1 | (20.0) | 4 | (10.5) | 5 | (11.6) |
| 3. | Death due to disease progression | 2 | (40.0) | 1 | (2.6) | 3 | (7.0) |
| 4. | Clear clinical deterioration where | 0 | (0.0) | 1 | (2.6) | 1 | (2.3) |
| | continued study treatment would not be in | | | | | | |
| | the best interest of the patient, as judged | | | | | | |
| | by the Investigator | | | | | | |
| 5. | Other | 0 | (0.0) | 1 | (2.6) | 1 | (2.3) |
| 6. | Withdrawal of consent | 0 | (0.0) | 1 | (2.6) | 1 | (2.3) |
| In Survival follow-up period after treatment | | 2 | (40.0) | 6 | (15.8) | 8 | (18.6) |
| Discontinued study | | 3 | (60.0) | 2 | (5.3) | 5 | (11.6) |
| 1. | Death due to disease progression | 2 | (40.0) | 1 | (2.6) | 3 | (7.0) |
| 2. | Withdrawal of consent | 1 | (20.0) | 1 | (2.6) | 2 | (4.7) |

Results show number of patients and percentage of patients in brackets relative to N for overall parameters. Possible reasons are displayed only if applicable.

TABLE 26
Patient disposition (FAS)

| | | 900 μg/kg | |
|---|---|---|---|
| | | Mitazalimab | |
| | | (N = 23) | |
| Ongoing treatment | | 18 | (78.3) |
| Discontinued treatment | | 5 | (21.7) |
| 1. | Clear clinical deterioration where continued study treatment would | 1 | (4.3) |
| | not be in the best interest of the patient, as judged by the | | |
| | Investigator | | |
| 2. | Progressive disease | 2 | (8.7) |
| 3. | Death due to disease progression | 1 | (4.3) |
| 4. | Unacceptable toxicity | 1 | (4.3) |
| In Survival follow-up period after treatment | | 3 | (13.0) |
| Discontinued study | | 1 | (4.3) |
| 1. | Death due to disease progression | 1 | (4.3) |

Results show number of patients and percentage of patients in brackets relative to N for overall parameters. Possible reasons are displayed only if applicable.
Data cut-off: 2022 Dec. 8
Program: t_fut_disp_fas.sas

TABLE 27
Patient demographics (FAS)

| | | 900 μg/kg Mitazalimab | |
|---|---|---|---|
| | | (N = 23) | |
| Age (years) | n | 23 | |
| | Mean | 63.8 | |
| | SD | 7.8 | |
| | Minimum | 43 | |
| | Median | 64.0 | |
| | Maximum | 77 | |
| Age Group | n | 23 | |
| | 65 or above | 11 | (47.8) |
| | Below 65 | 12 | (52.2) |
| Gender | n | 23 | |
| | Female | 14 | (60.9) |
| | Male | 9 | (39.1) |
| ECOG Performance Status | n | 23 | |
| | Grade 1 | 7 | (30.4) |
| | Grade 0 | 16 | (69.6) |

Abbreviations: n = number of patients with non-missing value, SD = standard deviation, ECOG = Eastern Cooperative Oncology Group. Categoric results show number of patients and percentage of patients in brackets relative to n of the respective category.
Data cut-off: 2022 Dec. 8
Program: t_fut_dm_fas.sas

TABLE 28
Futility analysis (FAS)

| | | | | | | | | Disease |
|---|---|---|---|---|---|---|---|---|
| | Complete | Partial | Stable | Progressive | Not | No | | control |
| | response | response | disease | disease | evaluable | imaging^{b} | Responders^{c} | rate^{d} |
| Best | 0 | 12 | 9 | 2 | 0 | 0 | 12 | 21 |
| Overall | | (52.2) | (39.1) | (8.7) | | | (52.2) | (91.3) |
| Response^{a} | | | | | | | | |
| n (%); | | | | | | | | |
| N = 23 | | | | | | | | |

n = number of patients included in calculations
^{a}Responses do not have to be confirmed by a subsequent scan for the futility analysis
^{b}No tumor response assessment available for futility analysis, these patients are regarded as non-responders
^{c}Responder is defined as having complete or partial response, as per RECIST 1.1 criteria
^{d}Disease control is defined as having complete response, partial response or stable disease

The futility analysis (Table 28) shows a promising disease control rate of 91.3% of the subjects involved who were treated with the 900 μg/kg dose of mitazalimab and mFOLFIRINOX cotreatment. Therefore, these preliminary data show an improvement in the treatment of cancer for the combination therapy. In patients with progressive disease, this likely demonstrates that the patients were unresponsive to the combination therapy, and not that the combination therapy provoked progression of the disease.

Importantly, these data show that the mitazalimab RP2D of 900 μg/kg showed promising efficacy. These are the first reported data on a (second generation) CD40 antibody in combination with FOLFIRINOX in this patient population. The ORR of 52.2% is substantially better than the historically reported ORR of 31.6% with FOLFIRINOX alone. Modified FOLFIRINOX applies a lower dose of irinotecan, implying reduced toxicity and better adherence to therapy, thereby improving efficacy.

A substantial proportion of patients (18 out of 23) were still ongoing with treatment at the point of this analysis, indicating promising clinical activity as well as likely manageable safety profiles. Only one patient (Patient ID 002-024) had to discontinue mitazalimab treatment due to an adverse event (pneumonia), and even this patient demonstrated a partial response at the end-of-treatment scan. 8 out of 12 responders in this analysis had a confirmed response via a subsequent scan, and 3 out of 4 unconfirmed responders were ongoing with treatment at the analysis date. One patient, SC-22, had a near-complete response with disappearance of one target lesion (it is only due to the persistent non-target lesion that this patient had to formally be considered as “partial response” instead of “complete response”).

Furthermore, one patient who had a delayed start of FOLFIRINOX showed a borderline disease progression at the first post-baseline assessment. While this patient was initially regarded as a non-responder, the subsequent scans showed tumour shrinkage. Steady tumour shrinkage was observed during treatment for this patient.

The patients used in the clinical study were assigned patient IDs, all of which were included in the safety set, but some of which were excluded from the FAS set data for each treatment arm, as shown in Table 29 (450 μg/kg mitazalimab and mFOLFIRINOX cotreatment) and Table 30 (900 μg/kg mitazalimab and mFOLFIRINOX cotreatment).

TABLE 29
Listing 1 Patient disposition (Safety set) - Treatment
arm = 450 μg/kg mitazalimab + mFOLFIRINOX

| | | Reason for | | |
|---|---|---|---|---|
| | | Exclusion | | |
| Patient | Safety | from Safety | | |
| ID | Set | Set | FAS | Reason for Exclusion from FAS |
| 001-001 | Yes | — | No | Did not receive the combination |
| | | | | of mitazalimab at the RP2D |
| 002-001 | Yes | — | No | Did not receive the combination |
| | | | | of mitazalimab at the RP2D |
| 002-002 | Yes | — | No | Did not receive the combination |
| | | | | of mitazalimab at the RP2D |
| 003-001 | Yes | — | No | Did not receive the combination |
| | | | | of mitazalimab at the RP2D |
| 103-001 | Yes | — | No | Did not receive the combination |
| | | | | of mitazalimab at the RP2D |

Abbreviations: FAS = Full Analysis Set

TABLE 30
Listing 1 Patient disposition (Safety set) - Treatment
arm = 900 μg/kg mitazalimab + mFOLFIRINOX

| | | Reason for | | |
|---|---|---|---|---|
| | | Exclusion | | |
| Patient | Safety | from Safety | | |
| ID | Set | Set | FAS | Reason for Exclusion from FAS |
| 001-006 | Yes | — | Yes | — |
| 001-009 | Yes | — | No | Did not complete two cycles of |
| | | | | treatment |
| 001-011 | Yes | — | Yes | — |
| 001-013 | Yes | — | Yes | — |
| 001-016 | Yes | — | Yes | — |
| 001-023 | Yes | — | Yes | — |
| 001-026 | Yes | — | Yes | — |
| 001-032 | Yes | — | Yes | — |
| 001-036 | Yes | — | No | Beyond required sample size of |
| | | | | 23 patients for futility analysis |
| 002-008 | Yes | — | Yes | — |
| 002-010 | Yes | — | No | Did not complete two cycles of |
| | | | | treatment |
| 002-024 | Yes | — | Yes | — |
| 002-034 | Yes | — | No | Beyond required sample size of |
| | | | | 23 patients for futility analysis |
| 002-044 | Yes | — | No | Did not complete two cycles of |
| | | | | treatment |
| 003-017 | Yes | — | Yes | — |
| 003-018 | Yes | — | Yes | — |
| 003-025 | Yes | — | Yes | — |
| 003-031 | Yes | — | Yes | — |
| 003-035 | Yes | — | No | Beyond required sample size of |
| | | | | 23 patients for futility analysis |
| 004-015 | Yes | — | Yes | — |
| 004-019 | Yes | — | Yes | — |
| 004-029 | Yes | — | Yes | — |
| 101-007 | Yes | — | Yes | — |
| 101-022 | Yes | — | Yes | — |
| 101-028 | Yes | — | No | Did not complete two cycles of |
| | | | | treatment |
| 101-039 | Yes | — | No | Beyond required sample size of |
| | | | | 23 patients for futility analysis |
| 101-042 | Yes | — | No | Beyond required sample size of |
| | | | | 23 patients for futility analysis |
| 103-012 | Yes | — | Yes | — |
| 103-030 | Yes | — | No | Did not complete two cycles of |
| | | | | treatment |
| 103-033 | Yes | — | No | Beyond required sample size of |
| | | | | 23 patients for futility analysis |
| 104-020 | Yes | — | Yes | — |
| 104-021 | Yes | — | Yes | — |
| 105-038 | Yes | — | No | Beyond required sample size of |
| | | | | 23 patients for futility analysis |
| 105-040 | Yes | — | No | Beyond required sample size of |
| | | | | 23 patients for futility analysis |
| 106-014 | Yes | — | Yes | — |
| 106-027 | Yes | — | Yes | — |
| 201-041 | Yes | — | No | Did not complete two cycles of |
| | | | | treatment |
| 203-037 | Yes | — | No | Beyond required sample size of |
| | | | | 23 patients for futility analysis |

Abbreviations: FAS = Full Analysis Set

The tumour response assessments were also included in the futility analysis, as shown in Table 31. The visits made to the patients are outlined (the details of the treatment cycles are as previously described) along with the target lesion(s) response and non-target lesion(s) response. The best overall response corresponds to the most positive outcome for the target lesion(s) response observed in that particular patient, wherein the order of response from best to worst is: (i) complete response (CR); (ii) partial response (PR); (iii) stable disease (SD); and (iv) progressive disease (PD). The target lesion(s) and non-target lesion(s) responses are as previously described in Example 3.

TABLE 31
Listing 2 Tumour response assessments included in futility analysis

| | | | Target | Non-target | |
|---|---|---|---|---|---|
| Patient | | Date of | lesion(s) | lesion(s) | Overall |
| ID | Visit | imaging | response | response | response |
| 001-006 | Cycle 4 | 2022 Mar. 3 | PR | Non-CR/non-PD | PR |
| | Cycle 8 | 2022 Apr. 28 | PR | Non-CR/non-PD | PR |
| | Best Overall | | | | PR |
| | Response^{a} | | | | |
| 001-011 | Cycle 4 | 2022 Apr. 1 | SD | Non-CR/non-PD | SD |
| | Cycle 8 | 2022 Jun. 9 | SD | Non-CR/non-PD | SD |
| | Best Overall | | | | SD |
| | Response^{a} | | | | |
| 001-013 | Cycle 4 | 2022 Jun. 30 | SD | Non-CR/non-PD | SD |
| | Cycle 8 | 2022 Aug. 25 | PR | Non-CR/non-PD | PR |
| | Best Overall | | | | PR |
| | Response^{a} | | | | |
| 001-016 | Cycle 4 | 2022 Jul. 7 | SD | Non-CR/non-PD | SD |
| | Cycle 8 | 2022 Sep. 1 | PD | Non-CR/non-PD | PD |
| | End of Treatment | 2022 Sep. 1 | PD | Non-CR/non-PD | PD |
| | visit | | | | |
| | Best Overall | | | | SD |
| | Response^{a} | | | | |
| 001-023 | Cycle 4 | 2022 Sep. 1 | PR | Non-CR/non-PD | PR |
| | Cycle 8 | 2022 Nov. 10 | PR | Non-CR/non-PD | PR |
| | Best Overall | | | | PR |
| | Response^{a} | | | | |
| 001-026 | Cycle 4 | 2022 Sep. 15 | PR | Non-CR/non-PD | PR |
| | Unscheduled visit | 2022 Nov. 10 | PR | Non-CR/non-PD | PR |
| | Best Overall | | | | PR |
| | Response^{a} | | | | |
| 001-032 | Cycle 4 | 2022 Oct. 13 | SD | Non-CR/non-PD | SD |
| | Unscheduled visit | 2022 Dec. 8 | SD | Non-CR/non-PD | SD |
| | Best Overall | | | | SD |
| | Response^{a} | | | | |
| 002-008 | Cycle 4 | 2022 Mar. 15 | SD | Non-CR/non-PD | SD |
| | Cycle 8 | 2022 May 11 | PR | Non-CR/non-PD | PR |
| | Best Overall | | | | PR |
| | Response^{a} | | | | |
| 002-024 | Cycle 4 | 2022 Aug. 30 | SD | Not evaluable (NE) | SD |
| | End of Treatment | 2022 Oct. 19 | PR | Not evaluable (NE) | PR |
| | visit | | | | |
| | Best Overall | | | | PR |
| | Response^{a} | | | | |
| 003-017 | Cycle 4 | 2022 Jul. 27 | PR | Non-CR/non-PD | PR |
| | Cycle 8 | 2022 Sep. 21 | PR | Non-CR/non-PD | PR |
| | Best Overall | | | | PR |
| | Response^{a} | | | | |
| 003-018 | Cycle 4 | 2022 Aug. 10 | SD | Non-CR/non-PD | SD |
| | Cycle 8 | 2022 Oct. 3 | SD | Non-CR/non-PD | SD |
| | Best Overall | | | | SD |
| | Response^{a} | | | | |
| 003-025 | Cycle 4 | 2022 Sep. 5 | SD | Non-CR/non-PD | SD |
| | Cycle 8 | 2022 Oct. 31 | PR | CR | PR |
| | Best Overall | | | | PR |
| | Response^{a} | | | | |
| 003-031 | Cycle 4 | 2022 Sep. 28 | PR | Non-CR/non-PD | PR |
| | Unscheduled visit | 2022 Nov. 21 | SD | Non-CR/non-PD | SD |
| | Best Overall | | | | SD |
| | Response^{a} | | | | |
| 004-015 | End of Treatment | 2022 Jun. 28 | PD | PD | PD |
| | visit | | | | |
| | Best Overall | | | | PD |
| | Response^{a} | | | | |
| 004-019 | Cycle 4 | 2022 Aug. 17 | SD | Non-CR/non-PD | SD |
| | Cycle 8 | 2022 Oct. 20 | SD | Non-CR/non-PD | SD |
| | Best Overall | | | | SD |
| | Response^{a} | | | | |
| 004-029 | Cycle 4 | 2022 Sep. 26 | PR | Non-CR/non-PD | PR |
| | Cycle 8 | 2022 Nov. 15 | PR | Non-CR/non-PD | PR |
| | Best Overall | | | | PR |
| | Response^{a} | | | | |
| 101-007 | Cycle 4 | 2022 Mar. 17 | SD | Non-CR/non-PD | SD |
| | Cycle 8 | 2022 May 12 | PR | Non-CR/non-PD | PR |
| | Best Overall | | | | PR |
| | Response^{a} | | | | |
| 101-022 | Cycle 4 | 2022 Aug. 26 | SD | Non-CR/non-PD | SD |
| | Cycle 8 | 2022 Oct. 20 | PR | Non-CR/non-PD | PR |
| | Best Overall | | | | PR |
| | Response^{a} | | | | |
| 103-012 | Cycle 4 | 2022 Jun. 30 | PR | Non-CR/non-PD | PR |
| | Cycle 8 | 2022 Aug. 26 | PR | CR | PR |
| | Best Overall | | | | PR |
| | Response^{a} | | | | |
| 104-020 | Cycle 4 | 2022 Aug. 16 | SD | Non-CR/non-PD | SD |
| | Unscheduled visit | 2022 Oct. 20 | SD | Non-CR/non-PD | SD |
| | Best Overall | | | | SD |
| | Response^{a} | | | | |
| 104-021 | Cycle 4 | 2022 Aug. 23 | SD | Non-CR/non-PD | SD |
| | Cycle 8 | 2022 Oct. 18 | SD | Non-CR/non-PD | SD |
| | Best Overall | | | | SD |
| | Response^{a} | | | | |
| 106-014 | Unscheduled visit | 2022 Jul. 8 | SD | Non-CR/non-PD | SD |
| | Cycle 8 | 2022 Sep. 2 | SD | Non-CR/non-PD | SD |
| | Best Overall | | | | SD |
| | Response^{a} | | | | |
| 106-027 | Cycle 4 | | | | No imaging |
| | Cycle 8 | | | | No imaging |
| | Unscheduled visit | 2022 Sep. 23 | PD | PD | PD |
| | Unscheduled visit | 2022 Oct. 18 | SD | Non-CR/non-PD | PD |
| | Unscheduled visit | 2022 Nov. 15 | SD | Non-CR/non-PD | PD |
| | Best Overall | | | | PD |
| | Response^{a} | | | | |

^{a}Responses do not have to be confirmed for this (futility) analysis

An unscheduled visit may have occurred for a number of reasons, but was most often due to administration reasons or patient preference. At other times, it may have been due to a delay in resuming treatment, for example due to an adverse event or the patient's preference. Regarding Patient ID 004-015, this patient received treatment as planned, but the disease progressed rapidly, which resulted in the premature end of treatment.

Patient ID 106-027 was started on FOLFIRINOX relatively late due to safety issues. The first scan showed borderline progressive disease, and so a decision was made to continue treatment. Following treatment with the combination, the disease is responding, with two later unscheduled scans showing a stable disease.

### Example 11: Mitazalimab and mFOLFIRINOX Safety Data and Recommended Dose for Phase 2 from OPTIMIZE-1 (a Phase 1b/2 Study)

The primary objective for the Phase 1b aspect was to determine the recommended Phase 2 dose. Secondary objectives include the assessment of clinical activity (overall response rate (ORR), disease control rate and time to next anti-cancer therapy) and survival outcome.

The primary objective for the Phase 2 aspect was to assess the clinical activity of mitazalimab in combination with chemotherapy as determined by ORR. Secondary objectives include survival outcomes, Best Overall Response (BOR), Duration of Response (DoR), Duration of SD, Disease control rate, and time to next anti-cancer therapy.

The patient demographics for the data generated in Example 11 is shown in Table 32, with an analysis of Treatment Emergent Adverse Events (TEAEs) and Mitazalimab Related Adverse Events shown in Tables 33 and 34, respectively.

TABLE 32
Patient demographics

| | Overall | |
|---|---|---|
| Number of patients | 11 | |
| Median (range) age, years | 62 | (57-70) |
| Age Group, n (%) | | |
| 18-64 | 7 | (63.6) |
| 65+ | 4 | (36.4) |
| ECOG Performance status, n (%) | | |
| 1 | 5 | (45.5) |
| 0 | 6 | (54.4) |

| | |
|---|---|
| Median time since mPDAC diagnosis, days | 25 |

TABLE 33
Treatment Emergent Adverse Events (TEAEs)

| | Mitazalimab dose level (μg/kg) | | | |
|---|---|---|---|---|
| | 450 | | 900 | Overall |
| Number of patients | 5 | | 6 | 11 |
| Patients with any TEAEs, n (%) | 5 | (100) | 4 (66.7) | 9 (81.8) |
| Grade 1 or 2 TEAEs, n (%) | 5 | (100) | 4 (66.7) | 9 (81.8) |
| Grade 3 TEAEs, n (%) | 3 | (60) | 1 (16.7) | 4 (36.4) |

| | | | |
|---|---|---|---|
| Grade ≥4 TEAEs, n (%) | 0 | 0 | 0 |

TABLE 34
Mitazalimab Related Adverse Events

| | Mitazalimab dose level (μg/kg) | | | |
|---|---|---|---|---|
| | 450 | 900 | Overall | |
| Number of patients | 5 | 6 | 11 | |
| Patients with any TEAEs, n (%) | 4 (80) | 4 (66.7) | 8 | (72.7) |
| Grade 1 or 2 TEAEs, n (%) | 4 (80) | 4 (66.7) | 8 | (72.7) |
| Grade 3 TEAEs, n (%) | 0 | 1 (16.7) | 1 | (9.1) |
| DLTs | 0 | 1 (16.7) | 1 | (9.1) |

The five patients at 450 μg/kg correspond to SC-01, SC-03, SC-04, SC-05 and SC-06; and the six patients at 900 μg/kg correspond to SC-09, SC-10, SC-11, SC-12, SC-13 and SC-14. The treatment duration for these patients is shown in FIG. 8. Patient SC-13 withdrew from the trial for administration reasons after the first mitazalimab infusion, prior to receiving mFOLFIRINOX, and was not included in RP2D determination.

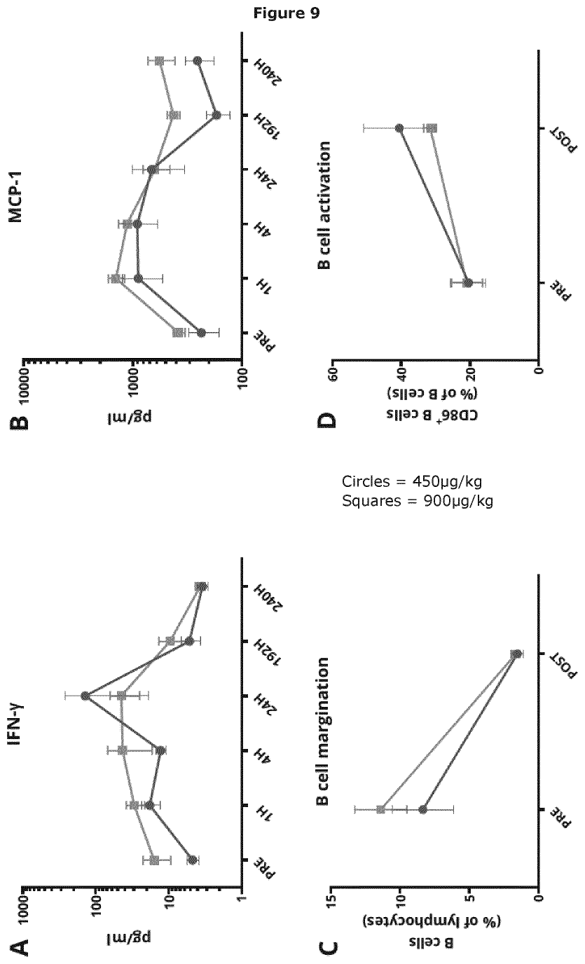

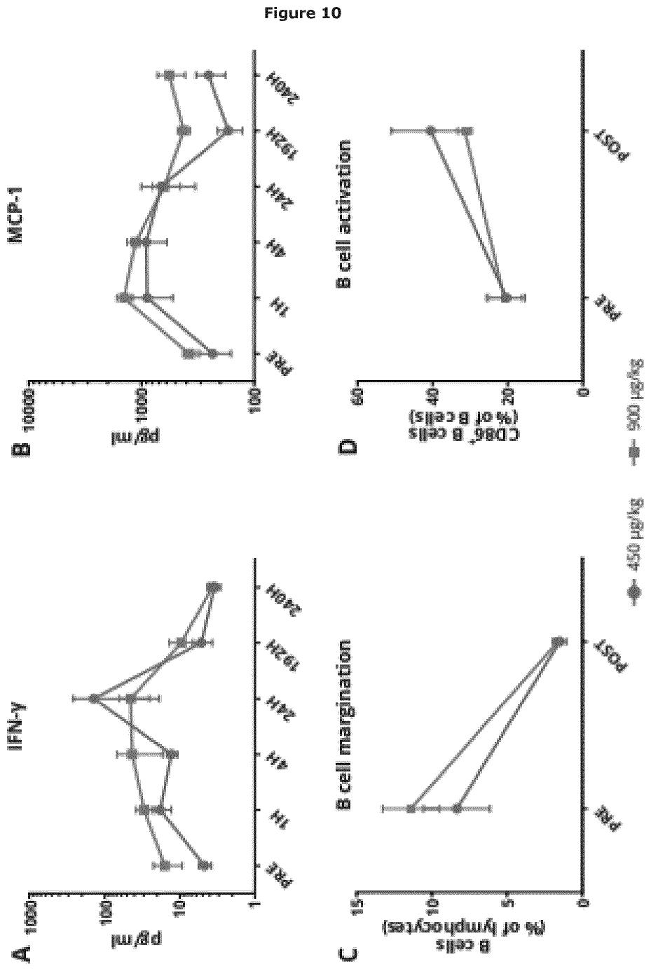

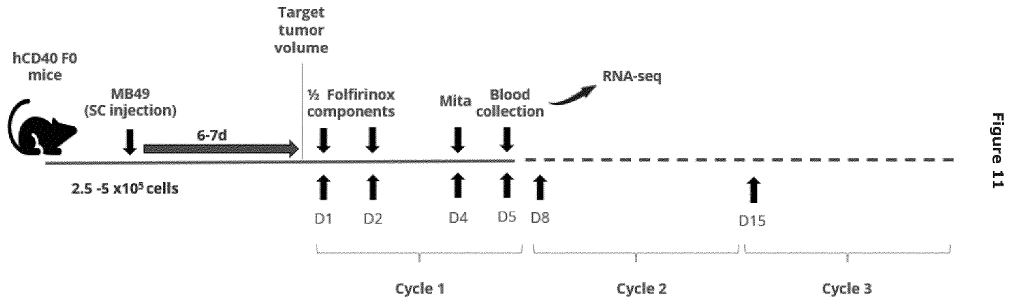

Several peripheral blood assessments were also conducted to assess IFN-γ, MCP-1, B cell margination, and B cell activation (see FIGS. 9A-D and 10A-D). Cytokine and chemokine increases in peripheral blood were observed, which confirms activation of T cells and myeloid cells at both doses of the mitazalimab tested (see FIGS. 9A and 9B and 10A and 10B). Furthermore, B cell margination and B cell activation for remaining B cells was observed in peripheral blood post-treatment with mitazalimab (see FIGS. 9C and 9D and 10C and 10D).

### Conclusion

These data support that the combination of mitazalimab with mFOLFIRINOX is safe and well tolerated. Mitazalimab related adverse events (AEs) that occurred in more than 1 patient included fever (60%), muscle pain (50%) and fatigue (20%). The cytokine profiles in peripheral blood confirm activation of T cells and myeloid cells, which is in accordance with mitazalimab's mode of action. Based on these data, the 900 μg/kg dose of mitazalimab was selected as the RP2D.

### Example 12: Efficacy and Pharmacodynamic Biomarkers of Mitazalimab in Combination with Chemotherapy in Preclinical Mouse Models

The present study demonstrates the synergistic potential of mitazalimab and FOLFIRINOX in vivo in a mouse pre-clinical model, and investigates pharmacodynamic biomarkers to assess effects of the combination in peripheral blood at early time points.

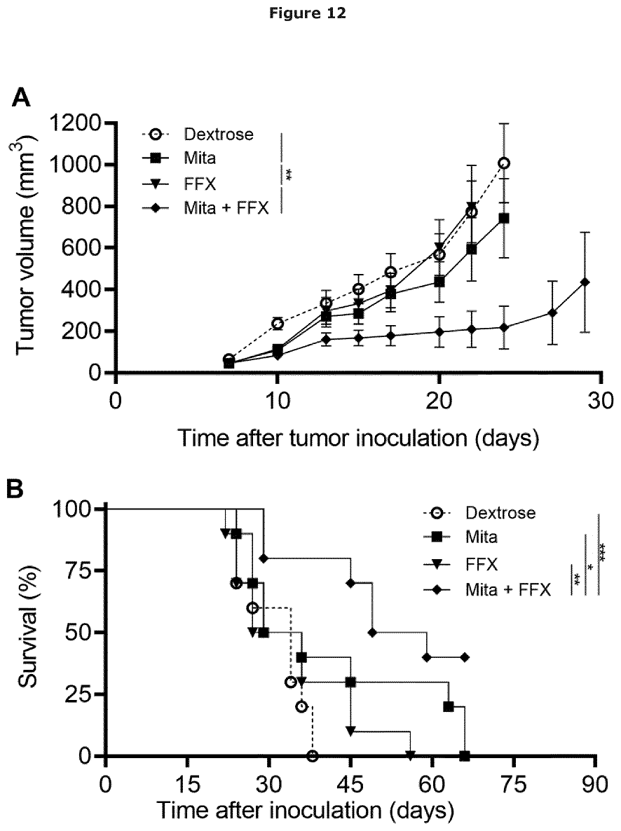

The ability of mitazalimab to augment the response to chemotherapy was demonstrated in human CD40 transgenic (hCD40tg) mice bearing MB49 syngeneic tumors (see FIG. 11). hCD40tg mice were subjected to a dosage regimen of three weekly cycles, containing either vehicle (dextrose), mitazalimab, FOLFIRINOX (oxaliplatin, irinotecan, 5-fluorouracil, and folinic acid), or mitazalimab and FOLFIRINOX in combination. As FOLFIRINOX is composed of four components: oxaliplatin, irinotecan, folinic acid and 5-fluorouracil, these were administered over the course of two days. All components were diluted in 5% glucose and administered i.p. at 100 μl, except for folinic acid which was administered at 200 μl. On day 6 post-inoculation, the mice received 2.5 mg/kg oxaliplatin followed by 25 mg/kg irinotecan. The next day, day 7, the mice received 50 mg/kg folinic acid followed by 25 mg/kg 5-fluorouracil 2 hrs later. Mitazalimab was dosed on day 9. Mitazalimab was diluted in 5% glucose and administered i.p. at a dose of 5 mg/kg (100 μg) in 100 μl. Repeated administration of mitazalimab together with FOLFIRINOX induced long-term survival (see FIG. 12B), and tumor volume control (see FIG. 12A).

Peripheral blood was collected from the mice at the end of the first cycle and subjected to RNA-sequencing to identify pharmacodynamic biomarkers. RNA was extracted from whole blood after red blood cell lysis using GLOBINclear™ Kit, mouse/rat (AM1981, Invitrogen), and Mouse RiboPure™-Blood RNA Isolation Kit (AM1951, Invitrogen) according to the manufacturer's instruction. Purified RNA was stored in RNase-free microfuge tubes (AM12425, Invitrogen), and frozen at −80 C until further use.

RNA library preparation and sequencing was performed by the Center for Translational Genomics (CTG, Lund University). RNA integrity was analyzed with the TapeStation 4200 (Agilent Technologies), using Agilent RNA ScreenTape and reagents (5067-5576, 5067-5577, Agilent Technologies). RNA concentration was analyzed with the Qubit Flex (Q33327 Invitrogen Thermo Fisher Scientific), using the QuantIT RNA HS Assay Kit (Q33140, Life Technologies). cDNA sequencing libraries were prepared using TruSeq® Stranded mRNA Library Prep kit (20020594, Illumina). The libraries were sequenced in a NovaSeq 6000 System (20012850, Illumina), using the NovaSeq 6000 SP Reagent Kit, 300 cycles v1.5 20028400). The reads were aligned to the mouse reference genome GRCm38 (mm10) using the STAR software (v 2.5.0a.13).

R (v4.0.3) and R studio (v1.4.1103) were used to perform quality control and thresholding, normalization, dimensionality reduction, and visualization as well as differential gene expression analysis. Protein coding genes with a minimum of 5 reads in 3 or more samples were kept for analysis. For data visualization, filtered raw counts were normalized using the variance-stabilizing-transformation method in the DESeq2 package (version 1.38.3).

Principal component analysis (PCA) was used to visualize the data, and assess the effects of the treatment at transcriptomic level.

To identify differentially expressed genes among treatment groups, filtered raw counts were normalized using the median of ratios method in the DESeq2 package. Treatment groups were compared using the negative binomial Wald's test (adjusted-p.value [adj-p.val]<0.05, fold change>2).

Pathway enrichment analysis was performed using EnrichR (v3.0) by querying differentially expressed genes among treatment groups onto the gene ontology database (GO, GO_bioprocess_2018). Re-occurring and highly ranked pathways (adj.p-value<0.05) were selected and harmonized throughout the samples using gene set variation transformation (GSVA). The resulting GSVA scores were plotted as a spider plot for pathway activity comparison among the groups.

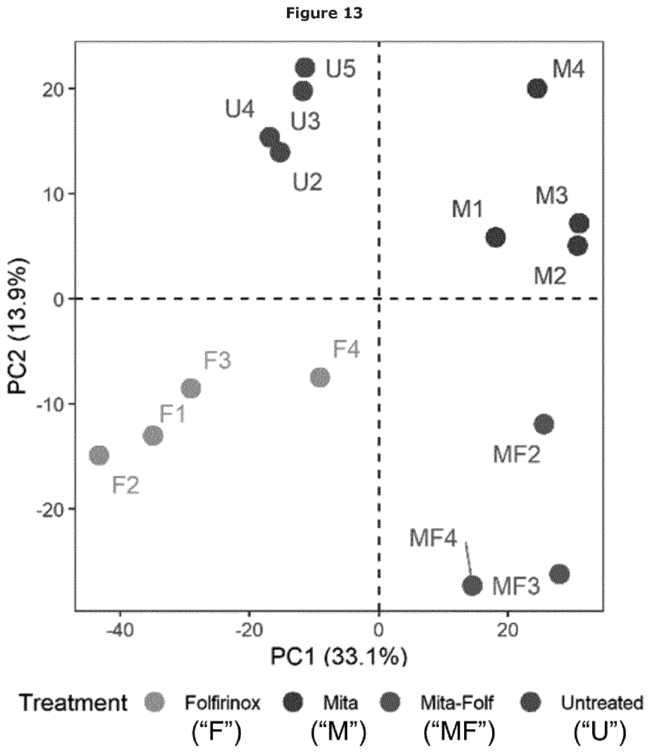

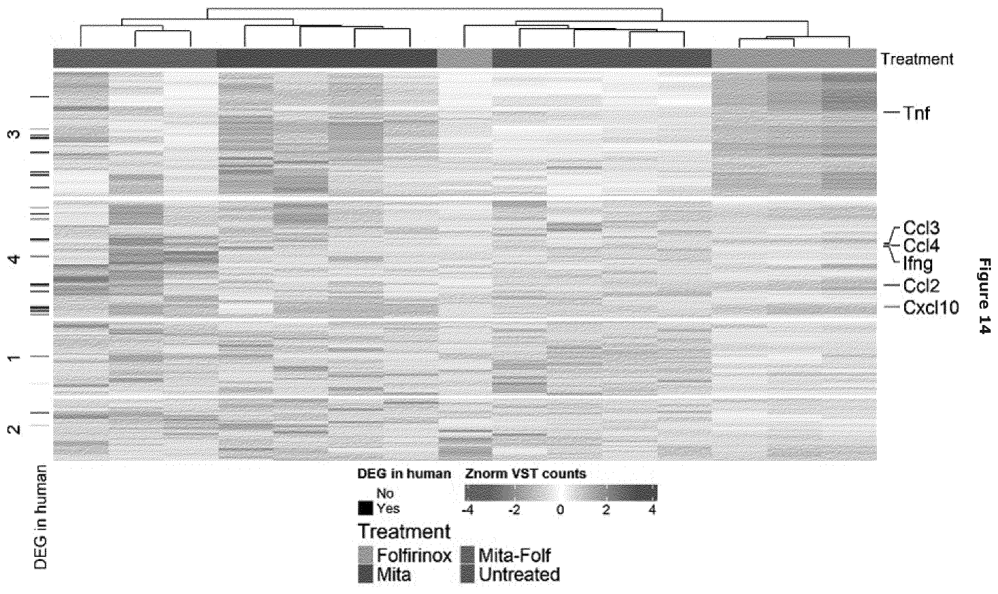

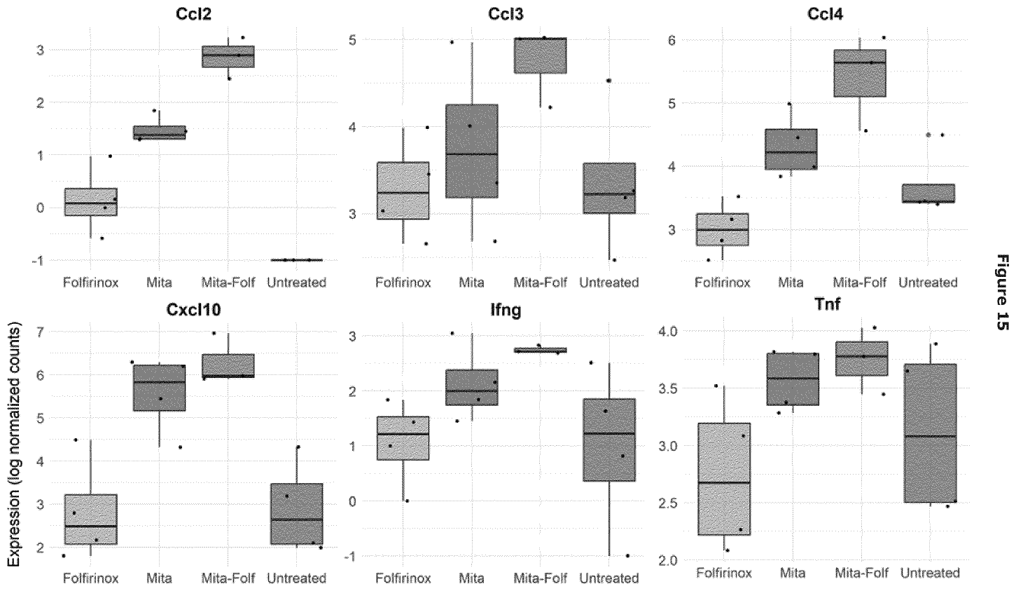

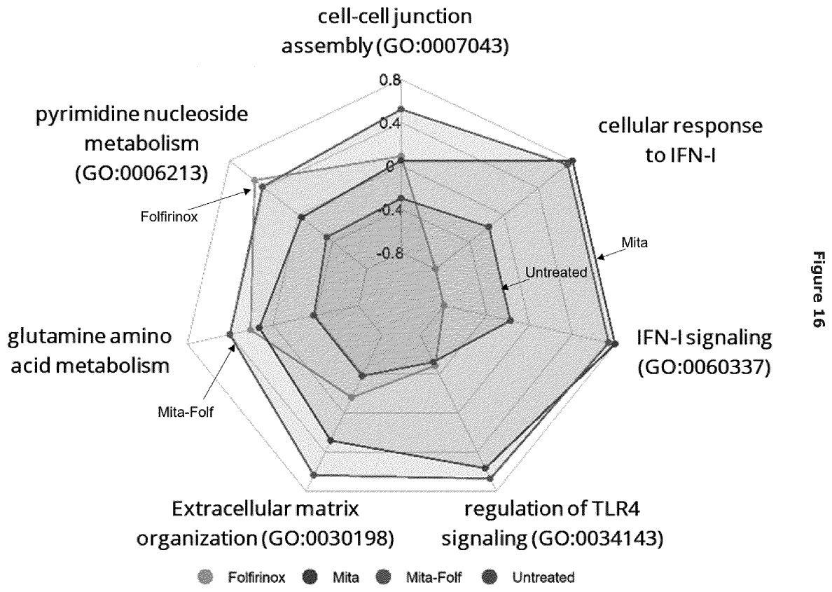

Exploratory data analysis confirmed that samples clustered according to treatment regimen (see FIG. 13), where the groups containing mitazalimab displayed the highest transcriptomic similarity. Differential gene expression profiles were observed for the different treatment groups (see FIG. 14).

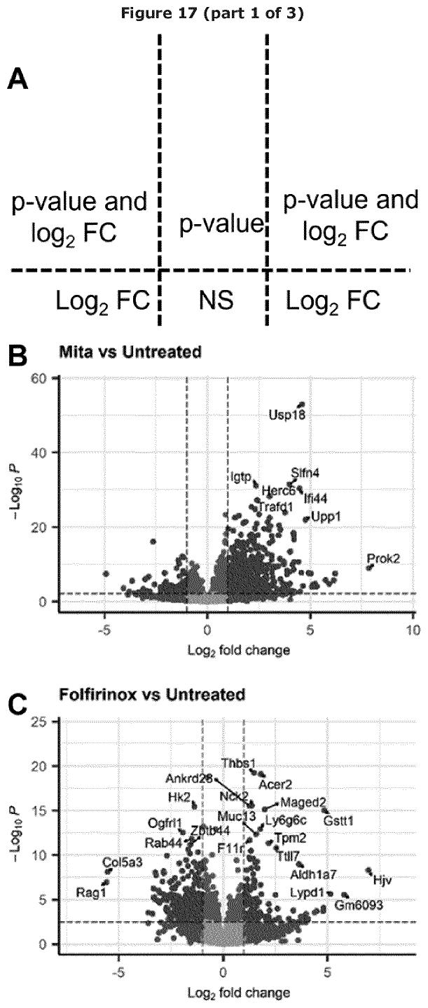

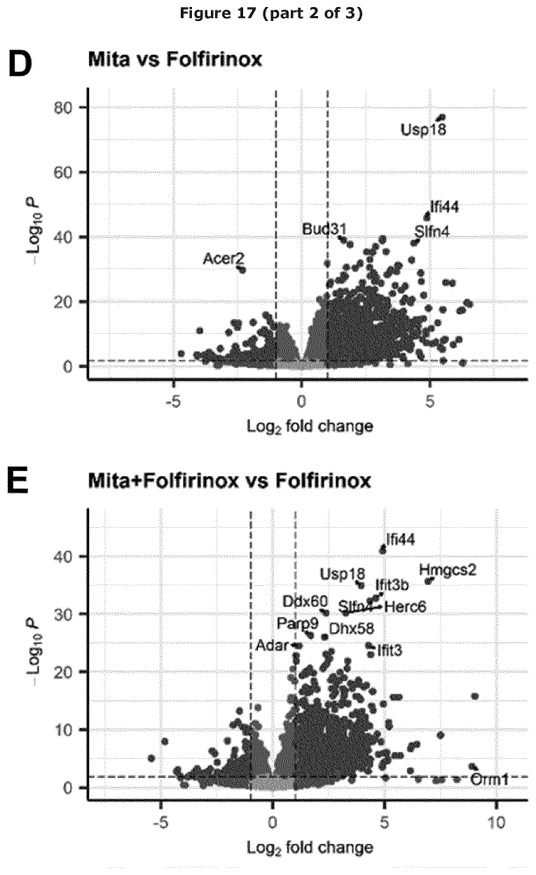

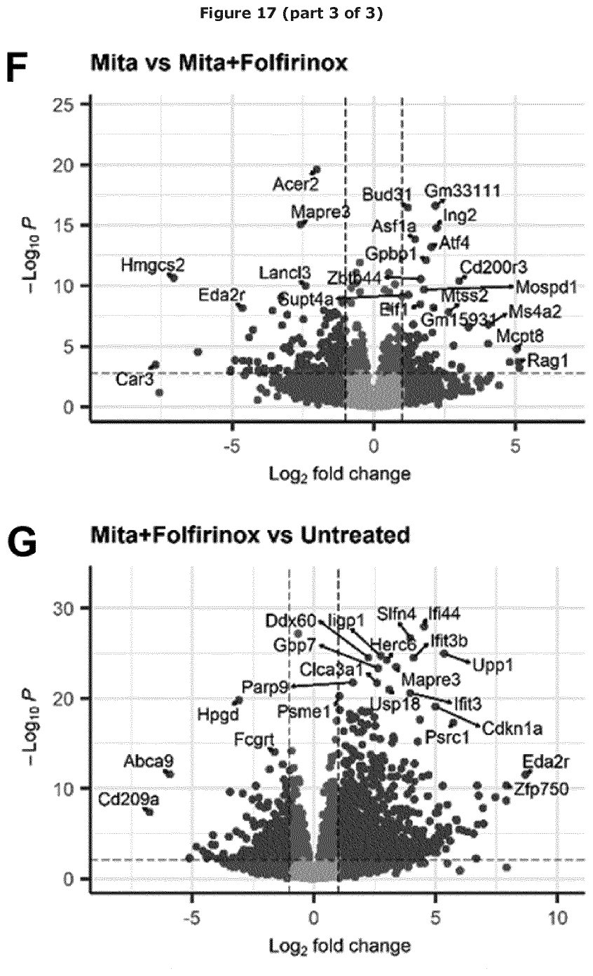

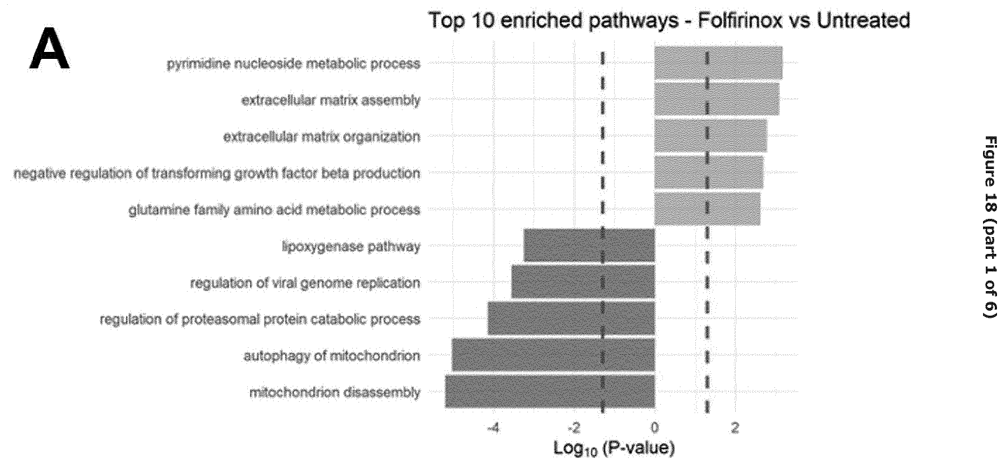

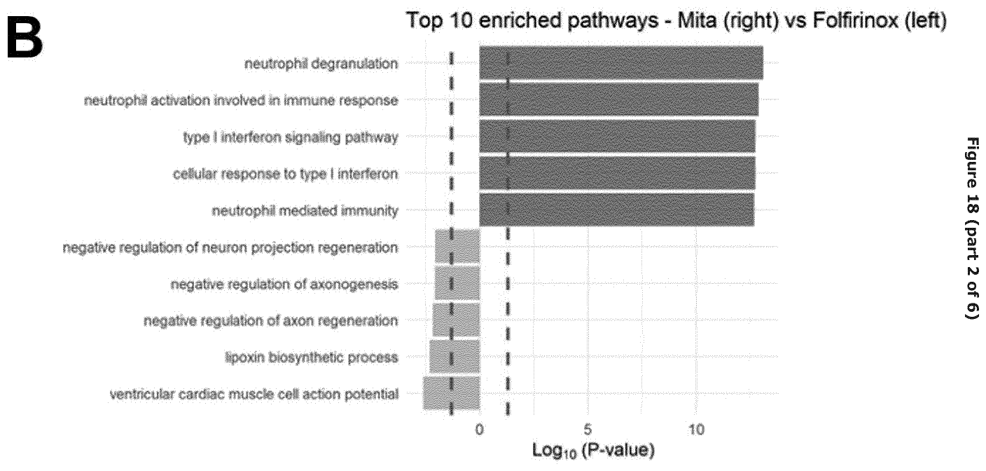

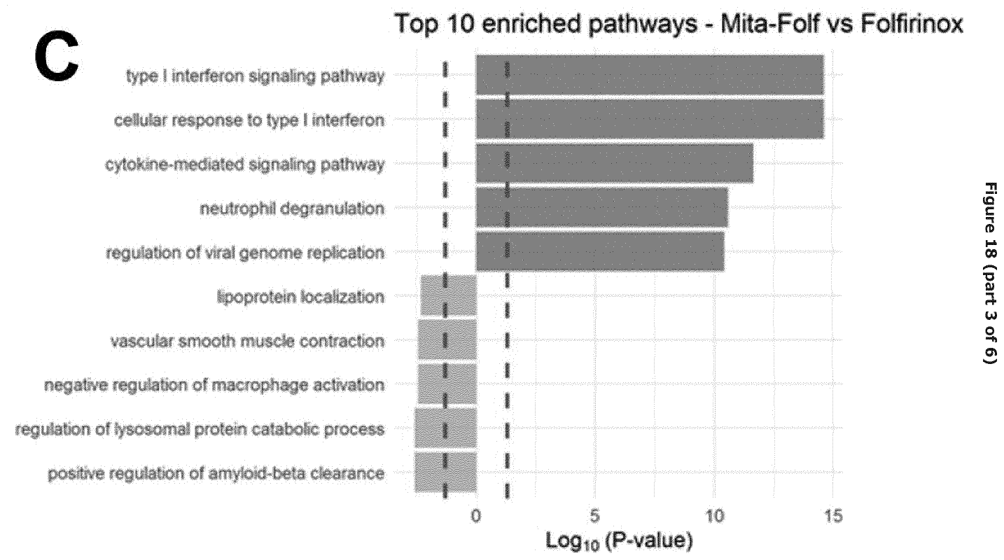

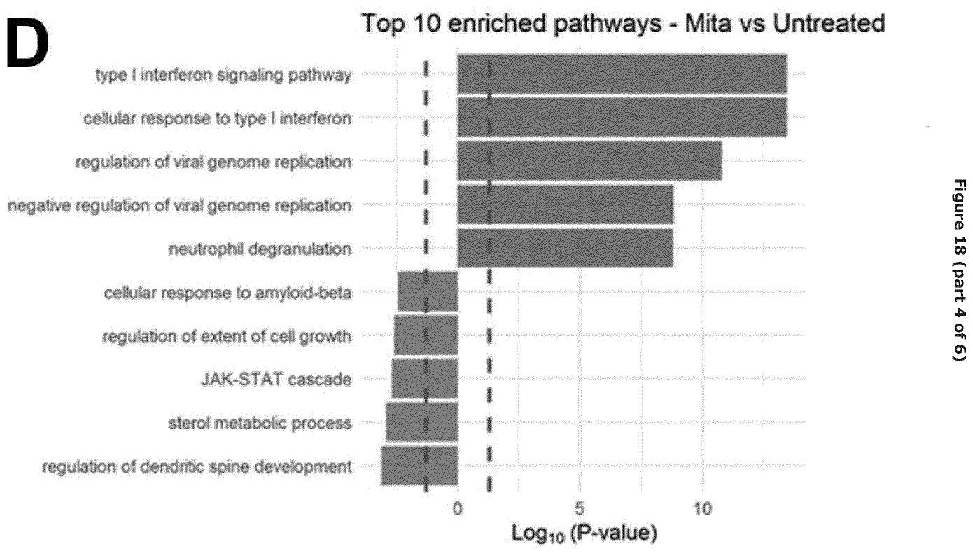

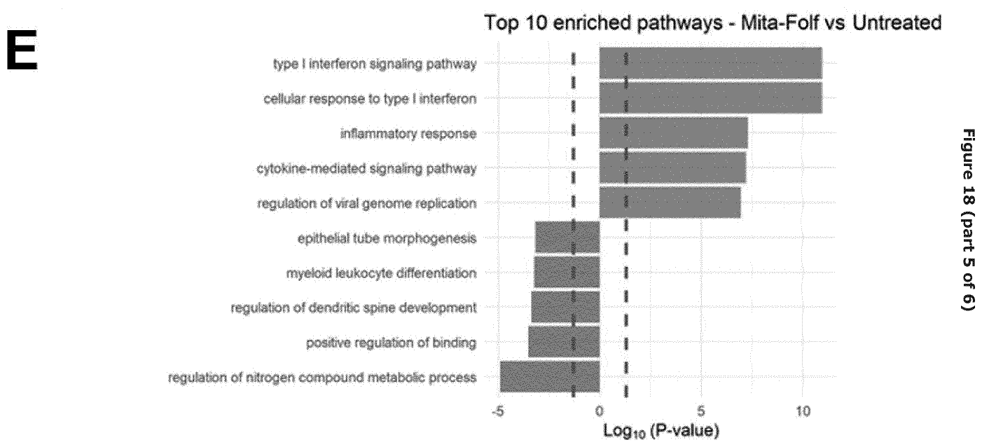

The analysis revealed 3356 differentially expressed genes between the groups, and 221 differentially expressed genes between the two mitazalimab containing groups (Table 35). Mitazalimab administered in combination with FOLFIRINOX induced the highest expression of Ccl2/3/4, Cxcl10 and Ifng (see FIG. 15). The expression of these cytokines suggests an early response of myeloid and T-cell compartments in peripheral blood, reinforcing the mode of action of mitazalimab. Similarly, pathway enrichment and gene set variation analyses yielded examples of the interplay between immune- and chemo-therapeutic agents (see FIGS. 16 and 18). Mitazalimab, alone or in combination, induced high expression of genes involved in response to type-1 IFN and TLR signaling (see FIGS. 15 and 17). In addition, the combination treatment boosted extracellular matrix organization, as well as pyrimidine metabolism; processes that are associated with CD40-mediated myeloid activation and 5-fluorouracil, respectively (see FIGS. 16 and 18).

TABLE 35
Comparison of differentially expressed genes between treatment groups

| | | | | | Mita- | Mita- | |
|---|---|---|---|---|---|---|---|
| | | | Mita vs | Folfirinox | folfirinox | folfirinox | |
| | Mita vs | Mita vs | Mita- | vs | vs | vs | Multigroup |
| | untreated | Folfirinox | Folfirinox | untreated | Folfirinox | untreated | comparison |
| Total | 14485 | 14485 | 14485 | 14485 | 14485 | 14485 | 14485 |
| genes | | | | | | | |
| p. adj < | 2114 | 3953 | 453 | 850 | 3391 | 2516 | 4719 |
| 0.05 | | | | | | | |
| p. adj < | 849 | 1717 | 221 | 425 | 1628 | 1156 | 3356 |
| 0.05 & | | | | | | | |
| FC > 2 | | | | | | | |

In conclusion, mitazalimab synergizes effectively with FOLFIRINOX, inducing long-term survival in a preclinical tumour model. The pharmacodynamic biomarkers of mitazalimab identified here concord with the data from the Phase 1 study of mitazalimab in patients with advanced stage tumours (NCT02829099). Together, these data support the ongoing clinical Phase 1b/2 study (NCT04888312) of mitazalimab in combination with mFOLFIRINOX.

These data also support particular biomarkers that correlate with efficacy of the combination therapy, depending on the control in question, as summarised in Tables 36, 37 and 38. These biomarkers are determined based on being above a particular Log₂ fold change threshold, and either above or below a particular p-value threshold. The biomarkers (whether upregulated or downregulated) indicate that the biomarker may be used to indicate efficacy of the combination therapy versus the respective control (chemotherapy alone, mitazalimab alone, or untreated). I.e. an upregulated biomarker means that the gene has higher expression in the combination therapy and lower expression in the control; and a downregulated biomarker means that the gene has lower expression in the combination therapy and lower expression in the control.

TABLE 36
Biomarkers, specified as MGI symbols, for the combination
therapy versus FOLFIRINOX alone (based on FIG. 17E)
Upregulated Biomarkers in the combination

Adar
Parp9
Dhx58
Dhx60
Slfn4
Ifit3
Ifit3b
Herc6
Usp18
Ifi44
Hmgcs2
Orm1

TABLE 37
Biomarkers, specified as Mouse Genome Informatics
(MGI) symbols, for the combination therapy versus
mitazalimab alone (based on FIG. 17F)

Downregulated Biomarkers in the combination
Bud31
Gm33111
Ing2
Asf1a
Atf4
Gpbp1
Cd200r3
Zbtb44
Mtss2
Mospd1
Supt4a
Eif1
Gm15931
Ms4a2
Mcpt8
Rag1
Upregulated Biomarkers in the combination
Acer2
Mapre3
Lancl3
Eda2r
Hmgcs2
Car3

TABLE 38
Biomarkers, specified as MGI symbols, for the combination
therapy versus untreated control (based on FIG. 17G)

Downregulated Biomarkers in the combination
Cd209a
Abca9
Hpgd
Fcgrt
Upregulated Biomarkers in the combination
Zfp750
Eda2r
Psrc1
Cdkn1a
Ifit3
Ifit3b
Usp18
Mapre3
Upp1
Psme1
Parp9
Clca3a1
Gbp7
Ddx60
Ligp1
Herc6
Slfn4
Ifi44

### Example 13: Association of Patient Response with Cell Abundance and Activation in the Blood of Pancreatic Cancer Patients

### Association Tests

The aim of this analysis was to identify Pharmacodynamic (PD) biomarkers markers for which baseline (C1D1) abundance, or change in abundance from C1D1 at subsequent time points (Fold change), display an association with patient response. PD biomarkers were assessed in the blood of patients enrolled in the clinical study Optimize-1, including counts, percentage of immune cell populations and plasma cytokines, within the first cycle of treatment (C1).

Baseline (C1D1) abundances of each PD biomarker (Tables 39 and 40), as well as change from baseline in abundances of each PD biomarker (Table 41), were assessed for association with patient overall response OR (PR vs SD/PD) and disease control DC (PR/SD vs PD) using a t-test. For each comparison, a positive fold change indicates greater abundance in responders in comparison to non-responders, and vice versa. Abundance of PD biomarkers, namely cell counts and percentages, at baseline (C1D1), or change in abundance from C1D1 at Day 8, were associated with patient response.

Of note, prior to statistical testing (via t-test) at Baseline (C1D1), cell percentages were subjected to arcsine transformation (Tables 39b and 40b).

TABLE 39
Baseline at C1D1: PR vs SD/PD (OR)

| | | | |
|---|---|---|---|
| Cell type | Unit | FC | p-value |

a. Cell counts

| | | | |
|---|---|---|---|
| CD38+ NKT cells | cells/μl | 0.213 | 0.00374 |
| DN T cells | cells/μl | 0.425 | 0.00747 |
| CD38+ CD4+ TEM cells | cells/μl | 0.477 | 0.0106 |
| CD38+ CD8+ TEMRA cells | cells/μl | 0.253 | 0.0142 |
| CD38+ DN T cells | cells/μl | 0.467 | 0.0143 |
| CD4+ TEM cells | cells/μl | 0.585 | 0.0506 |
| CD38+ CD8 T cells | cells/μl | 0.565 | 0.0543 |

b. Percentages

| | | | |
|---|---|---|---|
| CD38+ NKT cells | % of NK T cell | −22.480 | 0.00104 |
| CD54+ monocytes | % of monocyte | 0.154 | 0.0121 |
| CD54+ Int monocytes | % of int monocytes | 0.098 | 0.0274 |
| Ki67+ DN T cells | % of DN T cells | 3.188 | 0.0401 |
| PD-1+ NKT cells | % of NK T cell | 16.443 | 0.0408 |
| CD54+ Class monocytes | % of class monocytes | 0.079 | 0.0505 |

TABLE 40
Baseline at C1D1: PR/SD vs PD (DC)

| | | | |
|---|---|---|---|
| Cell type | Unit | FC | p-value |

a. Cell counts

| | | | |
|---|---|---|---|
| CD38+ CD8+ TCM cells | cells/μl | 0.422 | 0.000897 |
| Ki67+ CD8+ TCM cells | cells/μl | 0.586 | 0.00121 |
| Class switched memory B | cells/μl | 0.339 | 0.00846 |
| cells | | | |
| CD54+ Sw mem B cells | cells/μl | 0.401 | 0.00944 |
| CD86+ pDC | cells/μl | 1.275 | 0.0153 |
| HLA-DR+ Sw mem B cells | cells/μl | 0.404 | 0.0253 |
| CD54+ Plasmablasts | cells/μl | 1.264 | 0.0298 |
| CD86+ Plasmablasts | cells/μl | 1.264 | 0.0298 |
| HLA-DR+ Plasmablasts | cells/μl | 1.264 | 0.0298 |
| CD83+ Sw mem B cells | cells/μl | 1.105 | 0.041 |
| Plasmablasts | cells/μl | 1.239 | 0.048 |
| Double neg B cells | cells/μl | 0.347 | 0.0482 |

b. Percentages

| | | | |
|---|---|---|---|
| Naive CD4 T cells | % of CD4 T cells | 20.410 | 0.000365 |
| CD54+ pDC | % of pDCs | −0.917 | 0.000467 |
| PD-1+ NKT cells | % of NK T cell | 22.525 | 0.00107 |
| PD-1+ NK cells | % of NK cells | −0.559 | 0.00251 |
| HLA-DR+ pDC | % of pDCs | −0.187 | 0.00254 |
| PD-1+ DN T cells | % of DN T cells | 13.307 | 0.00526 |
| PD-1+ CD8+ TEMRA cells | % of CD8 Temra cell | 15.047 | 0.00948 |
| CD4+ TCM cells | % of CD4 T cells | −14.148 | 0.0101 |
| HLA-DR+ Int monocytes | % of int monocytes | −1.823 | 0.0287 |
| Plasmablasts (% of Sw | % of Sw mem B cells | 2.139 | 0.0318 |
| mem B cells) | | | |
| CD38+ CD4+ TEMRA | % of CD4 Temra cell | 16.690 | 0.0377 |
| cells | | | |
| CD86+ pDC | % of pDCs | 5.401 | 0.039 |
| PD-1+ CD8 T cells | % of CD8 T cells | 6.576 | 0.0413 |
| CD86+ Class monocytes | % of class monocytes | −0.359 | 0.0474 |

TABLE 41
Fold change at C1D8: PR vs SD/PD (OR)

| | | | |
|---|---|---|---|
| Cell type | Unit | FC | p-value |

a. Cell counts

| | | | |
|---|---|---|---|
| CD38+ NKT cells | cells/μl | 3.033 | 0.000243 |
| CD38+ CD8+ TEM cells | cells/μl | 3.165 | 0.0016 |
| Ki67+ CD8+ TEM cells | cells/μl | 2.277 | 0.00199 |
| Ki67+ CD4+ TEM cells | cells/μl | 2.250 | 0.00307 |
| CD38+ CD4+ TEM cells | cells/μl | 2.157 | 0.00402 |
| PD-1+ CD8+ TEM cells | cells/μl | 3.308 | 0.00404 |
| CD38+ CD8+ TEMRA | cells/μl | 2.332 | 0.00438 |
| cells | | | |
| CD4+ TEM cells | cells/μl | 1.708 | 0.0049 |
| PD-1+ CD4+ TEM cells | cells/μl | 1.759 | 0.00627 |
| CD8+ TEM cells | cells/μl | 2.274 | 0.0121 |
| Ki67+ CD4+ TCM cells | cells/μl | 1.533 | 0.0133 |
| Ki67+ T cells | cells/μl | 1.468 | 0.0145 |
| Ki67+ CD4 T cells | cells/μl | 1.467 | 0.0151 |
| Ki67+ CD8 T cells | cells/μl | 1.597 | 0.0225 |
| PD-1+ DP T cells | cells/μl | 1.741 | 0.0271 |
| PD-1+ CD8+ TEMRA cells | cells/μl | 2.117 | 0.037 |
| CD8+ TEMRA cells | cells/μl | 1.655 | 0.0381 |
| CD38+ CD8 T cells | cells/μl | 1.556 | 0.0439 |
| PD-1+ CD8 T cells | cells/μl | 1.521 | 0.0553 |

b. Percentages

| | | | |
|---|---|---|---|
| CD38+ NKT cells | % of NK T cell | 30.527 | 0.000993 |
| Naive CD8 T cells | % of CD8 T cells | −9.219 | 0.00512 |
| CD8+ TEM cells | % of CD8 T cells | 7.510 | 0.00695 |
| PD-1+ DP T cells | % of DP T cells | 9.333 | 0.0107 |
| HLA-DR+ pDC | % of pDCs | 0.364 | 0.0114 |
| PD-1+ NKT cells | % of NK T cell | 7.538 | 0.0179 |
| PD-1+ CD8+ TEM cells | % of CD8 Tem cell | 11.257 | 0.0187 |
| CD38+ CD8+ TEM cells | % of CD8 Tem cell | 15.665 | 0.0263 |
| PD-1+ CD8+ TEMRA cells | % of CD8 Temra cell | 7.382 | 0.0421 |
| Ki67+ CD4+ TEM cells | % of CD4 Tem cell | 2.732 | 0.0493 |

### Non-Significant Associate Test and Confounding Analysis

Of note, levels of activation markers or plasma cytokines were not statistically associated with OR or DC (data not included).

Furthermore, a confounding factors analysis was performed to investigate associations between the clinical variables (clinical variables including: Change in tumour lesion size from baseline, Modified Glasgow prognostic score at baseline, Neutrophil to lymphocyte ratio (NLR) at baseline, CA19-9 abundance at baseline, and Change in CA19-9 abundance from baseline). As may be expected, significant (adjusted P<0.05) inter-relationships were observed within response-related variables (RECIST response, OR, DC and change in tumour size), but not between response-variables and other clinical variables.

### Data Quality Control (QC)

The “Cell counts” data set was filtered to retain cell types with counts greater than zero in at least five samples. Of the 138 features, 9 were excluded, and 129 were retained for further analysis. A total of 148 samples and 129 cell count features were taken forward for further analysis. Missing data were imputed using the random forest method from the R package missForest.

The “Percentages” data set was filtered to retain cell types with relative abundance greater than 0% in at least five samples and less than 100% in at least five samples. Of the 153 features, 3 were excluded, and 150 were retained for further analysis. The data set was then filtered to exclude the 13 samples with no cell percentage information available, or missing data for more than 25% of features. Subsequently, two features with missing data in more than 20% of samples were excluded. A total of 149 samples and 148 cell percentage features were taken forward for further analysis. Missing data were imputed using the random forest method from the R package missForest.

### Example 14: Anti-Tumour Efficacy of Mitazalimab in Combination with Chemotherapy in KPCY Pancreatic Mouse Tumor Model

To support the combination of mitazalimab with FOLFIRINOX in a pancreatic mouse tumour model, anti-tumour efficacy was evaluated using the KPCY pancreatic mouse tumour cell line in human CD40 transgenic mice (hCD40tg mice). The KPCY pancreatic mouse tumour cell line was derived from a spontaneous autochthonous mouse model of PDAC (KPC) having major features of the human disease, including mutated Kras and p53 (Li et al., 2018, Immunity, 49, 178-193).

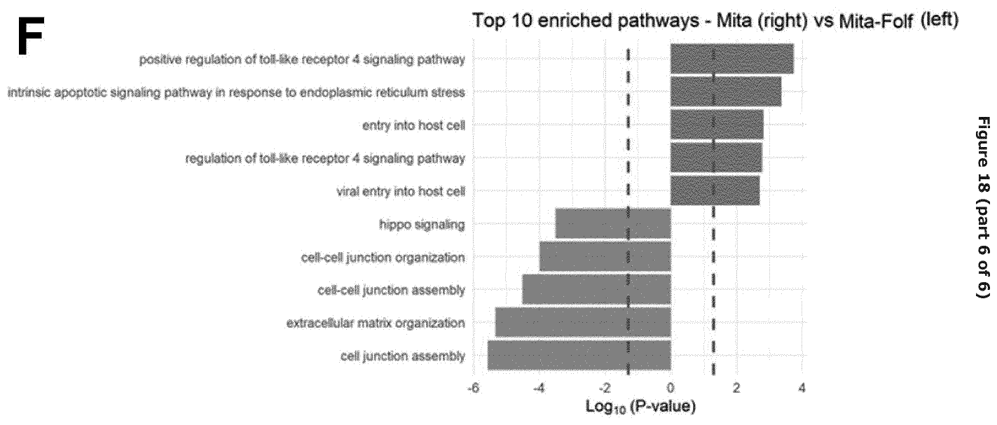

The anti-tumour efficacy of mitazalimab in combination with FOLFIRINOX was evaluated in KPCY tumour-bearing hCD40tg mice. Human CD40-transgenic mice were inoculated subcutaneously in the right flank with KPCY (3×10⁵ cells, clone 2838c3) mouse pancreatic tumour cells (Day 0). On Day 6, one day before initiation of treatment, mice were randomised by tumour size into treatment groups. The average tumour size within each group at the time of treatment initiation was 20 mm³. Mice were treated intraperitoneally once weekly for 3 weeks with mitazalimab (5 mg/kg, Day 10, 17, and 24) and/or FOLFIRINOX (oxaliplatin 2.5 mg/kg), irinotecan (25 mg/kg), 5-fluorouracil (25 mg/kg) and calcium folate (50 mg/kg); (Day 7-8, 14-15, and 21-22) or vehicle (5% glucose, Day 7-8, 10, 14, 15, 17, 21-22 and 24) (n=10 per treatment group) (FIG. 19). Each FOLFIRINOX treatment was administered over 2 days where oxaliplatin and irinotecan was given the first day and calcium folate followed by 5-fluorouracil 2 hours later were administered on the second day.

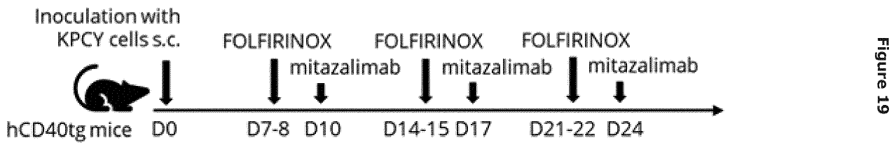

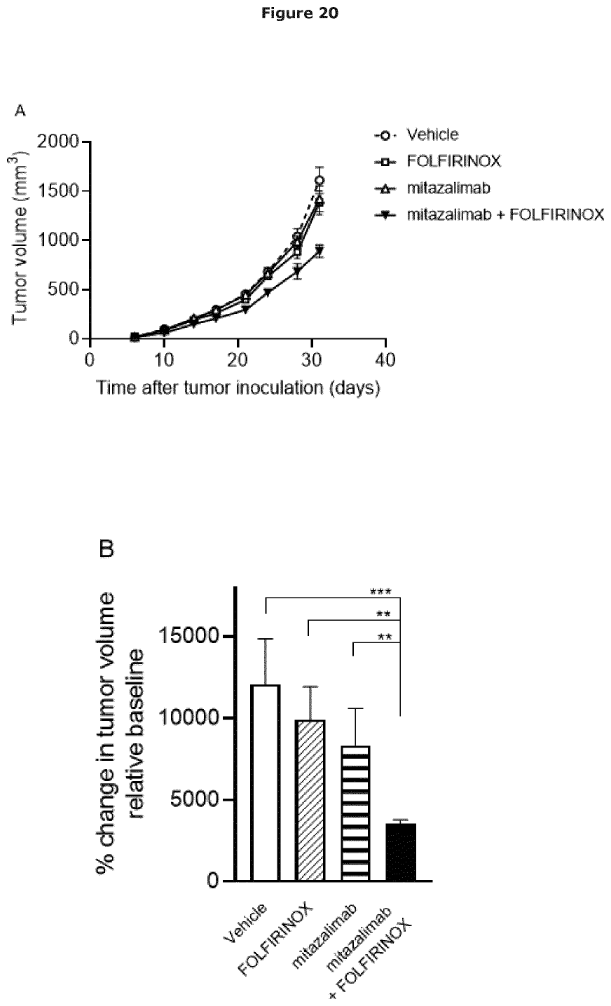

The data demonstrate that a significant synergistic effect on reduction in tumour growth rate was seen for mitazalimab in combination with FOLFIRINOX compared with each treatment alone (FIG. 20).

### Example 15: Mitazalimab and mFOLFIRINOX Efficacy Data from Phase 2 of OPTIMIZE-1 (a Phase 1b/2 Study)

The primary objective for the Phase 2 aspect was to assess the clinical activity of mitazalimab in combination with chemotherapy as determined by ORR. Secondary objectives include survival outcomes, Best Overall Response (BOR), Duration of Response (DoR), Progression Free Survival (PFS), Overall Survival (OS) Duration of SD, Disease control rate, and time to next anti-cancer therapy.

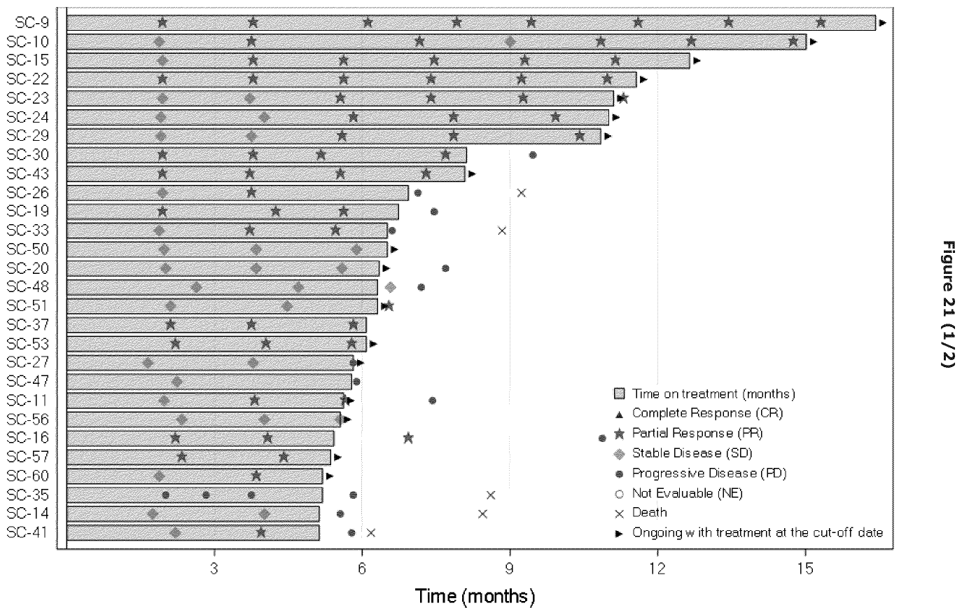

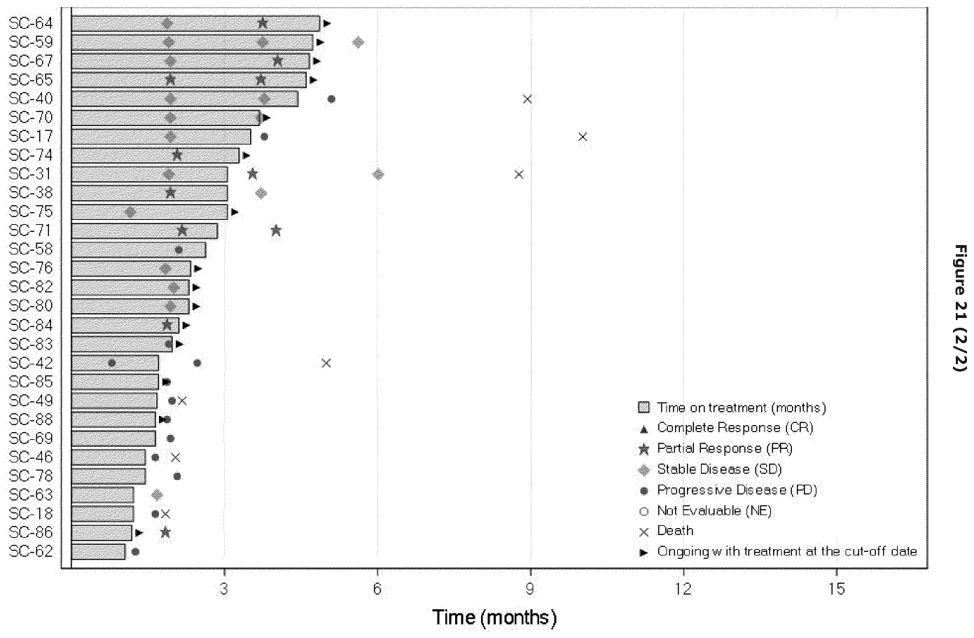

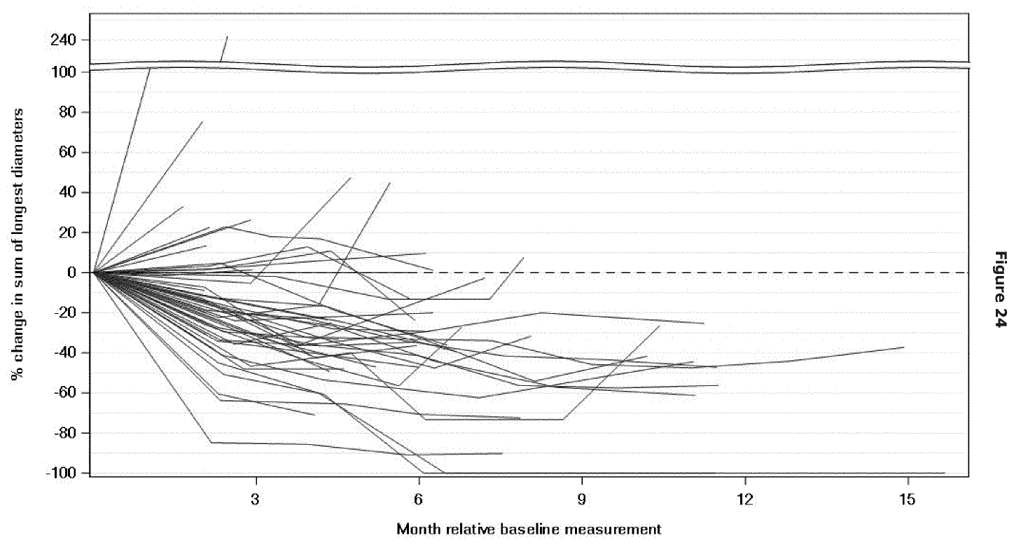

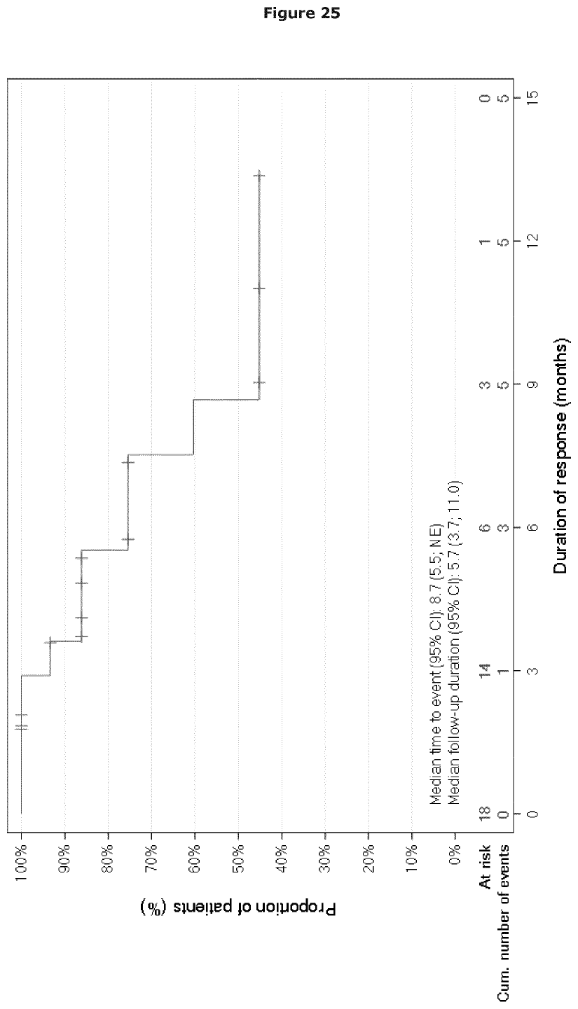

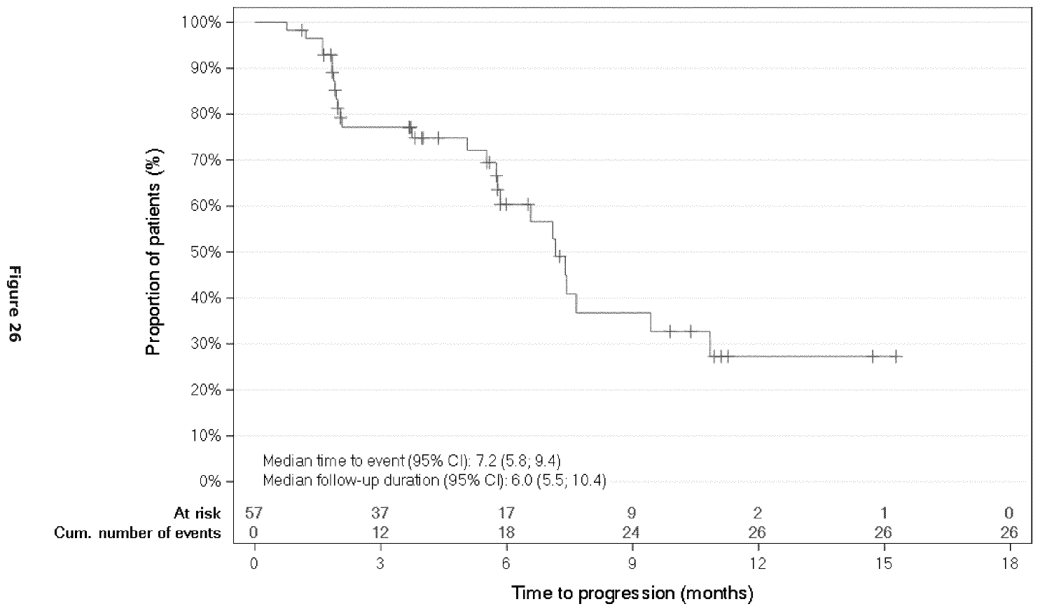

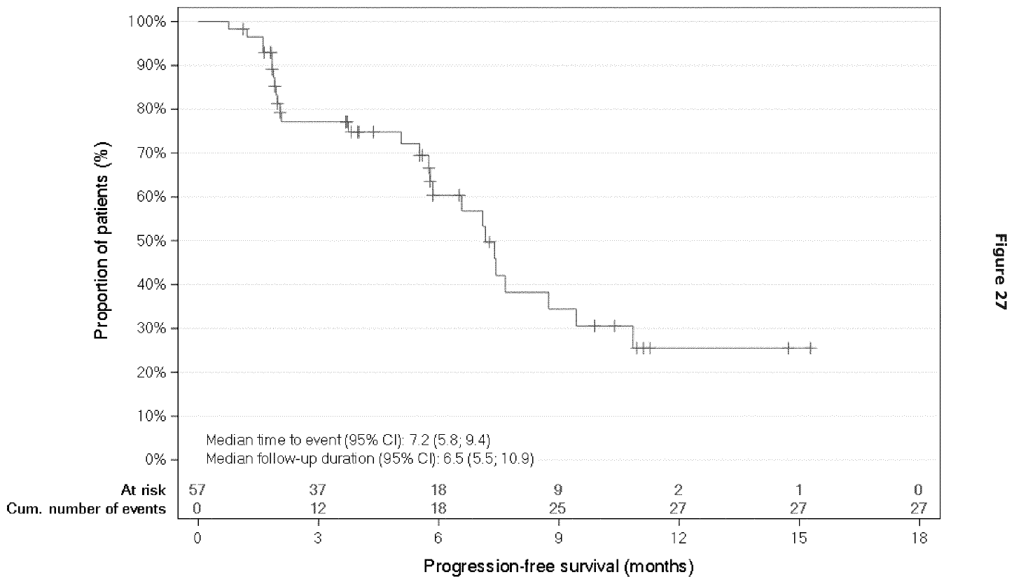

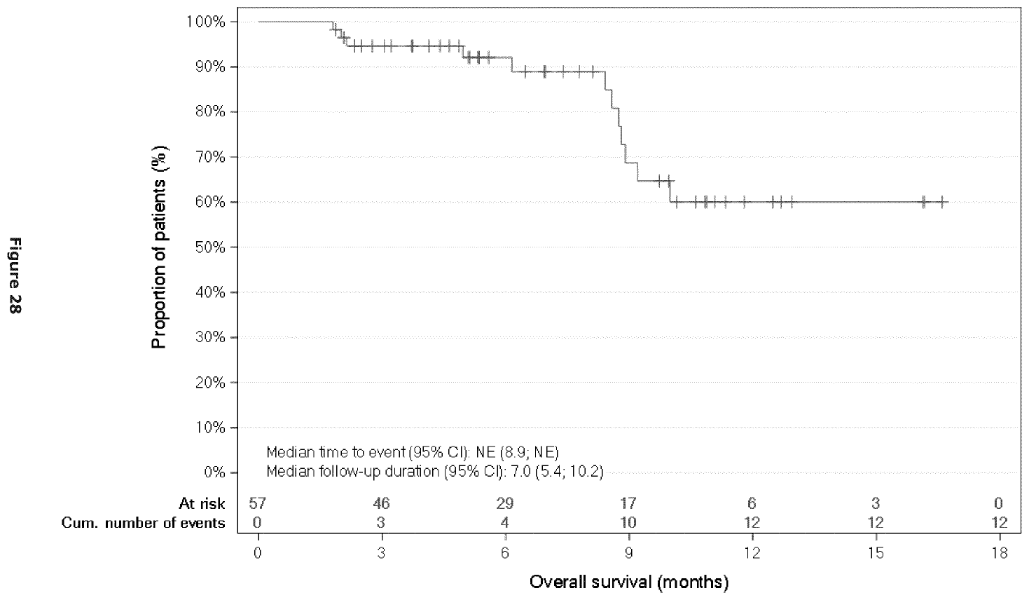

The patient demographics for the data generated in Example 15 is shown in Tables 42-44. Table 45 shows ORR and BOR including unconfirmed responses (FAS) (see also FIG. 21), Table 46 shows Disease control rate (FAS), Table 47 shows duration of response and time to response (FAS) (see also FIGS. 25 and 26), Table 48 shows time to progression (FAS) (see also FIG. 26), Table 49 shows Progression-free survival and overall survival (FAS) (see also FIGS. 27 and 28). FIGS. 22-24 show changes in tumour size and diameter.

TABLE 42
Disposition of patients -

| | 450 μg/kg | 900 μg/kg | Not | |
|---|---|---|---|---|
| | Mitazalimab | Mitazalimab | dosed | Overall |
| | (N = 5) | (N = 65) | (N = 15) | (N = 85) |
| Screened subjects | 5 (100.0) | 65 (100.0) | 15 (100.0) | 85 (100.0) |
| Included in safety set | 5 (100.0) | 65 (100.0) | 0 (0.0) | 70 (82.4) |
| Excluded from safety set | 0 (0.0) | 0 (0.0) | 15 (100.0) | 15 (17.6) |
| Not exposed to Mitazalimab | 0 (0.0) | 0 (0.0) | 15 (100.0) | 15 (100.0) |
| Included in full analysis | 0 (0.0) | 57 (87.7) | — | 57 (81.4) |
| set (Efficacy) | | | | |
| Excluded from full analysis set | 5 (100.0) | 8 (12.3) | — | 13 (18.6) |
| Did not complete two cycles | 0 (0.0) | 8 (100.0) | — | 8 (61.5) |
| of treatment | | | | |
| Did not receive the combination | 5 (100.0) | 0 (0.0) | — | 5 (38.5) |
| of mitazalimab at the RP2D | | | | |
| Ongoing treatment¹ | 0 (0.0) | 32 (49.2) | — | 32 (45.1) |
| Discontinued treatment | 5 (100.0) | 33 (50.8) | — | 39 (54.9) |
| Progressive disease | 4 (80.0) | 19 (57.6) | — | 24 (61.5) |
| Unacceptable toxicity | 1 (20.0) | 5 (15.2) | — | 6 (15.4) |
| Other | 0 (0.0) | 3 (9.1) | — | 3 (7.7) |
| Non-compliance with study protocol | 0 (0.0) | 1 (3.0) | — | 1 (2.6) |
| Withdrawal of consent | 0 (0.0) | 1 (3.0) | — | 1 (2.6) |
| No longer clinically benefiting | 0 (0.0) | 3 (9.1) | — | 3 (7.7) |
| Death | 0 (0.0) | 1 (3.0) | — | 1 (2.6) |
| In Survival follow-up period | 0 (0.0) | 17 (26.2) | — | 17 (20.0) |
| after treatment | | | | |
| Ongoing study | 0 (0.0) | 47 (72.3) | 2 (13.3) | 49 (57.6) |
| Discontinued study | 5 (100.0) | 18 (27.7) | 13 (86.7) | 36 (42.4) |
| Other | 0 (0.0) | 1 (5.6) | 0 (0.0) | 1 (2.8) |
| Withdrawal of consent | 1 (20.0) | 1 (5.6) | 0 (0.0) | 2 (5.6) |
| Screen failure | 0 (0.0) | 0 (0.0) | 12 (92.3) | 12 (33.3) |
| Death due to disease progression | 4 (80.0) | 15 (83.3) | 1 (7.7) | 20 (55.6) |
| Non-compliance with | 0 (0.0) | 1 (5.6) | 0 (0.0) | 1 (2.8) |
| clinical study protocol | | | | |

Abbreviations: N = number of patients in analysis set and treatment group
¹Includes all patients that started treatment and for which no discontinuation was reported. Results show the number of patients and percentage of patients in brackets relative to N for overall parameters and relative to number of excluded/discontinued for reasons for exclusion/discontinuation. Possible reasons are displayed only if applicable.

TABLE 43
Age, weight, height, BMI (FAS) -

| | | 900 μg/kg Mitazalimab |
|---|---|---|
| | | (N = 57) |
| Age (years) | n | 57 |
| | Mean | 60.6 |
| | SD | 8.8 |
| | Minimum | 43 |
| | Median | 62.0 |
| | Maximum | 77 |
| Age group | n | 57 |
| | Under 65 | 35 (61.4) |
| | 65 or above | 22 (38.6) |
| Weight (kg) | n | 57 |
| | Mean | 66.8 |
| | SD | 14.7 |
| | Minimum | 44 |
| | Median | 66.0 |
| | Maximum | 95 |
| BMI (kg/m²) | n | 57 |
| | Mean | 23.4 |
| | SD | 4.5 |
| | Minimum | 16 |
| | Median | 23.3 |
| | Maximum | 37 |
| Height (cm) | n | 57 |
| | Mean | 168.8 |
| | SD | 8.5 |
| | Minimum | 152 |
| | Median | 167.0 |
| | Maximum | 186 |

Abbreviations:
N = number of patients in analysis set and treatment group,
n = number of patients with non-missing value,
SD = standard deviation

TABLE 44
Gender, race, ethnic origin (FAS) -

| | 900 μg/kg Mitazalimab | |
|---|---|---|
| | (N = 57) | |
| n | 57 | |
| Female | 33 | (57.9) |
| Male | 24 | (42.1) |
| n | 57 | |
| American Indian or Alaska Native | 0 | (0.0) |
| Asian | 0 | (0.0) |
| Black or African American | 3 | (5.3) |
| Native Hawaiian or Other Pacific Islander | 0 | (0.0) |
| Not reported | 16 | (28.1) |
| Other | 1 | (1.8) |
| Unknown | 0 | (0.0) |
| White | 37 | (64.9) |
| n | 57 | |
| Unknown | 0 | (0.0) |
| Hispanic or Latino | 3 | (5.3) |
| Not Hispanic or Latino | 35 | (61.4) |
| Not Reported | 19 | (33.3) |

Abbreviations:
N = number of patients in analysis set and treatment group,
n = number of patients with non-missing value
Results are given as number of patients and percentage of patients relative to n in brackets.

TABLE 45
ORR and BOR including unconfirmed responses
(FAS) - (see also FIG. 21) -

| | 900 μg/kg Mitazalimab | |
|---|---|---|
| | (N = 57) | |
| n | 57 | |
| Complete response | 0 | (0.0) |
| Partial response | 25 | (43.9) |
| Stable disease | 19 | (33.3) |
| Progressive disease | 12 | (21.1) |
| Not evaluable | 1 | (1.8) |
| n | 57 | |
| Responders | 25 | (43.9) |

| | |
|---|---|
| Confidence Interval | 32.6-55.6 |

Abbreviations:
n = number of patients with non-missing value
Results are given as number of patients and percentage of patients relative to n in brackets. ORR is defined as the proportion of patients that achieved a complete or partial response as BOR according to RECIST v. 1.1 or without confirmation if not available. Patients not having evaluable results from tumour imaging are included in n and the denominator for ORR. The two-sided exact binomial 90% confidence interval of responders is presented.

TABLE 46
Disease control rate (FAS) -

| | 900 μg/kg Mitazalimab |
|---|---|
| | (N = 57) |
| n | 57 |
| Disease control rate | 44 (77.2) |

Abbreviations:
n = number of patients with non-missing value
Disease control is defined as having complete or partial response or stable disease as best overall response according to RECIST 1.1.

TABLE 47
Duration of response and time to response
(FAS) (see also FIGS. 25 and 26) -

| | | 900 μg/kg Mitazalimab | |
|---|---|---|---|
| | | (N = 57) | |
| Duration of | n | 18 | |
| response | Patients with event, n (%) | 5 | (27.8) |
| (months) | Censored patients, n (%) | 13 | (72.2) |
| | Median time to event (95% CI) | 8.7 | (5.5; NE) |
| | Median follow-up duration (95% | 5.7 | (3.7-11.0) |
| | CI) | | |
| Time to | n | 18 | |
| response | Median time to event (95% CI) | 2.2 | (1.9-3.7) |
| (months) | | | |

Abbreviations:
n = number of patients with non-missing value,
CI = confidence interval,
NE = not estimable
Duration of response is defined as number of months from initial response of CR or PR to progressive disease or death due to underlying disease, whichever was first. Date of last CT/MRI scan is used as censoring date. Time to response is defined as number of months from first dose of mitazalimab to disease response with no censoring.

TABLE 48
Time to progression (FAS) (see also FIG. 26)

| | | 900 μg/kg Mitazalimab | |
|---|---|---|---|
| | | (N = 57) | |
| Time to | n | 57 | |
| progression | Patients with event, n (%) | 26 | (45.6) |
| (months) | Censored patients, n (%) | 31 | (54.4) |
| | Median time to event (95% CI) | 7.2 | (5.8-9.4) |
| | Median follow-up duration (95% | 6.0 | (5.5-10.4) |
| | CI) | | |

Abbreviations:
n = number of patients with non-missing value,
CI = confidence interval,
NE = not estimable
Time to progression is defined as number of months from first dose of mitazalimab to progressive disease. The last known date of being free of disease progression, i.e., date of last CT/MRI scan, is used as censoring date. A patient not having any post-baseline CT/MRI scan available is censored at day of first dose of mitazalimab, i.e. censored with a duration of 0.

TABLE 49
Progression-free survival and overall survival
(FAS) (see also FIGS. 27 and 28)

900 μg/kg Mitazalimab
(N = 57)

| n | 57 | |
|---|---|---|
| Patients with event, n (%) | 27 | (47.4) |
| Censored patients, n (%) | 30 | (52.6) |
| Median time to event (95% CI) | 7.2 | (5.8-9.4) |
| Median follow-up duration (95% CI) | 6.5 | (5.5-10.9) |
| n | 57 | |
| Patients with event, n (%) | 12 | (21.1) |
| Censored patients, n (%) | 45 | (78.9) |
| Median time to event (95% CI) | NE | (8.9-NE) |
| Median follow-up duration (95% CI) | 7.0 | (5.4-10.2) |

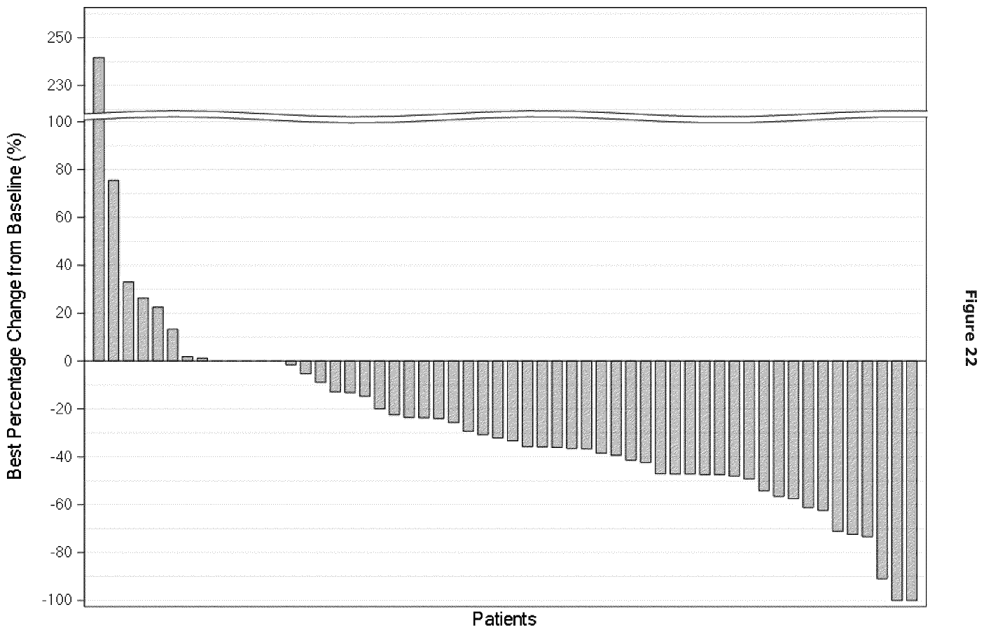

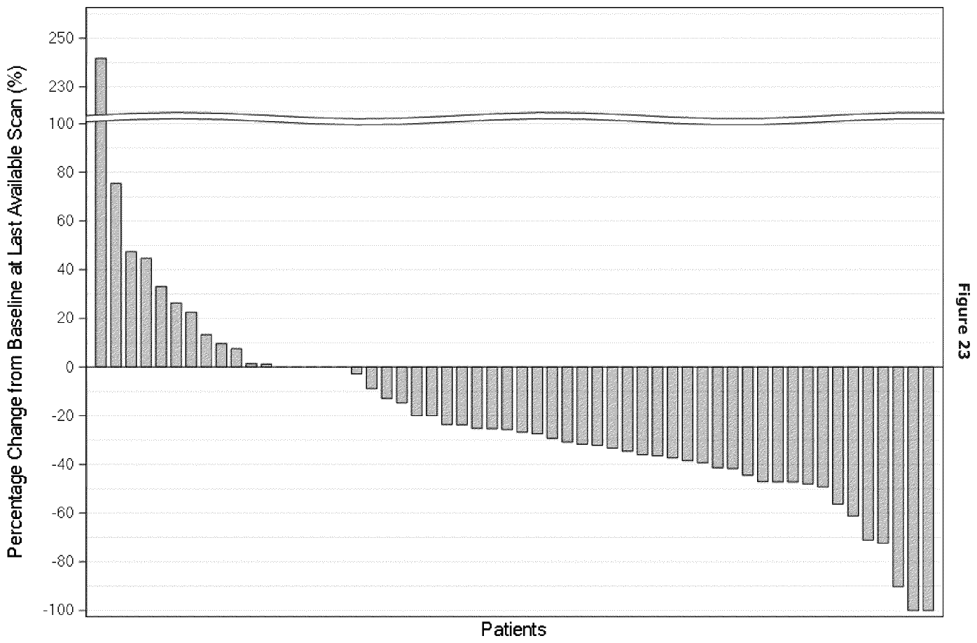

Abbreviations:
n = number of patients with non-missing value,
CI = confidence interval,
NE = not estimable
Progression-free survival is defined as number of months from first dose of mitazalimab to progressive disease or death due to any cause, whichever came first. Date of last CT/MRI scan is used as censoring date. Overall survival is defined as number of months from first dose of mitazalimab to death from any cause. Last known date being alive while on study, date of withdrawal of consent or cut-off date for the analysis, as applicable, is used as censoring date.
Conclusion
These data support that the combination of mitazalimab with mFOLFIRINOX is safe and well tolerated, and produces robust clinical
responses with a positive impact on ORR, BOR, tumour volume (see FIGS. 23 and 24), overall survival, and progression-free survival.

### REFERENCES

1. Puckett, Y. and K. Garfield, Pancreatic Cancer, in StatPearls. 2020: Treasure Island (FL).
2. Rawla, P., T. Sunkara, and V. Gaduputi, Epidemiology of Pancreatic Cancer: Global Trends, Etiology and Risk Factors. World J Oncol, 2019. 10(1): p. 10-27.
3. Ducreux, M., et al., Cancer of the pancreas: ESMO Clinical Practice Guidelines for diagnosis, treatment and follow-up. Ann Oncol, 2015. 26 Suppl 5: p. v56-68.
4. Conroy, T.e.a., FOLFIRINOX versus Gemcitabine for Metastatic Pancreatic Cancer. N Engl J Med, 2011(364): p. 1817-1825.
5. Von Hoff, D. D., et al., Increased survival in pancreatic cancer with nab-paclitaxel plus gemcitabine. N Engl J Med, 2013. 369(18): p. 1691-703.
6. Elgueta, R., et al., Molecular mechanism and function of CD40/CD40L engagement in the immune system. Immunol Rev, 2009. 229(1): p. 152-72.
7. Korniluk, A., H. Kemona, and V. Dymicka-Piekarska, Multifunctional CD40L: pro-and anti-neoplastic activity. Tumour Biol, 2014. 35(10): p. 9447-57.
8. Peters, A. L., L. L. Stunz, and G. A. Bishop, CD40 and autoimmunity: the dark side of a great activator. Semin Immunol, 2009. 21(5): p. 293-300.
9. Eliopoulos, A. G. and L. S. Young, The role of the CD40 pathway in the pathogenesis and treatment of cancer. Curr Opin Pharmacol, 2004. 4(4): p. 360-7.
10. Vonderheide, R. H. and M. J. Glennie, Agonistic CD40 antibodies and cancer therapy. Clin Cancer Res, 2013. 19(5): p. 1035-43.
11. Ma, D. Y. and E. A. Clark, The role of CD40 and CD154/CD40L in dendritic cells.

Semin Immunol, 2009. 21(5): p. 265-72.
12. Moran, A. E., M. Kovacsovics-Bankowski, and A. D. Weinberg, The TNFRs OX40, 4-1BB, and CD40 as targets for cancer immunotherapy. Curr Opin Immunol, 2013. 25(2): p. 230-7.
13. Piechutta, M. and A. S. Berghoff, New emerging targets in cancer immunotherapy: the role of Cluster of Differentiation 40 (CD40/TNFR5). ESMO Open, 2019. 4(Suppl 3): p. e000510.
14. Zarnegar, B., et al., Unique CD40-mediated biological program in B cell activation requires both type 1 and type 2 NF-kappaB activation pathways. Proc Natl Acad Sci USA, 2004. 101(21): p. 8108-13.
15. Rakhmilevich, A. L., K. L. Alderson, and P. M. Sondel, T-cell-independent antitumour effects of CD40 ligation. Int Rev Immunol, 2012. 31(4): p. 267-78.
16. Calvo, E., et al., A phase I study to assess safety, pharmacokinetics (PK), and pharmacodynamics (PD) of JNJ-64457107, a CD40 agonistic monoclonal antibody, in patients (pts) with advanced solid tumours. Journal of Clinical Oncology, 2019. 37(15_suppl): p. 2527-2527.
17. Vonderheide, R. H., et al., Clinical activity and immune modulation in cancer patients treated with CP-870,893, a novel CD40 agonist monoclonal antibody. J Clin Oncol, 2007. 25(7): p. 876-83.
18. Vonderheide, R. H., CD40 Agonist Antibodies in Cancer Immunotherapy. Annu Rev Med, 2020. 71: p. 47-58.
19. Beatty, G. L., et al., A phase I study of an agonist CD40 monoclonal antibody (CP-870,893) in combination with gemcitabine in patients with advanced pancreatic ductal adenocarcinoma. Clin Cancer Res, 2013. 19(22): p. 6286-95.
20. O'Hara, M. H., et al., Abstract CT004: A Phase Ib study of CD40 agonistic monoclonal antibody APX005M together with gemcitabine (Gem) and nab-paclitaxel (NP) with or without nivolumab (Nivo) in untreated metastatic ductal pancreatic adenocarcinoma (PDAC) patients. Cancer Research, 2019. 79(13 Supplement): p. CT004-CT004.
21. Yao, W., A. Maitra, and H. Ying, Recent insights into the biology of pancreatic cancer. EBioMedicine, 2020. 53: p. 102655.
22. Vonderheide, R. H., The Immune Revolution: A Case for Priming, Not Checkpoint. Cancer Cell, 2018. 33(4): p. 563-569.
23. Nowak, A. K., B. W. Robinson, and R. A. Lake, Synergy between chemotherapy and immunotherapy in the treatment of established murine solid tumours. Cancer Res, 2003. 63(15): p. 4490-6.
24. Byrne, K. T. and R. H. Vonderheide, CD40 Stimulation Obviates Innate Sensors and Drives T Cell Immunity in Cancer. Cell Rep, 2016. 15(12): p. 2719-32.
25. Beatty, G. L., et al., CD40 agonists alter tumour stroma and show efficacy against pancreatic carcinoma in mice and humans. Science (New York, NY), 2011. 331.
26. Beatty, G. L., Y. Li, and K. B. Long, Cancer immunotherapy: activating innate and adaptive immunity through CD40 agonists. Expert Rev Anticancer Ther, 2017. 17(2): p. 175-186.
27. Long, K. B., et al., IFNgamma and CCL2 Cooperate to Redirect Tumour-Infiltrating Monocytes to Degrade Fibrosis and Enhance Chemotherapy Efficacy in Pancreatic Carcinoma. Cancer Discov, 2016. 6(4): p. 400-413.
28. Byrne, K. T., et al., CSF-1R-Dependent Lethal Hepatotoxicity When Agonistic CD40 Antibody Is Given before but Not after Chemotherapy. J Immunol, 2016. 197(1): p. 179-87.
29. Mayes, P. A., K. W. Hance, and A. Hoos, The promise and challenges of immune agonist antibody development in cancer. Nat Rev Drug Discov, 2018. 17(7): p. 509-527.
30. Lowery, M. A., et al., Activity of front-line FOLFIRINOX (FFX) in stage III/IV pancreatic adenocarcinoma (PC) at Memorial Sloan-Kettering Cancer Center (MSKCC). Journal of Clinical Oncology, 2012. 30(15_suppl): p. 4057-4057.
31. Stein, S. M., et al., Final analysis of a phase II study of modified FOLFIRINOX in locally advanced and metastatic pancreatic cancer. Br J Cancer, 2016. 114(7): p. 737-43.
32. Ko, A. H., et al., A Phase I Study of FOLFIRINOX Plus IPI-926, a Hedgehog Pathway Inhibitor, for Advanced Pancreatic Adenocarcinoma. Pancreas, 2016. 45(3): p. 370-5.
33. Philip, P. A., et al., Avenger 500, a phase III open-label randomized trial of the combination of CPI-613 with modified FOLFIRINOX (mFFX) versus FOLFIRINOX (FFX) in patients with metastatic adenocarcinoma of the pancreas. Journal of Clinical Oncology, 2019. 37(4_suppl): p. TPS479-TPS479.
34. Conroy, T., et al., FOLFIRINOX or Gemcitabine as Adjuvant Therapy for Pancreatic Cancer. N Engl J Med, 2018. 379(25): p. 2395-2406.
35. Oken, M. M., et al., Toxicity and response criteria of the Eastern Cooperative Oncology Group. Am J Clin Oncol, 1982. 5(6): p. 649-55.
36. Mander, A. P. and S. G. Thompson, Two-stage designs optimal under the alternative hypothesis for phase II cancer clinical trials. Contemp Clin Trials, 2010. 31(6): p. 572-8.
37. Liu, S. and Y. Yuan, Bayesian optimal interval designs for phase I clinical trials. J R Stat Soc Ser C Appl Stat, 2015(64): p. 507-23.
38. Yuan, Y., et al., Bayesian Optimal Interval Design: A Simple and Well-Performing Design for Phase I Oncology Trials. Clin Cancer Res, 2016. 22(17): p. 4291-301.
39. Boulanger, J., et al., Management of the extravasation of anti-neoplastic agents. Support Care Cancer, 2015. 23(5): p. 1459-71.
40. Hongxuan Tong, Zhu Fan, Biyuan Liu & Tao Lu, The benefits of modified FOLFIRINOX for advanced pancreatic cancer and its induced adverse events: a systematic review and meta-analysis, Scientific Reports, 2018, 8L8666.
41. Stein, S. M. et al. Final analysis of a phase II study of modified FOLFIRINOX in locally advanced and metastatic pancreatic cancer. British journal of cancer 114, 737-743, https://doi.org/10.1038/bjc.2016.45 (2016).
42. Vivaldi, C. et al. First-line treatment with FOLFOXIRI for advanced pancreatic cancer in clinical practice: Patients' outcome and analysis of prognostic factors. International journal of cancer 139, 938-945 (2016).
43. Mahaseth, H. et al. Modified FOLFIRINOX regimen with improved safety and maintained efficacy in pancreatic adenocarcinoma. Pancreas 42, 1311-1315 (2013).
44. Ghorani, E. et al. Safety and Efficacy of Modified FOLFIRINOX for Advanced Pancreatic Adenocarcinoma: A UK Single-Centre Experience. Oncology 89(5), 281-287 (2015).
45. Nanda, R. H., El-Rayes, B., Maithel, S. K. & Landry, J. Neoadjuvant modified FOLFIRINOX and chemoradiation therapy for locally advanced pancreatic cancer improves resectability. Journal of surgical oncology 111, 1028-1034 (2015).
46. Vocka, M. & Petruzelka, L. Modified FOLFIRINOX in the treatment of pancreatic cancer-efficiency and toxicity. Gastroenterologie a Hepatologie 70, 413-417 (2016).
47. Li, X. et al. Modified-FOLFIRINOX in metastatic pancreatic cancer: A prospective study in Chinese population. Cancer letters 406, 22-26, https://doi.org/10.1016/j.canlet.2017.07.012 (2017).
48. Chllamma, M. K. et al. FOLFIRINOX for advanced pancreatic cancer: the Princess Margaret Cancer Centre experience. British journal of cancer 115, 649-654, https://doi.org/10.1038/bjc.2016.222 (2016).
49. Takeda, Y. et al. FOLFIRINOX Combination Chemotherapy in Patients with Metastatic or Recurrent Pancreatic Cancer-A Single Institution Experience. Gan to kagaku ryoho. Cancer & chemotherapy 42, 2360-2363 (2015).
50. Yoshida, K. et al. A multicenter prospective phase II study of first-line modified FOLFIRINOX for unresectable advanced pancreatic cancer. Oncotarget 8, 111346-111355, https://doi.org/10.18632/oncotarget.22795 (2017).
51. Blazer, M. et al. Neoadjuvant modified (m) FOLFIRINOX for locally advanced unresectable (LAPC) and borderline resectable (BRPC) adenocarcinoma of the pancreas. Annals of surgical oncology 22, 1153-1159, https://doi.org/10.1245/s10434-014-4225-1 (2015).
52. Jandu H, Aluzaite K, Fogh L, Thrane S W, Noer J B, Proszek J, et al. Molecular characterization of irinotecan (SN-38) resistant human breast cancer cell lines. BMC Cancer. 2016; 16:34.
53. Jensen N F, Stenvang J, Beck M K, Hanakova B, Belling K C, Do K N, et al. Establishment and characterization of models of chemotherapy resistance in colorectal cancer: Towards a predictive signature of chemoresistance. Mol Oncol. 2015; 9(6):1169-85.
54. Hansen S N, Westergaard D, Thomsen M B, Vistesen M, Do K N, Fogh L, et al. Acquisition of docetaxel resistance in breast cancer cells reveals upregulation of ABCB1 expression as a key mediator of resistance accompanied by discrete upregulation of other specific genes and pathways. Tumour Biol. 2015; 36(6):4327-38.

1. A combination therapy for use in treating cancer, optionally chemotherapy-resistant cancer, in a subject comprising:
a. an antibody or antigen-binding portion thereof that specifically binds to CD40, and
b. chemotherapy.

2-4. (canceled)

5. A method of treating cancer,
optionally chemotherapeutic-resistant cancer, in a subject, the method comprising administering to the subject:
a. a therapeutically effective amount of an antibody or antigen-binding portion thereof that specifically binds to CD40 and chemotherapy; or
b. a therapeutically effective amount of a combination therapy comprising an antibody or antigen-binding portion thereof that specifically binds to CD40, and chemotherapy.

6. A pharmaceutical composition comprising the combination therapy of claim 1.

7. A kit comprising an antibody or antigen-binding portion that specifically binds to CD40 and chemotherapy.

8. The method of treatment according to claim 5, wherein the cancer, optionally chemotherapy-resistant cancer, is pancreatic cancer;
optionally wherein the pancreatic cancer is an exocrine tumour, such as an exocrine adenocarcinoma or pancreatic ductal adenocarcinoma (PDAC), or an endocrine tumour.

9. The method of treatment according to claim 5, wherein the chemotherapy is selected from the group consisting of FOLFIRINOX or variants thereof, gemcitabine, nab-paclitaxel, and combinations thereof.

10. The method of treatment according to claim 5, wherein the chemotherapy is FOLFIRINOX or variants thereof, and the antibody or antigen-binding portion thereof is mitazalimab.

11. The method of treatment according to claim 10, wherein the FOLFIRINOX or variants thereof comprises:
a. oxaliplatin infusion, optionally wherein the oxaliplatin is administered:
i. at a dose of 85 mg/m²,
ii. intravenously, and/or
iii. for 2 hours;
b. a folinate infusion, optionally wherein the folinate is administered:
i. at a dose of 400 mg/m²,
ii. intravenously, and/or
iii. for 2 hours;
c. irinotecan infusion, optionally wherein the irinotecan is administered:
i. at a dose of 150 mg/m²,
ii. intravenously, and/or
iii. 30 minutes after the end of the leucovorin infusion; and/or
d. 5-fluorouracil infusion, optionally wherein the 5-fluorouracil is administered:
i. at a dose of 2400 mg/m²,
ii. intravenously, and/or
iii. for a duration of 46-48 hours (e.g. 2.4 g/m²/day);
optionally wherein the FOLFIRINOX or variants thereof is administered for multiple treatment cycles, preferably for 4 treatment cycles or 8 treatment cycles.

12. The method of treatment according to claim 11, wherein the oxaliplatin is administered over 2 hours, immediately followed by the folinate over 2 hours; and wherein the irinotecan is administered over 90 minutes, starting 30 minutes after the start of the leucovorin; following which the 5-fluorouracil is administered over 46-48 hours.

13. The method of treatment according to claim 11, further comprising:
a. premedication, optionally wherein the premedication comprises:
i. NK1-receptor antagonist, 125 mg PO, 60 minutes prior to infusion and/or continuously during days with chemotherapy,
ii. 5-HT3 receptor antagonist, 8 mg PO, 30 minutes prior to infusion and/or continuously during days with chemotherapy, and/or
iii. corticosteroid, 8 mg IV or PO, 30 minutes prior to infusion;
and/or
b. post-medication, optionally wherein the post-medication comprises:
i. G-CSF, 6 mg SC, on the fourth day following the start of the FOLFORINOX regimen or variant thereof, and/or at least 24 hours after the end of the continuous 5-fluorouracil infusion.

14. The method of treatment according to claim 5, wherein the antibody or antigen-binding portion thereof is administered at a dose:
a. from 50 μg/kg to 1200 μg/kg, such as from 450 μg/kg to 900 μg/kg;
b. of 50 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 950 μg/kg, 1000 μg/kg, 1050 μg/kg, 1100 μg/kg, 1150 μg/kg, 1200 μg/kg or higher;
c. of 450 μg/kg; and/or
d. of 900 μg/kg.

15. The method of treatment according to claim 5, wherein the antibody or antigen-binding portion thereof is administered more than once.

16. The method of treatment according to claim 5, wherein the antibody or antigen-binding portion thereof comprises the following CDRs:
V_{L} CDR1:
[SEQ ID NO: 1]
CTGSSSNIGAGYNVY;
V_{L} CDR2:
[SEQ ID NO: 2]
GNINRPS;
V_{L} CDR3:
[SEQ ID NO: 3]
CAAWDKSISGLV;
VH CDR1:
[SEQ ID NO: 4]
GFTFSTYGMH;
V_{H} CDR2:
[SEQ ID NO: 5]
GKGLEWLSYISGGSSYIFYADSVRGR;
and
V_{H} CDR3:
[SEQ ID NO: 6]
CARILRGGSGMDL.

17. The method of treatment according to claim 5, wherein the antibody or antigen-binding portion thereof comprises:
a. the light chain variable region of SEQ ID NO: 7 and/or the heavy chain variable region of SEQ ID NO: 8;
b. the light chain constant region of SEQ ID NO: 11 and/or the heavy chain constant region of SEQ ID NO: 12; or
c. the light chain of SEQ ID NO: 7 plus SEQ ID NO: 11, and/or the heavy chain of SEQ ID NO: 8 plus SEQ ID NO: 12.

18. The method of treatment according to claim 5, wherein the antibody or antigen-binding portion thereof that specifically binds to CD40 comprises or consists of an intact antibody.

19. The method of treatment according to claim 5, wherein the antibody or antigen-binding portion thereof comprises or consists of an antigen-binding fragment selected from the group consisting of: an Fv fragment and a Fab-like fragment.

20. The method of treatment combination therapy, use, method, pharmaceutical composition, or kit according to claim 5, wherein the antibody or antigen-binding portion thereof is human or humanised.

21. The method of treatment according to claim 5, wherein the antibody or antigen-binding portion thereof and the chemotherapy are administered simultaneously, sequentially, or subsequently to each other.

22. The method of treatment according to claim 5, wherein the antibody or antigen-binding portion thereof and/or the chemotherapy are administered locally to the tumour site.

23. The method of treatment according to claim 5, wherein the antibody or antigen-binding portion thereof is administered on multiple separate occasions and the chemotherapy is administered continuously for the duration of the method.
